SEQUENCE LISTING (1288 sequences)

SEQ ID NO: 1 - protein, 9 aa, Artificial Sequence
  Description of Artificial Sequence Synthetic peptide
GACGVGKSA

SEQ ID NO: 2 - protein, 9 aa, Artificial Sequence
  Description of Artificial Sequence Synthetic peptide
GAVGVGKSA

SEQ ID NO: 3 - protein, 10 aa, Artificial Sequence
  Description of Artificial Sequence Synthetic peptide
VVVGAVGVGK

SEQ ID NO: 4 - protein, 9 aa, Artificial Sequence
  Description of Artificial Sequence Synthetic peptide
ALNSEALSV

SEQ ID NO: 5 - protein, 10 aa, Artificial Sequence
  Description of Artificial Sequence Synthetic peptide
ILDTAGHEEY

SEQ ID NO: 6 - protein, 11 aa, Artificial Sequence
  Description of Artificial Sequence Synthetic peptide
DTAGHEEYSAM

SEQ ID NO: 7 - protein, 8 aa, Artificial Sequence
  Description of Artificial Sequence Synthetic peptide
GHEEYSAM

SEQ ID NO: 8 - protein, 8 aa, Artificial Sequence
  Description of Artificial Sequence Synthetic peptide
DTAGHEEY

SEQ ID NO: 9 - protein, 9 aa, Artificial Sequence
  Description of Artificial Sequence Synthetic peptide
ILDTAGHEE

SEQ ID NO: 10 - protein, 9 aa, Artificial Sequence
  Description of Artificial Sequence Synthetic peptide
AGHEEYSAM

SEQ ID NO: 11 - protein, 9 aa, Artificial Sequence
  Description of Artificial Sequence Synthetic peptide
HEEYSAMRD

SEQ ID NO: 12 - protein, 9 aa, Artificial Sequence
  Description of Artificial Sequence Synthetic peptide
TAGHEEYSA

SEQ ID NO: 13 - protein, 9 aa, Artificial Sequence
  Description of Artificial Sequence Synthetic peptide
DTAGHEEYS

SEQ ID NO: 14 - protein, 12 aa, Artificial Sequence
  Description of Artificial Sequence Synthetic peptide
DTAGHEEYSAMR

SEQ ID NO: 15 - protein, 9 aa, Artificial Sequence
  Description of Artificial Sequence Synthetic peptide
LDTAGHEEY

SEQ ID NO: 16 - protein, 10 aa, Artificial Sequence
  Description of Artificial Sequence Synthetic peptide
LLDILDTAGH

SEQ ID NO: 17 - protein, 10 aa, Artificial Sequence
  Description of Artificial Sequence Synthetic peptide
TAGHEEYSAM

SEQ ID NO: 18 - protein, 9 aa, Artificial Sequence
  Description of Artificial Sequence Synthetic peptide
DILDTAGHE

SEQ ID NO: 19 - protein, 11 aa, Artificial Sequence
  Description of Artificial Sequence Synthetic peptide
DILDTAGHEEY

SEQ ID NO: 20 - protein, 8 aa, Artificial Sequence
  Description of Artificial Sequence Synthetic peptide
DILDTAGH

SEQ ID NO: 21 - protein, 8 aa, Artificial Sequence
  Description of Artificial Sequence Synthetic peptide
ILDTAGHE

SEQ ID NO: 22 - protein, 10 aa, Artificial Sequence
  Description of Artificial Sequence Synthetic peptide
DTAGHEEYSA

SEQ ID NO: 23 - protein, 10 aa, Artificial Sequence
  Description of Artificial Sequence Synthetic peptide
ILDTAGREEY

SEQ ID NO: 24 - protein, 11 aa, Artificial Sequence
  Description of Artificial Sequence Synthetic peptide
DTAGREEYSAM

SEQ ID NO: 25 - protein, 8 aa, Artificial Sequence
  Description of Artificial Sequence Synthetic peptide
DILDTAGR

SEQ ID NO: 26 - protein, 8 aa, Artificial Sequence
  Description of Artificial Sequence Synthetic peptide
DTAGREEY

SEQ ID NO: 27 - protein, 11 aa, Artificial Sequence
  Description of Artificial Sequence Synthetic peptide
CLLDILDTAGR

SEQ ID NO: 28 - protein, 9 aa, Artificial Sequence
  Description of Artificial Sequence Synthetic peptide
REEYSAMRD

SEQ ID NO: 29 - protein, 9 aa, Artificial Sequence
  Description of Artificial Sequence Synthetic peptide
GREEYSAMR

SEQ ID NO: 30 - protein, 9 aa, Artificial Sequence
  Description of Artificial Sequence Synthetic peptide
ILDTAGREE

SEQ ID NO: 31 - protein, 9 aa, Artificial Sequence
  Description of Artificial Sequence Synthetic peptide
AGREEYSAM

SEQ ID NO: 32 - protein, 9 aa, Artificial Sequence
  Description of Artificial Sequence Synthetic peptide
DILDTAGRE

SEQ ID NO: 33 - protein, 8 aa, Artificial Sequence
  Description of Artificial Sequence Synthetic peptide
GREEYSAM

SEQ ID NO: 34 - protein, 9 aa, Artificial Sequence
  Description of Artificial Sequence Synthetic peptide
TAGREEYSA

SEQ ID NO: 35 - protein, 9 aa, Artificial Sequence
  Description of Artificial Sequence Synthetic peptide
DTAGREEYS

SEQ ID NO: 36 - protein, 8 aa, Artificial Sequence
  Description of Artificial Sequence Synthetic peptide
ILDTAGRE

SEQ ID NO: 37 - protein, 12 aa, Artificial Sequence
  Description of Artificial Sequence Synthetic peptide
DTAGREEYSAMR

SEQ ID NO: 38 - protein, 10 aa, Artificial Sequence
  Description of Artificial Sequence Synthetic peptide
LLDILDTAGR

SEQ ID NO: 39 - protein, 10 aa, Artificial Sequence
  Description of Artificial Sequence Synthetic peptide
TAGREEYSAM

SEQ ID NO: 40 - protein, 11 aa, Artificial Sequence
  Description of Artificial Sequence Synthetic peptide
DILDTAGREEY

SEQ ID NO: 41 - protein, 10 aa, Artificial Sequence
  Description of Artificial Sequence Synthetic peptide
AGREEYSAMR

SEQ ID NO: 42 - protein, 9 aa, Artificial Sequence
  Description of Artificial Sequence Synthetic peptide
LDILDTAGR

SEQ ID NO: 43 - protein, 9 aa, Artificial Sequence
  Description of Artificial Sequence Synthetic peptide
LDTAGREEY

SEQ ID NO: 44 - protein, 11 aa, Artificial Sequence
  Description of Artificial Sequence Synthetic peptide
REEYSAMRDQY

SEQ ID NO: 45 - protein, 10 aa, Artificial Sequence
  Description of Artificial Sequence Synthetic peptide
ILDTAGKEEY

SEQ ID NO: 46 - protein, 11 aa, Artificial Sequence
  Description of Artificial Sequence Synthetic peptide
DTAGKEEYSAM

SEQ ID NO: 47 - protein, 11 aa, Artificial Sequence
  Description of Artificial Sequence Synthetic peptide
CLLDILDTAGK

SEQ ID NO: 48 - protein, 8 aa, Artificial Sequence
  Description of Artificial Sequence Synthetic peptide
DTAGKEEY

SEQ ID NO: 49 - protein, 9 aa, Artificial Sequence
  Description of Artificial Sequence Synthetic peptide
AGKEEYSAM

SEQ ID NO: 50 - protein, 9 aa, Artificial Sequence
  Description of Artificial Sequence Synthetic peptide
DILDTAGKE

SEQ ID NO: 51 - protein, 9 aa, Artificial Sequence
  Description of Artificial Sequence Synthetic peptide
KEEYSAMRD

SEQ ID NO: 52 - protein, 8 aa, Artificial Sequence
  Description of Artificial Sequence Synthetic peptide
GKEEYSAM

SEQ ID NO: 53 - protein, 9 aa, Artificial Sequence
  Description of Artificial Sequence Synthetic peptide
ILDTAGKEE

SEQ ID NO: 54 - protein, 9 aa, Artificial Sequence
  Description of Artificial Sequence Synthetic peptide
DTAGKEEYS

SEQ ID NO: 55 - protein, 9 aa, Artificial Sequence
  Description of Artificial Sequence Synthetic peptide
LDTAGKEEY

SEQ ID NO: 56 - protein, 9 aa, Artificial Sequence
  Description of Artificial Sequence Synthetic peptide
TAGKEEYSA

SEQ ID NO: 57 - protein, 8 aa, Artificial Sequence
  Description of Artificial Sequence Synthetic peptide
DILDTAGK

SEQ ID NO: 58 - protein, 12 aa, Artificial Sequence
  Description of Artificial Sequence Synthetic peptide
DTAGKEEYSAMR

SEQ ID NO: 59 - protein, 10 aa, Artificial Sequence
  Description of Artificial Sequence Synthetic peptide
LLDILDTAGK

SEQ ID NO: 60 - protein, 8 aa, Artificial Sequence
  Description of Artificial Sequence Synthetic peptide
ILDTAGKE

SEQ ID NO: 61 - protein, 10 aa, Artificial Sequence
  Description of Artificial Sequence Synthetic peptide
AGKEEYSAMR

SEQ ID NO: 62 - protein, 10 aa, Artificial Sequence
  Description of Artificial Sequence Synthetic peptide
TAGKEEYSAM

SEQ ID NO: 63 - protein, 10 aa, Artificial Sequence
  Description of Artificial Sequence Synthetic peptide
ILDTAGLEEY

SEQ ID NO: 64 - protein, 10 aa, Artificial Sequence
  Description of Artificial Sequence Synthetic peptide
LLDILDTAGL

SEQ ID NO: 65 - protein, 12 aa, Artificial Sequence
  Description of Artificial Sequence Synthetic peptide
GLEEYSAMRDQY

SEQ ID NO: 66 - protein, 8 aa, Artificial Sequence
  Description of Artificial Sequence Synthetic peptide
DTAGLEEY

SEQ ID NO: 67 - protein, 11 aa, Artificial Sequence
  Description of Artificial Sequence Synthetic peptide
DTAGLEEYSAM

SEQ ID NO: 68 - protein, 9 aa, Artificial Sequence
  Description of Artificial Sequence Synthetic peptide
ILDTAGLEE

SEQ ID NO: 69 - protein, 11 aa, Artificial Sequence
  Description of Artificial Sequence Synthetic peptide
CLLDILDTAGL

SEQ ID NO: 70 - protein, 8 aa, Artificial Sequence
  Description of Artificial Sequence Synthetic peptide
DILDTAGL

SEQ ID NO: 71 - protein, 11 aa, Artificial Sequence
  Description of Artificial Sequence Synthetic peptide
DILDTAGLEEY

SEQ ID NO: 72 - protein, 9 aa, Artificial Sequence
  Description of Artificial Sequence Synthetic peptide
DTAGLEEYS

SEQ ID NO: 73 - protein, 9 aa, Artificial Sequence
  Description of Artificial Sequence Synthetic peptide
GLEEYSAMR

SEQ ID NO: 74 - protein, 8 aa, Artificial Sequence
  Description of Artificial Sequence Synthetic peptide
ILDTAGLE

SEQ ID NO: 75 - protein, 9 aa, Artificial Sequence
  Description of Artificial Sequence Synthetic peptide
LDTAGLEEY

SEQ ID NO: 76 - protein, 12 aa, Artificial Sequence
  Description of Artificial Sequence Synthetic peptide
DTAGLEEYSAMR

SEQ ID NO: 77 - protein, 9 aa, Artificial Sequence
  Description of Artificial Sequence Synthetic peptide
AGLEEYSAM

SEQ ID NO: 78 - protein, 10 aa, Artificial Sequence
  Description of Artificial Sequence Synthetic peptide
DTAGLEEYSA

SEQ ID NO: 79 - protein, 9 aa, Artificial Sequence
  Description of Artificial Sequence Synthetic peptide
LDILDTAGL

SEQ ID NO: 80 - protein, 10 aa, Artificial Sequence
  Description of Artificial Sequence Synthetic peptide
AGLEEYSAMR

SEQ ID NO: 81 - protein, 9 aa, Artificial Sequence
  Description of Artificial Sequence Synthetic peptide
LEEYSAMRD

SEQ ID NO: 82 - protein, 11 aa, Artificial Sequence
  Description of Artificial Sequence Synthetic peptide
LLDILDTAGLE

SEQ ID NO: 83 - protein, 9 aa, Artificial Sequence
  Description of Artificial Sequence Synthetic peptide
DILDTAGLE

SEQ ID NO: 84 - protein, 9 aa, Artificial Sequence
  Description of Artificial Sequence Synthetic peptide
TAGLEEYSA

SEQ ID NO: 85 - protein, 8 aa, Artificial Sequence
  Description of Artificial Sequence Synthetic peptide
GLEEYSAM

SEQ ID NO: 86 - protein, 10 aa, Artificial Sequence
  Description of Artificial Sequence Synthetic peptide
VVVGACGVGK

SEQ ID NO: 87 - protein, 9 aa, Artificial Sequence
  Description of Artificial Sequence Synthetic peptide
VVGACGVGK

SEQ ID NO: 88 - protein, 9 aa, Artificial Sequence
  Description of Artificial Sequence Synthetic peptide
ACGVGKSAL

SEQ ID NO: 89 - protein, 8 aa, Artificial Sequence
  Description of Artificial Sequence Synthetic peptide
CGVGKSAL

SEQ ID NO: 90 - protein, 10 aa, Artificial Sequence
  Description of Artificial Sequence Synthetic peptide
KLVVVGACGV

SEQ ID NO: 91 - protein, 10 aa, Artificial Sequence
  Description of Artificial Sequence Synthetic peptide
GACGVGKSAL

SEQ ID NO: 92 - protein, 9 aa, Artificial Sequence
  Description of Artificial Sequence Synthetic peptide
YKLVVVGAC

SEQ ID NO: 93 - protein, 9 aa, Artificial Sequence
  Description of Artificial Sequence Synthetic peptide
LVVVGACGV

SEQ ID NO: 94 - protein, 9 aa, Artificial Sequence
  Description of Artificial Sequence Synthetic peptide
VVGAVGVGK

SEQ ID NO: 95 - protein, 9 aa, Artificial Sequence
  Description of Artificial Sequence Synthetic peptide
LVVVGAVGV

SEQ ID NO: 96 - protein, 9 aa, Artificial Sequence
  Description of Artificial Sequence Synthetic peptide
AVGVGKSAL

SEQ ID NO: 97 - protein, 10 aa, Artificial Sequence
  Description of Artificial Sequence Synthetic peptide
KLVVVGAVGV

SEQ ID NO: 98 - protein, 10 aa, Artificial Sequence
  Description of Artificial Sequence Synthetic peptide
GAVGVGKSAL

SEQ ID NO: 99 - protein, 8 aa, Artificial Sequence
  Description of Artificial Sequence Synthetic peptide
VGVGKSAL

SEQ ID NO: 100 - protein, 8 aa, Artificial Sequence
  Description of Artificial Sequence Synthetic peptide
VVVGAVGV

SEQ ID NO: 101 - protein, 10 aa, Artificial Sequence
  Description of Artificial Sequence Synthetic peptide
VGVGKSALTI

SEQ ID NO: 102 - protein, 8 aa, Artificial Sequence
  Description of Artificial Sequence Synthetic peptide
AVGVGKSA

SEQ ID NO: 103 - protein, 9 aa, Artificial Sequence
  Description of Artificial Sequence Synthetic peptide
YKLVVVGAV

SEQ ID NO: 104 - protein, 62 aa, Artificial Sequence
  Description of Artificial Sequence Synthetic polypeptide
MTEYKLVVVGACGVGKSALTIQLIQNHFVDEYDPTIEDSYRKQVVIDGETCLLDILDTAGQE

SEQ ID NO: 105 - protein, 62 aa, Artificial Sequence
  Description of Artificial Sequence Synthetic polypeptide
MTEYKLVVVGADGVGKSALTIQLIQNHFVDEYDPTIEDSYRKQVVIDGETCLLDILDTAGQE

SEQ ID NO: 106 - protein, 62 aa, Artificial Sequence
  Description of Artificial Sequence Synthetic polypeptide
MTEYKLVVVGAVGVGKSALTIQLIQNHFVDEYDPTIEDSYRKQVVIDGETCLLDILDTAGQE

SEQ ID NO: 107 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGHEEYSAMRDQYMRTGEGFLCVFAINNTKSFEDIHHYREQIKRVKDSEDVPM

SEQ ID NO: 108 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGLEEYSAMRDQYMRTGEGFLCVFAINNTKSFEDIHHYREQIKRVKDSEDVPM

SEQ ID NO: 109 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGKEEYSAMRDQYMRTGEGFLCVFAINNSKSFADINLYREQIKRVKDSDDVPM

SEQ ID NO: 110 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGREEYSAMRDQYMRTGEGFLCVFAINNSKSFADINLYREQIKRVKDSDDVPM

SEQ ID NO: 111 - protein, 101 aa, Artificial Sequence
  Description of Artificial Sequence Synthetic polypeptide
MIKEGSMSEDEFIEEAKVMMNLSHEKLVQLYGVCTKQRPIFIITEYMANGSLLNYLREMRHRFQTQQLLEMCKDVCEAMEYLESKQFLHRDLAARNCLVND

SEQ ID NO: 112 - protein, 101 aa, Artificial Sequence
  Description of Artificial Sequence Synthetic polypeptide
SLNITSLGLRSLKEISDGDVIISGNKNLCYANTINWKKLFGTSGQKTKIIRNRGENSCKATGQVCHALCSPEGCWGPEPRDCVSCRNVSRGRECVDKCNLL

SEQ ID NO: 113 - protein, 101 aa, Artificial Sequence
  Description of Artificial Sequence Synthetic polypeptide
IPVAIKELREATSPKANKEILDEAYVMASVDNPHVCRLLGICLTSTVQLIMQLMPFGCLLDYVREHKDNIGSQYLLNWCVQIAKGMNYLEDRRLVHRDLAA

SEQ ID NO: 114 - protein, 101 aa, Artificial Sequence
  Description of Artificial Sequence Synthetic polypeptide
VADGLITTLHYPAPKRNKPTVYGVSPNYDKWEMERTDITMKHKLGGGQYGKVYEGVWKKYSLTVAVKTLKEDTMEVEEFLKEAAVMKEIKHPNLVQLLGVC

SEQ ID NO: 115 - protein, 101 aa, Artificial Sequence
  Description of Artificial Sequence Synthetic polypeptide
VADGLITTLHYPAPKRNKPTVYGVSPNYDKWEMERTDITMKHKLGGGQYGVVYEGVWKKYSLTVAVKTLKEDTMEVEEFLKEAAVMKEIKHPNLVQLLGVC

SEQ ID NO: 116 - protein, 101 aa, Artificial Sequence
  Description of Artificial Sequence Synthetic polypeptide
LLGVCTREPPFYIITEFMTYGNLLDYLRECNRQEVNAVVLLYMATQISSATEYLEKKNFIHRDLAARNCLVGENHLVKVADFGLSRLMTGDTYTAHAGAKF

SEQ ID NO: 117 - protein, 101 aa, Artificial Sequence
  Description of Artificial Sequence Synthetic polypeptide
SLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLA

SEQ ID NO: 118 - protein, 101 aa, Artificial Sequence
  Description of Artificial Sequence Synthetic polypeptide
STVADGLITTLHYPAPKRNKPTVYGVSPNYDKWEMERTDITMKHKLGGGQHGEVYEGVWKKYSLTVAVKTLKEDTMEVEEFLKEAAVMKEIKHPNLVQLLG

SEQ ID NO: 119 - protein, 101 aa, Artificial Sequence
  Description of Artificial Sequence Synthetic polypeptide
SSLAMLDLLHVARDIACGCQYLEENHFIHRDIAARNCLLTCPGPGRVAKIADFGMARDIYRASYYRKGGCAMLPVKWMPPEAFMEGIFTSKTDTWSFGVLL

SEQ ID NO: 120 - protein, 101 aa, Artificial Sequence
  Description of Artificial Sequence Synthetic polypeptide
QVAVKTLPEVCSEQDELDFLMEALIISKFNHQNIVRCIGVSLQSLPRFILMELMAGGDLKSFLRETRPRPSQPSSLAMLDLLHVARDIACGCQYLEENHFI

SEQ ID NO: 121 - protein, 101 aa, Artificial Sequence
  Description of Artificial Sequence Synthetic polypeptide
MIKLIDIARQTAQGMDYLHAKSIIHRDLKSNNIFLHEDLTVKIGDFGLATEKSRWSGSHQFEQLSGSILWMAPEVIRMQDKNPYSFQSDVYAFGIVLYELM

SEQ ID NO: 122 - protein, 93 aa, Artificial Sequence
  Description of Artificial Sequence Synthetic polypeptide
MGFGDLKSPAGLQVLNDYLADKSYIEGYVPSQADVAVFEAVSGPPPADLCHALRWYNHIKSYEKEKASLPGVKKALGKYGPADVEDTTGSGAT

SEQ ID NO: 123 - protein, 101 aa, Artificial Sequence
  Description of Artificial Sequence Synthetic polypeptide
ERCEVVMGNLEIVLTGHNADLSFLQWIREVTGYVLVAMNEFSTLPLPNLRMVRGTQVYDGKFAIFVMLNYNTNSSHALRQLRLTQLTEILSGGVYIEKNDK

SEQ ID NO: 124 - protein, 101 aa, Artificial Sequence
  Description of Artificial Sequence Synthetic polypeptide
HLMAKAGLTLQQQHQRLAQLLLILSHIRHMSNKGMEHLYSMKCKNVVPLYGLLLEMLDAHRLHAPTSRGGASVEETDQSHLATAGSTSSHSLQKYYITGEA

SEQ ID NO: 125 - protein, 101 aa, Artificial Sequence
  Description of Artificial Sequence Synthetic polypeptide
NQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSH

SEQ ID NO: 126 - protein, 101 aa, Artificial Sequence
  Description of Artificial Sequence Synthetic polypeptide
IHLMAKAGLTLQQQHQRLAQLLLILSHIRHMSNKGMEHLYSMKCKNVVPLCDLLLEMLDAHRLHAPTSRGGASVEETDQSHLATAGSTSSHSLQKYYITGE

SEQ ID NO: 127 - protein, 101 aa, Artificial Sequence
  Description of Artificial Sequence Synthetic polypeptide
IHLMAKAGLTLQQQHQRLAQLLLILSHIRHMSNKGMEHLYSMKCKNVVPLNDLLLEMLDAHRLHAPTSRGGASVEETDQSHLATAGSTSSHSLQKYYITGE

SEQ ID NO: 128 - protein, 101 aa, Artificial Sequence
  Description of Artificial Sequence Synthetic polypeptide
IHLMAKAGLTLQQQHQRLAQLLLILSHIRHMSNKGMEHLYSMKCKNVVPLSDLLLEMLDAHRLHAPTSRGGASVEETDQSHLATAGSTSSHSLQKYYITGE

SEQ ID NO: 129 - protein, 101 aa, Artificial Sequence
  Description of Artificial Sequence Synthetic polypeptide
HRIGGIKLRHQQWSLVMESVVPSDRGNYTCVVENKFGSIRQTYTLDVLERCPHRPILQAGLPANQTAVLGSDVEFHCKVYSDAQPHIQWLKHVEVNGSKVG

SEQ ID NO: 130 - protein, 101 aa, Artificial Sequence
  Description of Artificial Sequence Synthetic polypeptide
AVKLSDSRIALKSGYGKYLGINSDELVGHSDAIGPREQWEPVFQNGKMALSASNSCFIRCNEAGDIEAKSKTAGEEEMIKIRSCAEKETKKKDDIPEEDKG

SEQ ID NO: 131 - protein, 101 aa, Artificial Sequence
  Description of Artificial Sequence Synthetic polypeptide
GSGAFGTVYKGIWIPDGENVKIPVAIKVLRENTSPKANKEILDEAYVMAGLGSPYVSRLLGICLTSTVQLVTQLMPYGCLLDHVRENRGRLGSQDLLNWCM

SEQ ID NO: 132 - protein, 101 aa, Artificial Sequence
  Description of Artificial Sequence Synthetic polypeptide
RVEEFKLKQMWKSPNGTIRNILGGTVFREAIICKNIPRLVSGWVKPIIIGHHAYGDQYRATDFVVPGPGKVEITYTPSDGTQKVTYLVHNFEEGGGVAMGM

SEQ ID NO: 133 - protein, 101 aa, Artificial Sequence
  Description of Artificial Sequence Synthetic polypeptide
RVEEFKLKQMWKSPNGTIRNILGGTVFREAIICKNIPRLVSGWVKPIIIGCHAYGDQYRATDFVVPGPGKVEITYTPSDGTQKVTYLVHNFEEGGGVAMGM

SEQ ID NO: 134 - protein, 101 aa, Artificial Sequence
  Description of Artificial Sequence Synthetic polypeptide
RVEEFKLKQMWKSPNGTIRNILGGTVFREAIICKNIPRLVSGWVKPIIIGGHAYGDQYRATDFVVPGPGKVEITYTPSDGTQKVTYLVHNFEEGGGVAMGM

SEQ ID NO: 135 - protein, 101 aa, Artificial Sequence
  Description of Artificial Sequence Synthetic polypeptide
RVEEFKLKQMWKSPNGTIRNILGGTVFREAIICKNIPRLVSGWVKPIIIGSHAYGDQYRATDFVVPGPGKVEITYTPSDGTQKVTYLVHNFEEGGGVAMGM

SEQ ID NO: 136 - protein, 101 aa, Artificial Sequence
  Description of Artificial Sequence Synthetic polypeptide
VAVKMLKPSAHLTEREALMSELKVLSYLGNHMNIVNLLGACTIGGPTLVIIEYCCYGDLLNFLRRKRDSFICSKQEDHAEAALYKNLLHSKESSCSDSTNE

SEQ ID NO: 137 - protein, 101 aa, Artificial Sequence
  Description of Artificial Sequence Synthetic polypeptide
VEATAYGLIKSDAAMTVAVKMLKPSAHLTEREALMSELKVLSYLGNHMNIANLLGACTIGGPTLVITEYCCYGDLLNFLRRKRDSFICSKQEDHAEAALYK

SEQ ID NO: 138 - protein, 101 aa, Artificial Sequence
  Description of Artificial Sequence Synthetic polypeptide
ISELGAGNGGVVFKVSHKPSGLVMARKLIHLEIKPAIRNQIIRELQVLHESNSPYIVGFYGAFYSDGEISICMEHMDGGSLDQVLKKAGRIPEQILGKVSI

SEQ ID NO: 139 - protein, 101 aa, Artificial Sequence
  Description of Artificial Sequence Synthetic polypeptide
LGAGNGGVVFKVSHKPSGLVMARKLIHLEIKPAIRNQIIRELQVLHECNSLYIVGFYGAFYSDGEISICMEHMDGGSLDQVLKKAGRIPEQILGKVSIAVI

SEQ ID NO: 140 - protein, 89 aa, Artificial Sequence
  Description of Artificial Sequence Synthetic polypeptide
MPLNVSFTNRNYDLDYDSVQPYFYCDEEENFYQQQQQSDLQPPAPSEDIWKKFELLPTPPLSPSRRSGLCSPSYVAVTPFSLRGDNDGG

SEQ ID NO: 141 - protein, 101 aa, Artificial Sequence
  Description of Artificial Sequence Synthetic polypeptide
FTNRNYDLDYDSVQPYFYCDEEENFYQQQQQSELQPPAPSEDIWKKFELLSTPPLSPSRRSGLCSPSYVAVTPFSLRGDNDGGGGSFSTADQLEMVTELLG

SEQ ID NO: 142 - protein, 101 aa, Artificial Sequence
  Description of Artificial Sequence Synthetic polypeptide
TNRNYDLDYDSVQPYFYCDEEENFYQQQQQSELQPPAPSEDIWKKFELLPIPPLSPSRRSGLCSPSYVAVTPFSLRGDNDGGGGSFSTADQLEMVTELLGG

SEQ ID NO: 143 - protein, 101 aa, Artificial Sequence
  Description of Artificial Sequence Synthetic polypeptide
VAVKMLKPTARSSEKQALMSELKIMTHLGPHLNIVNLLGACTKSGPIYIIIEYCFYGDLVNYLHKNRDSFLSHHPEKPKKELDIFGLNPADESTRSYVILS

SEQ ID NO: 144 - protein, 101 aa, Artificial Sequence
  Description of Artificial Sequence Synthetic polypeptide
IEEHANWSVSREAGFSYSHAGLSNRLARDNELRENDKEQLKAISTRDPLSKITEQEKDFLWSHRHYCVTIPEILPKLLLSVKWNSRDEVAQMYCLVKDWPP

SEQ ID NO: 145 - protein, 101 aa, Artificial Sequence
  Description of Artificial Sequence Synthetic polypeptide
HANWSVSREAGFSYSHAGLSNRLARDNELRENDKEQLKAISTRDPLSEITKQEKDFLWSHRHYCVTIPEILPKLLLSVKWNSRDEVAQMYCLVKDWPPIKP

SEQ ID NO: 146 - protein, 72 aa, Artificial Sequence
  Description of Artificial Sequence Synthetic polypeptide
LFINLFSMMLGSGMPELQSFDDIAYIRKTLALDKTEQEALEYFMKQMNDARHGGWTTKMDWIFHTIKQHALN

SEQ ID NO: 147 - protein, 101 aa, Artificial Sequence
  Description of Artificial Sequence Synthetic polypeptide
QRGGVITDEEETSKKIADQLDNIVDMREYDVPYHIRLSIDIETTKLPLKFRDAETDQIMMISYMIDGQGYLITNREIVSEDIEDFEFTPKPEYEGPFCVFN

SEQ ID NO: 148 - protein, 101 aa, Artificial Sequence
  Description of Artificial Sequence Synthetic polypeptide
KFNCRVAQYPFEDHNPPQLELIKPFCEDLDQWLSEDDNHVAAIHCKAGKGQTGVMICAYLLHRGKFLKAQEALDFYGEVRTRDKKGVTIPSQRRYVYYYSY

SEQ ID NO: 149 - protein, 79 aa, Artificial Sequence
  Description of Artificial Sequence Synthetic polypeptide
MQAIKCVVVGDGAVGKTCLLISYTTNAFSGEYIPTVFDNYSANVMVDGKPVNLGLWDTAGQEDYDRLRPLSYPQTVGET

SEQ ID NO: 150 - protein, 101 aa, Artificial Sequence
  Description of Artificial Sequence Synthetic polypeptide
IRVEGNLRVEYLDDRNTFRHSVVVPYEPPEVGSDCTTIHYNYMCNSSCMGSMNRRPILTIITLEDSSGNLLGRNSFEVRVCACPGRDRRTEEENLRKKGEP

SEQ ID NO: 151 - protein, 101 aa, Artificial Sequence
  Description of Artificial Sequence Synthetic polypeptide
TYSPALNKMFCQLAKTCPVQLWVDSTPPPGTRVRAMAIYKQSQHMTEVVRHCPHHERCSDSDGLAPPQHLIRVEGNLRVEYLDDRNTFRHSVVVPYEPPEV

SEQ ID NO: 152 - protein, 101 aa, Artificial Sequence
  Description of Artificial Sequence Synthetic polypeptide
EGNLRVEYLDDRNTFRHSVVVPYEPPEVGSDCTTIHYNYMCNSSCMGGMNQRPILTIITLEDSSGNLLGRNSFEVRVCACPGRDRRTEEENLRKKGEPHHE

SEQ ID NO: 153 - protein, 101 aa, Artificial Sequence
  Description of Artificial Sequence Synthetic polypeptide
EGNLRVEYLDDRNTFRHSVVVPYEPPEVGSDCTTIHYNYMCNSSCMGGMNWRPILTIITLEDSSGNLLGRNSFEVRVCACPGRDRRTEEENLRKKGEPHHE

SEQ ID NO: 154 - protein, 101 aa, Artificial Sequence
  Description of Artificial Sequence Synthetic polypeptide
PEVGSDCTTIHYNYMCNSSCMGGMNRRPILTIITLEDSSGNLLGRNSFEVCVCACPGRDRRTEEENLRKKGEPHHELPPGSTKRALPNNTSSSPQPKKKPL

SEQ ID NO: 155 - protein, 53 aa, Artificial Sequence
  Description of Artificial Sequence Synthetic polypeptide
GVEPCYGDKDKRRHCFATWKNISGSIEIVKQGCWLDDINCYDRTDCVEKKRQP

SEQ ID NO: 156 - protein, 103 aa, Artificial Sequence
  Description of Artificial Sequence Synthetic polypeptide
GVEPCYGDKDKRRHCFATWKNISGSIEIVKQGCWLDDINCYDRTDCVEKKTALKYIFVAVRAICVMKSFLIFRRWKSHSPLQIQLHLSHPITTSCSIPWCHLC

SEQ ID NO: 157 - protein, 118 aa, Artificial Sequence
  Description of Artificial Sequence Synthetic polypeptide
TAEAVNVAIAAPPSEGEANAELCRYLSKVLELRKSDVVLDKVGLALFFFFFETKSCSVAQAGVQWRSLGSLQPPPPGFKLFSCLSFLSSWDYRRMPPCLANFCIFNRDGVSPCWSGWS

SEQ ID NO: 158 - protein, 71 aa, Artificial Sequence
  Description of Artificial Sequence Synthetic polypeptide
LSVIIFFFVYIWHWALPLILNNHHICLMSSIILDCNSVRQSIMSVCFFFFSVIFSTRCLTDSRYPNICWFK

SEQ ID NO: 159 - protein, 60 aa, Artificial Sequence
  Description of Artificial Sequence Synthetic polypeptide
LSVIIFFFVYIWHWALPLILNNHHICLMSSIILDCNSVRQSIMSVCFFFFCYILNTMFDR

SEQ ID NO: 160 - protein, 106 aa, Artificial Sequence
  Description of Artificial Sequence Synthetic polypeptide
VLVLSCDLITDVALHEVVDLFRAYDASLAMLMRKGQDSIEPVPGQKGKKKQWSSVTSLEWTAQERGCSSWLMKQTWMKSWSLRDPSYRSILEYVSTRVLWMPTSTV

SEQ ID NO: 161 - protein, 112 aa, Artificial Sequence
  Description of Artificial Sequence Synthetic polypeptide
SIQVMRAQMNQIQSVEGQPLARRPRATGRTKRCQPRDVTKKTCNSNDGKKREWEKRKQILGGGGKYKEYFLKRILIRKAMTVLAGDKKGLGRFMRCVQSETKAVSLQLPLGR

SEQ ID NO: 162 - protein, 57 aa, Artificial Sequence
  Description of Artificial Sequence Synthetic polypeptide
LDFLGEFATDIRTHGVHMVLNHQGRPSGDAFIQMKSADRAFMAAQKCHKKKHEGQIC

SEQ ID NO: 163 - protein, 51 aa, Artificial Sequence
  Description of Artificial Sequence Synthetic polypeptide
LDFLGEFATDIRTHGVHMVLNHQGRPSGDAFIQMKSADRAFMAAQKCHKKT

SEQ ID NO: 164 - protein, 75 aa, Artificial Sequence
  Description of Artificial Sequence Synthetic polypeptide
GALCKDGRFRSDIGEFEWKLKEGHKKIYGKQSMVDEVSGKVLEMDISKKKHYNRKISIKKLNRMKVPLMKLITRV

SEQ ID NO: 165 - protein, 58 aa, Artificial Sequence
  Description of Artificial Sequence Synthetic polypeptide
RERAQLLEEQEKTLTSKLQEQARVLKERCQGESTQLQNEIQKLQKTLKKKPRDICRIS

SEQ ID NO: 166 - protein, 53 aa, Artificial Sequence
  Description of Artificial Sequence Synthetic polypeptide
VNTLKEGKRLPCPPNCPDEVYQLMRKCWEFQPSNRTSFQNLIEGFEALLKTSN

SEQ ID NO: 167 - protein, 65 aa, Artificial Sequence
  Description of Artificial Sequence Synthetic polypeptide
CRPVTPSCKELADLMTRCMNYDPNQRPFFRAIMRDINKLEEQNPDIVSEKNQQLKWTPHILKSAS

SEQ ID NO: 168 - protein, 69 aa, Artificial Sequence
  Description of Artificial Sequence Synthetic polypeptide
DDHDVLSFLTFQLTEPGKEPPTPDKEISEKEKEKYQEEFEHFQQELDKKKRGIPEGPPRPPRAACGGNI

SEQ ID NO: 169 - protein, 71 aa, Artificial Sequence
  Description of Artificial Sequence Synthetic polypeptide
DDHDVLSFLTFQLTEPGKEPPTPDKEISEKEKEKYQEEFEHFQQELDKKKRNSRRATPTSKGSLRRKYLRV

SEQ ID NO: 170 - protein, 68 aa, Artificial Sequence
  Description of Artificial Sequence Synthetic polypeptide
TKSTLIGEDVNPLIKLDDAVNVDEIMTDTSTSYLLCISENKENVRDKKKGQHFYWHCGSAACHRRGCV

SEQ ID NO: 171 - protein, 81 aa, Artificial Sequence
  Description of Artificial Sequence Synthetic polypeptide
LYTKSTLIGEDVNPLIKLDDAVNVDEIMTDTSTSYLLCISENKENVRDKKRATFLLALWECSLPQARLCLIVSRTLLLVQS

SEQ ID NO: 172 - protein, 58 aa, Artificial Sequence
  Description of Artificial Sequence Synthetic polypeptide
LPPPKLTDPRLLYIGFLGYCSGLIDNLIRRRPIATAGLHRQLLYITAFFFCWILSCKT

SEQ ID NO: 173 - protein, 56 aa, Artificial Sequence
  Description of Artificial Sequence Synthetic polypeptide
SLPPPKLTDPRLLYIGFLGYCSGLIDNLIRRRPIATAGLHRQLLYITAFFLLDIIL

SEQ ID NO: 174 - protein, 112 aa, Artificial Sequence
  Description of Artificial Sequence Synthetic polypeptide
NQSGGAGEDCQIFSTPGHPKMIYSSSNLKTPSKLCSGSKSHDVQEVLKKKTGSNEVTTRYEEKKTGSVRKANRMPKDVNIQVRKKQKHETRRKSKYNEDFERAWREDLTIKR

SEQ ID NO: 175 - protein, 23 aa, Artificial Sequence
  Description of Artificial Sequence Synthetic peptide
MAPVKKLVVKGGKKKEASSEVHS

SEQ ID NO: 176 - protein, 18 aa, Artificial Sequence
  Description of Artificial Sequence Synthetic peptide
MAPVKKLVVKGGKKRSKF

SEQ ID NO: 177 - protein, 51 aa, Artificial Sequence
  Description of Artificial Sequence Synthetic polypeptide
MPSHQGAEQQQQQHHVFISQVVTEKEFLSRSDQLQQAVQSQGFINYCQKKN

SEQ ID NO: 178 - protein, 65 aa, Artificial Sequence
  Description of Artificial Sequence Synthetic polypeptide
MPSHQGAEQQQQQHHVFISQVVTEKEFLSRSDQLQQAVQSQGFINYCQKKLMLLRLNLRKMCGPF

SEQ ID NO: 179 - protein, 79 aa, Artificial Sequence
  Description of Artificial Sequence Synthetic polypeptide
AEVFEKEQSICAAEEQPAEDGQGETNKNRTKGGWQQKSKGPKKTAKSKKKETFKKKTYTCAITTVKATETKAGKWSRWE

SEQ ID NO: 180 - protein, 53 aa, Artificial Sequence
  Description of Artificial Sequence Synthetic polypeptide
MAEVFEKEQSICAAEEQPAEDGQGETNKNRTKGGWQQKSKGPKKTAKSKKRNL

SEQ ID NO: 181 - protein, 71 aa, Artificial Sequence
  Description of Artificial Sequence Synthetic polypeptide
NIMEIRQLPSSHALEAKLSRMSYPVKEQESILKTVGKLTATQVAKISFFFALCGFWQICHIKKHFQTHKLL

SEQ ID NO: 182 - protein, 68 aa, Artificial Sequence
  Description of Artificial Sequence Synthetic polypeptide
YEKKKYYDKNAIAITNISSSDAPLQPLVSSPSLQAAVDKNKLEKEKEKKKGREKERKGARKAGKTTYS

SEQ ID NO: 183 - protein, 97 aa, Artificial Sequence
  Description of Artificial Sequence Synthetic polypeptide
KYEKKKYYDKNAIAITNISSSDAPLQPLVSSPSLQAAVDKNKLEKEKEKKRKRKREKRSQKSRQNHLQLKSCRRKISNWSLKKVPALKKLRSPLWIF

SEQ ID NO: 184 - protein, 65 aa, Artificial Sequence
  Description of Artificial Sequence Synthetic polypeptide
IYQDAYRAEWQVYKEEISRFKEQLTPSQIMSLEKEIMDKHLKRKAMTKKKRVNTAWKTKKTSFSL

SEQ ID NO: 185 - protein, 51 aa, Artificial Sequence
  Description of Artificial Sequence Synthetic polypeptide
IYQDAYRAEWQVYKEEISRFKEQLTPSQIMSLEKEIMDKHLKRKAMTKKKS

SEQ ID NO: 186 - protein, 52 aa, Artificial Sequence
  Description of Artificial Sequence Synthetic polypeptide
KPQEVCVAVWRKNDENITLETVCHDPKLPYHDFILEDAASPKCIMKEKKKAW

SEQ ID NO: 187 - protein, 84 aa, Artificial Sequence
  Description of Artificial Sequence Synthetic polypeptide
EKPQEVCVAVWRKNDENITLETVCHDPKLPYHDFILEDAASPKCIMKEKKSLVRLSSCVPVALMSAMTTSSSQKNITPAILTCC

SEQ ID NO: 188 - protein, 74 aa, Artificial Sequence
  Description of Artificial Sequence Synthetic polypeptide
VPSKYQFLCSDHFTPDSLDIRWGIRYLKQTAVPTIFSLPEDNQGKDPSKKNPRRKTWKMRKKYAQKPSQKNHLY

SEQ ID NO: 189 - protein, 88 aa, Artificial Sequence
  Description of Artificial Sequence Synthetic polypeptide
GTTEEMKYVLGQLVGLNSPNSILKAAKTLYEHYSGGESHNSSSSKTFEKKGEKNDLQLFVMSDTTYKIYWTVILLNPCGNLHLKTTSL

SEQ ID NO: 190 - protein, 55 aa, Artificial Sequence
  Description of Artificial Sequence Synthetic polypeptide
QQLIRETLISWLQAQMLNPQPEKTFIRNKAAQVFALLFVTEYLTKWPKFFLTFSQ

SEQ ID NO: 191 - protein, 93 aa, Artificial Sequence
  Description of Artificial Sequence Synthetic polypeptide
AKFQQCHSTLEPNPADCRVLVYLQNQPGTKLLNFLQERNLPPKVVLRHPKVHLNTMFRRPHSCLADVLLSVHLIVLRVVRLPAPFRVNHAVEW

SEQ ID NO: 192 - protein, 53 aa, Artificial Sequence
  Description of Artificial Sequence Synthetic polypeptide
APVIFQIALDKPCHQAEVKHLHHLLKQLKPSEKYLKIKHLLLKRERVDLSKLQ

SEQ ID NO: 193 - protein, 33 aa, Artificial Sequence
  Description of Artificial Sequence Synthetic polypeptide
MLQFRGSRFFQMLILYYILPRKVLQMDFLVHPA

SEQ ID NO: 194 - protein, 179 aa, Artificial Sequence
  Description of Artificial Sequence Synthetic polypeptide
ALGPHSRISCLPTQTRGCILLAATPRSSSSSSSNDMIPMAISSPPKAPLLAAPSPASRLQCINSNSRITSGQWMAHMALLPSGTKGRCTACHTALGRGSLSSSSCPQPSPSLPASNKLPSLPLSKMYTTSMAMPILPLPQLLLSADQQAAPRTNFHSSLAETVSLHPLAPMPSKTCHHK

SEQ ID NO: 195 - protein, 79 aa, Artificial Sequence
  Description of Artificial Sequence Synthetic polypeptide
AHQGFPAAKESRVIQLSLLSLLIPPLTCLASEALPRPLLALPPVLLSLAQDHSRLLQCQATRCHLGHPVASRTASCILP

SEQ ID NO: 196 - protein, 222 aa, Artificial Sequence
  Description of Artificial Sequence Synthetic polypeptide
PILAATGTSVRTAARTWVPRAAIRVPDPAAVPDDHAGPGAECHGRPLLYTADSSLWTTRPQRVWSTGPDSILQPAKSSPSAAAATLLPATTVPDPSCPTFVSAAATVSTTTAPVLSASILPAAIPASTSAVPGSIPLPAVDDTAAPPEPAPLLTATGSVSLPAAATSAASTLDALPAGCVSSAPVSAVPANCLFPAALPSTAGAISRFIWVSGILSPLNDLQ

SEQ ID NO: 197 - protein, 185 aa, Artificial Sequence
  Description of Artificial Sequence Synthetic polypeptide
PCRAGRRVPWAASLIHSRFLLMDNKAPAGMVNRARLHITTSKVLTLSSSSHPTPSNHRPRPLMPNLRISSSHSLNHHSSSPLSLHTPSSHPSLHISSPRLHTPPSSRRHSSTPRASPPTHSHRLSLLTSSSNLSSQHPRRSPSRLRILSPSLSSPSKLPIPSSASLHRRSYLKIHLGLRHPQPPQ

SEQ ID NO: 198 - protein, 58 aa, Artificial Sequence
  Description of Artificial Sequence Synthetic polypeptide
RTNPTVRMRPHCVPFWTGRILLPSAASVCPIPFEACHLCQAMTLRCPNTQGCCSSWAS

SEQ ID NO: 199 - protein, 90 aa, Artificial Sequence
  Description of Artificial Sequence Synthetic polypeptide
TNQALPKIEVICRGTPRCPSTVPPSPAQPYLRVSLPEDRYTQAWAPTSRTPWGAMVPRGVSMAHKVATPGSQTIMPCPMPTTPVQAWLEA

SEQ ID NO: 200 - protein, 41 aa, Artificial Sequence
  Description of Artificial Sequence Synthetic polypeptide
RMERELKKWSIQTCLSARTGLSISCTTLNSPPLKKMSMPAV

SEQ ID NO: 201 - protein, 35 aa, Artificial Sequence
  Description of Artificial Sequence Synthetic polypeptide
LCSRYSLFLAWRLSSVLQRFRFTHVIQQRMESQIS

SEQ ID NO: 202 - protein, 39 aa, Artificial Sequence
  Description of Artificial Sequence Synthetic polypeptide
RSACVTVKGPLASVGRHSLSKQDCKFLPFWGFLEEFLLC

SEQ ID NO: 203 - protein, 97 aa, Artificial Sequence
  Description of Artificial Sequence Synthetic polypeptide
IQWGTTTAPRPIRPPFLESKQNCSHFPTPLLASEDRRETGLFLPSAAQKMKKAHFLKTWFRSNPTKTKKARFSTASLAKELTHPLLVSLLLKEKQDG

SEQ ID NO: 204 - protein, 73 aa, Artificial Sequence
  Description of Artificial Sequence Synthetic polypeptide
PTDPFLGLRLGLHLQKVFHQSHAEYSGAPPPPPAPSGLRFWNPSRIAHISQLLSWPQKTEERLGYSSHQLPRK

SEQ ID NO: 205 - protein, 45 aa, Artificial Sequence
  Description of Artificial Sequence Synthetic polypeptide
FCCSCCFFGGERWSKSPYCPQRMTPGTTFITMMKKEAEKRTRTLT

SEQ ID NO: 206 - protein, 95 aa, Artificial Sequence
  Description of Artificial Sequence Synthetic polypeptide
WRRNCKAPVSLRKSVQTPARSSPARPDRTRRLPSLGVPGQPWALGAAASRRCCCCCRSPLGSARSRSPATLALTPRATRSRCPGATWREAASWAE

SEQ ID NO: 207 - protein, 113 aa, Artificial Sequence
  Description of Artificial Sequence Synthetic polypeptide
PGRPLQTHVLPEPHLALQPLQPHADHAHADAPAIQPVLWTTPPLQHGHRHGLEPCSMLTGPPARVPAVPFDLHFCRSSIMKPKRDGYMFLKAESKIMFATLQRSSLWCLCSNH

SEQ ID NO: 208 - protein, 57 aa, Artificial Sequence
  Description of Artificial Sequence Synthetic polypeptide
PRPRRCTRHPACPLDHTTPPAWSPPWVRALLDAHRAPSESPCSPFRLAFLQEQYHEA

SEQ ID NO: 209 - protein, 494 aa, Artificial Sequence
  Description of Artificial Sequence Synthetic polypeptide
TRRCHCCPHLRSHPCPHHLRNHPRPHHLRHHACHHHLRNCPHPHFLRHCTCPGRWRNRPSLRRLRSLLCLPHLNHHLFLHWRSRPCLHRKSHPHLLHLRRLYPHHLKHRPCPHHLKNLLCPRHLRNCPLPRHLKHLACLHHLRSHPCPLHLKSHPCLHHRRHLVCSHHLKSLLCPLHLRSLPFPHHLRHHACPHHLRTRLCPHHLKNHLCPPHLRYRAYPPCLWCHACLHRLRNLPCPHRLRSLPRPLHLRLHASPHHLRTPPHPHHLRTHLLPHHRRTRSCPCRWRSHPCCHYLRSRNSAPGPRGRTCHPGLRSRTCPPGLRSHTYLRRLRSHTCPPSLRSHAYALCLRSHTCPPRLRDHICPLSLRNCTCPPRLRSRTCLLCLRSHACPPNLRNHTCPPSLRSHACPPGLRNRICPLSLRSHPCPLGLKSPLRSQANALHLRSCPCSLPLGNHPYLPCLESQPCLSLGNHLCPLCPRSCRCPHLGSHPCRLS

SEQ ID NO: 210 - protein, 53 aa, Artificial Sequence
  Description of Artificial Sequence Synthetic polypeptide
SWKGTNWCNDMCIFITSGQIFKGTRGPRFLWGSKDQRQKGSNYSQSEALCVLL

SEQ ID NO: 211 - protein, 9 aa, Artificial Sequence
  Description of Artificial Sequence Synthetic peptide
KRTKCFTFG

SEQ ID NO: 212 - protein, 27 aa, Artificial Sequence
  Description of Artificial Sequence Synthetic peptide
PIFIQTLLLWDFLQKDLKAYTGTILMM

SEQ ID NO: 213 - protein, 22 aa, Artificial Sequence
  Description of Artificial Sequence Synthetic peptide
QKMILTKQIKTKPTDTFLQILR

SEQ ID NO: 214 - protein, 43 aa, Artificial Sequence
  Description of Artificial Sequence Synthetic polypeptide
GFWIQSIKTITRYTIFVLKDIMTPPNLIAELHNILLKTITHHS

SEQ ID NO: 215 - protein, 40 aa, Artificial Sequence
  Description of Artificial Sequence Synthetic polypeptide
NYSNVQWRNLQSSVCGLPAKGEDIFLQFRTHTTGRQVHVL

SEQ ID NO: 216 - protein, 44 aa, Artificial Sequence
  Description of Artificial Sequence Synthetic polypeptide
YQSRVLPQTEQDAKKGQNVSLLGKYILHTRTRGNLRKSRKWKSM

SEQ ID NO: 217 - protein, 85 aa, Artificial Sequence
  Description of Artificial Sequence Synthetic polypeptide
SSQNARGCSPRGPCTSSSYTGGPCTSPLLAPVIFCPFPENLPGQLRFPSGLLAFWDSQVCDLHVLPCPQQDVLPTGQDLPCAAVG

SEQ ID NO: 218 - protein, 72 aa, Artificial Sequence
  Description of Artificial Sequence Synthetic polypeptide
GAAPTMSAAQIAMVWPLLSILSEWKEICVWSIWMTETLFDIVWWCPMSRLRLALTVPPSTTTTCVTVPAWAA

SEQ ID NO: 219 - protein, 97 aa, Artificial Sequence
  Description of Artificial Sequence Synthetic polypeptide
TGGPSSPSSHWKTPVVIYWDGTALRCVFVPVLGETGAQRKRISARKGSLTTSCPQGALSEHCPTTPAPLPSQRRNHWMENISPFRSVGVSASRCSES

SEQ ID NO: 220 - protein, 31 aa, Artificial Sequence
  Description of Artificial Sequence Synthetic polypeptide
FHTPARHPRPRHGHLQAVTAHDGGCEALPPP

SEQ ID NO: 221 - protein, 78 aa, Artificial Sequence
  Description of Artificial Sequence Synthetic polypeptide
CCPRTILNNGSLKTQVQMKLPECQRLLPPWPLHQQLLHRRPLHQPPPGPCHLLSLPRKPTRAATVSVWASCILGQPSL

SEQ ID NO: 222 - protein, 28 aa, Artificial Sequence
  Description of Artificial Sequence Synthetic peptide
VRKHFQTYGNYFLKTTFCPPCRPKQWMI

SEQ ID NO: 223 - protein, 46 aa, Artificial Sequence
  Description of Artificial Sequence Synthetic polypeptide
LARTPLPSTRCFANWPRPALCSCGLIPHPRPAPASAPWPSTSSHST

SEQ ID NO: 224 - protein, 81 aa, Artificial Sequence
  Description of Artificial Sequence Synthetic polypeptide
ELQETGHRQVALRRSGRPPKCAERPGAADTGAHCTSTDGRLKISVETYTVSSQLLMVLMSLDLDTGLVPSLVSKCLILRVK

SEQ ID NO: 225 - protein, 10 aa, Artificial Sequence
  Description of Artificial Sequence Synthetic peptide
KSDASRLSGA

SEQ ID NO: 226 - protein, 29 aa, Artificial Sequence
  Description of Artificial Sequence Synthetic peptide
RTAYFCQYHTASVYSERAMPPGCPEPSQA

SEQ ID NO: 227 - protein, 91 aa, Artificial Sequence
  Description of Artificial Sequence Synthetic polypeptide
TRASPPRSSSAIAVRASCCPYGSTSTASRSPTQRCRLARAAASTATEVTFGSSEMQGHTMGFWLTKLNYLCHLSMLTDSLFLPISHCQCIL

SEQ ID NO: 228 - protein, 31 aa, Artificial Sequence
  Description of Artificial Sequence Synthetic polypeptide
SSLRITGDWTSSGRSTKIWKTTQMCRKTWSG

SEQ ID NO: 229 - protein, 104 aa, Artificial Sequence
  Description of Artificial Sequence Synthetic polypeptide
RRRRGGVGRRGVRPGRVRPGGTGRRGGDGGRAAAARAALGELARALPGHLLQSQSARRAARMAQLRRRAAALPNAAAWHGPPHPQLPRSPLALQRCRDTRWASG

SEQ ID NO: 230 - protein, 66 aa, Artificial Sequence
  Description of Artificial Sequence Synthetic polypeptide
SCKVFFKRAAEGKQKYLCASRNDCTIDKFRRKNCPSCRLRKCYEAGMTLGEKFRVGNCKHLKMTRP

SEQ ID NO: 231 - protein, 49 aa, Artificial Sequence
  Description of Artificial Sequence Synthetic polypeptide
MAGAPPPASLPPCSLISDCCASNQRDSVGVGPSEPGNNIKICNESASRK

SEQ ID NO: 232 - protein, 61 aa, Artificial Sequence
  Description of Artificial Sequence Synthetic polypeptide
HGWRPFLPVRARSRWNRRLDVTVANGRSWKYGWSLLRVPQVNGIQVLNVSLKSSSNVISYE

SEQ ID NO: 233 - protein, 65 aa, Artificial Sequence
  Description of Artificial Sequence Synthetic polypeptide
RLKEVFQTKIQEFRKACYTLTGYQIDITTENQYRLTSLYAEHPGDCLIFKLRVPGSSVLVTVPGL

SEQ ID NO: 234 - protein, 107 aa, Artificial Sequence
  Description of Artificial Sequence Synthetic polypeptide
AEVLKVIRQSAGQKTTCGQGLEGPWERPPPLDESERDGGSEDQVEDPALSALLLRPRPPRPEVGAHQDEQAAQGADPRLGAQPACRGLPGLLTVPQPEPLLAPPSAA

SEQ ID NO: 235 - protein, 100 aa, Artificial Sequence
  Description of Artificial Sequence Synthetic polypeptide
ERAEWRENIREQQKKCFRSFSLTSVELQMLTNSCVKLQTVHSIPLTINKEEALQRPVASDFEPQGLSEAARWNSKENLLAGPSENDPNLFVALYDFVASG

SEQ ID NO: 236 - protein, 101 aa, Artificial Sequence
  Description of Artificial Sequence Synthetic polypeptide
ELQMLTNSCVKLQTVHSIPLTINKEDDESPGLYGFLNVIVHSATGFKQSSKALQRPVASDFEPQGLSEAARWNSKENLLAGPSENDPNLFVALYDFVASGD

SEQ ID NO: 237 - protein, 101 aa, Artificial Sequence
  Description of Artificial Sequence Synthetic polypeptide
ISNSWDAHLGLGACGEAEGLGVQGAEEEEEEEEEEEEEGAGVPACPPKGPELFPLIFPAEPAQASGPYVEIIEQPKQRGMRFRYKCEGRSAGSIPGERSTD

SEQ ID NO: 238 - protein, 100 aa, Artificial Sequence
  Description of Artificial Sequence Synthetic polypeptide
LQRLDGMGCLEFDEERAQQEDALAQQAFEEARRRTREFEDRDRSHREEMEVHELEKSKRALETQMEEMKTQLEELEDELQATEDAKLRLEVNMQALKGQF

SEQ ID NO: 239 - protein, 100 aa, Artificial Sequence
  Description of Artificial Sequence Synthetic polypeptide
KGSFPENLRHLKNTMETIDWKVFESWMHHWLLFEMSRHSLEQKPTDAPPKAGVPNKPGIPKLLEGSKNSIQWEKAEDNGCRITYYILEIRKSTSNNLQNQ

SEQ ID NO: 240 - protein, 80 aa, Artificial Sequence
  Description of Artificial Sequence Synthetic polypeptide
MRPSGTAGAALLALLAALCPASRALEEKKGNYVVTDHGSCVRACGADSYEMEEDGVRKCKKCEGPCRKVCNGIGIGEFKD

SEQ ID NO: 241 - protein, 100 aa, Artificial Sequence
  Description of Artificial Sequence Synthetic polypeptide
LPQPPICTIDVYMIMVKCWMIDADSRPKFRELIIEFSKMARDPQRYLVIQLQDKFEHLKMIQQEEIRKLEEEKKQLEGEIIDFYKMKAASEALQTQLSTD

SEQ ID NO: 242 - protein, 101 aa, Artificial Sequence
  Description of Artificial Sequence Synthetic polypeptide
SWENSDDSRNKLSKIPSTPKLIPKVTKTADKHKDVIINQAKMSTREKNSQVYRRKHQELQAMQMELQSPEYKLSKLRTSTIMTDYNPNYCFAGKTSSISDL

SEQ ID NO: 243 - protein, 100 aa, Artificial Sequence
  Description of Artificial Sequence Synthetic polypeptide
EGHRMDKPANCTHDLYMIMRECWHAAPSQRPTFKQLVEDLDRVLTVTSTDVKATQEENRELRSRCEELHGKNLELGKIMDRFEEVVYQAMEEVQKQKELS

SEQ ID NO: 244 - protein, 107 aa, Artificial Sequence
  Description of Artificial Sequence Synthetic polypeptide
RDNTLLLRRVELFSLSRQVARESTYLSSLKGSRLHPEELGGPPLKKLKQEATSKSQIMSLWGLVSKMPPEKVQRLYVDFPQHLRHLLGDWLESQPWEFLVGSDAFCC

SEQ ID NO: 245 - protein, 83 aa, Artificial Sequence
  Description of Artificial Sequence Synthetic polypeptide
MSREMQDVDLAEVKPLVEKGETITGLLQEFDVQEALSVVSEDQSLFECAYGTPHLAKTEMTASSSSDYGQTSKMSPRVPQQDW

SEQ ID NO: 246 - protein, 101 aa, Artificial Sequence
  Description of Artificial Sequence Synthetic polypeptide
VLDMHGFLRQALCRLRQEEPQSLQAAVRTDGFDEFKVRLQDLSSCITQGKAIETQSSSSEEIVPSPPSPPPLPRIYKPCFVCQDKSSGYHYGVSACEGCKG

SEQ ID NO: 247 - protein, 101 aa, Artificial Sequence
  Description of Artificial Sequence Synthetic polypeptide
RSSPEQPRPSTSKAVSPPHLDGPPSPRSPVIGSEVFLPNSNHVASGAGEAAIETQSSSSEEIVPSPPSPPPLPRIYKPCFVCQDKSSGYHYGVSACEGCKG

SEQ ID NO: 248 - protein, 101 aa, Artificial Sequence
  Description of Artificial Sequence Synthetic polypeptide
VARFNDLRFVGRSGRGKSFTLTITVFTNPPQVATYHRAIKITVDGPREPRNRTEKHSTMPDSPVDVKTQSRLTPPTMPPPPTTQGAPRTSSFTPTTLTNGT

SEQ ID NO: 249 - protein, 55 aa, Artificial Sequence
  Description of Artificial Sequence Synthetic polypeptide
MALNSEALSVVSEDQSLFECAYGTPHLAKTEMTASSSSDYGQTSKMSPRVPQQDW

SEQ ID NO: 250 - protein, 10 aa, Homo sapiens
KLVVVGACGV

SEQ ID NO: 251 - protein, 9 aa, Homo sapiens
LVVVGACGV

SEQ ID NO: 252 - protein, 9 aa, Homo sapiens
VVGACGVGK

SEQ ID NO: 253 - protein, 10 aa, Homo sapiens
VVVGACGVGK

SEQ ID NO: 254 - protein, 9 aa, Homo sapiens
VVGADGVGK

SEQ ID NO: 255 - protein, 10 aa, Homo sapiens
VVVGADGVGK

SEQ ID NO: 256 - protein, 10 aa, Homo sapiens
KLVVVGADGV

SEQ ID NO: 257 - protein, 9 aa, Homo sapiens
LVVVGADGV

SEQ ID NO: 258 - protein, 10 aa, Homo sapiens
KLVVVGAVGV

SEQ ID NO: 259 - protein, 9 aa, Homo sapiens
LVVVGAVGV

SEQ ID NO: 260 - protein, 9 aa, Homo sapiens
VVGAVGVGK

SEQ ID NO: 261 - protein, 10 aa, Homo sapiens
VVVGAVGVGK

SEQ ID NO: 262 - protein, 10 aa, Homo sapiens
ILDTAGHEEY

SEQ ID NO: 263 - protein, 10 aa, Homo sapiens
ILDTAGLEEY

SEQ ID NO: 264 - protein, 10 aa, Homo sapiens
LLDILDTAGL

SEQ ID NO: 265 - protein, 10 aa, Homo sapiens
ILDTAGKEEY

SEQ ID NO: 266 - protein, 10 aa, Homo sapiens
ILDTAGREEY

SEQ ID NO: 267 - protein, 9 aa, Homo sapiens
EYMANGSLL

SEQ ID NO: 268 - protein, 9 aa, Homo sapiens
MANGSLLNY

SEQ ID NO: 269 - protein, 10 aa, Homo sapiens
MANGSLLNYL

SEQ ID NO: 270 - protein, 9 aa, Homo sapiens
SLLNYLREM

SEQ ID NO: 271 - protein, 9 aa, Homo sapiens
YMANGSLLN

SEQ ID NO: 272 - protein, 10 aa, Homo sapiens
YMANGSLLNY

SEQ ID NO: 273 - protein, 11 aa, Homo sapiens
IIRNRGENSCK

SEQ ID NO: 274 - protein, 10 aa, Homo sapiens
CLTSTVQLIM

SEQ ID NO: 275 - protein, 9 aa, Homo sapiens
IMQLMPFGC

SEQ ID NO: 276 - protein, 10 aa, Homo sapiens
IMQLMPFGCL

SEQ ID NO: 277 - protein, 9 aa, Homo sapiens
LIMQLMPFG

SEQ ID NO: 278 - protein, 10 aa, Homo sapiens
LIMQLMPFGC

SEQ ID NO: 279 - protein, 9 aa, Homo sapiens
LTSTVQLIM

SEQ ID NO: 280 - protein, 9 aa, Homo sapiens
MQLMPFGCL

SEQ ID NO: 281 - protein, 10 aa, Homo sapiens
MQLMPFGCLL

SEQ ID NO: 282 - protein, 9 aa, Homo sapiens
QLIMQLMPF

SEQ ID NO: 283 - protein, 10 aa, Homo sapiens
QLIMQLMPFG

SEQ ID NO: 284 - protein, 9 aa, Homo sapiens
STVQLIMQL

SEQ ID NO: 285 - protein, 10 aa, Homo sapiens
VQLIMQLMPF

SEQ ID NO: 286 - protein, 9 aa, Homo sapiens
GQYGKVYEG

SEQ ID NO: 287 - protein, 10 aa, Homo sapiens
GQYGKVYEGV

SEQ ID NO: 288 - protein, 9 aa, Homo sapiens
KLGGGQYGK

SEQ ID NO: 289 - protein, 10 aa, Homo sapiens
KLGGGQYGKV

SEQ ID NO: 290 - protein, 9 aa, Homo sapiens
KVYEGVWKK

SEQ ID NO: 291 - protein, 10 aa, Homo sapiens
KVYEGVWKKY

SEQ ID NO: 292 - protein, 9 aa, Homo sapiens
QYGKVYEGV

SEQ ID NO: 293 - protein, 10 aa, Homo sapiens
QYGKVYEGVW

SEQ ID NO: 294 - protein, 9 aa, Homo sapiens
GQYGVVYEG

SEQ ID NO: 295 - protein, 10 aa, Homo sapiens
GQYGVVYEGV

SEQ ID NO: 296 - protein, 9 aa, Homo sapiens
KLGGGQYGV

SEQ ID NO: 297 - protein, 10 aa, Homo sapiens
KLGGGQYGVV

SEQ ID NO: 298 - protein, 9 aa, Homo sapiens
QYGVVYEGV

SEQ ID NO: 299 - protein, 10 aa, Homo sapiens
QYGVVYEGVW

SEQ ID NO: 300 - protein, 9 aa, Homo sapiens
VVYEGVWKK

SEQ ID NO: 301 - protein, 10 aa, Homo sapiens
VVYEGVWKKY

SEQ ID NO: 302 - protein, 10 aa, Homo sapiens
ATQISSATEY

SEQ ID NO: 303 - protein, 10 aa, Homo sapiens
ISSATEYLEK

SEQ ID NO: 304 - protein, 9 aa, Homo sapiens
SSATEYLEK

SEQ ID NO: 305 - protein, 10 aa, Homo sapiens
TQISSATEYL

SEQ ID NO: 306 - protein, 10 aa, Homo sapiens
YMATQISSAT

SEQ ID NO: 307 - protein, 10 aa, Homo sapiens
FYIIIEFMTY

SEQ ID NO: 308 - protein, 10 aa, Homo sapiens
IIEFMTYGNL

SEQ ID NO: 309 - protein, 9 aa, Homo sapiens
IIIEFMTYG

SEQ ID NO: 310 - protein, 10 aa, Homo sapiens
IIIEFMTYGN

SEQ ID NO: 311 - protein, 10 aa, Homo sapiens
YIIIEFMTYG

SEQ ID NO: 312 - protein, 10 aa, Homo sapiens
GQHGEVYEGV

SEQ ID NO: 313 - protein, 10 aa, Homo sapiens
KLGGGQHGEV

SEQ ID NO: 314 - protein, 9 aa, Homo sapiens
KIADFGMAR

SEQ ID NO: 315 - protein, 10 aa, Homo sapiens
RVAKIADFGM

SEQ ID NO: 316 - protein, 10 aa, Homo sapiens
FILMELMAGG

SEQ ID NO: 317 - protein, 9 aa, Homo sapiens
ILMELMAGG

SEQ ID NO: 318 - protein, 10 aa, Homo sapiens
ILMELMAGGD

SEQ ID NO: 319 - protein, 10 aa, Homo sapiens
LMELMAGGDL

SEQ ID NO: 320 - protein, 9 aa, Homo sapiens
LPRFILMEL

SEQ ID NO: 321 - protein, 10 aa, Homo sapiens
LPRFILMELM

SEQ ID NO: 322 - protein, 10 aa, Homo sapiens
LQSLPRFILM

SEQ ID NO: 323 - protein, 10 aa, Homo sapiens
SLPRFILMEL

SEQ ID NO: 324 - protein, 9 aa, Homo sapiens
LATEKSRWS

SEQ ID NO: 325 - protein, 10 aa, Homo sapiens
LATEKSRWSG

SEQ ID NO: 326 - protein, 11 aa, Homo sapiens
GPPPADLCHAL

SEQ ID NO: 327 - protein, 9 aa, Homo sapiens
GLLLEMLDA

SEQ ID NO: 328 - protein, 9 aa, Homo sapiens
LYGLLLEML

SEQ ID NO: 329 - protein, 9 aa, Homo sapiens
NVVPLYGLL

SEQ ID NO: 330 - protein, 9 aa, Homo sapiens
PLYGLLLEM

SEQ ID NO: 331 - protein, 10 aa, Homo sapiens
PLYGLLLEML

SEQ ID NO: 332 - protein, 10 aa, Homo sapiens
VPLYGLLLEM

SEQ ID NO: 333 - protein, 9 aa, Homo sapiens
VVPLYGLLL

SEQ ID NO: 334 - protein, 9 aa, Homo sapiens
FLPSTLKSL

SEQ ID NO: 335 - protein, 9 aa, Homo sapiens
GVYTFLPST

SEQ ID NO: 336 - protein, 10 aa, Homo sapiens
GVYTFLPSTL

SEQ ID NO: 337 - protein, 10 aa, Homo sapiens
TFLPSTLKSL

SEQ ID NO: 338 - protein, 9 aa, Homo sapiens
VYTFLPSTL

SEQ ID NO: 339 - protein, 9 aa, Homo sapiens
YTFLPSTLK

SEQ ID NO: 340 - protein, 9 aa, Homo sapiens
NVVPLCDLL

SEQ ID NO: 341 - protein, 10 aa, Homo sapiens
NVVPLCDLLL

SEQ ID NO: 342 - protein, 9 aa, Homo sapiens
PLCDLLLEM

SEQ ID NO: 343 - protein, 10 aa, Homo sapiens
PLCDLLLEML

SEQ ID NO: 344 - protein, 10 aa, Homo sapiens
VPLCDLLLEM

SEQ ID NO: 345 - protein, 9 aa, Homo sapiens
VVPLCDLLL

SEQ ID NO: 346 - protein, 9 aa, Homo sapiens
NVVPLNDLL

SEQ ID NO: 347 - protein, 10 aa, Homo sapiens
NVVPLNDLLL

SEQ ID NO: 348 - protein, 9 aa, Homo sapiens
PLNDLLLEM

SEQ ID NO: 349 - protein, 10 aa, Homo sapiens
PLNDLLLEML

SEQ ID NO: 350 - protein, 10 aa, Homo sapiens
VPLNDLLLEM

SEQ ID NO: 351 - protein, 9 aa, Homo sapiens
NVVPLSDLL

SEQ ID NO: 352 - protein, 10 aa, Homo sapiens
NVVPLSDLLL

SEQ ID NO: 353 - protein, 9 aa, Homo sapiens
PLSDLLLEM

SEQ ID NO: 354 - protein, 10 aa, Homo sapiens
PLSDLLLEML

SEQ ID NO: 355 - protein, 10 aa, Homo sapiens
VPLSDLLLEM

SEQ ID NO: 356 - protein, 9 aa, Homo sapiens
VVPLSDLLL

SEQ ID NO: 357 - protein, 10 aa, Homo sapiens
VLERCPHRPI

SEQ ID NO: 358 - protein, 9 aa, Homo sapiens
YTLDVLERC

SEQ ID NO: 359 - protein, 9 aa, Homo sapiens
FQNGKMALS

SEQ ID NO: 360 - protein, 10 aa, Homo sapiens
VMAGLGSPYV

SEQ ID NO: 361 - protein, 9 aa, Homo sapiens
KPIIIGHHA

SEQ ID NO: 362 - protein, 9 aa, Homo sapiens
KPIIIGCHA

SEQ ID NO: 363 - protein, 9 aa, Homo sapiens
KPIIIGGHA

SEQ ID NO: 364 - protein, 9 aa, Homo sapiens
KPIIIGSHA

SEQ ID NO: 365 - protein, 10 aa, Homo sapiens
IIEYCCYGDL

SEQ ID NO: 366 - protein, 10 aa, Homo sapiens
TIGGPTLVII

SEQ ID NO: 367 - protein, 9 aa, Homo sapiens
VIIEYCCYG

SEQ ID NO: 368 - protein, 10 aa, Homo sapiens
HMNIANLLGA

SEQ ID NO: 369 - protein, 10 aa, Homo sapiens
IANLLGACTI

SEQ ID NO: 370 - protein, 9 aa, Homo sapiens
MNIANLLGA

SEQ ID NO: 371 - protein, 9 aa, Homo sapiens
YLGNHMNIA

SEQ ID NO: 372 - protein, 10 aa, Homo sapiens
YLGNHMNIAN

SEQ ID NO: 373 - protein, 9 aa, Homo sapiens
VLHESNSPY

SEQ ID NO: 374 - protein, 10 aa, Homo sapiens
VLHESNSPYI

SEQ ID NO: 375 - protein, 10 aa, Homo sapiens
LQVLHECNSL

SEQ ID NO: 376 - protein, 10 aa, Homo sapiens
LYIVGFYGAF

SEQ ID NO: 377 - protein, 9 aa, Homo sapiens
NSLYIVGFY

SEQ ID NO: 378 - protein, 9 aa, Homo sapiens
QVLHECNSL

SEQ ID NO: 379 - protein, 9 aa, Homo sapiens
SLYIVGFYG

SEQ ID NO: 380 - protein, 10 aa, Homo sapiens
SLYIVGFYGA

SEQ ID NO: 381 - protein, 9 aa, Homo sapiens
VLHECNSLY

SEQ ID NO: 382 - protein, 10 aa, Homo sapiens
VLHECNSLYI

SEQ ID NO: 383 - protein, 10 aa, Homo sapiens
FYQQQQQSDL

SEQ ID NO: 384 - protein, 10 aa, Homo sapiens
QQQSDLQPPA

SEQ ID NO: 385 - protein, 9 aa, Homo sapiens
QQSDLQPPA

SEQ ID NO: 386 - protein, 9 aa, Homo sapiens
YQQQQQSDL

SEQ ID NO: 387 - protein, 9 aa, Homo sapiens
FELLSTPPL

SEQ ID NO: 388 - protein, 10 aa, Homo sapiens
LLSTPPLSPS

SEQ ID NO: 389 - protein, 9 aa, Homo sapiens
FELLPIPPL

SEQ ID NO: 390 - protein, 10 aa, Homo sapiens
IWKKFELLPI

SEQ ID NO: 391 - protein, 10 aa, Homo sapiens
LLPIPPLSPS

SEQ ID NO: 392 - protein, 9 aa, Homo sapiens
LPIPPLSPS

SEQ ID NO: 393 - protein, 10 aa, Homo sapiens
IIEYCFYGDL

SEQ ID NO: 394 - protein, 9 aa, Homo sapiens
IIIEYCFYG

SEQ ID NO: 395 - protein, 9 aa, Homo sapiens
IYIIIEYCF

SEQ ID NO: 396 - protein, 10 aa, Homo sapiens
IYIIIEYCFY

SEQ ID NO: 397 - protein, 10 aa, Homo sapiens
YIIIEYCFYG

SEQ ID NO: 398 - protein, 10 aa, Homo sapiens
KITEQEKDFL

SEQ ID NO: 399 - protein, 11 aa, Homo sapiens
STRDPLSEITK

SEQ ID NO: 400 - protein, 8 aa, Homo sapiens
DPLSEITK

SEQ ID NO: 401 - protein, 10 aa, Homo sapiens
LPLKFRDAET

SEQ ID NO: 402 - protein, 10 aa, Homo sapiens
QTGVMICAYL

SEQ ID NO: 403 - protein, 10 aa, Homo sapiens
AFSGEYIPTV

SEQ ID NO: 404 - protein, 9 aa, Homo sapiens
SMNRRPILT

SEQ ID NO: 405 - protein, 10 aa, Homo sapiens
YMCNSSCMGS

SEQ ID NO: 406 - protein, 9 aa, Homo sapiens
GMNQRPILT

SEQ ID NO: 407 - protein, 9 aa, Homo sapiens
GMNWRPILT

SEQ ID NO: 408 - protein, 10 aa, Homo sapiens
LLGRNSFEVC

SEQ ID NO: 409 - protein, 9 aa, Homo sapiens
ALKYIFVAV

SEQ ID NO: 410 - protein, 10 aa, Homo sapiens
ALKYIFVAVR

SEQ ID NO: 411 - protein, 9 aa, Homo sapiens
AVRAICVMK

SEQ ID NO: 412 - protein, 10 aa, Homo sapiens
AVRAICVMKS

SEQ ID NO: 413 - protein, 9 aa, Homo sapiens
CVEKKTALK

SEQ ID NO: 414 - protein, 10 aa, Homo sapiens
CVEKKTALKY

SEQ ID NO: 415 - protein, 9 aa, Homo sapiens
CVMKSFLIF

SEQ ID NO: 416 - protein, 10 aa, Homo sapiens
CVMKSFLIFR

SEQ ID NO: 417 - protein, 9 aa, Homo sapiens
FLIFRRWKS

SEQ ID NO: 418 - protein, 10 aa, Homo sapiens
FRRWKSHSPL

SEQ ID NO: 419 - protein, 9 aa, Homo sapiens
FVAVRAICV

SEQ ID NO: 420 - protein, 10 aa, Homo sapiens
FVAVRAICVM

SEQ ID NO: 421 - protein, 9 aa, Homo sapiens
IQLHLSHPI

SEQ ID NO: 422 - protein, 10 aa, Homo sapiens
KSFLIFRRWK

SEQ ID NO: 423 - protein, 9 aa, Homo sapiens
KTALKYIFV

SEQ ID NO: 424 - protein, 10 aa, Homo sapiens
KYIFVAVRAI

SEQ ID NO: 425 - protein, 10 aa, Homo sapiens
RWKSHSPLQI

SEQ ID NO: 426 - protein, 10 aa, Homo sapiens
TALKYIFVAV

SEQ ID NO: 427 - protein, 10 aa, Homo sapiens
VAVRAICVMK

SEQ ID NO: 428 - protein, 9 aa, Homo sapiens
VMKSFLIFR

SEQ ID NO: 429 - protein, 10 aa, Homo sapiens
VMKSFLIFRR

SEQ ID NO: 430 - protein, 9 aa, Homo sapiens
YIFVAVRAI

SEQ ID NO: 431 - protein, 9 aa, Homo sapiens
ALFFFFFET

SEQ ID NO: 432 - protein, 10 aa, Homo sapiens
ALFFFFFETK

SEQ ID NO: 433 - protein, 10 aa, Homo sapiens
AQAGVQWRSL

SEQ ID NO: 434 - protein, 10 aa, Homo sapiens
CLANFCIFNR

SEQ ID NO: 435 - protein, 10 aa, Homo sapiens
CLSFLSSWDY

SEQ ID NO: 436 - protein, 9 aa, Homo sapiens
FFETKSCSV

SEQ ID NO: 437 - protein, 10 aa, Homo sapiens
FFFETKSCSV

SEQ ID NO: 438 - protein, 10 aa, Homo sapiens
FKLFSCLSFL

SEQ ID NO: 439 - protein, 10 aa, Homo sapiens
FLSSWDYRRM

SEQ ID NO: 440 - protein, 10 aa, Homo sapiens
GFKLFSCLSF

SEQ ID NO: 441 - protein, 9 aa, Homo sapiens
KLFSCLSFL

SEQ ID NO: 442 - protein, 10 aa, Homo sapiens
KLFSCLSFLS

SEQ ID NO: 443 - protein, 10 aa, Homo sapiens
LALFFFFFET

SEQ ID NO: 444 - protein, 9 aa, Homo sapiens
LFFFFFETK

SEQ ID NO: 445 - protein, 9 aa, Homo sapiens
LSFLSSWDY

SEQ ID NO: 446 - protein, 10 aa, Homo sapiens
LSFLSSWDYR

SEQ ID NO: 447 - protein, 9 aa, Homo sapiens
RMPPCLANF

SEQ ID NO: 448 - protein, 10 aa, Homo sapiens
RRMPPCLANF

SEQ ID NO: 449 - protein, 10 aa, Homo sapiens
SLQPPPPGFK

SEQ ID NO: 450 - protein, 9 aa, Homo sapiens
VQWRSLGSL

SEQ ID NO: 451 - protein, 9 aa, Homo sapiens
FFFSVIFST

SEQ ID NO: 452 - protein, 10 aa, Homo sapiens
MSVCFFFFSV

SEQ ID NO: 453 - protein, 9 aa, Homo sapiens
SVCFFFFSV

SEQ ID NO: 454 - protein, 10 aa, Homo sapiens
SVCFFFFSVI

SEQ ID NO: 455 - protein, 10 aa, Homo sapiens
FFCYILNTMF

SEQ ID NO: 456 - protein, 10 aa, Homo sapiens
MSVCFFFFCY

SEQ ID NO: 457 - protein, 10 aa, Homo sapiens
SVCFFFFCYI

SEQ ID NO: 458 - protein, 9 aa, Homo sapiens
KQWSSVTSL

SEQ ID NO: 459 - protein, 9 aa, Homo sapiens
VLWMPTSTV

SEQ ID NO: 460 - protein, 9 aa, Homo sapiens
ILIRKAMTV

SEQ ID NO: 461 - protein, 8 aa, Homo sapiens
RMKVPLMK

SEQ ID NO: 462 - protein, 9 aa, Homo sapiens
TLKKKPRDI

SEQ ID NO: 463 - protein, 10 aa, Homo sapiens
LIEGFEALLK

SEQ ID NO: 464 - protein, 9 aa, Homo sapiens
QQLKWTPHI

SEQ ID NO: 465 - protein, 10 aa, Homo sapiens
QLKWTPHILK

SEQ ID NO: 466 - protein, 10 aa, Homo sapiens
IVSEKNQQLK

SEQ ID NO: 467 - protein, 11 aa, Homo sapiens
NQQLKWTPHIL

SEQ ID NO: 468 - protein, 10 aa, Homo sapiens
NQQLKWTPHI

SEQ ID NO: 469 - protein, 9 aa, Homo sapiens
QLKWTPHIL

SEQ ID NO: 470 - protein, 10 aa, Homo sapiens
GPPRPPRAAC

SEQ ID NO: 471 - protein, 9 aa, Homo sapiens
PPRPPRAAC

SEQ ID NO: 472 - protein, 9 aa, Homo sapiens
SLRRKYLRV

SEQ ID NO: 473 - protein, 10 aa, Homo sapiens
SAACHRRGCV

SEQ ID NO: 474 - protein, 10 aa, Homo sapiens
ALWECSLPQA

SEQ ID NO: 475 - protein, 9 aa, Homo sapiens
CLIVSRTLL

SEQ ID NO: 476 - protein, 10 aa, Homo sapiens
CLIVSRTLLL

SEQ ID NO: 477 - protein, 9 aa, Homo sapiens
FLLALWECS

SEQ ID NO: 478 - protein, 10 aa, Homo sapiens
FLLALWECSL

SEQ ID NO: 479 - protein, 9 aa, Homo sapiens
IVSRTLLLV

SEQ ID NO: 480 - protein, 9 aa, Homo sapiens
LIVSRTLLL

SEQ ID NO: 481 - protein, 10 aa, Homo sapiens
LIVSRTLLLV

SEQ ID NO: 482 - protein, 9 aa, Homo sapiens
LLALWECSL

SEQ ID NO: 483 - protein, 9 aa, Homo sapiens
LPQARLCLI

SEQ ID NO: 484 - protein, 10 aa, Homo sapiens
LPQARLCLIV

SEQ ID NO: 485 - protein, 10 aa, Homo sapiens
NVRDKKRATF

SEQ ID NO: 486 - protein, 10 aa, Homo sapiens
SLPQARLCLI

SEQ ID NO: 487 - protein, 9 aa, Homo sapiens
FFCWILSCK

SEQ ID NO: 488 - protein, 10 aa, Homo sapiens
FFFCWILSCK

SEQ ID NO: 489 - protein, 9 aa, Homo sapiens
ITAFFFCWI

SEQ ID NO: 490 - protein, 10 aa, Homo sapiens
LYITAFFFCW

SEQ ID NO: 491 - protein, 10 aa, Homo sapiens
YITAFFFCWI

SEQ ID NO: 492 - protein, 9 aa, Homo sapiens
ITAFFLLDI

SEQ ID NO: 493 - protein, 9 aa, Homo sapiens
LLYITAFFL

SEQ ID NO: 494 - protein, 10 aa, Homo sapiens
LLYITAFFLL

SEQ ID NO: 495 - protein, 9 aa, Homo sapiens
LYITAFFLL

SEQ ID NO: 496 - protein, 10 aa, Homo sapiens
LYITAFFLLD

SEQ ID NO: 497 - protein, 10 aa, Homo sapiens
YITAFFLLDI

SEQ ID NO: 498 - protein, 9 aa, Homo sapiens
GSNEVTTRY

SEQ ID NO: 499 - protein, 9 aa, Homo sapiens
MPKDVNIQV

SEQ ID NO: 500 - protein, 10 aa, Homo sapiens
TGSNEVTTRY

SEQ ID NO: 501 - protein, 10 aa, Homo sapiens
KKLMLLRLNL

SEQ ID NO: 502 - protein, 9 aa, Homo sapiens
KLMLLRLNL

SEQ ID NO: 503 - protein, 10 aa, Homo sapiens
KLMLLRLNLR

SEQ ID NO: 504 - protein, 9 aa, Homo sapiens
LLRLNLRKM

SEQ ID NO: 505 - protein, 8 aa, Homo sapiens
LMLLRLNL

SEQ ID NO: 506 - protein, 10 aa, Homo sapiens
LMLLRLNLRK

SEQ ID NO: 507 - protein, 10 aa, Homo sapiens
LNLRKMCGPF

SEQ ID NO: 508 - protein, 9 aa, Homo sapiens
MLLRLNLRK

SEQ ID NO: 509 - protein, 10 aa, Homo sapiens
MLLRLNLRKM

SEQ ID NO: 510 - protein, 9 aa, Homo sapiens
NLRKMCGPF

SEQ ID NO: 511 - protein, 10 aa, Homo sapiens
NYCQKKLMLL

SEQ ID NO: 512 - protein, 9 aa, Homo sapiens
YCQKKLMLL

SEQ ID NO: 513 - protein, 10 aa, Homo sapiens
FKKKTYTCAI

SEQ ID NO: 514 - protein, 10 aa, Homo sapiens
ITTVKATETK

SEQ ID NO: 515 - protein, 9 aa, Homo sapiens
KSKKKETFK

SEQ ID NO: 516 - protein, 10 aa, Homo sapiens
KSKKKETFKK

SEQ ID NO: 517 - protein, 10 aa, Homo sapiens
KTYTCAITTV

SEQ ID NO: 518 - protein, 9 aa, Homo sapiens
TFKKKTYTC

SEQ ID NO: 519 - protein, 9 aa, Homo sapiens
TYTCAITTV

SEQ ID NO: 520 - protein, 10 aa, Homo sapiens
TYTCAITTVK

SEQ ID NO: 521 - protein, 9 aa, Homo sapiens
YTCAITTVK

SEQ ID NO: 522 - protein, 9 aa, Homo sapiens
TAKSKKRNL

SEQ ID NO: 523 - protein, 9 aa, Homo sapiens
FALCGFWQI

SEQ ID NO: 524 - protein, 8 aa, Homo sapiens
LKKLRSPL

SEQ ID NO: 525 - protein, 8 aa, Homo sapiens
SLKKVPAL

SEQ ID NO: 526 - protein, 10 aa, Homo sapiens
RKISNWSLKK

SEQ ID NO: 527 - protein, 11 aa, Homo sapiens
VPALKKLRSPL

SEQ ID NO: 528 - protein, 10 aa, Homo sapiens
KRVNTAWKTK

SEQ ID NO: 529 - protein, 10 aa, Homo sapiens
MTKKKRVNTA

SEQ ID NO: 530 - protein, 9 aa, Homo sapiens
RVNTAWKTK

SEQ ID NO: 531 - protein, 10 aa, Homo sapiens
RVNTAWKTKK

SEQ ID NO: 532 - protein, 9 aa, Homo sapiens
TKKKRVNTA

SEQ ID NO: 533 - protein, 10 aa, Homo sapiens
WKTKKTSFSL

SEQ ID NO: 534 - protein, 9 aa, Homo sapiens
ALMSAMTTS

SEQ ID NO: 535 - protein, 9 aa, Homo sapiens
AMTTSSSQK

SEQ ID NO: 536 - protein, 10 aa, Homo sapiens
AMTTSSSQKN

SEQ ID NO: 537 - protein, 9 aa, Homo sapiens
CIMKEKKSL

SEQ ID NO: 538 - protein, 10 aa, Homo sapiens
CIMKEKKSLV

SEQ ID NO: 539 - protein, 8 aa, Homo sapiens
IMKEKKSL

SEQ ID NO: 540 - protein, 9 aa, Homo sapiens
IMKEKKSLV

SEQ ID NO: 541 - protein, 10 aa, Homo sapiens
KSLVRLSSCV

SEQ ID NO: 542 - protein, 10 aa, Homo sapiens
LVRLSSCVPV

SEQ ID NO: 543 - protein, 9 aa, Homo sapiens
RLSSCVPVA

SEQ ID NO: 544 - protein, 10 aa, Homo sapiens
RLSSCVPVAL

SEQ ID NO: 545 - protein, 10 aa, Homo sapiens
SAMTTSSSQK

SEQ ID NO: 546 - protein, 9 aa, Homo sapiens
SLVRLSSCV

SEQ ID NO: 547 - protein, 9 aa, Homo sapiens
VPVALMSAM

SEQ ID NO: 548 - protein, 10 aa, Homo sapiens
VRLSSCVPVA

SEQ ID NO: 549 - protein, 9 aa, Homo sapiens
KMRKKYAQK

SEQ ID NO: 550 - protein, 9 aa, Homo sapiens
FVMSDTTYK

SEQ ID NO: 551 - protein, 10 aa, Homo sapiens
FVMSDTTYKI

SEQ ID NO: 552 - protein, 9 aa, Homo sapiens
KTFEKKGEK

SEQ ID NO: 553 - protein, 10 aa, Homo sapiens
LFVMSDTTYK

SEQ ID NO: 554 - protein, 9 aa, Homo sapiens
MSDTTYKIY

SEQ ID NO: 555 - protein, 9 aa, Homo sapiens
VMSDTTYKI

SEQ ID NO: 556 - protein, 10 aa, Homo sapiens
VMSDTTYKIY

SEQ ID NO: 557 - protein, 10 aa, Homo sapiens
YLTKWPKFFL

SEQ ID NO: 558 - protein, 9 aa, Homo sapiens
FLQERNLPP

SEQ ID NO: 559 - protein, 9 aa, Homo sapiens
FRRPHSCLA

SEQ ID NO: 560 - protein, 9 aa, Homo sapiens
LIVLRVVRL

SEQ ID NO: 561 - protein, 9 aa, Homo sapiens
LLSVHLIVL

SEQ ID NO: 562 - protein, 9 aa, Homo sapiens
EVKHLHHLL

SEQ ID NO: 563 - protein, 10 aa, Homo sapiens
HLHHLLKQLK

SEQ ID NO: 564 - protein, 9 aa, Homo sapiens
HLLLKRERV

SEQ ID NO: 565 - protein, 9 aa, Homo sapiens
KIKHLLLKR

SEQ ID NO: 566 - protein, 9 aa, Homo sapiens
KPSEKYLKI

SEQ ID NO: 567 - protein, 9 aa, Homo sapiens
KYLKIKHLL

SEQ ID NO: 568 - protein, 10 aa, Homo sapiens
KYLKIKHLLL

SEQ ID NO: 569 - protein, 10 aa, Homo sapiens
LLKQLKPSEK

SEQ ID NO: 570 - protein, 9 aa, Homo sapiens
LLKRERVDL

SEQ ID NO: 571 - protein, 10 aa, Homo sapiens
LLLKRERVDL

SEQ ID NO: 572 - protein, 10 aa, Homo sapiens
QLKPSEKYLK

SEQ ID NO: 573 - protein, 9 aa, Homo sapiens
YLKIKHLLL

SEQ ID NO: 574 - protein, 10 aa, Homo sapiens
YLKIKHLLLK

SEQ ID NO: 575 - protein, 9 aa, Homo sapiens
ILPRKVLQM

SEQ ID NO: 576 - protein, 9 aa, Homo sapiens
KVLQMDFLV

SEQ ID NO: 577 - protein, 10 aa, Homo sapiens
LPRKVLQMDF

SEQ ID NO: 578 - protein, 10 aa, Homo sapiens
LQMDFLVHPA

SEQ ID NO: 579 - protein, 9 aa, Homo sapiens
QMDFLVHPA

SEQ ID NO: 580 - protein, 10 aa, Homo sapiens
YILPRKVLQM

SEQ ID NO: 581 - protein, 10 aa, Homo sapiens
APSPASRLQC

SEQ ID NO: 582 - protein, 10 aa, Homo sapiens
HPLAPMPSKT

SEQ ID NO: 583 - protein, 9 aa, Homo sapiens
ILPLPQLLL

SEQ ID NO: 584 - protein, 9 aa, Homo sapiens
LLLSADQQA

SEQ ID NO: 585 - protein, 9 aa, Homo sapiens
LPTQTRGCI

SEQ ID NO: 586 - protein, 10 aa, Homo sapiens
LPTQTRGCIL

SEQ ID NO: 587 - protein, 10 aa, Homo sapiens
RISCLPTQTR

SEQ ID NO: 588 - protein, 9 aa, Homo sapiens
SLAETVSLH

SEQ ID NO: 589 - protein, 9 aa, Homo sapiens
TPRSSSSSS

SEQ ID NO: 590 - protein, 10 aa, Homo sapiens
TPRSSSSSSS

SEQ ID NO: 591 - protein, 9 aa, Homo sapiens
ALPPVLLSL

SEQ ID NO: 592 - protein, 10 aa, Homo sapiens
ALPPVLLSLA

SEQ ID NO: 593 - protein, 9 aa, Homo sapiens
ALPRPLLAL

SEQ ID NO: 594 - protein, 9 aa, Homo sapiens
ASRTASCIL

SEQ ID NO: 595 - protein, 10 aa, Homo sapiens
EALPRPLLAL

SEQ ID NO: 596 - protein, 9 aa, Homo sapiens
HLGHPVASR

SEQ ID NO: 597 - protein, 9 aa, Homo sapiens
HPVASRTAS

SEQ ID NO: 598 - protein, 10 aa, Homo sapiens
HPVASRTASC

SEQ ID NO: 599 - protein, 10 aa, Homo sapiens
IIQLSLLSLL

SEQ ID NO: 600 - protein, 9 aa, Homo sapiens
IQLSLLSLL

SEQ ID NO: 601 - protein, 10 aa, Homo sapiens
IQLSLLSLLI

SEQ ID NO: 602 - protein, 9 aa, Homo sapiens
LLALPPVLL

SEQ ID NO: 603 - protein, 9 aa, Homo sapiens
LLIPPLTCL

SEQ ID NO: 604 - protein, 10 aa, Homo sapiens
LLIPPLTCLA

SEQ ID NO: 605 - protein, 9 aa, Homo sapiens
LLSLLIPPL

SEQ ID NO: 606 - protein, 10 aa, Homo sapiens
LLSLLIPPLT

SEQ ID NO: 607 - protein, 10 aa, Homo sapiens
LPRPLLALPP

SEQ ID NO: 608 - protein, 9 aa, Homo sapiens
QLSLLSLLI

SEQ ID NO: 609 - protein, 9 aa, Homo sapiens
RLLQCQATR

SEQ ID NO: 610 - protein, 9 aa, Homo sapiens
RPLLALPPV

SEQ ID NO: 611 - protein, 10 aa, Homo sapiens
RPLLALPPVL

SEQ ID NO: 612 - protein, 9 aa, Homo sapiens
SLAQDHSRL

SEQ ID NO: 613 - protein, 10 aa, Homo sapiens
SLAQDHSRLL

SEQ ID NO: 614 - protein, 10 aa, Homo sapiens
SLLIPPLTCL

SEQ ID NO: 615 - protein, 9 aa, Homo sapiens
SLLSLLIPP

SEQ ID NO: 616 - protein, 10 aa, Homo sapiens
SLLSLLIPPL

SEQ ID NO: 617 - protein, 10 aa, Homo sapiens
AAATSAASTL

SEQ ID NO: 618 - protein, 10 aa, Homo sapiens
AAIPASTSAV

SEQ ID NO: 619 - protein, 9 aa, Homo sapiens
AIPASTSAV

SEQ ID NO: 620 - protein, 10 aa, Homo sapiens
ALPAGCVSSA

SEQ ID NO: 621 - protein, 10 aa, Homo sapiens
APLLTATGSV

SEQ ID NO: 622 - protein, 9 aa, Homo sapiens
APVLSASIL

SEQ ID NO: 623 - protein, 9 aa, Homo sapiens
ATLLPATTV

SEQ ID NO: 624 - protein, 10 aa, Homo sapiens
ATVSTTTAPV

SEQ ID NO: 625 - protein, 10 aa, Homo sapiens
AVPANCLFPA

SEQ ID NO: 626 - protein, 10 aa, Homo sapiens
CLFPAALPST

SEQ ID NO: 627 - protein, 9 aa, Homo sapiens
CPTFVSAAA

SEQ ID NO: 628 - protein, 9 aa, Homo sapiens
FPAALPSTA

SEQ ID NO: 629 - protein, 10 aa, Homo sapiens
FPAALPSTAG

SEQ ID NO: 630 - protein, 9 aa, Homo sapiens
GAECHGRPL

SEQ ID NO: 631 - protein, 9 aa, Homo sapiens
GAISRFIWV

SEQ ID NO: 632 - protein, 10 aa, Homo sapiens
ILPAAIPAST

SEQ ID NO: 633 - protein, 10 aa, Homo sapiens
IWVSGILSPL

SEQ ID NO: 634 - protein, 10 aa, Homo sapiens
LLTATGSVSL

SEQ ID NO: 635 - protein, 9 aa, Homo sapiens
LLYTADSSL

SEQ ID NO: 636 - protein, 9 aa, Homo sapiens
LPAAATSAA

SEQ ID NO: 637 - protein, 10 aa, Homo sapiens
LPAAATSAAS

SEQ ID NO: 638 - protein, 9 aa, Homo sapiens
LPAAIPAST

SEQ ID NO: 639 - protein, 9 aa, Homo sapiens
LPAGCVSSA

SEQ ID NO: 640 - protein, 10 aa, Homo sapiens
LPAGCVSSAP

SEQ ID NO: 641 - protein, 9 aa, Homo sapiens
LYTADSSLW

SEQ ID NO: 642 - protein, 9 aa, Homo sapiens
QPAKSSPSA

SEQ ID NO: 643 - protein, 10 aa, Homo sapiens
QPAKSSPSAA

SEQ ID NO: 644 - protein, 9 aa, Homo sapiens
RFIWVSGIL

SEQ ID NO: 645 - protein, 9 aa, Homo sapiens
RPQRVWSTG

SEQ ID NO: 646 - protein, 10 aa, Homo sapiens
RVWSTGPDSI

SEQ ID NO: 647 - protein, 9 aa, Homo sapiens
SAVPGSIPL

SEQ ID NO: 648 - protein, 9 aa, Homo sapiens
SILPAAIPA

SEQ ID NO: 649 - protein, 9 aa, Homo sapiens
SLPAAATSA

SEQ ID NO: 650 - protein, 10 aa, Homo sapiens
SLPAAATSAA

SEQ ID NO: 651 - protein, 9 aa, Homo sapiens
SLWTTRPQR

SEQ ID NO: 652 - protein, 10 aa, Homo sapiens
SLWTTRPQRV

SEQ ID NO: 653 - protein, 9 aa, Homo sapiens
SPSAAAATL

SEQ ID NO: 654 - protein, 10 aa, Homo sapiens
SPSAAAATLL

SEQ ID NO: 655 - protein, 10 aa, Homo sapiens
TLDALPAGCV

SEQ ID NO: 656 - protein, 9 aa, Homo sapiens
TVSTTTAPV

SEQ ID NO: 657 - protein, 9 aa, Homo sapiens
VLSASILPA

SEQ ID NO: 658 - protein, 10 aa, Homo sapiens
VLSASILPAA

SEQ ID NO: 659 - protein, 9 aa, Homo sapiens
VPANCLFPA

SEQ ID NO: 660 - protein, 10 aa, Homo sapiens
VPANCLFPAA

SEQ ID NO: 661 - protein, 9 aa, Homo sapiens
VPDPSCPTF

SEQ ID NO: 662 - protein, 9 aa, Homo sapiens
VPGSIPLPA

SEQ ID NO: 663 - protein, 10 aa, Homo sapiens
VPGSIPLPAV

SEQ ID NO: 664 - protein, 9 aa, Homo sapiens
WVSGILSPL

SEQ ID NO: 665 - protein, 10 aa, Homo sapiens
YTADSSLWTT

SEQ ID NO: 666 - protein, 9 aa, Homo sapiens
APAGMVNRA

SEQ ID NO: 667 - protein, 9 aa, Homo sapiens
ASLHRRSYL

SEQ ID NO: 668 - protein, 10 aa, Homo sapiens
ASLHRRSYLK

SEQ ID NO: 669 - protein, 10 aa, Homo sapiens
FLLMDNKAPA

SEQ ID NO: 670 - protein, 9 aa, Homo sapiens
HPRRSPSRL

SEQ ID NO: 671 - protein, 9 aa, Homo sapiens
HPSLHISSP

SEQ ID NO: 672 - protein, 10 aa, Homo sapiens
HRRSYLKIHL

SEQ ID NO: 673 - protein, 10 aa, Homo sapiens
HSRFLLMDNK

SEQ ID NO: 674 - protein, 10 aa, Homo sapiens
KLPIPSSASL

SEQ ID NO: 675 - protein, 10 aa, Homo sapiens
KVLTLSSSSH

SEQ ID NO: 676 - protein, 9 aa, Homo sapiens
LIHSRFLLM

SEQ ID NO: 677 - protein, 9 aa, Homo sapiens
LLMDNKAPA

SEQ ID NO: 678 - protein, 10 aa, Homo sapiens
LMDNKAPAGM

SEQ ID NO: 679 - protein, 9 aa, Homo sapiens
LPIPSSASL

SEQ ID NO: 680 - protein, 9 aa, Homo sapiens
MPNLRISSS

SEQ ID NO: 681 - protein, 10 aa, Homo sapiens
MPNLRISSSH

SEQ ID NO: 682 - protein, 10 aa, Homo sapiens
NLRISSSHSL

SEQ ID NO: 683 - protein, 10 aa, Homo sapiens
PPTHSHRLSL

SEQ ID NO: 684 - protein, 10 aa, Homo sapiens
RAGRRVPWAA

SEQ ID NO: 685 - protein, 10 aa, Homo sapiens
RARLHITTSK

SEQ ID NO: 686 - protein, 10 aa, Homo sapiens
RISSSHSLNH

SEQ ID NO: 687 - protein, 9 aa, Homo sapiens
RLHTPPSSR

SEQ ID NO: 688 - protein, 10 aa, Homo sapiens
RLHTPPSSRR

SEQ ID NO: 689 - protein, 9 aa, Homo sapiens
RLRILSPSL

SEQ ID NO: 690 - protein, 9 aa, Homo sapiens
RPLMPNLRI

SEQ ID NO: 691 - protein, 9 aa, Homo sapiens
RPRPLMPNL

SEQ ID NO: 692 - protein, 10 aa, Homo sapiens
SASLHRRSYL

SEQ ID NO: 693 - protein, 9 aa, Homo sapiens
SLHISSPRL

SEQ ID NO: 694 - protein, 9 aa, Homo sapiens
SLHRRSYLK

SEQ ID NO: 695 - protein, 10 aa, Homo sapiens
SLHRRSYLKI

SEQ ID NO: 696 - protein, 9 aa, Homo sapiens
SLIHSRFLL

SEQ ID NO: 697 - protein, 10 aa, Homo sapiens
SLIHSRFLLM

SEQ ID NO: 698 - protein, 9 aa, Homo sapiens
SLLTSSSNL

SEQ ID NO: 699 - protein, 10 aa, Homo sapiens
SLNHHSSSPL

SEQ ID NO: 700 - protein, 10 aa, Homo sapiens
SLSSPSKLPI

SEQ ID NO: 701 - protein, 9 aa, Homo sapiens
SPLSLHTPS

SEQ ID NO: 702 - protein, 10 aa, Homo sapiens
SPLSLHTPSS

SEQ ID NO: 703 - protein, 9 aa, Homo sapiens
SPPTHSHRL

SEQ ID NO: 704 - protein, 9 aa, Homo sapiens
SPRLHTPPS

SEQ ID NO: 705 - protein, 10 aa, Homo sapiens
SPRLHTPPSS

SEQ ID NO: 706 - protein, 10 aa, Homo sapiens
SPSLSSPSKL

SEQ ID NO: 707 - protein, 9 aa, Homo sapiens
SYLKIHLGL

SEQ ID NO: 708 - protein, 10 aa, Homo sapiens
TPSNHRPRPL

SEQ ID NO: 709 - protein, 10 aa, Homo sapiens
TPSSHPSLHI

SEQ ID NO: 710 - protein, 10 aa, Homo sapiens
CVPFWTGRIL

SEQ ID NO: 711 - protein, 11 aa, Homo sapiens
HCVPFWTGRIL

SEQ ID NO: 712 - protein, 9 aa, Homo sapiens
ILLPSAASV

SEQ ID NO: 713 - protein, 10 aa, Homo sapiens
ILLPSAASVC

SEQ ID NO: 714 - protein, 11 aa, Homo sapiens
LLPSAASVCPI

SEQ ID NO: 715 - protein, 10 aa, Homo sapiens
LPSAASVCPI

SEQ ID NO: 716 - protein, 8 aa, Homo sapiens
MRPHCVPF

SEQ ID NO: 717 - protein, 10 aa, Homo sapiens
RILLPSAASV

SEQ ID NO: 718 - protein, 9 aa, Homo sapiens
RMRPHCVPF

SEQ ID NO: 719 - protein, 10 aa, Homo sapiens
RMRPHCVPFW

SEQ ID NO: 720 - protein, 9 aa, Homo sapiens
RTNPTVRMR

SEQ ID NO: 721 - protein, 9 aa, Homo sapiens
SVCPIPFEA

SEQ ID NO: 722 - protein, 9 aa, Homo sapiens
TVRMRPHCV

SEQ ID NO: 723 - protein, 11 aa, Homo sapiens
TVRMRPHCVPF

SEQ ID NO: 724 - protein, 9 aa, Homo sapiens
VPFWTGRIL

SEQ ID NO: 725 - protein, 10 aa, Homo sapiens
VPFWTGRILL

SEQ ID NO: 726 - protein, 10 aa, Homo sapiens
VRMRPHCVPF

SEQ ID NO: 727 - protein, 9 aa, Homo sapiens
AMVPRGVSM

SEQ ID NO: 728 - protein, 10 aa, Homo sapiens
AMVPRGVSMA

SEQ ID NO: 729 - protein, 10 aa, Homo sapiens
AWAPTSRTPW

SEQ ID NO: 730 - protein, 10 aa, Homo sapiens
CPMPTTPVQA

SEQ ID NO: 731 - protein, 10 aa, Homo sapiens
CPSTVPPSPA

SEQ ID NO: 732 - protein, 10 aa, Homo sapiens
GAMVPRGVSM

SEQ ID NO: 733 - protein, 10 aa, Homo sapiens
MPCPMPTTPV

SEQ ID NO: 734 - protein, 9 aa, Homo sapiens
MPTTPVQAW

SEQ ID NO: 735 - protein, 10 aa, Homo sapiens
MPTTPVQAWL

SEQ ID NO: 736 - protein, 10 aa, Homo sapiens
SLPEDRYTQA

SEQ ID NO: 737 - protein, 9 aa, Homo sapiens
SPAQPYLRV

SEQ ID NO: 738 - protein, 10 aa, Homo sapiens
SPAQPYLRVS

SEQ ID NO: 739 - protein, 9 aa, Homo sapiens
TIMPCPMPT

SEQ ID NO: 740 - protein, 9 aa, Homo sapiens
TPVQAWLEA

SEQ ID NO: 741 - protein, 9 aa, Homo sapiens
TSRTPWGAM

SEQ ID NO: 742 - protein, 10 aa, Homo sapiens
VPPSPAQPYL

SEQ ID NO: 743 - protein, 9 aa, Homo sapiens
VPRGVSMAH

SEQ ID NO: 744 - protein, 10 aa, Homo sapiens
CLSARTGLSI

SEQ ID NO: 745 - protein, 10 aa, Homo sapiens
CTTLNSPPLK

SEQ ID NO: 746 - protein, 9 aa, Homo sapiens
GLSISCTTL

SEQ ID NO: 747 - protein, 9 aa, Homo sapiens
SPPLKKMSM

SEQ ID NO: 748 - protein, 9 aa, Homo sapiens
TLNSPPLKK

SEQ ID NO: 749 - protein, 9 aa, Homo sapiens
TTLNSPPLK

SEQ ID NO: 750 - protein, 10 aa, Homo sapiens
TTLNSPPLKK

SEQ ID NO: 751 - protein, 9 aa, Homo sapiens
LQRFRFTHV

SEQ ID NO: 752 - protein, 10 aa, Homo sapiens
LQRFRFTHVI

SEQ ID NO: 753 - protein, 9 aa, Homo sapiens
RLSSVLQRF

SEQ ID NO: 754 - protein, 10 aa, Homo sapiens
RLSSVLQRFR

SEQ ID NO: 755 - protein, 10 aa, Homo sapiens
VLQRFRFTHV

SEQ ID NO: 756 - protein, 10 aa, Homo sapiens
ASVGRHSLSK

SEQ ID NO: 757 - protein, 9 aa, Homo sapiens
KFLPFWGFL

SEQ ID NO: 758 - protein, 9 aa, Homo sapiens
LASVGRHSL

SEQ ID NO: 759 - protein, 10 aa, Homo sapiens
LPFWGFLEEF

SEQ ID NO: 760 - protein, 9 aa, Homo sapiens
PFWGFLEEF

SEQ ID NO: 761 - protein, 9 aa, Homo sapiens
SVGRHSLSK

SEQ ID NO: 762 - protein, 9 aa, Homo sapiens
APRPIRPPF

SEQ ID NO: 763 - protein, 10 aa, Homo sapiens
APRPIRPPFL

SEQ ID NO: 764 - protein, 10 aa, Homo sapiens
AQKMKKAHFL

SEQ ID NO: 765 - protein, 9 aa, Homo sapiens
FLPSAAQKM

SEQ ID NO: 766 - protein, 10 aa, Homo sapiens
GLFLPSAAQK

SEQ ID NO: 767 - protein, 9 aa, Homo sapiens
HPLLVSLLL

SEQ ID NO: 768 - protein, 10 aa, Homo sapiens
KAHFLKTWFR

SEQ ID NO: 769 - protein, 9 aa, Homo sapiens
KARFSTASL

SEQ ID NO: 770 - protein, 9 aa, Homo sapiens
KMKKAHFLK

SEQ ID NO: 771 - protein, 10 aa, Homo sapiens
KTWFRSNPTK

SEQ ID NO: 772 - protein, 9 aa, Homo sapiens
LAKELTHPL

SEQ ID NO: 773 - protein, 10 aa, Homo sapiens
LAKELTHPLL

SEQ ID NO: 774 - protein, 10 aa, Homo sapiens
NPTKTKKARF

SEQ ID NO: 775 - protein, 9 aa, Homo sapiens
QKMKKAHFL

SEQ ID NO: 776 - protein, 9 aa, Homo sapiens
RFSTASLAK

SEQ ID NO: 777 - protein, 10 aa, Homo sapiens
RPIRPPFLES

SEQ ID NO: 778 - protein, 9 aa, Homo sapiens
RSNPTKTKK

SEQ ID NO: 779 - protein, 10 aa, Homo sapiens
SLAKELTHPL

SEQ ID NO: 780 - protein, 9 aa, Homo sapiens
TKKARFSTA

SEQ ID NO: 781 - protein, 9 aa, Homo sapiens
GLRFWNPSR

SEQ ID NO: 782 - protein, 10 aa, Homo sapiens
ISQLLSWPQK

SEQ ID NO: 783 - protein, 9 aa, Homo sapiens
RIAHISQLL

SEQ ID NO: 784 - protein, 9 aa, Homo sapiens
RLGYSSHQL

SEQ ID NO: 785 - protein, 9 aa, Homo sapiens
SQLLSWPQK

SEQ ID NO: 786 - protein, 9 aa, Homo sapiens
SRIAHISQL

SEQ ID NO: 787 - protein, 9 aa, Homo sapiens
WPQKTEERL

SEQ ID NO: 788 - protein, 9 aa, Homo sapiens
YSSHQLPRK

SEQ ID NO: 789 - protein, 10 aa, Homo sapiens
CPQRMTPGTT

SEQ ID NO: 790 - protein, 9 aa, Homo sapiens
EAEKRTRTL

SEQ ID NO: 791 - protein, 9 aa, Homo sapiens
GTTFITMMK

SEQ ID NO: 792 - protein, 10 aa, Homo sapiens
GTTFITMMKK

SEQ ID NO: 793 - protein, 10 aa, Homo sapiens
ITMMKKEAEK

SEQ ID NO: 794 - protein, 9 aa, Homo sapiens
RMTPGTTFI

SEQ ID NO: 795 - protein, 9 aa, Homo sapiens
SPYCPQRMT

SEQ ID NO: 796 - protein, 9 aa, Homo sapiens
TMMKKEAEK

SEQ ID NO: 797 - protein, 9 aa, Homo sapiens
TPGTTFITM

SEQ ID NO: 798 - protein, 10 aa, Homo sapiens
TPGTTFITMM

SEQ ID NO: 799 - protein, 9 aa, Homo sapiens
TTFITMMKK

SEQ ID NO: 800 - protein, 9 aa, Homo sapiens
CPGATWREA

SEQ ID NO: 801 - protein, 10 aa, Homo sapiens
CPGATWREAA

SEQ ID NO: 802 - protein, 10 aa, Homo sapiens
RSRCPGATWR

SEQ ID NO: 803 - protein, 9 aa, Homo sapiens
TPRATRSRC

SEQ ID NO: 804 - protein, 10 aa, Homo sapiens
HVLPEPHLAL

SEQ ID NO: 805 - protein, 10 aa, Homo sapiens
RPLQTHVLPE

SEQ ID NO: 806 - protein, 10 aa, Homo sapiens
VLWTTPPLQH

SEQ ID NO: 807 - protein, 10 aa, Homo sapiens
APSESPCSPF

SEQ ID NO: 808 - protein, 10 aa, Homo sapiens
CPLDHTTPPA

SEQ ID NO: 809 - protein, 9 aa, Homo sapiens
FLQEQYHEA

SEQ ID NO: 810 - protein, 10 aa, Homo sapiens
RLAFLQEQYH

SEQ ID NO: 811 - protein, 10 aa, Homo sapiens
SPCSPFRLAF

SEQ ID NO: 812 - protein, 9 aa, Homo sapiens
SPPWVRALL

SEQ ID NO: 813 - protein, 10 aa, Homo sapiens
YPACPLDHTT

SEQ ID NO: 814 - protein, 9 aa, Homo sapiens
ALHLRSCPC

SEQ ID NO: 815 - protein, 9 aa, Homo sapiens
CLHHRRHLV

SEQ ID NO: 816 - protein, 10 aa, Homo sapiens
CLHHRRHLVC

SEQ ID NO: 817 - protein, 10 aa, Homo sapiens
CLHRKSHPHL

SEQ ID NO: 818 - protein, 9 aa, Homo sapiens
CLRSHACPP

SEQ ID NO: 819 - protein, 9 aa, Homo sapiens
CLRSHTCPP

SEQ ID NO: 820 - protein, 9 aa, Homo sapiens
CLWCHACLH

SEQ ID NO: 821 - protein, 9 aa, Homo sapiens
CPHHLKNHL

SEQ ID NO: 822 - protein, 9 aa, Homo sapiens
CPHHLKNLL

SEQ ID NO: 823 - protein, 9 aa, Homo sapiens
CPHHLRTRL

SEQ ID NO: 824 - protein, 10 aa, Homo sapiens
CPLHLRSLPF

SEQ ID NO: 825 - protein, 10 aa, Homo sapiens
CPLPRHLKHL

SEQ ID NO: 826 - protein, 10 aa, Homo sapiens
CPLSLRSHPC

SEQ ID NO: 827 - protein, 10 aa, Homo sapiens
CPRHLRNCPL

SEQ ID NO: 828 - protein, 9 aa, Homo sapiens
FPHHLRHHA

SEQ ID NO: 829 - protein, 10 aa, Homo sapiens
FPHHLRHHAC

SEQ ID NO: 830 - protein, 9 aa, Homo sapiens
GLRSRTCPP

SEQ ID NO: 831 - protein, 10 aa, Homo sapiens
HACLHRLRNL

SEQ ID NO: 832 - protein, 9 aa, Homo sapiens
HLACLHHLR

SEQ ID NO: 833 - protein, 9 aa, Homo sapiens
HLCPPHLRY

SEQ ID NO: 834 - protein, 10 aa, Homo sapiens
HLCPPHLRYR

SEQ ID NO: 835 - protein, 9 aa, Homo sapiens
HLKHLACLH

SEQ ID NO: 836 - protein, 9 aa, Homo sapiens
HLKHRPCPH

SEQ ID NO: 837 - protein, 9 aa, Homo sapiens
HLKNHLCPP

SEQ ID NO: 838 - protein, 9 aa, Homo sapiens
HLKSHPCLH

SEQ ID NO: 839 - protein, 9 aa, Homo sapiens
HLKSLLCPL

SEQ ID NO: 840 - protein, 9 aa, Homo sapiens
HLLHLRRLY

SEQ ID NO: 841 - protein, 9 aa, Homo sapiens
HLRNCPLPR

SEQ ID NO: 842 - protein, 10 aa, Homo sapiens
HLRNCPLPRH

SEQ ID NO: 843 - protein, 10 aa, Homo sapiens
HLRRLYPHHL

SEQ ID NO: 844 - protein, 9 aa, Homo sapiens
HLRSHPCPL

SEQ ID NO: 845 - protein, 10 aa, Homo sapiens
HLRSHPCPLH

SEQ ID NO: 846 - protein, 9 aa, Homo sapiens
HLRSLPFPH

SEQ ID NO: 847 - protein, 9 aa, Homo sapiens
HLRTRLCPH

SEQ ID NO: 848 - protein, 9 aa, Homo sapiens
HLVCSHHLK

SEQ ID NO: 849 - protein, 10 aa, Homo sapiens
HPCLHHRRHL

SEQ ID NO: 850 - protein, 9 aa, Homo sapiens
HPGLRSRTC

SEQ ID NO: 851 - protein, 10 aa, Homo sapiens
HPHLLHLRRL

SEQ ID NO: 852 - protein, 9 aa, Homo sapiens
HRKSHPHLL

SEQ ID NO: 853 - protein, 9 aa, Homo sapiens
HRRTRSCPC

SEQ ID NO: 854 - protein, 10 aa, Homo sapiens
KSHPHLLHLR

SEQ ID NO: 855 - protein, 10 aa, Homo sapiens
KSLLCPLHLR

SEQ ID NO: 856 - protein, 10 aa, Homo sapiens
LLCPLHLRSL

SEQ ID NO: 857 - protein, 10 aa, Homo sapiens
LLHLRRLYPH

SEQ ID NO: 858 - protein, 9 aa, Homo sapiens
LPRHLKHLA

SEQ ID NO: 859 - protein, 10 aa, Homo sapiens
LPRHLKHLAC

SEQ ID NO: 860 - protein, 9 aa, Homo sapiens
LRRLRSHTC

SEQ ID NO: 861 - protein, 9 aa, Homo sapiens
LRRLYPHHL

SEQ ID NO: 862 - protein, 10 aa, Homo sapiens
LVCSHHLKSL

SEQ ID NO: 863 - protein, 10 aa, Homo sapiens
NLRNHTCPPS

SEQ ID NO: 864 - protein, 9 aa, Homo sapiens
PLHLRSLPF

SEQ ID NO: 865 - protein, 10 aa, Homo sapiens
RLCPHHLKNH

SEQ ID NO: 866 - protein, 9 aa, Homo sapiens
RLYPHHLKH

SEQ ID NO: 867 - protein, 10 aa, Homo sapiens
RLYPHHLKHR

SEQ ID NO: 868 - protein, 10 aa, Homo sapiens
RPCPHHLKNL

SEQ ID NO: 869 - protein, 10 aa, Homo sapiens
RSHPCPLHLK

SEQ ID NO: 870 - protein, 10 aa, Homo sapiens
RSLPFPHHLR

SEQ ID NO: 871 - protein, 9 aa, Homo sapiens
RTRLCPHHL

SEQ ID NO: 872 - protein, 10 aa, Homo sapiens
RTRLCPHHLK

SEQ ID NO: 873 - protein, 9 aa, Homo sapiens
SLLCPLHLR

SEQ ID NO: 874 - protein, 9 aa, Homo sapiens
SLRSHACPP

SEQ ID NO: 875 - protein, 9 aa, Homo sapiens
SPLRSQANA

SEQ ID NO: 876 - protein, 9 aa, Homo sapiens
YLRRLRSHT

SEQ ID NO: 877 - protein, 10 aa, Homo sapiens
YPHHLKHRPC

SEQ ID NO: 878 - protein, 9 aa, Homo sapiens
FITSGQIFK

SEQ ID NO: 879 - protein, 9 aa, Homo sapiens
IFITSGQIF

SEQ ID NO: 880 - protein, 9 aa, Homo sapiens
SQSEALCVL

SEQ ID NO: 881 - protein, 10 aa, Homo sapiens
SQSEALCVLL

SEQ ID NO: 882 - protein, 9 aa, Homo sapiens
AYTGTILMM

SEQ ID NO: 883 - protein, 10 aa, Homo sapiens
DLKAYTGTIL

SEQ ID NO: 884 - protein, 9 aa, Homo sapiens
ILTKQIKTK

SEQ ID NO: 885 - protein, 9 aa, Homo sapiens
KMILTKQIK

SEQ ID NO: 886 - protein, 9 aa, Homo sapiens
KPTDTFLQI

SEQ ID NO: 887 - protein, 10 aa, Homo sapiens
KPTDTFLQIL

SEQ ID NO: 888 - protein, 10 aa, Homo sapiens
MILTKQIKTK

SEQ ID NO: 889 - protein, 10 aa, Homo sapiens
ITRYTIFVLK

SEQ ID NO: 890 - protein, 9 aa, Homo sapiens
LIAELHNIL

SEQ ID NO: 891 - protein, 10 aa, Homo sapiens
LIAELHNILL

SEQ ID NO: 892 - protein, 10 aa, Homo sapiens
MTPPNLIAEL

SEQ ID NO: 893 - protein, 9 aa, Homo sapiens
NLIAELHNI

SEQ ID NO: 894 - protein, 10 aa, Homo sapiens
NLIAELHNIL

SEQ ID NO: 895 - protein, 10 aa, Homo sapiens
RYTIFVLKDI

SEQ ID NO: 896 - protein, 10 aa, Homo sapiens
TITRYTIFVL

SEQ ID NO: 897 - protein, 9 aa, Homo sapiens
TPPNLIAEL

SEQ ID NO: 898 - protein, 9 aa, Homo sapiens
FLQFRTHTT

SEQ ID NO: 899 - protein, 10 aa, Homo sapiens
LPAKGEDIFL

SEQ ID NO: 900 - protein, 10 aa, Homo sapiens
LQFRTHTTGR

SEQ ID NO: 901 - protein, 9 aa, Homo sapiens
NLQSSVCGL

SEQ ID NO: 902 - protein, 9 aa, Homo sapiens
SSVCGLPAK

SEQ ID NO: 903 - protein, 10 aa, Homo sapiens
VQWRNLQSSV

SEQ ID NO: 904 - protein, 9 aa, Homo sapiens
GQNVSLLGK

SEQ ID NO: 905 - protein, 10 aa, Homo sapiens
HTRTRGNLRK

SEQ ID NO: 906 - protein, 10 aa, Homo sapiens
ILHTRTRGNL

SEQ ID NO: 907 - protein, 10 aa, Homo sapiens
KGQNVSLLGK

SEQ ID NO: 908 - protein, 9 aa, Homo sapiens
LLGKYILHT

SEQ ID NO: 909 - protein, 10 aa, Homo sapiens
LRKSRKWKSM

SEQ ID NO: 910 - protein, 9 aa, Homo sapiens
SLLGKYILH

SEQ ID NO: 911 - protein, 10 aa, Homo sapiens
SLLGKYILHT

SEQ ID NO: 912 - protein, 9 aa, Homo sapiens
CTSPLLAPV

SEQ ID NO: 913 - protein, 9 aa, Homo sapiens
FPENLPGQL

SEQ ID NO: 914 - protein, 10 aa, Homo sapiens
GLLAFWDSQV

SEQ ID NO: 915 - protein, 9 aa, Homo sapiens
IFCPFPENL

SEQ ID NO: 916 - protein, 9 aa, Homo sapiens
LLAFWDSQV

SEQ ID NO: 917 - protein, 9 aa, Homo sapiens
LLAPVIFCP

SEQ ID NO: 918 - protein, 10 aa, Homo sapiens
LLAPVIFCPF

SEQ ID NO: 919 - protein, 9 aa, Homo sapiens
LPCPQQDVL

SEQ ID NO: 920 - protein, 9 aa, Homo sapiens
RFPSGLLAF

SEQ ID NO: 921 - protein, 10 aa, Homo sapiens
RFPSGLLAFW

SEQ ID NO: 922 - protein, 9 aa, Homo sapiens
SPLLAPVIF

SEQ ID NO: 923 - protein, 9 aa, Homo sapiens
SPRGPCTSS

SEQ ID NO: 924 - protein, 10 aa, Homo sapiens
SPRGPCTSSS

SEQ ID NO: 925 - protein, 9 aa, Homo sapiens
SQVCDLHVL

SEQ ID NO: 926 - protein, 10 aa, Homo sapiens
VIFCPFPENL

SEQ ID NO: 927 - protein, 9 aa, Homo sapiens
AMVWPLLSI

SEQ ID NO: 928 - protein, 10 aa, Homo sapiens
AMVWPLLSIL

SEQ ID NO: 929 - protein, 9 aa, Homo sapiens
AQIAMVWPL

SEQ ID NO: 930 - protein, 10 aa, Homo sapiens
AQIAMVWPLL

SEQ ID NO: 931 - protein, 9 aa, Homo sapiens
CPMSRLRLA

SEQ ID NO: 932 - protein, 10 aa, Homo sapiens
CPMSRLRLAL

SEQ ID NO: 933 - protein, 10 aa, Homo sapiens
IAMVWPLLSI

SEQ ID NO: 934 - protein, 10 aa, Homo sapiens
ILSEWKEICV

SEQ ID NO: 935 - protein, 9 aa, Homo sapiens
IVWWCPMSR

SEQ ID NO: 936 - protein, 10 aa, Homo sapiens
IVWWCPMSRL

SEQ ID NO: 937 - protein, 10 aa, Homo sapiens
IWMTETLFDI

SEQ ID NO: 938 - protein, 9 aa, Homo sapiens
LLSILSEWK

SEQ ID NO: 939 - protein, 9 aa, Homo sapiens
MSAAQIAMV

SEQ ID NO: 940 - protein, 9 aa, Homo sapiens
MSRLRLALT

SEQ ID NO: 941 - protein, 10 aa, Homo sapiens
MSRLRLALTV

SEQ ID NO: 942 - protein, 9 aa, Homo sapiens
MVWPLLSIL

SEQ ID NO: 943 - protein, 10 aa, Homo sapiens
RLALTVPPST

SEQ ID NO: 944 - protein, 9 aa, Homo sapiens
TLFDIVWWC

SEQ ID NO: 945 - protein, 10 aa, Homo sapiens
TLFDIVWWCP

SEQ ID NO: 946 - protein, 10 aa, Homo sapiens
TMSAAQIAMV

SEQ ID NO: 947 - protein, 10 aa, Homo sapiens
VWSIWMTETL

SEQ ID NO: 948 - protein, 9 aa, Homo sapiens
WMTETLFDI

SEQ ID NO: 949 - protein, 10 aa, Homo sapiens
WMTETLFDIV

SEQ ID NO: 950 - protein, 9 aa, Homo sapiens
ALRCVFVPV

SEQ ID NO: 951 - protein, 10 aa, Homo sapiens
ALRCVFVPVL

SEQ ID NO: 952 - protein, 9 aa, Homo sapiens
ALSEHCPTT

SEQ ID NO: 953 - protein, 10 aa, Homo sapiens
AQRKRISARK

SEQ ID NO: 954 - protein, 9 aa, Homo sapiens
GAQRKRISA

SEQ ID NO: 955 - protein, 9 aa, Homo sapiens
HWMENISPF

SEQ ID NO: 956 - protein, 9 aa, Homo sapiens
LPSQRRNHW

SEQ ID NO: 957 - protein, 10 aa, Homo sapiens
LPSQRRNHWM

SEQ ID NO: 958 - protein, 10 aa, Homo sapiens
NISPFRSVGV

SEQ ID NO: 959 - protein, 9 aa, Homo sapiens
RISARKGSL

SEQ ID NO: 960 - protein, 10 aa, Homo sapiens
SPFRSVGVSA

SEQ ID NO: 961 - protein, 10 aa, Homo sapiens
SPSSHWKTPV

SEQ ID NO: 962 - protein, 10 aa, Homo sapiens
TALRCVFVPV

SEQ ID NO: 963 - protein, 9 aa, Artificial Sequence
  Description of Artificial Sequence Synthetic peptide
VIYWDGTAL

SEQ ID NO: 964 - protein, 10 aa, Homo sapiens
VIYWDGTALR

SEQ ID NO: 965 - protein, 10 aa, Homo sapiens
VLGETGAQRK

SEQ ID NO: 966 - protein, 9 aa, Homo sapiens
HPRPRHGHL

SEQ ID NO: 967 - protein, 10 aa, Homo sapiens
HPRPRHGHLQ

SEQ ID NO: 968 - protein, 9 aa, Homo sapiens
RPRHGHLQA

SEQ ID NO: 969 - protein, 10 aa, Homo sapiens
RPRHGHLQAV

SEQ ID NO: 970 - protein, 10 aa, Homo sapiens
GSLKTQVQMK

SEQ ID NO: 971 - protein, 10 aa, Homo sapiens
PPGPCHLLSL

SEQ ID NO: 972 - protein, 10 aa, Homo sapiens
RTILNNGSLK

SEQ ID NO: 973 - protein, 9 aa, Homo sapiens
SLKTQVQMK

SEQ ID NO: 974 - protein, 10 aa, Homo sapiens
SLKTQVQMKL

SEQ ID NO: 975 - protein, 9 aa, Homo sapiens
TILNNGSLK

SEQ ID NO: 976 - protein, 10 aa, Homo sapiens
CPPCRPKQWM

SEQ ID NO: 977 - protein, 10 aa, Homo sapiens
TTFCPPCRPK

SEQ ID NO: 978 - protein, 10 aa, Homo sapiens
CFANWPRPAL

SEQ ID NO: 979 - protein, 9 aa, Homo sapiens
FANWPRPAL

SEQ ID NO: 980 - protein, 9 aa, Homo sapiens
GLIPHPRPA

SEQ ID NO: 981 - protein, 9 aa, Homo sapiens
HPRPAPASA

SEQ ID NO: 982 - protein, 10 aa, Homo sapiens
HPRPAPASAP

SEQ ID NO: 983 - protein, 9 aa, Homo sapiens
IPHPRPAPA

SEQ ID NO: 984 - protein, 10 aa, Homo sapiens
IPHPRPAPAS

SEQ ID NO: 985 - protein, 9 aa, Homo sapiens
RPALCSCGL

SEQ ID NO: 986 - protein, 10 aa, Homo sapiens
RPALCSCGLI

SEQ ID NO: 987 - protein, 9 aa, Homo sapiens
TPLPSTRCF

SEQ ID NO: 988 - protein, 9 aa, Homo sapiens
WPRPALCSC

SEQ ID NO: 989 - protein, 10 aa, Homo sapiens
WPRPALCSCG

SEQ ID NO: 990 - protein, 10 aa, Homo sapiens
ALRRSGRPPK

SEQ ID NO: 991 - protein, 9 aa, Homo sapiens
GLVPSLVSK

SEQ ID NO: 992 - protein, 9 aa, Homo sapiens
KISVETYTV

SEQ ID NO: 993 - protein, 10 aa, Homo sapiens
LLMVLMSLDL

SEQ ID NO: 994 - protein, 10 aa, Homo sapiens
LMSLDLDTGL

SEQ ID NO: 995 - protein, 9 aa, Homo sapiens
LMVLMSLDL

SEQ ID NO: 996 - protein, 10 aa, Homo sapiens
LVSKCLILRV

SEQ ID NO: 997 - protein, 9 aa, Homo sapiens
QLLMVLMSL

SEQ ID NO: 998 - protein, 9 aa, Homo sapiens
RPGAADTGA

SEQ ID NO: 999 - protein, 10 aa, Homo sapiens
RPGAADTGAH

SEQ ID NO: 1000 - protein, 9 aa, Homo sapiens
SLDLDTGLV

SEQ ID NO: 1001 - protein, 9 aa, Homo sapiens
SLVSKCLIL

SEQ ID NO: 1002 - protein, 10 aa, Homo sapiens
SQLLMVLMSL

SEQ ID NO: 1003 - protein, 9 aa, Homo sapiens
TVSSQLLMV

SEQ ID NO: 1004 - protein, 9 aa, Homo sapiens
TYTVSSQLL

SEQ ID NO: 1005 - protein, 10 aa, Homo sapiens
TYTVSSQLLM

SEQ ID NO: 1006 - protein, 9 aa, Homo sapiens
VLMSLDLDT

SEQ ID NO: 1007 - protein, 9 aa, Homo sapiens
VPSLVSKCL

SEQ ID NO: 1008 - protein, 10 aa, Homo sapiens
VSKCLILRVK

SEQ ID NO: 1009 - protein, 9 aa, Homo sapiens
YTVSSQLLM

SEQ ID NO: 1010 - protein, 10 aa, Homo sapiens
YTVSSQLLMV

SEQ ID NO: 1011 - protein, 9 aa, Homo sapiens
FCQYHTASV

SEQ ID NO: 1012 - protein, 9 aa, Homo sapiens
CPYGSTSTA

SEQ ID NO: 1013 - protein, 10 aa, Homo sapiens
CPYGSTSTAS

SEQ ID NO: 1014 - protein, 10 aa, Homo sapiens
LARAAASTAT

SEQ ID NO: 1015 - protein, 9 aa, Homo sapiens
MLTDSLFLP

SEQ ID NO: 1016 - protein, 10 aa, Homo sapiens
PPRSSSAIAV

SEQ ID NO: 1017 - protein, 10 aa, Homo sapiens
RAAASTATEV

SEQ ID NO: 1018 - protein, 9 aa, Homo sapiens
SPPRSSSAI

SEQ ID NO: 1019 - protein, 10 aa, Homo sapiens
SPPRSSSAIA

SEQ ID NO: 1020 - protein, 9 aa, Homo sapiens
SPTQRCRLA

SEQ ID NO: 1021 - protein, 9 aa, Homo sapiens
TQRCRLARA

SEQ ID NO: 1022 - protein, 10 aa, Homo sapiens
TQRCRLARAA

SEQ ID NO: 1023 - protein, 10 aa, Homo sapiens
KIWKTTQMCR

SEQ ID NO: 1024 - protein, 9 aa, Homo sapiens
WTSSGRSTK

SEQ ID NO: 1025 - protein, 9 aa, Homo sapiens
ALGELARAL

SEQ ID NO: 1026 - protein, 9 aa, Homo sapiens
AQLRRRAAA

SEQ ID NO: 1027 - protein, 10 aa, Homo sapiens
AQLRRRAAAL

SEQ ID NO: 1028 - protein, 10 aa, Homo sapiens
ARRAARMAQL

SEQ ID NO: 1029 - protein, 9 aa, Homo sapiens
HPQLPRSPL

SEQ ID NO: 1030 - protein, 10 aa, Homo sapiens
HPQLPRSPLA

SEQ ID NO: 1031 - protein, 9 aa, Homo sapiens
LARALPGHL

SEQ ID NO: 1032 - protein, 10 aa, Homo sapiens
LARALPGHLL

SEQ ID NO: 1033 - protein, 9 aa, Homo sapiens
MAQLRRRAA

SEQ ID NO: 1034 - protein, 10 aa, Homo sapiens
MAQLRRRAAA

SEQ ID NO: 1035 - protein, 9 aa, Homo sapiens
QLRRRAAAL

SEQ ID NO: 1036 - protein, 10 aa, Homo sapiens
RAAALPNAAA

SEQ ID NO: 1037 - protein, 10 aa, Homo sapiens
RMAQLRRRAA

SEQ ID NO: 1038 - protein, 10 aa, Homo sapiens
SQSARRAARM

SEQ ID NO: 1039 - protein, 10 aa, Homo sapiens
GMTLGEKFRV

SEQ ID NO: 1040 - protein, 9 aa, Homo sapiens
RVGNCKHLK

SEQ ID NO: 1041 - protein, 9 aa, Homo sapiens
GPSEPGNNI

SEQ ID NO: 1042 - protein, 10 aa, Homo sapiens
KICNESASRK

SEQ ID NO: 1043 - protein, 10 aa, Homo sapiens
GIQVLNVSLK

SEQ ID NO: 1044 - protein, 9 aa, Homo sapiens
IQVLNVSLK

SEQ ID NO: 1045 - protein, 9 aa, Homo sapiens
KSSSNVISY

SEQ ID NO: 1046 - protein, 9 aa, Homo sapiens
KYGWSLLRV

SEQ ID NO: 1047 - protein, 9 aa, Homo sapiens
RSWKYGWSL

SEQ ID NO: 1048 - protein, 9 aa, Homo sapiens
SLKSSSNVI

SEQ ID NO: 1049 - protein, 9 aa, Homo sapiens
SWKYGWSLL

SEQ ID NO: 1050 - protein, 9 aa, Homo sapiens
TVANGRSWK

SEQ ID NO: 1051 - protein, 9 aa, Homo sapiens
VPQVNGIQV

SEQ ID NO: 1052 - protein, 10 aa, Homo sapiens
VPQVNGIQVL

SEQ ID NO: 1053 - protein, 10 aa, Homo sapiens
VTVANGRSWK

SEQ ID NO: 1054 - protein, 9 aa, Homo sapiens
WSLLRVPQV

SEQ ID NO: 1055 - protein, 10 aa, Homo sapiens
HPGDCLIFKL

SEQ ID NO: 1056 - protein, 9 aa, Homo sapiens
KLRVPGSSV

SEQ ID NO: 1057 - protein, 10 aa, Homo sapiens
KLRVPGSSVL

SEQ ID NO: 1058 - protein, 9 aa, Homo sapiens
RVPGSSVLV

SEQ ID NO: 1059 - protein, 9 aa, Homo sapiens
SVLVTVPGL

SEQ ID NO: 1060 - protein, 10 aa, Homo sapiens
VPGSSVLVTV

SEQ ID NO: 1061 - protein, 10 aa, Homo sapiens
ALLLRPRPPR

SEQ ID NO: 1062 - protein, 10 aa, Homo sapiens
ALSALLLRPR

SEQ ID NO: 1063 - protein, 9 aa, Homo sapiens
LTINKEEAL

SEQ ID NO: 1064 - protein, 9 aa, Homo sapiens
IVHSATGFK

SEQ ID NO: 1065 - protein, 9 aa, Homo sapiens
ATGFKQSSK

SEQ ID NO: 1066 - protein, 9 aa, Homo sapiens
ELFPLIFPA

SEQ ID NO: 1067 - protein, 9 aa, Homo sapiens
KGPELFPLI

SEQ ID NO: 1068 - protein, 10 aa, Homo sapiens
KGPELFPLIF

SEQ ID NO: 1069 - protein, 11 aa, Homo sapiens
KPTDAPPKAGV

SEQ ID NO: 1070 - protein, 10 aa, Homo sapiens
ALEEKKGNYV

SEQ ID NO: 1071 - protein, 10 aa, Homo sapiens
IQLQDKFEHL

SEQ ID NO: 1072 - protein, 9 aa, Homo sapiens
QLQDKFEHL

SEQ ID NO: 1073 - protein, 10 aa, Homo sapiens
QLQDKFEHLK

SEQ ID NO: 1074 - protein, 10 aa, Homo sapiens
YLVIQLQDKF

SEQ ID NO: 1075 - protein, 10 aa, Homo sapiens
QVYRRKHQEL

SEQ ID NO: 1076 - protein, 9 aa, Homo sapiens
STREKNSQV

SEQ ID NO: 1077 - protein, 9 aa, Homo sapiens
VYRRKHQEL

SEQ ID NO: 1078 - protein, 9 aa, Homo sapiens
VLTVTSTDV

SEQ ID NO: 1079 - protein, 10 aa, Homo sapiens
VLTVTSTDVK

SEQ ID NO: 1080 - protein, 9 aa, Homo sapiens
IMSLWGLVS

SEQ ID NO: 1081 - protein, 10 aa, Homo sapiens
IMSLWGLVSK

SEQ ID NO: 1082 - protein, 9 aa, Homo sapiens
KLKQEATSK

SEQ ID NO: 1083 - protein, 9 aa, Homo sapiens
QIMSLWGLV

SEQ ID NO: 1084 - protein, 9 aa, Homo sapiens
SQIMSLWGL

SEQ ID NO: 1085 - protein, 10 aa, Homo sapiens
SQIMSLWGLV

SEQ ID NO: 1086 - protein, 9 aa, Homo sapiens
TSKSQIMSL

SEQ ID NO: 1087 - protein, 10 aa, Homo sapiens
LLQEFDVQEA

SEQ ID NO: 1088 - protein, 10 aa, Homo sapiens
LQEFDVQEAL

SEQ ID NO: 1089 - protein, 9 aa, Homo sapiens
GPREPRNRT

SEQ ID NO: 1090 - protein, 10 aa, Homo sapiens
RNRTEKHSTM

SEQ ID NO: 1091 - protein, 9 aa, Homo sapiens
ALNSEALSV

SEQ ID NO: 1092 - protein, 10 aa, Homo sapiens
ALNSEALSVV

SEQ ID NO: 1093 - protein, 10 aa, Homo sapiens
MALNSEALSV

SEQ ID NO: 1094 - protein, 67 aa, Artificial Sequence
  Description of Artificial Sequence Synthetic polypeptide
MSDVAIVKEGWLHKRGKYIKTWRPRYFLLKNDGTFIGYKERPQDVDQREAPLNNFSVAQCQLMKTER

SEQ ID NO: 1095 - protein, 101 aa, Artificial Sequence
  Description of Artificial Sequence Synthetic polypeptide
TMLVLEGSGNLVLYTGVVRVGKVFIPGLPAPSLTMSNTMPRPSTPLDGVSAPKPLSKLLGSLDEVVLLSPVPELRDSSKLHDSLYNEDCTFQQLGTYIHSI

SEQ ID NO: 1096 - protein, 90 aa, Artificial Sequence
  Description of Artificial Sequence Synthetic polypeptide
MREPIYMHSTMVFLPWELHTKKGPSPPEQFMAVKLSDSRTALKSGYGKYLGINSDELVGHSDAIGPREQWEPVFQNGKMALLASNSCFIR

SEQ ID NO: 1097 - protein, 101 aa, Artificial Sequence
  Description of Artificial Sequence Synthetic polypeptide
AMPNQAQMRILKETELRKVKVLGSGAFGTVYKGIWIPDGENVKIPVAIKVSRENTSPKANKEILDEAYVMAGVGSPYVSRLLGICLTSTVQLVTQLMPYGC

SEQ ID NO: 1098 - protein, 101 aa, Artificial Sequence
  Description of Artificial Sequence Synthetic polypeptide
AVCKSKKSWQARHTGIKIVQQIAILMGCAILPHLRSLVEIIEHGLVDEQQEVRTISALAIAALAEAATPYGIESFDSVLKPLWKGIRQHRGKGLAAFLKAI

SEQ ID NO: 1099 - protein, 101 aa, Artificial Sequence
  Description of Artificial Sequence Synthetic polypeptide
YLSLYLLLVSCPKSEVRAKFKFSILNAKGEETKAMESQRAYRFVQGKDWGLKKFIRRDFLLDEANGLLPDDKLTLFCEVSVVQDSVNISGQNTMNMVKVPE

SEQ ID NO: 1100 - protein, 101 aa, Artificial Sequence
  Description of Artificial Sequence Synthetic polypeptide
YLSLYLLLVSCPKSEVRAKFKFSILNAKGEETKAMESQRAYRFVQGKDWGVKKFIRRDFLLDEANGLLPDDKLTLFCEVSVVQDSVNISGQNTMNMVKVPE

SEQ ID NO: 1101 - protein, 101 aa, Artificial Sequence
  Description of Artificial Sequence Synthetic polypeptide
PEVGSDCTTIHYNYMCNSSCMGGMNRRPILTIITLEDSSGNLLGRNSFEVHVCACPGRDRRTEEENLRKKGEPHHELPPGSTKRALPNNTSSSPQPKKKPL

SEQ ID NO: 1102 - protein, 101 aa, Artificial Sequence
  Description of Artificial Sequence Synthetic polypeptide
TEVVRRCPHHERCSDSDGLAPPQHLIRVEGNLRVEYLDDRNTFRHSVVVPCEPPEVGSDCTTIHYNYMCNSSCMGGMNRRPILTIITLEDSSGNLLGRNSF

SEQ ID NO: 1103 - protein, 54 aa, Artificial Sequence
  Description of Artificial Sequence Synthetic polypeptide
YNFDKNYKDWQSAVECIAVLDVLLCLANYSRGGDGPMCRPVILLPEDTPPLLRA

SEQ ID NO: 1104 - protein, 10 aa, Homo sapiens
KYIKTWRPRY

SEQ ID NO: 1105 - protein, 9 aa, Homo sapiens
WLHKRGKYI

SEQ ID NO: 1106 - protein, 10 aa, Homo sapiens
WLHKRGKYIK

SEQ ID NO: 1107 - protein, 9 aa, Homo sapiens
APKPLSKLL

SEQ ID NO: 1108 - protein, 10 aa, Homo sapiens
GVSAPKPLSK

SEQ ID NO: 1109 - protein, 9 aa, Homo sapiens
VSAPKPLSK

SEQ ID NO: 1110 - protein, 9 aa, Homo sapiens
CPHRPILQA

SEQ ID NO: 1111 - protein, 9 aa, Homo sapiens
KLSDSRTAL

SEQ ID NO: 1112 - protein, 10 aa, Homo sapiens
KLSDSRTALK

SEQ ID NO: 1113 - protein, 9 aa, Homo sapiens
LSDSRTALK

SEQ ID NO: 1114 - protein, 10 aa, Homo sapiens
RTALKSGYGK

SEQ ID NO: 1115 - protein, 9 aa, Homo sapiens
TALKSGYGK

SEQ ID NO: 1116 - protein, 9 aa, Homo sapiens
ALSASNSCF

SEQ ID NO: 1117 - protein, 10 aa, Homo sapiens
ALSASNSCFI

SEQ ID NO: 1118 - protein, 10 aa, Homo sapiens
FQNGKMALSA

SEQ ID NO: 1119 - protein, 10 aa, Homo sapiens
KVSRENTSPK

SEQ ID NO: 1120 - protein, 10 aa, Homo sapiens
KPIIIGGHAY

SEQ ID NO: 1121 - protein, 10 aa, Homo sapiens
AISTRDPLSK

SEQ ID NO: 1122 - protein, 9 aa, Homo sapiens
QTGVMICAY

SEQ ID NO: 1123 - protein, 9 aa, Homo sapiens
FSGEYIPTV

SEQ ID NO: 1124 - protein, 9 aa, Homo sapiens
TTNAFSGEY

SEQ ID NO: 1125 - protein, 10 aa, Homo sapiens
YTTNAFSGEY

SEQ ID NO: 1126 - protein, 9 aa, Homo sapiens
GLVDEQQEV

SEQ ID NO: 1127 - protein, 9 aa, Homo sapiens
FVQGKDWGL

SEQ ID NO: 1128 - protein, 9 aa, Homo sapiens
FVQGKDWGV

SEQ ID NO: 1129 - protein, 10 aa, Homo sapiens
CMGSMNRRPI

SEQ ID NO: 1130 - protein, 9 aa, Homo sapiens
GSMNRRPIL

SEQ ID NO: 1131 - protein, 9 aa, Homo sapiens
MGSMNRRPI

SEQ ID NO: 1132 - protein, 10 aa, Homo sapiens
MGSMNRRPIL

SEQ ID NO: 1133 - protein, 10 aa, Homo sapiens
SMNRRPILTI

SEQ ID NO: 1134 - protein, 10 aa, Homo sapiens
CMGGMNQRPI

SEQ ID NO: 1135 - protein, 10 aa, Homo sapiens
GMNQRPILTI

SEQ ID NO: 1136 - protein, 9 aa, Homo sapiens
NQRPILTII

SEQ ID NO: 1137 - protein, 10 aa, Homo sapiens
CMGGMNWRPI

SEQ ID NO: 1138 - protein, 10 aa, Homo sapiens
GMNWRPILTI

SEQ ID NO: 1139 - protein, 9 aa, Homo sapiens
MNWRPILTI

SEQ ID NO: 1140 - protein, 10 aa, Homo sapiens
MNWRPILTII

SEQ ID NO: 1141 - protein, 9 aa, Homo sapiens
NSFEVCVCA

SEQ ID NO: 1142 - protein, 9 aa, Homo sapiens
NSFEVHVCA

SEQ ID NO: 1143 - protein, 9 aa, Homo sapiens
VVPCEPPEV

SEQ ID NO: 1144 - protein, 10 aa, Homo sapiens
VVVPCEPPEV

SEQ ID NO: 1145 - protein, 10 aa, Homo sapiens
ILLPEDTPPL

SEQ ID NO: 1146 - protein, 9 aa, Homo sapiens
LLPEDTPPL

SEQ ID NO: 1147 - protein, 9 aa, Homo sapiens
APFRVNHAV

SEQ ID NO: 1148 - protein, 9 aa, Homo sapiens
CLADVLLSV

SEQ ID NO: 1149 - protein, 10 aa, Homo sapiens
FLQERNLPPK

SEQ ID NO: 1150 - protein, 10 aa, Homo sapiens
HLIVLRVVRL

SEQ ID NO: 1151 - protein, 9 aa, Homo sapiens
HPKVHLNTM

SEQ ID NO: 1152 - protein, 10 aa, Homo sapiens
HPKVHLNTMF

SEQ ID NO: 1153 - protein, 9 aa, Homo sapiens
KVHLNTMFR

SEQ ID NO: 1154 - protein, 10 aa, Homo sapiens
KVHLNTMFRR

SEQ ID NO: 1155 - protein, 10 aa, Homo sapiens
LPAPFRVNHA

SEQ ID NO: 1156 - protein, 9 aa, Homo sapiens
MFRRPHSCL

SEQ ID NO: 1157 - protein, 10 aa, Homo sapiens
MFRRPHSCLA

SEQ ID NO: 1158 - protein, 10 aa, Homo sapiens
NTMFRRPHSC

SEQ ID NO: 1159 - protein, 9 aa, Homo sapiens
RPHSCLADV

SEQ ID NO: 1160 - protein, 10 aa, Homo sapiens
RPHSCLADVL

SEQ ID NO: 1161 - protein, 10 aa, Homo sapiens
RVVRLPAPFR

SEQ ID NO: 1162 - protein, 9 aa, Homo sapiens
SVHLIVLRV

SEQ ID NO: 1163 - protein, 9 aa, Homo sapiens
TMFRRPHSC

SEQ ID NO: 1164 - protein, 10 aa, Homo sapiens
TMFRRPHSCL

SEQ ID NO: 1165 - protein, 9 aa, Homo sapiens
VLLSVHLIV

SEQ ID NO: 1166 - protein, 10 aa, Homo sapiens
VLLSVHLIVL

SEQ ID NO: 1167 - protein, 9 aa, Homo sapiens
VLRVVRLPA

SEQ ID NO: 1168 - protein, 9 aa, Homo sapiens
VVRLPAPFR

SEQ ID NO: 1169 - protein, 10 aa, Artificial Sequence
  Description of Artificial Sequence Synthetic peptide
YMANGSLLNY

SEQ ID NO: 1170 - protein, 9 aa, Artificial Sequence
  Description of Artificial Sequence Synthetic peptide
MANGSLLNY

SEQ ID NO: 1171 - protein, 9 aa, Artificial Sequence
  Description of Artificial Sequence Synthetic peptide
EYMANGSLL

SEQ ID NO: 1172 - protein, 9 aa, Artificial Sequence
  Description of Artificial Sequence Synthetic peptide
SLLNYLREM

SEQ ID NO: 1173 - protein, 10 aa, Artificial Sequence
  Description of Artificial Sequence Synthetic peptide
MANGSLLNYL

SEQ ID NO: 1174 - protein, 9 aa, Artificial Sequence
  Description of Artificial Sequence Synthetic peptide
YMANGSLLN

SEQ ID NO: 1175 - protein, 9 aa, Artificial Sequence
  Description of Artificial Sequence Synthetic peptide
LTSTVQLIM

SEQ ID NO: 1176 - protein, 10 aa, Artificial Sequence
  Description of Artificial Sequence Synthetic peptide
CLTSTVQLIM

SEQ ID NO: 1177 - protein, 10 aa, Artificial Sequence
  Description of Artificial Sequence Synthetic peptide
MQLMPFGCLL

SEQ ID NO: 1178 - protein, 9 aa, Artificial Sequence
  Description of Artificial Sequence Synthetic peptide
MQLMPFGCL

SEQ ID NO: 1179 - protein, 10 aa, Artificial Sequence
  Description of Artificial Sequence Synthetic peptide
LIMQLMPFGC

SEQ ID NO: 1180 - protein, 9 aa, Artificial Sequence
  Description of Artificial Sequence Synthetic peptide
QLIMQLMPF

SEQ ID NO: 1181 - protein, 10 aa, Artificial Sequence
  Description of Artificial Sequence Synthetic peptide
IMQLMPFGCL

SEQ ID NO: 1182 - protein, 9 aa, Artificial Sequence
  Description of Artificial Sequence Synthetic peptide
IMQLMPFGC

SEQ ID NO: 1183 - protein, 9 aa, Artificial Sequence
  Description of Artificial Sequence Synthetic peptide
LIMQLMPFG

SEQ ID NO: 1184 - protein, 10 aa, Artificial Sequence
  Description of Artificial Sequence Synthetic peptide
VQLIMQLMPF

SEQ ID NO: 1185 - protein, 10 aa, Artificial Sequence
  Description of Artificial Sequence Synthetic peptide
QLIMQLMPFG

SEQ ID NO: 1186 - protein, 9 aa, Artificial Sequence
  Description of Artificial Sequence Synthetic peptide
STVQLIMQL

SEQ ID NO: 1187 - protein, 10 aa, Artificial Sequence
  Description of Artificial Sequence Synthetic peptide
FTCQIPGIYY

SEQ ID NO: 1188 - protein, 8 aa, Artificial Sequence
  Description of Artificial Sequence Synthetic peptide
GSDGKPGY

SEQ ID NO: 1189 - protein, 8 aa, Artificial Sequence
  Description of Artificial Sequence Synthetic peptide
NAESNGLY

SEQ ID NO: 1190 - protein, 9 aa, Artificial Sequence
  Description of Artificial Sequence Synthetic peptide
LTENDQVWL

SEQ ID NO: 1191 - protein, 9 aa, Artificial Sequence
  Description of Artificial Sequence Synthetic peptide
GTHVWVGLY

SEQ ID NO: 1192 - protein, 9 aa, Artificial Sequence
  Description of Artificial Sequence Synthetic peptide
TYDEYTKGY

SEQ ID NO: 1193 - protein, 10 aa, Artificial Sequence
  Description of Artificial Sequence Synthetic peptide
YTYDEYTKGY

SEQ ID NO: 1194 - protein, 9 aa, Artificial Sequence
  Description of Artificial Sequence Synthetic peptide
FTCQIPGIY

SEQ ID NO: 1195 - protein, 12 aa, Artificial Sequence
  Description of Artificial Sequence Synthetic peptide
NAESNGLYSSEY

SEQ ID NO: 1196 - protein, 9 aa, Artificial Sequence
  Description of Artificial Sequence Synthetic peptide
YLDQASGSA

SEQ ID NO: 1197 - protein, 9 aa, Artificial Sequence
  Description of Artificial Sequence Synthetic peptide
FLLLVSLNL

SEQ ID NO: 1198 - protein, 10 aa, Artificial Sequence
  Description of Artificial Sequence Synthetic peptide
FLLLVSLNLV

SEQ ID NO: 1199 - protein, 9 aa, Artificial Sequence
  Description of Artificial Sequence Synthetic peptide
GLYKNGTPV

SEQ ID NO: 1200 - protein, 11 aa, Artificial Sequence
  Description of Artificial Sequence Synthetic peptide
GLDGPKGNPGL

SEQ ID NO: 1201 - protein, 9 aa, Artificial Sequence
  Description of Artificial Sequence Synthetic peptide
LLLVSLNLV

SEQ ID NO: 1202 - protein, 10 aa, Artificial Sequence
  Description of Artificial Sequence Synthetic peptide
SLSGTPLVSA

SEQ ID NO: 1203 - protein, 8 aa, Artificial Sequence
  Description of Artificial Sequence Synthetic peptide
GLYSSEYV

SEQ ID NO: 1204 - protein, 8 aa, Artificial Sequence
  Description of Artificial Sequence Synthetic peptide
SLSGTPLV

SEQ ID NO: 1205 - protein, 9 aa, Artificial Sequence
  Description of Artificial Sequence Synthetic peptide
MLPQIPFLL

SEQ ID NO: 1206 - protein, 10 aa, Artificial Sequence
  Description of Artificial Sequence Synthetic peptide
GLPGPPGPSA

SEQ ID NO: 1207 - protein, 9 aa, Artificial Sequence
  Description of Artificial Sequence Synthetic peptide
SAFTVILSK

SEQ ID NO: 1208 - protein, 9 aa, Artificial Sequence
  Description of Artificial Sequence Synthetic peptide
AVMPEGFIK

SEQ ID NO: 1209 - protein, 11 aa, Artificial Sequence
  Description of Artificial Sequence Synthetic peptide
GLYKNGTPVMY

SEQ ID NO: 1210 - protein, 10 aa, Artificial Sequence
  Description of Artificial Sequence Synthetic peptide
AIGTPIPFDK

SEQ ID NO: 1211 - protein, 9 aa, Artificial Sequence
  Description of Artificial Sequence Synthetic peptide
GLPGGPGAK

SEQ ID NO: 1212 - protein, 9 aa, Artificial Sequence
  Description of Artificial Sequence Synthetic peptide
ILYNRQQHY

SEQ ID NO: 1213 - protein, 11 aa, Artificial Sequence
  Description of Artificial Sequence Synthetic peptide
AGPPGPPGFGK

SEQ ID NO: 1214 - protein, 9 aa, Artificial Sequence
  Description of Artificial Sequence Synthetic peptide
GIPGFPGSK

SEQ ID NO: 1215 - protein, 10 aa, Artificial Sequence
  Description of Artificial Sequence Synthetic peptide
GTHVWVGLYK

SEQ ID NO: 1216 - protein, 9 aa, Artificial Sequence
  Description of Artificial Sequence Synthetic peptide
GVPGQPGIK

SEQ ID NO: 1217 - protein, 10 aa, Artificial Sequence
  Description of Artificial Sequence Synthetic peptide
VSAFTVILSK

SEQ ID NO: 1218 - protein, 10 aa, Artificial Sequence
  Description of Artificial Sequence Synthetic peptide
AVMPEGFIKA

SEQ ID NO: 1219 - protein, 9 aa, Artificial Sequence
  Description of Artificial Sequence Synthetic peptide
SSFSGFLVA

SEQ ID NO: 1220 - protein, 11 aa, Artificial Sequence
  Description of Artificial Sequence Synthetic peptide
PVSAFTVILSK

SEQ ID NO: 1221 - protein, 9 aa, Artificial Sequence
  Description of Artificial Sequence Synthetic peptide
GVPGMNGQK

SEQ ID NO: 1222 - protein, 11 aa, Artificial Sequence
  Description of Artificial Sequence Synthetic peptide
AYPAIGTPIPF

SEQ ID NO: 1223 - protein, 9 aa, Artificial Sequence
  Description of Artificial Sequence Synthetic peptide
IGPPGIPGF

SEQ ID NO: 1224 - protein, 9 aa, Artificial Sequence
  Description of Artificial Sequence Synthetic peptide
HYDPRTGIF

SEQ ID NO: 1225 - protein, 10 aa, Artificial Sequence
  Description of Artificial Sequence Synthetic peptide
EYVHSSFSGF

SEQ ID NO: 1226 - protein, 9 aa, Artificial Sequence
  Description of Artificial Sequence Synthetic peptide
AGPPGPPGF

SEQ ID NO: 1227 - protein, 9 aa, Artificial Sequence
  Description of Artificial Sequence Synthetic peptide
YYFSYHVHV

SEQ ID NO: 1228 - protein, 9 aa, Artificial Sequence
  Description of Artificial Sequence Synthetic peptide
AYPAIGTPI

SEQ ID NO: 1229 - protein, 9 aa, Artificial Sequence
  Description of Artificial Sequence Synthetic peptide
PLPNTKTQF

SEQ ID NO: 1230 - protein, 9 aa, Artificial Sequence
  Description of Artificial Sequence Synthetic peptide
CQIPGIYYF

SEQ ID NO: 1231 - protein, 9 aa, Artificial Sequence
  Description of Artificial Sequence Synthetic peptide
RPSLSGTPL

SEQ ID NO: 1232 - protein, 9 aa, Artificial Sequence
  Description of Artificial Sequence Synthetic peptide
LPQIPFLLL

SEQ ID NO: 1233 - protein, 9 aa, Artificial Sequence
  Description of Artificial Sequence Synthetic peptide
IPFLLLVSL

SEQ ID NO: 1234 - protein, 10 aa, Artificial Sequence
  Description of Artificial Sequence Synthetic peptide
LPGPPGPSAV

SEQ ID NO: 1235 - protein, 11 aa, Artificial Sequence
  Description of Artificial Sequence Synthetic peptide
GPIGPPGIPGF

SEQ ID NO: 1236 - protein, 9 aa, Artificial Sequence
  Description of Artificial Sequence Synthetic peptide
IPGPAGISV

SEQ ID NO: 1237 - protein, 10 aa, Artificial Sequence
  Description of Artificial Sequence Synthetic peptide
YPAIGTPIPF

SEQ ID NO: 1238 - protein, 10 aa, Artificial Sequence
  Description of Artificial Sequence Synthetic peptide
SPGPPGPAGI

SEQ ID NO: 1239 - protein, 9 aa, Artificial Sequence
  Description of Artificial Sequence Synthetic peptide
LPGPPGPSA

SEQ ID NO: 1240 - protein, 9 aa, Artificial Sequence
  Description of Artificial Sequence Synthetic peptide
SPGPPGPAG

SEQ ID NO: 1241 - protein, 9 aa, Artificial Sequence
  Description of Artificial Sequence Synthetic peptide
TIKSKGIAV

SEQ ID NO: 1242 - protein, 9 aa, Artificial Sequence
  Description of Artificial Sequence Synthetic peptide
HVHVKGTHV

SEQ ID NO: 1243 - protein, 8 aa, Artificial Sequence
  Description of Artificial Sequence Synthetic peptide
LPNTKTQF

SEQ ID NO: 1244 - protein, 8 aa, Artificial Sequence
  Description of Artificial Sequence Synthetic peptide
LPQIPFLL

SEQ ID NO: 1245 - protein, 8 aa, Artificial Sequence
  Description of Artificial Sequence Synthetic peptide
PFLLLVSL

SEQ ID NO: 1246 - protein, 8 aa, Artificial Sequence
  Description of Artificial Sequence Synthetic peptide
SLNLVHGV

SEQ ID NO: 1247 - protein, 8 aa, Artificial Sequence
  Description of Artificial Sequence Synthetic peptide
TGMPVSAF

SEQ ID NO: 1248 - protein, 9 aa, Artificial Sequence
  Description of Artificial Sequence Synthetic peptide
TPIPFDKIL

SEQ ID NO: 1249 - protein, 9 aa, Artificial Sequence
  Description of Artificial Sequence Synthetic peptide
SLQCVSLHL

SEQ ID NO: 1250 - protein, 9 aa, Artificial Sequence
  Description of Artificial Sequence Synthetic peptide
LVLSIALSV

SEQ ID NO: 1251 - protein, 9 aa, Artificial Sequence
  Description of Artificial Sequence Synthetic peptide
VILGVHLSV

SEQ ID NO: 1252 - protein, 9 aa, Artificial Sequence
  Description of Artificial Sequence Synthetic peptide
VLAPQESSV

SEQ ID NO: 1253 - protein, 10 aa, Artificial Sequence
  Description of Artificial Sequence Synthetic peptide
SLQCVSLHLL

SEQ ID NO: 1254 - protein, 9 aa, Artificial Sequence
  Description of Artificial Sequence Synthetic peptide
MLLRLSEPA

SEQ ID NO: 1255 - protein, 9 aa, Artificial Sequence
  Description of Artificial Sequence Synthetic peptide
LTMPALPMV

SEQ ID NO: 1256 - protein, 10 aa, Artificial Sequence
  Description of Artificial Sequence Synthetic peptide
FLTLSVTWIA

SEQ ID NO: 1257 - protein, 9 aa, Artificial Sequence
  Description of Artificial Sequence Synthetic peptide
KLQCVDLHV

SEQ ID NO: 1258 - protein, 10 aa, Artificial Sequence
  Description of Artificial Sequence Synthetic peptide
FLTPKKLQCV

SEQ ID NO: 1259 - protein, 10 aa, Artificial Sequence
  Description of Artificial Sequence Synthetic peptide
FLRPGDDSTL

SEQ ID NO: 1260 - protein, 9 aa, Artificial Sequence
  Description of Artificial Sequence Synthetic peptide
FLGYLILGV

SEQ ID NO: 1261 - protein, 9 aa, Artificial Sequence
  Description of Artificial Sequence Synthetic peptide
LLANDLMLI

SEQ ID NO: 1262 - protein, 9 aa, Artificial Sequence
  Description of Artificial Sequence Synthetic peptide
FQNSYTIGL

SEQ ID NO: 1263 - protein, 9 aa, Artificial Sequence
  Description of Artificial Sequence Synthetic peptide
MLIKLDESV

SEQ ID NO: 1264 - protein, 9 aa, Artificial Sequence
  Description of Artificial Sequence Synthetic peptide
VLQCVNVSV

SEQ ID NO: 1265 - protein, 10 aa, Artificial Sequence
  Description of Artificial Sequence Synthetic peptide
LLANGRMPTV

SEQ ID NO: 1266 - protein, 10 aa, Artificial Sequence
  Description of Artificial Sequence Synthetic peptide
ILNDTGCHYV

SEQ ID NO: 1267 - protein, 10 aa, Artificial Sequence
  Description of Artificial Sequence Synthetic peptide
FQYPEFSIEL

SEQ ID NO: 1268 - protein, 9 aa, Artificial Sequence
  Description of Artificial Sequence Synthetic peptide
ILGKYQLNV

SEQ ID NO: 1269 - protein, 10 aa, Artificial Sequence
  Description of Artificial Sequence Synthetic peptide
LLGNSVNFTV

SEQ ID NO: 1270 - protein, 9 aa, Artificial Sequence
  Description of Artificial Sequence Synthetic peptide
VLDCCISLL

SEQ ID NO: 1271 - protein, 9 aa, Artificial Sequence
  Description of Artificial Sequence Synthetic peptide
ILGSFELQL

SEQ ID NO: 1272 - protein, 9 aa, Artificial Sequence
  Description of Artificial Sequence Synthetic peptide
RLIWLVKMV

SEQ ID NO: 1273 - protein, 11 aa, Artificial Sequence
  Description of Artificial Sequence Synthetic peptide
VLLGNSVNFTV

SEQ ID NO: 1274 - protein, 9 aa, Artificial Sequence
  Description of Artificial Sequence Synthetic peptide
TLAIPLTDV

SEQ ID NO: 1275 - protein, 10 aa, Artificial Sequence
  Description of Artificial Sequence Synthetic peptide
  MOD_RES (1)..(1): May or may not be present
VVVGADGVGK

SEQ ID NO: 1276 - protein, 10 aa, Artificial Sequence
  Description of Artificial Sequence Synthetic peptide
  MOD_RES (10)..(10): May or may not be present
GADGVGKSAL

SEQ ID NO: 1277 - protein, 8 aa, Artificial Sequence
  Description of Artificial Sequence Synthetic peptide
DGVGKSAL

SEQ ID NO: 1278 - protein, 9 aa, Artificial Sequence
  Description of Artificial Sequence Synthetic peptide
ADGVGKSAL

SEQ ID NO: 1279 - protein, 8 aa, Artificial Sequence
  Description of Artificial Sequence Synthetic peptide
GADGVGKS

SEQ ID NO: 1280 - protein, 10 aa, Artificial Sequence
  Description of Artificial Sequence Synthetic peptide
  MOD_RES (1)..(1): May or may not be present
VVVGARGVGK

SEQ ID NO: 1281 - protein, 9 aa, Artificial Sequence
  Description of Artificial Sequence Synthetic peptide
  MOD_RES (1)..(1): May or may not be present
ARGVGKSAL

SEQ ID NO: 1282 - protein, 9 aa, Artificial Sequence
  Description of Artificial Sequence Synthetic peptide
GARGVGKSA

SEQ ID NO: 1283 - protein, 10 aa, Artificial Sequence
  Description of Artificial Sequence Synthetic peptide
EYKLVVVGAR

SEQ ID NO: 1284 - protein, 15 aa, Artificial Sequence
  Description of Artificial Sequence Synthetic peptide
TEYKLVVVGAVGVGK

SEQ ID NO: 1285 - protein, 20 aa, Artificial Sequence
  Description of Artificial Sequence Synthetic peptide
TEYKLVVVGAVGVGKSALTI

SEQ ID NO: 1286 - protein, 10 aa, Artificial Sequence
  Description of Artificial Sequence Synthetic peptide
  MOD_RES (1)..(1): May or may not be present
GAVGVGKSAL

SEQ ID NO: 1287 - protein, 10 aa, Artificial Sequence
  Description of Artificial Sequence Synthetic peptide
  MOD_RES (1)..(1): May or may not be present
VVVGACGVGK

SEQ ID NO: 1288 - protein, 10 aa, Artificial Sequence
  Description of Artificial Sequence Synthetic peptide
  MOD_RES (1)..(1): May or may not be present
VVVGAVGVGK

(19) United States
(12) Patent Application Publication
(10) Pub. No.: US 2023/0398218 A1
(43) Pub. Date: Dec. 14, 2023

(54) RAS NEOANTIGENS AND USES THEREOF

(71) Applicant: BioNTech US Inc.

(72) Inventor: Vikram Juneja, Newton, MA (US)

(21) Appl. No.: 18/007,096

(22) PCT Filed: Aug. 12, 2021
(86) PCT No.: PCT/US21/45808

§ 371 Date: Jan. 27, 2023

Related U.S. Application Data
(60) Provisional application No. 63/065,346, filed on Aug. 13, 2020.

(51) Int. Cl. A61K 39/00; C12N 5/0783; C07K 14/725; A61P 35/00

(52) U.S. Cl. CPC A61K 39/464401; C12N 5/0636; A61K 39/4611; C07K 14/7051; A61P 35/00; C12N 2502/1121; C12N 2501/2307; C12N 2501/2315; C12N 2501/26; C12N 2501/2301; C12N 2501/25

(57) ABSTRACT

Compositions and methods for preparing T cell compositions and uses thereof are described, including methods for treating cancer in a subject in need thereof by administering T cells induced with peptides comprising at least one of KRAS epitope having a sequence GACGVGKSA that binds to a protein encoded by an HLA allele C03:04; or having a sequence GAVGVGKSA that binds to a protein encoded by an HLA allele C03:03 wherein the respective protein encoded by the HLA allele is expressed in a cell of the subject. Also included are immunogenic compositions comprising peptide(s) comprising an epitope described above, or antigen presenting cells loaded with the peptide(s) comprising the epitope.

### CROSS-REFERENCE

This application claims the benefit of U.S. Provisional Application No. 63/065,346, filed on Aug. 13, 2020; which is incorporated herein by reference in its entirety.

### BACKGROUND

Cancer immunotherapy focuses on enhancing one's body's immune response to fight and eradicate cancer cells. This is achieved by administering antigenic peptide therapy, where the peptides comprise cancer antigens, or administering nucleic acid encoding the antigenic peptides comprising the cancer antigens, or using adoptive immunotherapy targeted towards the cancer. Adoptive immunotherapy or adoptive cellular therapy with lymphocytes (ACT) is the transfer of gene modified T lymphocytes to a subject for the therapy of disease. Adoptive immunotherapy has yet to realize its potential for treating a wide variety of diseases including cancer, infectious disease, autoimmune disease, inflammatory disease, and immunodeficiency. However, most, if not all adoptive immunotherapy strategies require T cell activation and expansion steps to generate a clinically effective, therapeutic dose of T cells. Accurate identification of cancer antigenic epitopes, and their potential to produce immune activation in a patient is necessary. Additionally, existing strategies of obtaining patient cells, and ex vivo activation, expansion and recovery of effective number of cells for ACT is a prolonged, cumbersome and an inherently complex process—and poses a serious challenge. Accordingly, there remains a need for developing compositions and methods for generating antigen-specific immunotherapy directed to a single patient having a cancer.

### SUMMARY

Provided herein is a method for treating cancer in a subject in need thereof, wherein the cancer contains a RAS mutation and the treatment is for a patient expressing a specific major histocompatibility complex (MHC) peptide encoded by a specific HLA allele. Also provided herein is an ex vivo method for preparing antigen-specific T cells, the method comprising contacting T cells with antigen presenting cells (APCs) comprising one or more peptides containing an epitope with a RAS mutation.

In some embodiments, the method for treating cancer described herein comprises administering a therapy, such as an immunotherapy, to a subject with a cancer comprising a RAS mutation. In some embodiments, the RAS mutation is a G12C mutation. In some embodiments, RAS mutation is a G12V mutation. In some embodiments, the method for treating cancer is for a subject that expresses an MHC protein encoded by HLA C03:04 allele. In some embodiments, the method for treating cancer is for a subject that expresses an MHC protein encoded by HLA C03:03 allele. In some embodiments, the therapy is a peptide, a polynucleotide encoding the peptide, APCs comprising the peptide or the polynucleotide, or T cells stimulated with APCs comprising the peptide or the polynucleotide.

In some embodiments the method comprises generating an antigen therapy comprising one or more peptides, wherein the one or more peptides comprise at least one epitope with a sequence GACGVGKSA, that is capable of activating an anti-cancer immune response in a suitable subject. A suitable subject expresses an MHC protein encoded by HLA C03:04 allele. In some embodiments, the method comprises administering to a subject an antigen therapy, wherein the subject expresses an MHC protein encoded by HLA C03:04 allele and has a cancer cell with a KRAS G12C mutation, wherein the antigen therapy comprises one or more peptides containing an epitope with a sequence GACGVGKSA. In some embodiments, the subject expressing an MHC protein encoded by HLA C03:04 allele is administered an anti-cancer therapy that comprises one or more nucleic acids encoding the peptide, wherein the peptide comprises an epitope with a sequence GACGVGKSA.

In some embodiments, the method comprises administering antigen presenting cells expressing one or more peptides comprising an epitope with a sequence GACGVGKSA or a polynucleotide sequence encoding the one or more peptides, and an MHC protein encoded by an HLA C03:04 allele to a subject who expresses the MHC protein encoded by the HLA C03:04 allele.

In some embodiments the method comprises generating T cell therapy for a subject that expresses an MHC protein encoded by HLA C03:04 allele and has a cancer cell with a KRAS G12C mutation, the method comprising, contacting allogeneic or autologous T cells with APCs comprising one or more peptides comprising an epitope with a sequence GACGVGKSA, expanding the T cells in culture under conditions that stimulate the T cells to be cytotoxic according to a cytotoxicity assay. In some embodiments, the method comprises administering the T cell therapy to the subject. In some embodiments, the method comprises preparing a T cell, wherein the T cell is responsive to an antigen comprising an amino acid sequence GACGVGKSA. In some embodiments, the method comprises preparing a T cell, the method comprising stimulating a T cell with a polypeptide comprising an amino acid sequence GACGVGKSA. In some embodiments, the method comprises administering to a subject in need thereof a T cell preparation comprising a T cell, wherein the T cell is responsive to an antigen comprising an amino acid sequence GACGVGKSA. In some embodiments, the subject is human. In some embodiments, the subject expresses an MHC protein encoded by HLA C03:04 allele.

In some embodiments, the method described herein targets a mutant RAS antigen comprising a G12V mutation. In some embodiments, the method is directed to a patient that expresses an MHC protein encoded by HLA C03:03 allele. In some embodiments, the method comprises generating an antigen therapy comprising one or more peptides, wherein the one or more peptides comprise at least one epitope with a sequence GAVGVGKSA that can activate an anti-cancer immune response in a suitable patient. A suitable patient can express an MHC protein encoded by HLA C03:03 allele. In some embodiments, the method comprises administering to a patient the antigen therapy, wherein the patient an MHC protein encoded by HLA C03:03 allele and has a cancer cell with a KRAS G12V mutation, wherein the antigen therapy comprises a one or more peptides containing an epitope with a sequence GAVGVGKSA. In some embodiments, the patient expressing an MHC protein encoded by HLA C03:03 allele is administered an anti-cancer therapy that comprises one or more nucleic acids encoding the peptide, wherein the peptide comprises an epitope with a sequence GAVGVGKSA.

In some embodiments, the method comprises administering antigen presenting cells expressing one or more peptides comprising an epitope with a sequence GACGVGKSA or a polynucleotide sequence encoding the one or more peptides, and an MHC protein encoded by an HLA C03:04 allele to a subject who expresses the MHC protein encoded by the HLA C03:04 allele.

In some embodiments the method comprises generating T cell therapy for a patient that expresses an MHC protein encoded by HLA C03:03 allele and has a cancer cell with a KRAS G12V mutation, the method comprising, contacting allogeneic or an autologous T cells derived from the patient with APCs comprising one or more peptides containing an epitope with a sequence GAVGVGKSA, expanding the T cells in culture under conditions that stimulate the T cells to be cytotoxic according to a cytotoxicity assay. In some embodiments, the method comprises administering the T cell therapy to the patient. In some embodiments, the method comprises preparing a T cell, wherein the T cell is responsive to an antigen comprising an amino acid sequence GAVGVGKSA. In some embodiments, the method comprises preparing a T cell, the method comprising stimulating a T cell with a polypeptide comprising an amino acid sequence GAVGVGKSA. In some embodiments, the method comprises administering to a subject in need thereof a T cell preparation comprising a T cell, wherein the T cell is responsive to an antigen comprising an amino acid sequence GAVGVGKSA. In some embodiments, the subject is human. In some embodiments, the subject expresses an MHC protein encoded by HLA C03:03 allele.

In one aspect, provided herein is an ex vivo method for preparing antigen-specific T cells, the method comprising contacting T cells with APCs comprising one or more peptides containing an epitope with a sequence selected from GACGVGKSA and GAVGVGKSA, wherein the APCs express a protein encoded by an HLA-C03:04 allele and the epitope sequence is GACGVGKSA; and wherein the APCs express a protein encoded by an HLA-C03:03 allele and the epitope sequence is GAVGVGKSA.

In some embodiments, the T cells are from a subject with cancer. In some embodiments, the T cells are allogeneic T cells.

In some embodiments, the method further comprises administering the T cells to a subject in need thereof, wherein the subject expresses a protein encoded by an HLA-C03:04 allele, and the T cells have been contacted with APCs comprising one or more peptides containing the epitope GACGVGKSA.

In some embodiments, the method further comprises administering the T cells to a subject in need thereof, wherein the subject expresses one or more protein encoded by an HLA-C03:03 allele and the T cells have been contacted with APCs comprising a peptide containing the epitope GAVGVGKSA.

In some embodiments, the APCs are from a subject with cancer. In some embodiments, the APCs are allogeneic APCs.

In some embodiments, the method comprises obtaining a biological sample comprising T cells and/or APCs from a subject. In some embodiments, the biological sample is peripheral blood mononuclear cell (PBMC) sample. In some embodiments, the method comprises depleting CD14+ cells from the biological sample comprising T cells and/or APCs. In some embodiments, the method comprises depleting CD25+ cells from the biological sample comprising T cells and/or APCs. In some embodiments, the method comprises incubating the T cells and APCs in the presence of FMS-like tyrosine kinase 3 receptor ligand (FLT3L).

In some embodiments, the method comprises stimulating or expanding the T cells in the presence of the APCs for at least 3, 4, 5, 6, 7, 8, 9, 10, 11, 12, 13, 14, 15, 16, 7, 18, 19 or 20 or more days. In some embodiments, the method comprises expanding the T cells at least 5-fold, 10-fold, 20, fold, 50-fold, 100-fold, 500-fold or 1,000-fold or more in the presence of the APCs. In some embodiments, the antigen-specific T cells are prepared in less than 28 days.

In some embodiments, the epitope binds to a protein encoded by an HLA allele of the subject, is immunogenic according to an immunogenicity assay, is presented by APCs according to a mass spectrometry assay, and/or stimulates T cells to be cytotoxic according to a cytotoxicity assay.

In one aspect, provided herein is a method of treating a subject with cancer comprising administering to the subject a peptide, a polynucleotide encoding the peptide, APCs comprising the peptide or the polynucleotide encoding the peptide, or T cells stimulated with APCs comprising the peptide or the polynucleotide encoding the peptide; wherein the peptide comprises an epitope with a sequence GACGVGKSA, and wherein the subject expresses a protein encoded by an HLA-C03:04 allele.

In one aspect, provided herein is a method of treating a subject with cancer comprising administering to the subject a peptide, a polynucleotide encoding the peptide, APCs comprising the peptide or the polynucleotide encoding the peptide, or T cells stimulated with APCs comprising the peptide or the polynucleotide encoding the peptide; wherein the peptide comprises an epitope with a sequence GAVGVGKSA, and wherein the subject expresses a protein encoded by an HLA-C03:03 allele.

In some embodiments, the epitope with the sequence GACGVGKSA binds to the protein encoded by the HLA-C03:04 allele. In one embodiment, said epitope is presented by the protein encoded by the HLA-C03:04 allele.

In some embodiments, the epitope with the sequence GAVGVGKSA binds to the protein encoded by the HLA-C03:03 allele. In one embodiment, said epitope is presented by the protein encoded by the HLA-C03:03 allele.

In some embodiments, the cancer is selected from the group consisting of pancreatic ductal adenocarcinoma, non-small cell lung cancer, colorectal cancer and cholangiocarcinoma.

In some embodiments, the peptide comprises one or more additional epitopes.

In some embodiments, the method further comprises administering an additional anti-cancer therapy to the subject.

In some embodiments, the APCs are from the subject with cancer. In some embodiments of the method of treating described herein, the APCs are allogeneic APCs.

In some embodiments, the T cells are from the subject with cancer. In some embodiments, the T cells are allogeneic. In some embodiments, the T cells are stimulated with the APCs in vitro or ex vivo. In some embodiments, the T cells have been stimulated with the APCs for at least 3, 4, 5, 6, 7, 8, 9, 10, 11, 12, 13, 14, 15, 16, 7, 18, 19 or 20 or more days. In some embodiments, the T cells are expanded in the presence of the APCs in vitro or ex vivo. In some embodiments, the T cells have been expanded in the presence the APCs for at least 3, 4, 5, 6, 7, 8, 9, 10, 11, 12, 13, 14, 15, 16, 7, 18, 19 or 20 or more days In some embodiments, the T cells have been expanded at least 5-fold, 10-fold, 20, fold, 50-fold, 100-fold, 500-fold or 1,000-fold or more in the presence of the APCs.

In some embodiments, the T cells are assayed for expression of a T cell activation marker. In some embodiments, the T cells are assayed for cytokine production. In some embodiments, the T cell activation marker or cytokine is selected from the group consisting of cell surface expression of CD107a and/or CD107b, IL2, IFN-γ, TNFα, TNFβ and any combination thereof.

In some embodiments, the T cells are antigen-specific T cells.

In one aspect, provided herein is a pharmaceutical composition comprising T cells comprising a population of T cells expressing a T cell receptor (TCR) that binds to a complex of an MHC protein encoded by an HLA-C03:04 allele and an epitope with a sequence GACGVGKSA.

In one aspect, provided herein is a pharmaceutical composition comprising T cells comprising a population of T cells expressing a T cell receptor (TCR) that binds to a complex of an MHC protein encoded by an HLA-C03:03 allele and an epitope with a sequence GAVGVGKSA.

In one aspect, provided herein is a pharmaceutical composition comprising APCs expressing an MHC protein encoded by an HLA-C03:04 allele, wherein the APCs comprise a peptide having an epitope with a sequence GACGVGKSA or a polynucleotide encoding the peptide.

In one aspect, provided herein is a pharmaceutical composition comprising APCs expressing an MHC protein encoded by an HLA-C03:03 allele, wherein the APCs comprise a peptide having an epitope with a sequence GAVGVGKSA or a polynucleotide encoding the peptide.

In some embodiments, the APCs are from a subject with cancer. In some embodiments, the APCs are allogeneic APCs.

In some embodiments, the T cells are from a subject with cancer.

In some embodiments, the T cells are allogeneic.

In some embodiments, the population of T cells comprises CD8+ T cells.

In some embodiments, at least 0.1% of the CD8+ T cells in the population of T cells are derived from naïve CD8+ T cells

In some embodiments, the population of T cells comprises CD4+ T cells.

In some embodiments, at least 0.1% of the CD4+ T cells in the population of T cells are derived from naïve CD4+ T cells.

In some embodiments, provided herein is a TCR comprising a TCR alpha chain and a TCR beta chain, wherein the TCR is capable of binding to a mutated RAS epitope comprising a GACGVGKSA when presented in complex with an MHC encoded by C03:04 allele. In some embodiments, provided herein is a TCR comprising a TCR alpha chain and a TCR beta chain, wherein the TCR is capable of binding to a mutated RAS epitope comprising a GAVGVGKSA when presented in complex with an MHC encoded by C03:03 allele.

### BRIEF DESCRIPTION OF THE DRAWINGS

FIG. 1A is schematic of an exemplary method provided herein to prime, activate and expand antigen-specific T cells.

FIG. 1B is schematic of an exemplary method provided herein to prime, activate and expand antigen-specific T cells.

FIG. 2 is schematic of an exemplary method for offline characterization of shared epitopes.

FIG. 3A depicts data illustrating that in silico epitope prediction identified multiple neoantigens derived from RAS G12D mutations that are presented according to mass spectrometry.

FIG. 3B depicts data illustrating that in silico epitope prediction identified multiple neoantigens derived from RAS G12V mutations that are presented according to mass spectrometry.

FIG. 3C depicts data illustrating that in silico epitope prediction identified multiple neoantigens derived from RAS G12C mutations that are presented according to mass spectrometry.

FIG. 3D depicts data illustrating that in silico epitope prediction identified multiple neoantigens derived from RAS G12R mutations that are presented according to mass spectrometry

FIG. 4 depicts data illustrating that presentation of shared neoantigen epitopes can be directly confirmed by mass spectrometry and that RAS neoantigens are targetable in defined patient populations.

FIG. 5 shows an exemplary head-to-toe plot of MS/MS spectra for the endogenously processed mutant RAS peptide epitope VVVGAVGVGK (top) and its corresponding heavy isotopically labeled peptide (bottom). 293T cells were lentivirally transduced to make cells express RAS^{G12V} and HLA-A*03:01.

FIG. 6 depicts data illustrating that an exemplary method provided herein to prime, activate and expand antigen-specific T cells induces de novo CD8 T cell responses against RAS G12 neoantigens on HLA-A11:01 and HLA-A03:01.

FIG. 7 depicts exemplary data illustrating that RAS^{G12V}-activated T cells generated ex vivo can kill target cells. A375 target cells expressing GFP were loaded with 2 μM RAS^{G12V} antigen, wild-type RAS antigen, or no peptide as control GFP+ cells. RAS^{G12V}-specific CD8 T cells (effector cells) were incubated with control cells or target cells in a 0.05:1 ratio. In presence of the effector cells, target cells were lysed and depleted more readily that control cells which present either RAS WT antigen or no antigen. Graph of specific cell killing as normalized by target cell growth with no peptide is shown in the left diagram. Representative images are shown on the right.

FIG. 8 depicts data illustrating that an exemplary method provided herein to prime, activate and expand RAS G12V-specific T cells with RAS G12V neoantigens on HLA-11:01, but not the corresponding wild-type antigens, induces T cells to become cytotoxic using the indicated effector:target cell ratios and increasing peptide concentration.

FIG. 9 depicts an exemplary graph of AnnexinV positive cells over time after co-culturing NCI-H441 cells naturally expressing both the RAS^{G12V} mutation and the HLA-A*03:01 gene with T cells that had been primed and activated and expanded with a peptide containing an epitope with the RAS^{G12V} mutation at the indicated effector:target cell ratio.

FIG. 10A depicts a graph of IL2 concentration (μg/mL) by Jurkat cells transduced with a TCR that binds to the RAS-G12V epitope bound to an MHC encoded by the HLA-A11:01 allele, when cultured with A375 expressing HLA-A11:01 loaded with increasing concentration of RAS wild-type peptide or RAS-G12V mutant peptide.

FIG. 10B depicts exemplary graphs of AnnexinV positive cells over time after co-culturing TCR-transduced PBMCs with 5,000 SNGM cells with natural G12V and HLA-A11:01 across a range of effector:target cell ratios. PBMCs transduced with a TCR that does not recognize RAS G12V mutant are shown in the top panel. PBMCs transduced with a TCR that does not recognize RAS G12V mutant when presented by an HLA A11:01 are shown in the bottom panel.

FIG. 10C depicts an exemplary graph of IL2 concentration (μg/mL) released by Jurkat cells transduced with a TCR that binds to RAS-G12V when bound to an MHC encoded by the HLA-A03:01 allele cultured with RAS-wild-type peptide or RAS-G12V mutant peptide loaded target cells (A375-A03:01).

FIG. 10D depicts a graph of AnnexinV positive cells over time (top) after co-culturing TCR-transduced PBMCs with target cells that express RAS G12V and HLA-A03:01 using an effector:target cell ratio of 0.75:1. PBMCs were either transduced with the RAS mutant specific TCR, or with a TCR that does not bind to RAS (irrelevant, irr TCR). A graph of IFNγ concentration (μg/mL) after 24 hours of coculturing TCR-transduced PBMCs with target cells with natural G12V and HLA-A03:01 using an effector:target cell ratio of 0.75:1 is shown in the bottom panel.

FIG. 11A depicts a graph of IL2 concentration (μg/mL) released by Jurkat cells transduced with a TCR that binds to the underlined RAS-G12V epitope when bound to an MHC encoded by the HLA-A11:01 allele in the presence of FLT3L-treated PBMCs contacted with increasing amounts of the indicated RAS-G12V mutant peptides.

FIG. 11B depicts an exemplary data illustrating the immunogenicity of the indicated RAS-G12V mutant peptides in vitro using PBMCs from healthy donors (top) and in vivo using HLA-A11:01 transgenic mice immunized with the peptides (bottom).

### DETAILED DESCRIPTION

Although many epitopes have the potential to bind to an MHC molecule, few are capable of binding to an MHC molecule when tested experimentally. Although many epitopes also have the potential to potential to be presented by an MHC molecule that can, for example, be detected by mass spectrometry, only a select number of these epitopes can be presented and detected by mass spectrometry. Although many epitopes also have the potential to be immunogenic, when tested experimentally many of these epitopes are not immunogenic, despite being demonstrated to be presented by antigen presenting cells. Many epitopes also have the potential to activate T cells to become cytotoxic; however, many epitopes that have been demonstrated to be presented by antigen presenting cells and/or to be immunogenic are still not capable of activating T cells to become cytotoxic.

Provided herein are antigens containing T cell epitopes that have been identified and validated as binding to one or more MHC molecules, presented by the one or more MHC molecules, being immunogenic and capable of activating T cells to become cytotoxic. The validated antigens and polynucleotides encoding these antigens can be used in preparing antigen specific T cells for therapeutic uses. In some embodiments, the validated antigens and polynucleotides encoding these antigens can be pre-manufactured and stored for use in a method of manufacturing T cells for therapeutic uses. For example, the validated antigens and polynucleotides encoding these antigens can be pre-manufactured or manufactured quickly to prepare therapeutic T cell compositions for patients quickly. Using validated antigens with T cell epitopes, immunogens such as peptides having HLA binding activity or RNA encoding such peptides can be manufactured. Multiple immunogens can be identified, validated and pre-manufactured in a library. In some embodiments, peptides can be manufactured in a scale suitable for storage, archiving and use for pharmacological intervention on a suitable patient at a suitable time.

Mutations in KRAS have been known for more than 60 years to cause various forms of cancer, but developing successful therapy against KRAS cancers remains largely elusive. Described herein are new immunotherapeutic agents and uses thereof based on the discovery of neoantigens arising from mutational events unique to an individual's tumor. Accordingly, the present disclosure described herein provides peptides, polynucleotides encoding the peptides, and peptide binding agents that can be used, for example, to stimulate an immune response to a tumor associated antigen or neoepitope, to create an immunogenic composition or cancer vaccine for use in treating disease.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

### Definitions

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

“Major Histocompatibility Complex” or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, Fundamental Immunology, 3^{rd} Ed., Raven Press, New York (1993). “Proteins or molecules of the major histocompatibility complex (MHC)”, “MHC molecules”, “MHC proteins” or “HLA proteins” are to be understood as meaning proteins capable of binding peptides resulting from the proteolytic cleavage of protein antigens and representing potential lymphocyte epitopes, (e.g., T cell epitope and B cell epitope) transporting them to the cell surface and presenting them there to specific cells, in particular cytotoxic T-lymphocytes, T-helper cells, or B cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The cellular biology and the expression patterns of the two MHC classes are adapted to these different roles.

“Human Leukocyte Antigen” or “HLA” is a human class I or class II Major Histocompatibility Complex (MHC) protein (see, e.g., Stites, et al., Immunology, 8^{th} Ed., Lange Publishing, Los Altos, Calif. (1994).

“Polypeptide”, “peptide” and their grammatical equivalents as used herein refer to a polymer of amino acid residues, typically L-amino acids, connected one to the other, typically by peptide bonds between the α-amino and carboxyl groups of adjacent amino acids. Polypeptides and peptides include, but are not limited to, mutant peptides, “neoantigen peptides” and “neoantigenic peptides”. Polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. A “mature protein” is a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitination, addition of pyrrolidone carboxylic acid, formation of disulfide bridges, sulfation, myristoylation, palmitoylation, isoprenylation, farnesylation, geranylation, glypiation, lipoylation and iodination.

A peptide or polypeptide may comprise at least one flanking sequence. The term “flanking sequence” as used herein refers to a fragment or region of a peptide that is not a part of an epitope.

An “immunogenic” peptide or an “immunogenic” epitope or “peptide epitope” is a peptide that comprises an allele-specific motif such that the peptide will bind an HLA molecule and induce a cell-mediated or humoral response, for example, cytotoxic T lymphocyte (CTL (e.g., CD8⁺)), helper T lymphocyte (Th (e.g., CD4⁺)) and/or B lymphocyte response. Thus, immunogenic peptides described herein are capable of binding to an appropriate HLA molecule and thereafter inducing a CTL (cytotoxic) response, or a HTL (and humoral) response, to the peptide.

“Neoantigen” means a class of tumor antigens which arise from tumor-specific changes in proteins. Neoantigens encompass, but are not limited to, tumor antigens which arise from, for example, substitution in the protein sequence, frame shift mutation, fusion polypeptide, in-frame deletion, insertion, expression of endogenous retroviral polypeptides, and tumor-specific overexpression of polypeptides.

The term “residue” refers to an amino acid residue or amino acid mimetic residue incorporated into a peptide or protein by an amide bond or amide bond mimetic, or nucleic acid (DNA or RNA) that encodes the amino acid or amino acid mimetic.

A “neoepitope”, “tumor specific neoepitope” or “tumor antigen” refers to an epitope or antigenic determinant region that is not present in a reference, such as a non-diseased cell, e.g., a non-cancerous cell or a germline cell, but is found in a diseased cell, e.g., a cancer cell. This includes situations where a corresponding epitope is found in a normal non-diseased cell or a germline cell but, due to one or more mutations in a diseased cell, e.g., a cancer cell, the sequence of the epitope is changed so as to result in the neoepitope. The term “neoepitope” as used herein refers to an antigenic determinant region within the peptide or neoantigenic peptide. A neoepitope may comprise at least one “anchor residue” and at least one “anchor residue flanking region.” A neoepitope may further comprise a “separation region.” The term “anchor residue” refers to an amino acid residue that binds to specific pockets on HLAs, resulting in specificity of interactions with HLAs. In some cases, an anchor residue may be at a canonical anchor position. In other cases, an anchor residue may be at a non-canonical anchor position. Neoepitopes may bind to HLA molecules through primary and secondary anchor residues protruding into the pockets in the peptide-binding grooves. In the peptide-binding grooves, specific amino acids compose pockets that accommodate the corresponding side chains of the anchor residues of the presented neoepitopes. Peptide-binding preferences exist among different alleles of both of HLA I and HLA II molecules. HLA class I molecules bind short neoepitopes, whose N- and C-terminal ends are anchored into the pockets located at the ends of the neoepitope binding groove. While the majority of the HLA class I binding neoepitopes are of about 9 amino acids, longer neoepitopes can be accommodated by the bulging of their central portion, resulting in binding neoepitopes of about 8 to 12 amino acids. Neoepitopes binding to HLA class II proteins are not constrained in size and can vary from about 16 to 25 amino acids. The neoepitope binding groove in the HLA class II molecules is open at both ends, which enables binding of peptides with relatively longer length. Though the core 9 amino acid residues long segment contributes the most to the recognition of the neoepitope, the anchor residue flanking regions are also important for the specificity of the peptide to the HLA class II allele. In some cases, the anchor residue flanking region is N-terminus residues. In another case, the anchor residue flanking region is C-terminus residues. In yet another case, the anchor residue flanking region is both N-terminus residues and C-terminus residues. In some cases, the anchor residue flanking region is flanked by at least two anchor residues. An anchor residue flanking region flanked by anchor residues is a “separation region.” In some embodiments, an epitope may be interchangeably used with a neoepitope, wherein the epitope is described to be present in a cancer cell and comprises a mutation that is not present in a non-cancer cell.

A “reference” can be used to correlate and compare the results obtained in the methods of the present disclosure from a tumor specimen. Typically, the “reference” may be obtained on the basis of one or more normal specimens, in particular specimens which are not affected by a cancer disease, either obtained from a patient or one or more different individuals, for example, healthy individuals, in particular individuals of the same species. A “reference” can be determined empirically by testing a sufficiently large number of normal specimens.

An “epitope” is the collective features of a molecule, such as primary, secondary and tertiary peptide structure, and charge, that together form a site recognized by, for example, an immunoglobulin, T cell receptor, HLA molecule, or chimeric antigen receptor. Alternatively, an epitope can be defined as a set of amino acid residues which is involved in recognition by a particular immunoglobulin, or in the context of T cells, those residues necessary for recognition by T cell receptor proteins, chimeric antigen receptors, and/or Major Histocompatibility Complex (MHC) receptors. A “T cell epitope” is to be understood as meaning a peptide sequence which can be bound by the MHC molecules of class I or II in the form of a peptide-presenting MHC molecule or MHC complex and then, in this form, be recognized and bound by T cells, such as T-lymphocytes or T-helper cells. Epitopes can be prepared by isolation from a natural source, or they can be synthesized according to standard protocols in the art. Synthetic epitopes can comprise artificial amino acid residues, “amino acid mimetics,” such as D isomers of naturally-occurring L amino acid residues or non-naturally-occurring amino acid residues such as cyclohexylalanine. Throughout this disclosure, epitopes may be referred to in some cases as peptides or peptide epitopes. It is to be appreciated that proteins or peptides that comprise an epitope or an analog described herein as well as additional amino acid(s) are still within the bounds of the present disclosure. In certain embodiments, the peptide comprises a fragment of an antigen. In certain embodiments, there is a limitation on the length of a peptide of the present disclosure. The embodiment that is length-limited occurs when the protein or peptide comprising an epitope described herein comprises a region (i.e., a contiguous series of amino acid residues) having 100% identity with a native sequence. In order to avoid the definition of epitope from reading, e.g., on whole natural molecules, there is a limitation on the length of any region that has 100% identity with a native peptide sequence. Thus, for a peptide comprising an epitope described herein and a region with 100% identity with a native peptide sequence, the region with 100% identity to a native sequence generally has a length of: less than or equal to 600 amino acid residues, less than or equal to 500 amino acid residues, less than or equal to 400 amino acid residues, less than or equal to 250 amino acid residues, less than or equal to 100 amino acid residues, less than or equal to 85 amino acid residues, less than or equal to 75 amino acid residues, less than or equal to 65 amino acid residues, and less than or equal to 50 amino acid residues. In certain embodiments, an “epitope” described herein is comprised by a peptide having a region with less than 51 amino acid residues that has 100% identity to a native peptide sequence, in any increment down to 5 amino acid residues; for example 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues.

The nomenclature used to describe peptides or proteins follows the conventional practice wherein the amino group is presented to the left (the amino- or N-terminus) and the carboxyl group to the right (the carboxy- or C-terminus) of each amino acid residue. When amino acid residue positions are referred to in a peptide epitope they are numbered in an amino to carboxyl direction with position one being the residue located at the amino terminal end of the epitope, or the peptide or protein of which it can be a part. In the formula representing selected specific embodiments of the present disclosure, the amino- and carboxyl-terminal groups, although not specifically shown, are in the form they would assume at physiologic pH values, unless otherwise specified. In the amino acid structure formula, each residue is generally represented by standard three letter or single letter designations. The L-form of an amino acid residue is represented by a capital single letter or a capital first letter of a three-letter symbol, and the D-form for those amino acid residues having D-forms is represented by a lower case single letter or a lower case three letter symbol. However, when three letter symbols or full names are used without capitals, they can refer to L amino acid residues. Glycine has no asymmetric carbon atom and is simply referred to as “Gly” or “G”. The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine.)

The term “mutation” refers to a change of or difference in the nucleic acid sequence (nucleotide substitution, addition or deletion) compared to a reference. A “somatic mutation” can occur in any of the cells of the body except the germ cells (sperm and egg) and therefore are not passed on to children. These alterations can (but do not always) cause cancer or other diseases. In some embodiments, a mutation is a non-synonymous mutation. The term “non-synonymous mutation” refers to a mutation, for example, a nucleotide substitution, which does result in an amino acid change such as an amino acid substitution in the translation product. A “frameshift” occurs when a mutation disrupts the normal phase of a gene's codon periodicity (also known as “reading frame”), resulting in the translation of a non-native protein sequence. It is possible for different mutations in a gene to achieve the same altered reading frame.

A “conservative” amino acid substitution is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate peptide function are well-known in the art.

As used herein, the term “affinity” refers to a measure of the strength of binding between two members of a binding pair, for example, an HLA-binding peptide and a class I or II HLA. K D is the dissociation constant and has units of molarity. The affinity constant is the inverse of the dissociation constant. An affinity constant is sometimes used as a generic term to describe this chemical entity. It is a direct measure of the energy of binding. Affinity may be determined experimentally, for example by surface plasmon resonance (SPR) using commercially available Biacore SPR units. Affinity may also be expressed as the inhibitory concentration 50 (IC₅₀), that concentration at which 50% of the peptide is displaced. Likewise, ln(IC₅₀) refers to the natural log of the IC₅₀. K_{off} refers to the off-rate constant, for example, for dissociation of an HLA-binding peptide and a class I or II HLA. Throughout this disclosure, “binding data” results can be expressed in terms of “IC₅₀.” IC₅₀ is the concentration of the tested peptide in a binding assay at which 50% inhibition of binding of a labeled reference peptide is observed. Given the conditions in which the assays are run (i.e., limiting HLA protein and labeled reference peptide concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC₅₀, relative to the IC₅₀ of a reference standard peptide. Binding can also be determined using other assay systems including those using live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)). “Cross-reactive binding” indicates that a peptide is bound by more than one HLA molecule; a synonym is degenerate binding.

The term “derived” and its grammatical equivalents when used to discuss an epitope is a synonym for “prepared” and its grammatical equivalents. A derived epitope can be isolated from a natural source, or it can be synthesized according to standard protocols in the art. Synthetic epitopes can comprise artificial amino acid residues “amino acid mimetics,” such as D isomers of natural occurring L amino acid residues or non-natural amino acid residues such as cyclohexylalanine. A derived or prepared epitope can be an analog of a native epitope.

A “native” or a “wild type” sequence refers to a sequence found in nature. Such a sequence can comprise a longer sequence in nature.

A “receptor” is to be understood as meaning a biological molecule or a molecule grouping capable of binding a ligand. A receptor may serve, to transmit information in a cell, a cell formation or an organism. The receptor comprises at least one receptor unit, for example, where each receptor unit may consist of a protein molecule. The receptor has a structure which complements that of a ligand and may complex the ligand as a binding partner. The information is transmitted in particular by conformational changes of the receptor following complexation of the ligand on the surface of a cell. In some embodiments, a receptor is to be understood as meaning in particular proteins of MHC classes I and II capable of forming a receptor/ligand complex with a ligand, in particular a peptide or peptide fragment of suitable length.

A “ligand” is to be understood as meaning a molecule which has a structure complementary to that of a receptor and is capable of forming a complex with this receptor. In some embodiments, a ligand is to be understood as meaning a peptide or peptide fragment which has a suitable length and suitable binding motifs in its amino acid sequence, so that the peptide or peptide fragment is capable of forming a complex with proteins of MHC class I or MHC class II.

In some embodiments, a “receptor/ligand complex” is also to be understood as meaning a “receptor/peptide complex” or “receptor/peptide fragment complex”, including a peptide- or peptide fragment-presenting MHC molecule of class I or of class II.

“Synthetic peptide” refers to a peptide that is obtained from a non-natural source, e.g., is man-made. Such peptides can be produced using such methods as chemical synthesis or recombinant DNA technology. “Synthetic peptides” include “fusion proteins.”

The term “motif” refers to a pattern of residues in an amino acid sequence of defined length, for example, a peptide of less than about 15 amino acid residues in length, or less than about 13 amino acid residues in length, for example, from about 8 to about 13 amino acid residues (e.g., 8, 9, 10, 11, 12, or 13) for a class I HLA motif and from about 6 to about 25 amino acid residues (e.g., 6, 7, 8, 9, 10, 11, 12, 13, 14, 15, 16, 17, 18, 19, 20, 21, 22, 23, 24, or 25) for a class II HLA motif, which is recognized by a particular HLA molecule. Motifs are typically different for each HLA protein encoded by a given human HLA allele. These motifs differ in their pattern of the primary and secondary anchor residues. In some embodiments, an MHC class I motif identifies a peptide of 9, 10, or 11 amino acid residues in length.

The term “naturally occurring” and its grammatical equivalents as used herein refer to the fact that an object can be found in nature. For example, a peptide or nucleic acid that is present in an organism (including viruses) and can be isolated from a source in nature and which has not been intentionally modified by man in the laboratory is naturally occurring.

According to the present disclosure, the term “vaccine” relates to a pharmaceutical preparation (pharmaceutical composition) or product that upon administration induces an immune response, for example, a cellular or humoral immune response, which recognizes and attacks a pathogen or a diseased cell such as a cancer cell. A vaccine may be used for the prevention or treatment of a disease. The term “individualized cancer vaccine” or “personalized cancer vaccine” concerns a particular cancer patient and means that a cancer vaccine is adapted to the needs or special circumstances of an individual cancer patient.

A “protective immune response” or “therapeutic immune response” refers to a CTL and/or an HTL response to an antigen derived from an pathogenic antigen (e.g., a tumor antigen), which in some way prevents or at least partially arrests disease symptoms, side effects or progression. The immune response can also include an antibody response which has been facilitated by the stimulation of helper T cells.

“Antigen processing” or “processing” and its grammatical equivalents refers to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, for example, antigen presenting cells, to specific T cells.

“Antigen presenting cells” (APC) are cells which present peptide fragments of protein antigens in association with MHC molecules on their cell surface. Some APCs may activate antigen specific T cells. Professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen presenting cell. An additional co-stimulatory signal is then produced by the antigen presenting cell, leading to activation of the T cell. The expression of co-stimulatory molecules is a defining feature of professional antigen-presenting cells. The main types of professional antigen-presenting cells are dendritic cells, which have the broadest range of antigen presentation, and are probably the most important antigen presenting cells, macrophages, B-cells, and certain activated epithelial cells. Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes. However, this nomenclature should not be construed to exclude all possible intermediate stages of differentiation. Immature dendritic cells are characterized as antigen presenting cells with a high capacity for antigen uptake and processing, which correlates with the high expression of Fc receptor (FcR) and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1 BB).

The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci. U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can have 50%, 60%, 70%, 75%, 80%, 85%, 90%, 98%, 99% or 100% sequence identity to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure. In some embodiments, an “isolated polynucleotide” encompasses a PCR or quantitative PCR reaction comprising the polynucleotide amplified in the PCR or quantitative PCR reaction.

The term “isolated”, “biologically pure” or their grammatical equivalents refers to material which is substantially or essentially free from components which normally accompany the material as it is found in its native state. Thus, isolated peptides described herein do not contain some or all of the materials normally associated with the peptides in their in-situ environment. An “isolated” epitope refers to an epitope that does not include the whole sequence of the antigen from which the epitope was derived. Typically, the “isolated” epitope does not have attached thereto additional amino acid residues that result in a sequence that has 100% identity over the entire length of a native sequence. The native sequence can be a sequence such as a tumor-associated antigen from which the epitope is derived. Thus, the term “isolated” means that the material is removed from its original environment (e.g., the natural environment if it is naturally occurring). An “isolated” nucleic acid is a nucleic acid removed from its natural environment. For example, a naturally occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. Isolated RNA molecules include in vivo or in vitro RNA transcripts of the DNA molecules described herein, and further include such molecules produced synthetically.

The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

The term “substantially pure” as used herein refers to material which is at least 50% pure (i.e., free from contaminants), at least 90% pure, at least 95% pure, at least 98% pure, or at least 99% pure.

The terms “polynucleotide”, “nucleotide”, “nucleic acid”, “polynucleic acid” or “oligonucleotide” and their grammatical equivalents are used interchangeably herein and refer to polymers of nucleotides of any length, and include DNA and RNA, for example, mRNA. Thus, these terms include double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein may be introduced into a cell by, for example, transfection, transformation, or transduction. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered using the methods of the present disclosure is mRNA.

“Transfection,” “transformation,” or “transduction” as used herein refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder; and (2) prophylactic or preventative measures that prevent or slow the development of a targeted pathologic condition or disorder. Thus, those in need of treatment include those already with the disorder; those prone to have the disorder; and those in whom the disorder is to be prevented.

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

Unless otherwise stated, the term “TCR” should be understood to encompass full TCRs as well as antigen-binding portions or antigen-binding fragments (also called MHC-peptide binding fragments) thereof. In some embodiments, the TCR is an intact or full-length TCR. In some embodiments, the TCR is an antigen-binding portion that is less than a full-length TCR but that binds to a specific antigenic peptide bound to (i.e., in the context of) an MHC molecule, i.e., an MHC-peptide complex. In some cases, an antigen-binding portion or fragment of a TCR contains only a portion of the structural domains of a full-length or intact TCR, but yet is able to bind the epitope (e.g., MHC-peptide complex) to which the full TCR binds. In some cases, an antigen-binding portion or fragment of a TCR contains the variable domains of a TCR, such as variable α chain and variable β chain of a TCR, sufficient to form a binding site for a MHC-peptide complex, for example, each chain can contain three complementarity determining regions.

Some, if not all cancers have antigens that are potential targets for immunotherapy. Each peptide antigen may be presented for T cell activation on an antigen presenting cells in association with a specific HLA-encoded MHC molecule. On the other hand, provided herein is a potentially universal approach, where particular epitopes are pre-identified and pre-validated for particular HLAs, and these epitopes can be pre-manufactured for a cell therapy manufacturing process. For example, a number of KRAS epitopes with G12, G13 and Q61 mutations can be identified using a reliable T cell epitope presentation prediction model (see, e.g., PCT/US2018/017849, filed Feb. 12, 2018, and PCT/US2019/068084 filed Dec. 20, 2019, each of which are incorporated by reference in their entirety), with validation of immunogenicity of these epitopes, processing and presentation using mass spectrometry of these epitopes, and ability to generate cytotoxic T cells with TCRs against these epitopes and MHCs encoded by different HLAs. Each epitope is validated with its specific amino acid sequence and relevant HLA. Once these epitopes are validated, a library can be created containing pre-manufactured immunogens, such as peptides containing the epitopes or RNA encoding peptides containing these epitopes.

In some embodiments, antigenic peptides comprising an epitope may be identified by mass spectrometry to be bound to an MHC encoded by an HLA. In some embodiments, the antigenic peptide may further be identified as having immunogenic potential ex vivo, wherein, an antigen presenting cell expressing the MHC encoded by the HLA is loaded with a peptide comprising the epitope sequence and is contacted to a T cell ex vivo, and the T cell exhibits activation signals, such as cytokine generation and cytotoxicity.

Provided herein are KRAS epitopes each of which specifically can bind to the MHC protein encoded by the alleles indicated in right hand column in the same row of Table 1, that have been identified to bind to the MHC protein by mass spectrometry, and that each epitope is presented to T cells by association with the MHC protein in the respective right hand column for each row in Table 1. Provided herein are specific KRAS epitopes, each of which can specifically bind to a specific MHC encoded by an allele as indicated in Table 1, and is predetermined to be immunogenic by a suitable ex vivo validation assay. In one embodiment, the KRAS epitope has an amino acid sequence GACGVGKSA, and the epitope specifically binds to an MHC encoded by an HLA-C03:04 allele. In some embodiments, the KRAS epitope has an amino acid sequence GAVGVGKSA, and the epitope specifically binds to an MHC encoded by an HLA-C03:03 allele (Table 1).

TABLE 1

| Epitope | MHC protein encoded by allele |
|---|---|
| GACGVGKSA | C03: 04 |
| GAVGVGKSA | C03: 03 |

In some embodiments, provided herein is a method for preparing antigen-specific T cells, the method comprising contacting T cells from the subject or allogeneic T cells with APCs comprising one or more peptides containing an epitope with a sequence GACGVGKSA, wherein the APCs express a protein encoded by an HLA-C03:04 allele. In some embodiments, a method of treating a subject with cancer is provided herein, the method comprising administering to the subject, a therapy comprising: a peptide, a polynucleotide encoding the peptide, APCs comprising the peptide or polynucleotide, or T cells stimulated with the APCs; wherein the peptide comprises an epitope with a sequence GACGVGKSA; and wherein the subject expresses a protein encoded by an HLA-C03:04 allele.

In some embodiments, provided herein is a method for preparing antigen-specific T cells, the method comprising contacting T cells from the subject or allogeneic T cells with APCs comprising one or more peptides containing an epitope with a sequence GAVGVGKSA, wherein the APCs express a protein encoded by an HLA-C03:03 allele. In some embodiments, a method of treating a subject with cancer is provided herein, the method comprising administering to the subject, a therapy comprising: a peptide, a polynucleotide encoding the peptide, APCs comprising the peptide or polynucleotide, or T cells stimulated with the APCs; wherein the peptide comprises an epitope with a sequence GAVGVGKSA; and wherein the subject expresses a protein encoded by an HLA-C03:03 allele.

In some embodiments, the method described above comprising a peptide comprising an epitope of Table 1, or a polynucleotide encoding the peptide may be combined with 1, 2, 3, 4, 5, 6, 7, 8, 9, 10, 11, 12, 13, 14, 15, 16, 17, 18, 19, 20 or more additional peptides comprising at least one epitope sequence selected from a library of epitope sequences, or polynucleotides encoding the same or APCs comprising the additional peptides or polynucleotides, or T cells stimulated with these APCs; and can be administered to the subject. In some embodiments, antigen presenting cells loaded with a peptide comprising an epitope of Table 1, or polynucleotide encoding the peptide, combined with antigen presenting cells loaded with 1, 2, 3, 4, 5, 6, 7, 8, 9, 10, 11, 12, 13, 14, 15, 16, 17, 18, 19, 20 or more additional peptides or nucleotides encoding the same may be administered to the subject. In some embodiments, T cells stimulated with antigen presenting cells loaded with a peptide comprising an epitope of Table 1, or polynucleotide encoding the peptide, combined with stimulated with antigen presenting cells loaded with 1, 2, 3, 4, 5, 6, 7, 8, 9, 10, 11, 12, 13, 14, 15, 16, 17, 18, 19, 20 or more additional peptides may be administered to the subject, in addition to the therapy described in the preceding paragraphs.

In some embodiments, the peptide or the additional peptide may be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more number of amino acid residues in length.

In some embodiments, any combination of therapy or therapeutic modalities (e.g., peptide, RNA, APC or T cells) may be used for the 1, 2, 3, 4, 5, 6, 7, 8, 9, 10, 11, 12, 13, 14, 15, 16, 17, 18, 19, 20 or more additional peptides comprising at least one epitope sequence selected from a library of epitope sequences, along with using a peptide comprising the RAS mutation, a polynucleotide encoding the peptide, APCs comprising the peptide or polynucleotide, or T cells stimulated with the APCs.

In some embodiments, in addition to any of the treatment compositions described above, the compositions may comprise an adjuvant.

In some embodiments, in addition to any of the treatment compositions described above, the compositions may comprise an additional therapy such as a blocker of checkpoint inhibition. Examples include anti-PD1 antibody nivolumab or pembrolizumab.

In some embodiments, in addition to any of the treatment compositions described above, the compositions may comprise an additional therapy such as a small molecule drug.

### RAS and Mutations in Cancer

KRAS protein is a GTPase, and it converts GTP into another molecule called GDP. In this way the KRAS protein acts like a switch that is turned on and off by the GTP and GDP molecules. To transmit signals, it must be turned on by attaching (binding) to a molecule of GTP. The KRAS protein is turned off (inactivated) when it converts the GTP to GDP. When the protein is bound to GDP, it does not relay signals to the cell's nucleus.

The KRAS gene belongs to a class of genes known as oncogenes. When mutated, oncogenes have the potential to cause normal cells to become cancerous. The KRAS gene is in the Ras family of oncogenes, which also includes two other genes: HRAS and NRAS. These proteins play important roles in cell division, cell differentiation, and apoptosis. KRAS gain-of-function mutations occur in approximately 30% of all human cancers, including more than 90 percent of pancreatic cancers, 35 to 45 percent of colorectal cancers and approximately 25 percent of lung cancers. Mutation of glycine 12 (G12) causes RAS activation by interfering with GAP binding and GAP-stimulated GTP hydrolysis. There are currently no effective treatment for KRAS-positive cancers.

In one aspect, provided herein is a therapeutic composition for a subject having a cancer with a G12C KRAS mutation, wherein the subject express a protein encoded by an HLA-C03:04, and the therapeutic comprises of at least one peptide comprising an epitope having a sequence GACGVGKSA, or a polynucleotide encoding the at least one peptide. In some embodiments, provided herein is a method comprising, identifying whether a subject having a cancer related to a KRAS mutation comprises a KRAS G12C mutation by sequencing a biological sample from the subject; identifying the subject expresses a protein encoded by an HLA-C03:04 allele; and administering to the subject a composition comprising at least a peptide comprising an epitope GACGVGKSA.

In another aspect, provided herein is a therapeutic composition for a subject having a cancer with a G12V KRAS mutation, wherein the subject express a protein encoded by an HLA-C03:03, and the therapeutic comprises of at least one peptide comprising a the epitope having a sequence GAVGVGKSA, or a polynucleotide encoding the at least one peptide. In some embodiments, provided herein is a method comprising, identifying whether a subject having a cancer related to a KRAS mutation comprises a KRAS G12V mutation by sequencing a biological sample from the subject; identifying the subject expresses a protein encoded by an HLA-C03:03 allele; and administering to the subject a composition comprising at least one peptide comprising an epitope GAVGVGKSA.

In some aspects, the present disclosure provides a composition comprising at least two peptides, a polynucleotide encoding the at least two peptides, APCs comprising the at least two peptides or the polynucleotide encoding the at least two peptides, or T cells stimulated with APCs comprising the at least two peptides or the polynucleotide encoding the at least two peptides. In some embodiments, the peptides comprise at least two distinct peptides. In some embodiments, the first peptide comprises a first epitope GACGVGKSA, and the second peptide comprises a second neoepitope, wherein the composition is administered to a subject that has a cancer with KRAS G12C mutation, and expresses a protein encoded by an HLA-C03:04 allele. In some embodiments, the first peptide comprises a first epitope GAVGVGKSA, and the second peptide comprises a second neoepitope, wherein the composition is administered to a subject that has a cancer with KRAS G12V mutation, and expresses a protein encoded by an HLA-C03:03 allele.

In some embodiments, the first and second peptides are derived from the same protein. The at least two distinct peptides may vary by length, amino acid sequence or both. The peptides can be derived from any protein known to or have been found to contain a tumor specific mutation. In some embodiments, the composition described herein comprises a first peptide or a polynucleotide encoding the first peptide, the first peptide comprising a first neoepitope of a protein and a second peptide or a polynucleotide encoding the second peptide, the second peptide comprising a second neoepitope of the same protein, wherein the first peptide is different from the second peptide, and wherein the first epitope comprises a mutation and the second epitope comprises the same mutation. In some embodiments, the composition described herein comprises a first peptide comprising a first epitope of a first region of a protein and a second peptide comprising a second epitope of a second region of the same protein, wherein the first region comprises at least one amino acid of the second region, wherein the first peptide is different from the second peptide and wherein the first epitope comprises a first mutation and the second epitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof. In some embodiments, 1, 2, 3, 4, 5, 6, 7, 8, 9, 10, 11, 12, 13, 14, 15, 16, 17, 18, 19, 20 or more additional peptides comprising at least one epitope sequence selected from a library of epitope sequences, or polynucleotides encoding the 1, 2, 3, 4, 5, 6, 7, 8, 9, 10, 11, 12, 13, 14, 15, 16, 17, 18, 19, 20 or more additional peptides.

In some embodiments, a peptide can be derived from a protein with a substitution mutation, e.g., the KRAS G12C, G12D, G12V, Q61H or Q61L mutation, or the NRAS Q61K or Q61R mutation, provided that said mutation(s) have also been identified to be expressed in the tumor of the subject. The substitution may be positioned anywhere along the length of the peptide. For example, it can be located in the N terminal third of the peptide, the central third of the peptide or the C terminal third of the peptide. In another embodiment, the substituted residue is located 2-5 residues away from the N terminal end or 2-5 residues away from the C terminal end. The peptides can similarly derived from tumor specific insertion mutations where the peptide comprises one or more, or all of the inserted residues. In some embodiments, the MHC epitope prediction program implemented on a computer is an in-house prediction program (described in WO2018148671 publication, WO2017184590 publication) or NetMHCpan. In some embodiments, the MHC epitope prediction program implemented on a computer is NetMHCpan version 4.0.

In some embodiments, a peptide comprising an epitope of Table 1, a polynucleotide encoding a peptide comprising an epitope of Table 1, APCs comprising an epitope of Table 1 or a polynucleotide encoding a peptide comprising an epitope of Table 1, or T cells stimulated with APCs comprising an epitope of Table 1 or a polynucleotide encoding a peptide comprising an epitope of Table 1, can be administered to a subject that expresses an MHC protein encoded by an HLA-C03:04 or HLA-C03:03 allele.

Exemplary RAS epitope sequences comprising a Q61H mutation, corresponding HLA allele, and rank binding potential are listed in Table 2 below. In some embodiments, a peptide comprising an epitope of Table 1, a polynucleotide encoding a peptide comprising an epitope of Table 1, APCs comprising an epitope of Table 1 or a polynucleotide encoding a peptide comprising an epitope of Table 1, or T cells stimulated with APCs comprising an epitope of Table 1 or a polynucleotide encoding a peptide comprising an epitope of Table 1, can be administered to a subject that expresses an MHC protein encoded by an HLA-C03:04 or HLA-C03:03 allele; and a peptide comprising an epitope of Table 2, a polynucleotide encoding a peptide comprising an epitope of Table 2, APCs comprising an epitope of Table 2 or a polynucleotide encoding a peptide comprising an epitope of Table 2, or T cells stimulated with APCs comprising an epitope of Table 2 or a polynucleotide encoding a peptide comprising an epitope of Table 2, can be administered to the subject, for example, if the subject expresses an MHC protein encoded by a corresponding HLA allele in Table 2 and contains a cancer with a RAS Q61H mutation.

TABLE 2

| Peptide | Allele | Rank of Binding Potential |
|---|---|---|
| ILDTAGHEEY | HLA-A36: 01 | 1 |
| ILDTAGHEEY | HLA-A01: 01 | 2 |
| DTAGHEEYSAM | HLA-A26: 01 | 3 |
| DTAGHEEYSAM | HLA-A25: 01 | 4 |
| GHEEYSAM | HLA-B15: 09 | 4 |
| DTAGHEEY | HLA-A26: 01 | 5 |
| ILDTAGHEE | HLA-C08: 02 | 5 |
| AGHEEYSAM | HLA-C01: 02 | 6 |
| AGHEEYSAM | HLA-B46: 01 | 6 |
| DTAGHEEY | HLA-A25: 01 | 6 |
| DTAGHEEY | HLA-A01: 01 | 6 |
| DTAGHEEY | HLA-B18: 01 | 7 |
| DTAGHEEY | HLA-A36: 01 | 7 |
| ILDTAGHEE | HLA-C05: 01 | 7 |
| ILDTAGHEE | HLA-A02: 07 | 7 |
| ILDTAGHEEY | HLA-A29: 02 | 7 |
| ILDTAGHEEY | HLA-C08: 02 | 7 |
| HEEYSAMRD | HLA-B49: 01 | 8 |
| TAGHEEYSA | HLA-B35: 03 | 8 |
| DTAGHEEYS | HLA-A68: 02 | 9 |
| DTAGHEEYSAMR | HLA-A68: 01 | 9 |
| GHEEYSAM | HLA-B39: 01 | 9 |
| ILDTAGHEE | HLA-A01: 01 | 9 |
| LDTAGHEEY | HLA-B53: 01 | 9 |
| HEEYSAMRD | HLA-B41: 01 | 10 |
| ILDTAGHEE | HLA-A36: 01 | 10 |
| DTAGHEEY | HLA-B58: 01 | 11 |
| LLDILDTAGH | HLA-A01: 01 | 12 |
| TAGHEEYSAM | HLA-B35: 03 | 12 |
| LDTAGHEEY | HLA-B35: 01 | 13 |
| DILDTAGHE | HLA-A26: 01 | 14 |
| DTAGHEEY | HLA-C12: 03 | 14 |
| ILDTAGHEEY | HLA-C05: 01 | 14 |
| AGHEEYSAM | HLA-A30: 02 | 15 |
| DILDTAGHEEY | HLA-A25: 01 | 15 |
| DTAGHEEY | HLA-C02: 02 | 15 |
| ILDTAGHEE | HLA-C04: 01 | 15 |
| DILDTAGH | HLA-A26: 01 | 16 |
| ILDTAGHEE | HLA-A02: 01 | 16 |
| LDTAGHEEY | HLA-A29: 02 | 16 |
| ILDTAGHE | HLA-A01: 01 | 17 |
| LDTAGHEEY | HLA-B18: 01 | 17 |
| AGHEEYSAM | HLA-C14: 03 | 18 |
| DILDTAGHEEY | HLA-A29: 02 | 18 |
| DTAGHEEYS | HLA-A26: 01 | 18 |
| ILDTAGHEEY | HLA-B15: 01 | 18 |
| DTAGHEEYSA | HLA-A68: 02 | 19 |
| ILDTAGHE | HLA-C05: 01 | 19 |
| ILDTAGHEEY | HLA-A02: 07 | 19 |
| ILDTAGHEEY | HLA-A30: 02 | 19 |
| LDTAGHEEY | HLA-A36: 01 | 19 |
| AGHEEYSAM | HLA-C14: 02 | 20 |
| AGHEEYSAM | HLA-B15: 03 | 20 |
| LLDILDTAGH | HLA-A02: 07 | 20 |

Exemplary RAS epitope sequences comprising a Q61R mutation, corresponding HLA allele, and rank binding potential are listed in Table 3 below. In some embodiments, a peptide comprising an epitope of Table 1, a polynucleotide encoding a peptide comprising an epitope of Table 1, APCs comprising an epitope of Table 1 or a polynucleotide encoding a peptide comprising an epitope of Table 1, or T cells stimulated with APCs comprising an epitope of Table 1 or a polynucleotide encoding a peptide comprising an epitope of Table 1, can be administered to a subject that expresses an MHC protein encoded by an HLA-C03:04 or HLA-C03:03 allele; and a peptide comprising an epitope of Table 3, a polynucleotide encoding a peptide comprising an epitope of Table 3, APCs comprising an epitope of Table 3 or a polynucleotide encoding a peptide comprising an epitope of Table 3, or T cells stimulated with APCs comprising an epitope of Table 3 or a polynucleotide encoding a peptide comprising an epitope of Table 3, can be administered to the subject, for example, if the subject expresses an MHC protein encoded by a corresponding HLA allele in Table 3 and contains a cancer with a RAS Q61R mutation.

TABLE 3

| Peptide | Allele | Rank of Binding Potential |
|---|---|---|
| ILDTAGREEY | HLA-A36: 01 | 1 |
| ILDTAGREEY | HLA-A01: 01 | 2 |
| DTAGREEYSAM | HLA-A26: 01 | 3 |
| DILDTAGR | HLA-A33: 03 | 4 |
| DILDTAGR | HLA-A68: 01 | 5 |
| DTAGREEY | HLA-A26: 01 | 6 |
| DTAGREEYSAM | HLA-A25: 01 | 6 |
| CLLDILDTAGR | HLA-A74: 01 | 7 |
| DTAGREEY | HLA-A01: 01 | 7 |
| REEYSAMRD | HLA-B41: 01 | 7 |
| GREEYSAMR | HLA-B27: 05 | 8 |
| ILDTAGREE | HLA-C08: 02 | 8 |
| ILDTAGREEY | HLA-A29: 02 | 8 |
| REEYSAMRD | HLA-B49: 01 | 8 |
| AGREEYSAM | HLA-B46: 01 | 9 |
| DTAGREEY | HLA-B18: 01 | 9 |
| DTAGREEY | HLA-A25: 01 | 9 |
| DTAGREEY | HLA-A36: 01 | 9 |
| DILDTAGR | HLA-A74: 01 | 10 |
| DILDTAGRE | HLA-A26: 01 | 10 |
| ILDTAGREE | HLA-C05: 01 | 10 |
| DILDTAGR | HLA-A26: 01 | 11 |
| GREEYSAM | HLA-B39: 01 | 11 |
| AGREEYSAM | HLA-B15: 03 | 12 |
| GREEYSAM | HLA-C07: 02 | 12 |
| ILDTAGREE | HLA-A01: 01 | 12 |
| TAGREEYSA | HLA-B35: 03 | 12 |
| ILDTAGREEY | HLA-A30: 02 | 13 |
| DTAGREEYS | HLA-A68: 02 | 14 |
| ILDTAGRE | HLA-A01: 01 | 14 |
| CLLDILDTAGR | HLA-A31: 01 | 15 |
| DTAGREEYSAMR | HLA-A68: 01 | 15 |
| LLDILDTAGR | HLA-A01: 01 | 15 |
| DTAGREEY | HLA-B58: 01 | 16 |
| ILDTAGREEY | HLA-C08: 02 | 16 |
| DILDTAGR | HLA-A31: 01 | 17 |
| ILDTAGREE | HLA-C04: 01 | 17 |
| ILDTAGREEY | HLA-A32: 01 | 17 |
| LLDILDTAGR | HLA-A74: 01 | 17 |
| TAGREEYSAM | HLA-B35: 03 | 17 |
| DILDTAGREEY | HLA-A32: 01 | 18 |
| ILDTAGRE | HLA-C05: 01 | 18 |
| ILDTAGREE | HLA-A02: 07 | 18 |
| REEYSAMRD | HLA-B40: 01 | 18 |
| AGREEYSAM | HLA-B15: 01 | 19 |
| AGREEYSAMR | HLA-A31: 01 | 19 |
| ILDTAGRE | HLA-A36: 01 | 19 |
| LDILDTAGR | HLA-A68: 01 | 19 |
| LDTAGREEY | HLA-A29: 02 | 19 |
| LDTAGREEY | HLA-B35: 01 | 19 |
| REEYSAMRD | HLA-B45: 01 | 19 |
| REEYSAMRDQY | HLA-A36: 01 | 19 |
| DTAGREEY | HLA-C02: 02 | 20 |

Exemplary RAS epitope sequences comprising a Q61K mutation, corresponding HLA allele, and rank binding potential are listed in Table 4 below. In some embodiments, a peptide comprising an epitope of Table 1, a polynucleotide encoding a peptide comprising an epitope of Table 1, APCs comprising an epitope of Table 1 or a polynucleotide encoding a peptide comprising an epitope of Table 1, or T cells stimulated with APCs comprising an epitope of Table 1 or a polynucleotide encoding a peptide comprising an epitope of Table 1, can be administered to a subject that expresses an MHC protein encoded by an HLA- or HLA-C03:03 allele; and a peptide comprising an epitope of Table 4, a polynucleotide encoding a peptide comprising an epitope of Table 4, APCs comprising an epitope of Table 4 or a polynucleotide encoding a peptide comprising an epitope of Table 4, or T cells stimulated with APCs comprising an epitope of Table 4 or a polynucleotide encoding a peptide comprising an epitope of Table 4, can be administered to the subject, for example, if the subject expresses an MHC protein encoded by a corresponding HLA allele in Table 4 and contains a cancer with a RAS Q61K mutation.

TABLE 4

| Peptide | Allele | Rank of Binding Potential |
|---|---|---|
| ILDTAGKEEY | HLA-A36: 01 | 1 |
| ILDTAGKEEY | HLA-A01: 01 | 2 |
| DTAGKEEYSAM | HLA-A26: 01 | 3 |
| CLLDILDTAGK | HLA-A03: 01 | 4 |
| DTAGKEEY | HLA-A01: 01 | 5 |
| DTAGKEEY | HLA-A26: 01 | 5 |
| DTAGKEEYSAM | HLA-A25: 01 | 5 |
| AGKEEYSAM | HLA-B46: 01 | 6 |
| DILDTAGKE | HLA-A26: 01 | 7 |
| KEEYSAMRD | HLA-B41: 01 | 7 |
| DTAGKEEY | HLA-B18: 01 | 8 |
| GKEEYSAM | HLA-B15: 03 | 8 |
| ILDTAGKEE | HLA-C08: 02 | 8 |
| ILDTAGKEEY | HLA-A29: 02 | 8 |
| DTAGKEEYS | HLA-A68: 02 | 9 |
| LDTAGKEEY | HLA-B53: 01 | 9 |
| TAGKEEYSA | HLA-B35: 03 | 9 |
| DILDTAGK | HLA-A68: 01 | 10 |
| DTAGKEEY | HLA-A36: 01 | 10 |
| KEEYSAMRD | HLA-B49: 01 | 10 |
| LDTAGKEEY | HLA-C07: 01 | 10 |
| DTAGKEEYSAMR | HLA-A68: 01 | 11 |
| ILDTAGKEE | HLA-C05: 01 | 11 |
| ILDTAGKEEY | HLA-C08: 02 | 11 |
| LLDILDTAGK | HLA-A01: 01 | 12 |
| AGKEEYSAM | HLA-A30: 02 | 13 |
| DTAGKEEY | HLA-A25: 01 | 13 |
| DTAGKEEYS | HLA-A26: 01 | 13 |
| ILDTAGKE | HLA-C05: 01 | 13 |
| LDTAGKEEY | HLA-B35: 01 | 13 |
| AGKEEYSAMR | HLA-A31: 01 | 14 |
| DILDTAGK | HLA-A33: 03 | 14 |
| ILDTAGKE | HLA-A01: 01 | 14 |
| ILDTAGKEE | HLA-A01: 01 | 14 |
| ILDTAGKEE | HLA-A02: 07 | 14 |
| TAGKEEYSAM | HLA-B35: 03 | 14 |
| AGKEEYSAM | HLA-B15: 01 | 15 |
| ILDTAGKEEY | HLA-A30: 02 | 15 |
| LDTAGKEEY | HLA-B46: 01 | 15 |
| DTAGKEEY | HLA-B58: 01 | 16 |
| ILDTAGKEEY | HLA-C05: 01 | 17 |
| AGKEEYSAM | HLA-A30: 01 | 18 |
| AGKEEYSAM | HLA-B15: 03 | 18 |
| DTAGKEEY | HLA-C02: 02 | 18 |
| LDTAGKEEY | HLA-A29: 02 | 18 |

Exemplary RAS epitope sequences comprising a Q61L mutation, corresponding HLA allele, and rank binding potential are listed in Table 5 below. In some embodiments, a peptide comprising an epitope of Table 1, a polynucleotide encoding a peptide comprising an epitope of Table 1, APCs comprising an epitope of Table 1 or a polynucleotide encoding a peptide comprising an epitope of Table 1, or T cells stimulated with APCs comprising an epitope of Table 1 or a polynucleotide encoding a peptide comprising an epitope of Table 1, can be administered to a subject that expresses an MHC protein encoded by an HLA-C03:04 or HLA-C03:03 allele; and a peptide comprising an epitope of Table 5, a polynucleotide encoding a peptide comprising an epitope of Table 5, APCs comprising an epitope of Table 5 or a polynucleotide encoding a peptide comprising an epitope of Table 5, or T cells stimulated with APCs comprising an epitope of Table 5 or a polynucleotide encoding a peptide comprising an epitope of Table 5, can be administered to the subject, for example, if the subject expresses an MHC protein encoded by a corresponding HLA allele in Table 5 and contains a cancer with a RAS Q61L mutation.

TABLE 5

| Peptide | Allele | Rank of Binding Potential |
|---|---|---|
| ILDTAGLEEY | HLA-A36: 01 | 1 |
| ILDTAGLEEY | HLA-A01: 01 | 2 |
| LLDILDTAGL | HLA-A02: 07 | 3 |
| GLEEYSAMRDQY | HLA-A36: 01 | 4 |
| DTAGLEEY | HLA-A25: 01 | 5 |
| DTAGLEEY | HLA-A26: 01 | 5 |
| DTAGLEEYSAM | HLA-A26: 01 | 5 |
| DTAGLEEY | HLA-A01: 01 | 6 |
| ILDTAGLEE | HLA-C08: 02 | 6 |
| ILDTAGLEE | HLA-A01: 01 | 6 |
| CLLDILDTAGL | HLA-A02: 04 | 7 |
| ILDTAGLEE | HLA-A36: 01 | 7 |
| LLDILDTAGL | HLA-A01: 01 | 7 |
| DILDTAGL | HLA-B14: 02 | 8 |
| DILDTAGLEEY | HLA-A25: 01 | 8 |
| DTAGLEEYS | HLA-A68: 02 | 8 |
| DTAGLEEYSAM | HLA-A25: 01 | 8 |
| GLEEYSAMR | HLA-A74: 01 | 8 |
| ILDTAGLE | HLA-A01: 01 | 8 |
| DILDTAGLEEY | HLA-A26: 01 | 9 |
| DTAGLEEY | HLA-A36: 01 | 9 |
| ILDTAGLEEY | HLA-A29: 02 | 9 |
| DILDTAGL | HLA-B08: 01 | 10 |
| DTAGLEEY | HLA-B18: 01 | 10 |
| ILDTAGLEE | HLA-A02: 07 | 10 |
| LDTAGLEEY | HLA-B35: 01 | 10 |
| CLLDILDTAGL | HLA-A02: 01 | 11 |
| DTAGLEEY | HLA-C02: 02 | 11 |
| ILDTAGLEE | HLA-C05: 01 | 11 |
| ILDTAGLEEY | HLA-C08: 02 | 11 |
| ILDTAGLEEY | HLA-A02: 07 | 11 |
| LLDILDTAGL | HLA-C08: 02 | 11 |
| DILDTAGL | HLA-A26: 01 | 12 |
| LDTAGLEEY | HLA-B53: 01 | 12 |
| DTAGLEEY | HLA-C03: 02 | 13 |
| DTAGLEEY | HLA-B58: 01 | 13 |
| ILDTAGLEEY | HLA-A30: 02 | 13 |
| LLDILDTAGL | HLA-C05: 01 | 13 |
| LLDILDTAGL | HLA-C04: 01 | 13 |
| DTAGLEEYSAMR | HLA-A68: 01 | 14 |
| ILDTAGLE | HLA-A36: 01 | 15 |
| LLDILDTAGL | HLA-A02: 01 | 15 |
| AGLEEYSAM | HLA-B15: 03 | 16 |
| DTAGLEEYSA | HLA-A68: 02 | 16 |
| GLEEYSAMRDQY | HLA-A01: 01 | 16 |
| ILDTAGLE | HLA-C04: 01 | 16 |
| ILDTAGLEEY | HLA-B15: 01 | 16 |
| LDILDTAGL | HLA-B37: 01 | 16 |
| AGLEEYSAM | HLA-A30: 02 | 17 |
| AGLEEYSAM | HLA-B48: 01 | 17 |
| AGLEEYSAMR | HLA-A31: 01 | 17 |
| ILDTAGLEE | HLA-C04: 01 | 17 |
| LDTAGLEEY | HLA-C03: 02 | 17 |
| AGLEEYSAM | HLA-C14: 02 | 18 |
| GLEEYSAMR | HLA-A31: 01 | 18 |
| LEEYSAMRD | HLA-B41: 01 | 18 |
| LLDILDTAGLE | HLA-A01: 01 | 18 |
| AGLEEYSAM | HLA-C14: 03 | 19 |
| LDILDTAGL | HLA-B40: 02 | 19 |
| LDTAGLEEY | HLA-A29: 02 | 19 |
| DILDTAGLE | HLA-A26: 01 | 20 |
| DTAGLEEY | HLA-B15: 01 | 20 |
| ILDTAGLEEY | HLA-A02: 01 | 20 |
| LDTAGLEEY | HLA-A36: 01 | 20 |
| LDTAGLEEY | HLA-B46: 01 | 20 |
| DTAGLEEY | HLA-A68: 02 | 21 |
| DTAGLEEY | HLA-C12: 03 | 21 |
| ILDTAGLE | HLA-C05: 01 | 21 |
| LDTAGLEEY | HLA-B18: 01 | 21 |
| LEEYSAMRD | HLA-B49: 01 | 21 |
| TAGLEEYSA | HLA-B54: 01 | 21 |
| DILDTAGLEEY | HLA-A29: 02 | 22 |
| GLEEYSAM | HLA-C05: 01 | 22 |

Exemplary RAS epitope sequences comprising a G12C mutation, corresponding HLA allele, and rank binding potential are listed in Table 6 below. In some embodiments, a peptide comprising an epitope of Table 1, a polynucleotide encoding a peptide comprising an epitope of Table 1, APCs comprising an epitope of Table 1 or a polynucleotide encoding a peptide comprising an epitope of Table 1, or T cells stimulated with APCs comprising an epitope of Table 1 or a polynucleotide encoding a peptide comprising an epitope of Table 1, can be administered to a subject that expresses an MHC protein encoded by an HLA-C03:04 or HLA-C03:03 allele; and a peptide comprising an epitope of Table 6, a polynucleotide encoding a peptide comprising an epitope of Table 6, APCs comprising an epitope of Table 6 or a polynucleotide encoding a peptide comprising an epitope of Table 6, or T cells stimulated with APCs comprising an epitope of Table 6 or a polynucleotide encoding a peptide comprising an epitope of Table 6, can be administered to the subject, for example, if the subject expresses an MHC protein encoded by a corresponding HLA allele in Table 6 and contains a cancer with a RAS G12C mutation.

TABLE 6

| Peptide | Allele | Rank of Binding Potential |
|---|---|---|
| VVVGACGVGK | HLA-A11: 01 | 1 |
| VVGACGVGK | HLA-A03: 01 | 2 |
| VVGACGVGK | HLA-A11: 01 | 3 |
| VVVGACGVGK | HLA-A68: 01 | 4 |
| VVGACGVGK | HLA-A68: 01 | 5 |
| VVVGACGVGK | HLA-A03: 01 | 5 |
| VVGACGVGK | HLA-A30: 01 | 6 |
| ACGVGKSAL | HLA-B81: 01 | 7 |
| ACGVGKSAL | HLA-C01: 02 | 7 |
| ACGVGKSAL | HLA-C14: 03 | 8 |
| ACGVGKSAL | HLA-C03: 04 | 9 |
| VVVGACGVGK | HLA-A30: 01 | 9 |
| ACGVGKSAL | HLA-C14: 02 | 10 |
| CGVGKSAL | HLA-B08: 01 | 10 |
| KLVVVGACGV | HLA-A02: 01 | 10 |
| ACGVGKSAL | HLA-B07: 02 | 11 |
| GACGVGKSAL | HLA-B48: 01 | 12 |
| GACGVGKSAL | HLA-C03: 03 | 13 |
| ACGVGKSAL | HLA-B48: 01 | 14 |
| ACGVGKSAL | HLA-B40: 01 | 14 |
| YKLVVVGAC | HLA-B48: 01 | 14 |
| YKLVVVGAC | HLA-B15: 03 | 14 |
| GACGVGKSA | HLA-B46: 01 | 15 |
| GACGVGKSAL | HLA-C03: 04 | 15 |
| GACGVGKSAL | HLA-C01: 02 | 15 |
| LVVVGACGV | HLA-A68: 02 | 15 |
| CGVGKSAL | HLA-C03: 04 | 16 |
| GACGVGKSAL | HLA-C08: 02 | 16 |
| VVGACGVGK | HLA-A74: 01 | 16 |

Exemplary RAS epitope sequences comprising a G12V mutation, corresponding HLA allele, and rank binding potential are listed in Table 7 below. In some embodiments, a peptide comprising an epitope of Table 1, a polynucleotide encoding a peptide comprising an epitope of Table 1, APCs comprising an epitope of Table 1 or a polynucleotide encoding a peptide comprising an epitope of Table 1, or T cells stimulated with APCs comprising an epitope of Table 1 or a polynucleotide encoding a peptide comprising an epitope of Table 1, can be administered to a subject that expresses an MHC protein encoded by an HLA- or HLA-C03:03 allele; and a peptide comprising an epitope of Table 7, a polynucleotide encoding a peptide comprising an epitope of Table 7, APCs comprising an epitope of Table 7 or a polynucleotide encoding a peptide comprising an epitope of Table 7, or T cells stimulated with APCs comprising an epitope of Table 7 or a polynucleotide encoding a peptide comprising an epitope of Table 7, can be administered to the subject, for example, if the subject expresses an MHC protein encoded by a corresponding HLA allele in Table 7 and contains a cancer with a RAS G12V mutation.

TABLE 7

| Peptide | Allele | Rank of Binding Potential |
|---|---|---|
| VVGAVGVGK | HLA-A03: 01 | 1 |
| VVGAVGVGK | HLA-A11: 01 | 2 |
| VVVGAVGVGK | HLA-A11: 01 | 2 |
| VVVGAVGVGK | HLA-A68: 01 | 3 |
| VVGAVGVGK | HLA-A68: 01 | 4 |
| LVVVGAVGV | HLA-A68: 02 | 5 |
| VVGAVGVGK | HLA-A30: 01 | 5 |
| AVGVGKSAL | HLA-B81: 01 | 6 |
| KLVVVGAVGV | HLA-A02: 01 | 6 |
| AVGVGKSAL | HLA-B46: 01 | 7 |
| GAVGVGKSAL | HLA-C03: 03 | 7 |
| GAVGVGKSAL | HLA-B48: 01 | 7 |
| VVVGAVGVGK | HLA-A03: 01 | 7 |
| AVGVGKSAL | HLA-C03: 04 | 8 |
| GAVGVGKSAL | HLA-C03: 04 | 8 |
| KLVVVGAVGV | HLA-A02: 07 | 9 |
| VGVGKSAL | HLA-B08: 01 | 9 |
| VVVGAVGV | HLA-A68: 02 | 9 |
| AVGVGKSAL | HLA-C08: 02 | 10 |
| AVGVGKSAL | HLA-B07: 02 | 10 |
| GAVGVGKSAL | HLA-B35: 03 | 10 |
| AVGVGKSAL | HLA-C08: 01 | 11 |
| AVGVGKSAL | HLA-C01: 02 | 11 |
| GAVGVGKSA | HLA-B55: 01 | 11 |
| GAVGVGKSAL | HLA-B81: 01 | 11 |
| GAVGVGKSAL | HLA-C08: 01 | 11 |
| KLVVVGAVGV | HLA-B13: 02 | 11 |
| VGVGKSAL | HLA-C03: 04 | 11 |
| AVGVGKSAL | HLA-A32: 01 | 12 |
| GAVGVGKSA | HLA-B46: 01 | 12 |
| VGVGKSAL | HLA-C03: 02 | 12 |
| VGVGKSALTI | HLA-A23: 01 | 12 |
| GAVGVGKSA | HLA-B54: 01 | 13 |
| VGVGKSAL | HLA-C01: 02 | 3 |
| AVGVGKSAL | HLA-B48: 01 | 14 |
| AVGVGKSAL | HLA-C03: 03 | 14 |
| AVGVGKSAL | HLA-B42: 01 | 14 |
| LVVVGAVGV | HLA-B55: 01 | 14 |
| VGVGKSAL | HLA-C08: 01 | 14 |
| VVGAVGVGK | HLA-A74: 01 | 14 |
| AVGVGKSAL | HLA-C05: 01 | 15 |
| AVGVGKSAL | HLA-C03: 02 | 15 |
| GAVGVGKSA | HLA-C03: 04 | 15 |
| KLVVVGAVGV | HLA-A02: 04 | 15 |
| LVVVGAVGV | HLA-A02: 07 | 15 |
| VGVGKSAL | HLA-B14: 02 | 15 |
| VVVGAVGVGK | HLA-A30: 01 | 15 |
| VVGAVGVGK | HLA-B81: 01 | 16 |
| VVVGAVGV | HLA-B55: 01 | 16 |
| AVGVGKSAL | HLA-C14: 03 | 17 |
| AVGVGKSAL | HLA-B15: 01 | 17 |
| LVVVGAVGV | HLA-B54: 01 | 17 |
| AVGVGKSA | HLA-B55: 01 | 18 |
| AVGVGKSAL | HLA-C17: 01 | 18 |
| GAVGVGKSA | HLA-B50: 01 | 19 |
| GAVGVGKSAL | HLA-C17: 01 | 19 |
| YKLVVVGAV | HLA-A02: 04 | 19 |
| GAVGVGKSAL | HLA-B35: 01 | 20 |
| VVGAVGVGK | HLA-A31: 01 | 20 |
| YKLVVVGAV | HLA-B51: 01 | 20 |

In some embodiments, a first peptide comprising an epitope (or neoepitope) from Table 1, or a polynucleotide encoding the same may be combined with a second peptide comprising the same epitope but differing in peptide sequence and/or peptide length or a polynucleotide encoding the same and administered to a subject, expressing the MHC encoded by the HLA allele it binds to, as per Table 1.

In some embodiments, a first peptide comprising a first epitope (or neoepitope) from Table 1, or a polynucleotide encoding the same may be combined with a second peptide comprising a different epitope, or a polynucleotide encoding the same, wherein the second peptide comprises an epitope that is selected from Tables 2-7 as an epitope that can bind to an MHC protein encoded by an HLA allele that is expressed in the subject who expresses the MHC protein corresponding to the first epitope in Table 1.

In some embodiments, APCs are loaded with one or more peptides comprising an epitope selected from Table 1, and one or more peptides comprising epitopes selected from Tables 2-7, such that the APCs express the MHC protein encoded by the allele corresponding to the selected epitope in Table 1, and that the selected one or more epitopes from Tables 2-7 can bind to and be presented by the APCs.

In some embodiments, APCs loaded with one or more peptides comprising an epitope selected from Table 1 and one or more peptides comprising epitopes selected from Tables 2-7 may be contacted with T cells, such that the T cells are activated and primed against the epitopes, and the T cells are administered to a subject expressing an MHC protein corresponding to an HLA allele that can bind to the selected epitopes.

In some embodiments, the first peptide comprises at least one additional mutation. In some embodiments, one or more of the at least one additional mutation is not a mutation that is present in the first epitope. In some embodiments, one or more of the at least one additional mutation is a mutation in the first neoepitope.

In some aspects, the present disclosure provides a composition comprising a single polypeptide comprises the first peptide and the second peptide, or a single polynucleotide encodes the first peptide and the second peptide. In some embodiments, the composition provided herein comprises one or more additional peptides, wherein the one or more additional peptides comprise a third neoepitope. In some embodiments, the first peptide and the second peptide are encoded by a sequence transcribed from the same transcription start site. In some embodiments, the first peptide is encoded by a sequence transcribed from a first transcription start site and the second peptide is encoded by a sequence transcribed from a second transcription start site. In some embodiments, wherein the polypeptide has a length of at least 10; 15; 20; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the polypeptide comprises a first sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence; and a second sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence.

The antigens can be non-mutated antigens or mutated antigens. For example, the antigens can be tumor-associated antigens, mutated antigens, tissue-specific antigens or neoantigens. In some embodiments, the antigens are tumor-associated antigens. In some embodiments, the antigens are mutated antigens. In some embodiments, the antigens are tissue-specific antigens. In some embodiments, the antigens are neoantigens. Neoantigens are found in the cancer or the tumor in a subject and is not evident in the germline or expressed in the healthy tissue of the subject. Therefore, for a gene mutation in cancer to satisfy the criteria of generating a neoantigen, the gene mutation in the cancer must be a non-silent mutation that translates into an altered protein product. The altered protein product contains an amino acid sequence with a mutation that can be a mutated epitope for a T cell. The mutated epitope has the potential to bind to an MHC molecule. The mutated epitope also has the potential to be presented by an MHC molecule that can, for example, be detected by mass spectrometry. Furthermore, the mutated epitope has the potential to be immunogenic. Additionally, the mutated epitope has the potential to activate T cells to become cytotoxic.

In some embodiments, the present disclosure includes modified peptides. A modification can include a covalent chemical modification that does not alter the primary amino acid sequence of the antigenic peptide itself. Modifications can produce peptides with desired properties, for example, prolonging the in vivo half-life, increasing the stability, reducing the clearance, altering the immunogenicity or allergenicity, enabling the raising of particular antibodies, cellular targeting, antigen uptake, antigen processing, HLA affinity, HLA stability or antigen presentation. In some embodiments, a peptide may comprise one or more sequences that enhance processing and presentation of epitopes by APCs, for example, for generation of an immune response.

In some embodiments, the peptide may be modified to provide desired attributes. For instance, the ability of the peptides to induce CTL activity can be enhanced by linkage to a sequence which contains at least one epitope that is capable of inducing a T helper cell response. In some embodiments, immunogenic peptides/T helper conjugates are linked by a spacer molecule. In some embodiments, a spacer comprises relatively small, neutral molecules, such as amino acids or amino acid mimetics, which are substantially uncharged under physiological conditions. Spacers can be selected from, e.g., Ala, Gly, or other neutral spacers of nonpolar amino acids or neutral polar amino acids. It will be understood that the optionally present spacer need not be comprised of the same residues and thus may be a hetero- or homo-oligomer. The neoantigenic peptide may be linked to the T helper peptide either directly or via a spacer either at the amino or carboxy terminus of the peptide. The amino terminus of either the neoantigenic peptide or the T helper peptide may be acylated. Examples of T helper peptides include tetanus toxoid residues 830-843, influenza residues 307-319, and malaria circumsporozoite residues 382-398 and residues 378-389.

The peptide sequences of the present disclosure may optionally be altered through changes at the DNA level, particularly by mutating the DNA encoding the peptide at preselected bases such that codons are generated that will translate into the desired amino acids.

Provided herein is a method for treating cancer in a subject in need thereof comprising selecting at least one epitope sequence from a library of epitope sequences, wherein each epitope sequence in the library is matched to a protein encoded by an HLA allele of the subject; and contacting a T cell from the subject or an allogeneic T cell with one or more peptides comprising the at least one selected epitope sequence, wherein each of the at least one selected epitope sequence is pre-validated to satisfy at least two or three or four of the following criteria binds to a protein encoded by an HLA allele of the subject, is immunogenic according to an immunogenicity assay, is presented by APCs according to a mass spectrometry assay, and stimulates T cells to be cytotoxic according to a cytotoxicity assay. In some embodiments, the method further comprises administering the population of T cells to the subject.

In some embodiments, the at least one selected epitope sequence comprises a mutation and the method comprises identifying cancer cells of the subject to encode the epitope with the mutation. In some embodiments, the at least one selected epitope sequence is within a protein overexpressed by cancer cells of the subject and the method comprises identifying cancer cells of the subject to overexpress the protein containing the epitope. In some embodiments, the at least one epitope sequence comprises a protein expressed by a cell in a tumor microenvironment. In some embodiments, one or more of the least one selected epitope sequence comprises an epitope that is not expressed by cancer cells of the subject. In some embodiments, the epitope that is not expressed by cancer cells of the subject is expressed by cells in a tumor microenvironment of the subject. In some embodiments, the method comprises selecting the subject using a circulating tumor DNA assay. In some embodiments, the method comprises selecting the subject using a gene panel.

In some embodiments, the T cell is from a biological sample from the subject. In some embodiments, the T cell is from an apheresis or a leukapheresis sample from the subject. In some embodiments, the T cell is an allogeneic T cell.

In some embodiments, each of the at least one selected epitope sequence is pre-validated to satisfy one or more or each of the following criteria: binds to a protein encoded by an HLA allele of the subject, is immunogenic according to an immunogenicity assay, is presented by APCs according to a mass spectrometry assay, and stimulates T cells to be cytotoxic according to a cytotoxicity assay.

In some embodiments, an epitope that binds to a protein encoded by an HLA allele of the subject binds to an MHC molecule encoded by the HLA allele with an affinity of 500 nM or less according to a binding assay. For example, an epitope that binds to a protein encoded by an HLA allele of the subject can bind to an MHC molecule encoded by the HLA allele with an affinity of 400 nM, 300 nM, 200 nM, 150 nM, 100 nM, 75 nM, 50 nM, or 25 nM or less according to a binding assay. In some embodiments, an epitope that binds to a protein encoded by an HLA allele of the subject is predicted to bind to an MHC molecule encoded by the HLA allele with an affinity of 500 nM or less using an MHC epitope prediction program implemented on a computer. For example, an epitope that binds to a protein encoded by an HLA allele of the subject can be predicted to bind to an MHC molecule encoded by the HLA allele with an affinity of 400 nM, 300 nM, 200 nM, 150 nM, 100 nM, 75 nM, 50 nM, or 25 nM or less using an MHC epitope prediction program implemented on a computer. In some embodiments, the MHC epitope prediction program implemented on a computer is an in-house prediction program (described in WO2018148671 publication, WO2017184590 publication) or NetMHCpan. In some embodiments, the MHC epitope prediction program implemented on a computer is NetMHCpan version 4.0.

In some embodiments, the epitope that is presented by APCs according to a mass spectrometry assay is detected by mass spectrometry after elution from the APCs with a mass accuracy of the detected peptide to be less than 15 Da. For example, the epitope that is presented by APCs according to a mass spectrometry assay can be detected by mass spectrometry after elution from the APCs with a mass accuracy of the detected peptide to be less than 14 Da, 13 Da, 12 Da, 11 Da, 10 Da, 9 Da, 8 Da, 7 Da, 6 Da, 5 Da, 4 Da, 3 Da, 2 Da, or 1 Da. In some embodiments, the epitope that is presented by APCs according to a mass spectrometry assay is detected by mass spectrometry after elution from the APCs with a mass accuracy of the detected peptide to be less than 10,000 parts per million (ppm). For example, the epitope that is presented by APCs according to a mass spectrometry assay can be detected by mass spectrometry after elution from the APCs with a mass accuracy of the detected peptide to be less than 7,500 ppm; 5,000 ppm; 2,500 ppm; 1,000 ppm; 900 ppm; 800 ppm; 700 ppm; 600 ppm; 500 ppm; 400 ppm; 300 ppm; 200 ppm or 100 ppm.

In some embodiments, the epitope that is immunogenic according to an immunogenicity assay is immunogenic according to a multimer assay. In some embodiments, the multimer assay comprises flow cytometry analysis. In some embodiments, the multimer assay comprises detecting T cells bound to a peptide-MHC multimer comprising the at least one selected epitope sequence and the matched HLA allele, wherein the T cells have been stimulated with APCs comprising a peptide containing the at least one selected epitope sequence. In some embodiments, an epitope is immunogenic according to the multimer assay when (i) at least 10 T cells that have been stimulated with APCs comprising a peptide containing the at least one selected epitope sequence are detected, (ii) the detected T cells make up at least 0.005% of the CD8⁺ cells analyzed, and (iii) the percentage of detected T cells of CD8+ T cells is higher than the percentage of detected T cells of CD8+ T cells detected in a control sample. For example, an epitope can be immunogenic according to the multimer assay when (i) at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected, (ii) the detected T cells make up at least 0.005% of the CD8⁺ cells analyzed, and (iii) the percentage of detected T cells of CD8+ T cells is higher than the percentage of detected T cells of CD8+ T cells detected in a control sample. For example, an epitope can be immunogenic according to the multimer assay when (i) at least 10 T cells that have been stimulated with APCs comprising a peptide containing the at least one selected epitope sequence are detected, (ii) the detected T cells make up at least 0.01%, 0.05%, 0.1%, 0.2%, 0.3%, 0.4%, 0.6%, 0.7%, 0.8%, 0.9%, 1%, 1.5%, 2%, 3%, 4%, 5%, 6%, 7%, 8%, 9%, or 10% of the CD8⁺ cells analyzed, and (iii) the percentage of detected T cells of CD8+ T cells is higher than the percentage of detected T cells of CD8+ T cells detected in a control sample. For example, an epitope can be immunogenic according to the multimer assay when (i) at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected, (ii) the detected T cells make up at least 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD8⁺ cells analyzed, and (iii) the percentage of detected T cells of CD8+ T cells is higher than the percentage of detected T cells of CD8+ T cells detected in a control sample.

In some embodiments, the epitope is immunogenic according to the multimer assay when at least T cells that have been stimulated with APCs comprising a peptide containing the at least one selected epitope sequence are detected in at least one out of six stimulations from the same starting sample. For example, the epitope can be immunogenic according to the multimer assay when at least 10, 15, 20, 30, 40, 60, 70, 80, 90, 100, 150, 200, 250, 300, 400, 500, 600, 700, 800, 900 or more T cells that have been stimulated with APCs comprising a peptide containing the at least one selected epitope sequence are detected in at least one out of six stimulations from the same starting sample. For example, the epitope can be immunogenic according to the multimer assay when at least 10 T cells that have been stimulated with APCs comprising a peptide containing the at least one selected epitope sequence are detected in at least 2 out of 6, 7, 8, 9, 10, 11 or 12 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5 or 6 out of 6 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6 or 7 out of 7 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7 or 8 out of 8 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7, 8 or 9 out of 9 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7, 8, 9 or 10 out of 10 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7, 8, 9, 10 or 11 out of 11 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7, 8, 9, 11 or 12 out of 12 stimulations from the same starting sample. For example, the epitope can be immunogenic according to the multimer assay when at least 10 T cells that have been stimulated with APCs comprising a peptide containing the at least one selected epitope sequence are detected in at least 3 out of 6, 7, 8, 9, 10, 11, 12, 13, 14, 15, 16, 17 or 18 stimulations from the same starting sample. For example, the epitope can be immunogenic according to the multimer assay when at least 10 T cells that have been stimulated with APCs comprising a peptide containing the at least one selected epitope sequence are detected in at least 4 out of 6, 7, 8, 9, 10, 11, 12, 13, 14, 15, 16, 17, 18, 19, 20, 21, 22, 23 or 24 stimulations from the same starting sample. For example, the epitope can be immunogenic according to the multimer assay when at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected in at least one out of six stimulations from the same starting sample. For example, the epitope can be immunogenic according to the multimer assay when at least 10, 15, 20, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected in at least 2 out of 6, 7, 8, 9, 10, 11 or 12 stimulations from the same starting sample or in at least 3 out of 6, 7, 8, 9, 10, 11, 12, 13, 14, 15, 16, 17 or 18 stimulations from the same starting sample or in at least 4 out of 6, 7, 8, 9, 10, 11, 12, 13, 14, 15, 16, 17, 18, 19, 20, 21, 22, 23 or 24 stimulations from the same starting sample. In some embodiments, the control sample comprises T cells that have been stimulated with APCs that (i) do not comprise a peptide containing the at least one selected epitope sequence, (ii) comprise a peptide derived from a different protein than the at least one selected epitope sequence, or (iii) comprise a peptide with a random sequence. In some embodiments, the T cells have been stimulated with APCs comprising a peptide containing the at least one selected epitope sequence for at least 3, 4, 5, 6, 7, 8, 9, 10, 11, 12, 13, 14, 15, 16, 7, 18, 19, 20 or more days. In some embodiments, antigen-specific T cells have been expanded at least 5-fold, 10-fold, 20, fold, 50-fold, 100-fold, 500-fold or 1,000-fold or more in the presence of APCs comprising a peptide containing the at least one selected epitope sequence.

In some embodiments, the epitope that is immunogenic according to an immunogenicity assay is immunogenic according to a functional assay. In some embodiments, the functional assay comprises an immunoassay. In some embodiments, the functional assay comprises detecting T cells with intracellular staining of IFNγ or TNFα or cell surface expression of CD107a and/or CD107b, wherein the T cells have been stimulated with APCs comprising a peptide containing the at least one selected epitope sequence In some embodiments, the epitope is immunogenic according to the functional assay when (i) at least 10 T cells that have been stimulated with APCs comprising a peptide containing the at least one selected epitope sequence are detected, (ii) the detected T cells make up at least 0.005% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample. For example the epitope can be immunogenic according to the functional assay when (i) at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected, (ii) the detected T cells make up at least 0.005% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample. For example the epitope can be immunogenic according to the functional assay when (i) at least 10 T cells that have been stimulated with APCs comprising a peptide containing the at least one selected epitope sequence are detected, (ii) the detected T cells make up at least 0.01%, 0.05%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample. For example the epitope can be immunogenic according to the functional assay when (i) at least 10, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected, (ii) the detected T cells make up at least 0.01%, 0.05%, 0.1%, 0.2%, 0.3%, 0.4%, 0.6%, 0.7%, 0.8%, 0.9%, 1%, 1.5%, 2%, 3%, 4%, 5%, 6%, 7%, 8%, 9%, or 10% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample.

In some embodiments, the T cells stimulated to be cytotoxic according to the cytotoxicity assay are T cells that have been stimulated with APCs comprising a peptide containing the at least one selected epitope sequence that kill cells presenting the epitope. In some embodiments, a number of cells presenting the epitope that are killed by the T cells is at least 1.1, 2, 3, 4, 5, 6, 7, 8, 9, 10, 15, 20, 25, 30, 50, 100, 500, or 1,000 fold higher than a number of cells that do not present the epitope that are killed by the T cells. In some embodiments, a number of cells presenting the epitope that are killed by the T cells is at least 1.1, 2, 3, 4, 5, 6, 7, 8, 9, 10, 15, 20, 25, 30, 50, 100, 500, or 1,000 fold higher than a number of cells presenting the epitope killed by T cells that have been stimulated with APCs that (i) do not comprise a peptide containing the at least one selected epitope sequence, (ii) comprise a peptide derived from a different protein than the at least one selected epitope sequence, or (iii) comprise a peptide with a random sequence In some embodiments, a number of cells presenting a mutant epitope that are killed by the T cells is at least 1.1, 2, 3, 4, 5, 6, 7, 8, 9, 10, 15, 20, 25, 30, 50, 100, 500, or 1,000 fold higher than a number of cells presenting a corresponding wild-type epitope that are killed by the T cells. In some embodiments, the T cells stimulated to be cytotoxic according to the cytotoxicity assay are T cells stimulated to be specifically cytotoxic according to the cytotoxicity assay.

In some embodiments, at least one of the one or more peptides is a synthesized peptide or a peptide expressed from a nucleic acid sequence.

In some embodiments, the method comprises identifying a protein encoded by an HLA allele of the subject or identifying an HLA allele in the genome of the subject.

In some embodiments, the at least one selected epitope sequence is selected from one or more epitope sequences of Table 1-12.

In some embodiments, the method comprises expanding the T cell contacted with the one or more peptides in vitro or ex vivo to obtain a population of T cells specific to the at least one selected epitope sequence in complex with an MHC protein.

In some embodiments, a protein comprising the at least one selected epitope sequence is expressed by a cancer cell of the subject. In some embodiments, a protein comprising the at least one selected epitope sequence is expressed by cells in the tumor microenvironment of the subject.

In some embodiments, one or more of the at least one selected epitope sequence comprises a mutation. In some embodiments, one or more of the at least one selected epitope sequence comprises a tumor specific mutation. In some embodiments, one or more of the at least one selected epitope sequence is from a protein overexpressed by a cancer cell of the subject. In some embodiments, one or more of the at least one selected epitope sequence comprises a driver mutation. In some embodiments, one or more of the at least one selected epitope sequence comprises a drug resistance mutation. In some embodiments, one or more of the at least one selected epitope sequence is from a tissue-specific protein. In some embodiments, one or more of the at least one selected epitope sequence is from a cancer testes protein. In some embodiments, one or more of the at least one selected epitope sequence is a viral epitope. In some embodiments, one or more of the at least one selected epitope sequence is a minor histocompatibility epitope. In some embodiments, one or more of the at least one selected epitope sequence is from a RAS protein. In some embodiments, one or more of the at least one selected epitope sequence is from a GATA3 protein. In some embodiments, one or more of the at least one selected epitope sequence is from a EGFR protein. In some embodiments, one or more of the at least one selected epitope sequence is from a BTK protein. In some embodiments, one or more of the at least one selected epitope sequence is from a p53 protein. In some embodiments, one or more of the at least one selected epitope sequence is from aTMPRSS2::ERG fusion polypeptide. In some embodiments, one or more of the at least one selected epitope sequence is from a Myc protein. In some embodiments, at least one of the at least one selected epitope sequence is from a protein encoded by a gene selected from the group consisting of ANKRD30A, COL10A 1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGE5, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53, KIF2B, ADAD1, SPATA8, CCDC70, TPD52L3, ACTL7B, DMRTB1, SYCN, CELA2A, CELA2B, PNLIPRP1, CTRC, AMY2A, SERPINI2, RBPJL, AQP12A, IAPP, KIRREL2, G6PC2, AQP12B, CYP11B1, CYP11B2, STAR, CYP11A1, and MC2R.

In some embodiments, at least one of the at least one selected epitope sequence is from a tissue-specific protein that has an expression level in a target tissue of the subject that is at least 2 fold more than an expression level of the tissue-specific protein in each tissue of a plurality of non-target tissues that are different than the target tissue.

In some embodiments, contacting a T cell from the subject or an allogeneic T cell with one or more peptides comprising the at least one selected epitope sequence comprises contacting the T cell with APCs presenting the epitope.

In some embodiments, the APCs presenting the epitope comprises one or more peptides comprising the at least one selected epitope sequence or a polynucleic acid that encodes one or more peptides comprising the at least one selected epitope sequence. In some embodiments, the polypeptide comprises at least two of the selected epitope sequences, each expressed by cancer cells of a human subject with cancer.

In some embodiments, the method comprises depleting CD14+ cells and CD25+ cells from a population of immune cells comprising APCs and T cells, thereby forming a CD14/CD25 depleted population of immune cells comprising a first population of APCs and T cells. In some embodiments, the population of immune cells is from a biological sample from the subject. In some embodiments, the method further comprises incubating the CD14/CD25 depleted population of immune cells comprising a first population of APCs and T cells for a first time period in the presence of FMS-like tyrosine kinase 3 receptor ligand (FLT3L), and a polypeptide comprising the at least one selected epitope sequence, or a polynucleotide encoding the polypeptide; thereby forming a population of cells comprising stimulated T cells. In some embodiments, the method further comprises expanding the population of cells comprising stimulated T cells, thereby forming an expanded population of cells comprising tumor antigen-specific T cells, wherein the tumor antigen-specific T cells comprise T cells that are specific to a complex comprising the at least one selected epitope sequence and an MHC protein expressed by the cancer cells or APCs of the subject. In some embodiments, expanding is performed in less than 28 days. In some embodiments, incubating comprises incubating the CD14/CD25 depleted population of immune cells comprising a first population of APCs and T cells for a first time period in the presence of FLT3L and an RNA encoding the polypeptide. In some embodiments, depleting CD14+ cells and CD25+ cells from the population of immune cells comprising a first population of APCs and T cells comprises contacting the population of immune cells comprising a first population of APCs and T cells with a CD14 binding agent and a CD25 binding agent. In some embodiments, depleting further comprising depleting CD19+ cells from the population of immune cells comprising a first population of APCs and T cells. In some embodiments, depleting further comprising depleting CD11b+ cells from the population of immune cells comprising a first population of APCs and T cells.

In some embodiments, the method further comprises administering a pharmaceutical composition comprising the expanded population of cells comprising tumor antigen specific T cells to a human subject with cancer. In some embodiments, the human subject with cancer is the human subject from which the biological sample was obtained.

In some embodiments, the fraction of CD8+ tumor antigen-specific T cells of the total number of CD8+ T cells in the expanded population of cells comprising tumor antigen specific T cells is at least two-fold higher than the fraction of CD8+ tumor antigen-specific T cells of the total number of CD8+ T cells in the biological sample. In some embodiments, the fraction of CD4+ tumor antigen-specific T cells of the total number of CD4+ T cells in the expanded population of cells comprising tumor antigen specific T cells is at least two-fold higher than the fraction of CD4+ tumor antigen-specific T cells of the total number of CD4+ T cells in the biological sample. In some embodiments, at least 0.1%, 0.2%, 0.3%, 0.4%, 0.5%, 0.7%, 0.8%, 0.9%, 1%, 1.5%, 2%, 3%, 4%, 5%, 6%, 7%, 8%, 9%, or 10% of the CD8+ T cells in the expanded population of cells comprising tumor antigen specific T cells are CD8+ tumor antigen-specific T cells derived from naïve CD8+ T cells. In some embodiments, at least 0.1%, 0.2%, 0.3%, 0.4%, 0.6%, 0.7%, 0.8%, 0.9%, 1%, 1.5%, 2%, 3%, 4%, 5%, 6%, 7%, 8%, 9%, or 10% of the CD8+ T cells in the expanded population of cells comprising tumor antigen specific T cells are CD8+ tumor antigen-specific T cells derived from memory CD8+ T cells. In some embodiments, at least 0.1%, 0.2%, 0.3%, 0.5%, 0.6%, 0.7%, 0.8%, 0.9%, 1%, 1.5%, 2%, 3%, 4%, 5%, 6%, 7%, 8%, 9%, or 10% of the CD4+ T cells in the expanded population of cells comprising tumor antigen specific T cells are CD4+ tumor antigen-specific T cells derived from naïve CD4+ T cells. In some embodiments, at least 0.1%, 0.2%, 0.3%, 0.5%, 0.6%, 0.7%, 0.8%, 0.9%, 1%, 1.5%, 2%, 3%, 4%, 5%, 6%, 7%, 8%, 9%, or 10% of the CD4+ T cells in the expanded population of cells comprising tumor antigen specific T cells are CD4+ tumor antigen-specific T cells derived from memory CD4+ T cells.

In some embodiments, expanding comprises contacting the population of cells comprising stimulated T cells with a second population of mature APCs, wherein the second population of mature APCs have been incubated with FLT3L and present the at least one selected epitope sequence; and expanding the population of cells comprising stimulated T cells for a second time period, thereby forming an expanded population of T cells. In some embodiments, the second population of mature APCs has been incubated with FLT3L for at least 1 day prior to contacting the population of cells comprising stimulated T cells with the second population of mature APCs. In some embodiments, expanding further comprises contacting the expanded population of T cells with a third population of mature APCs, wherein the third population of mature APCs have been incubated with FLT3L and present the at least one selected epitope sequence; and expanding the expanded population of T cells for a third time period, thereby forming the expanded population of cells comprising tumor antigen-specific T cells. In some embodiments, the third population of mature APCs has been incubated with FLT3L for at least 1 day prior to contacting the expanded population of T cells with the third population of mature APCs. In some embodiments, the biological sample is a peripheral blood sample, a leukapheresis sample or an apheresis sample.

In some embodiments, the method further comprises harvesting the expanded population of cells comprising tumor antigen-specific T cells, cryopreserving the expanded population of cells comprising tumor antigen-specific T cells or preparing a pharmaceutical composition containing the expanded population of cells comprising tumor antigen-specific T cells.

In some embodiments, the method comprises generating cancer cell nucleic acids from a first biological sample comprising cancer cells obtained from a subject and generating non-cancer cell nucleic acids from a second biological sample comprising non-cancer cells obtained from the same subject.

In some embodiments, the protein encoded by an HLA allele of the subject is a protein encoded by an HLA allele selected from the group consisting of HLA-A01:01, HLA-A02:01, HLA-A03:01, HLA-A03:03, HLA-A03:04, HLA-A11:01, HLA-A24:01, HLA-A30:01, HLA-A31:01, HLA-A32:01, HLA-A33:01, HLA-A68:01, HLA-B07:02, HLA-B08:01, HLA-B15:01, HLA-B44:03, HLA-007:01 and HLA-007:02. In some embodiments, the protein encoded an HLA allele by the subject is a protein encoded by the HLA-A03:04. In some embodiments, the protein encoded an HLA allele by the subject is a protein encoded by the HLA-A03:03.

In some embodiments, the method comprises identifying one or two or more different proteins that comprise the at least one selected epitope sequence and that are expressed by cancer cells of the subject. In some embodiments, the method comprises identifying one or two or more different proteins that comprise the at least one selected epitope sequence and that are expressed by cancer cells of the subject by measuring levels of RNA encoding the one or two or more different proteins in the cancer cells. In some embodiments, the method comprises isolating genomic DNA or RNA from cancer cells and non-cancer cells of the subject.

In some embodiments, one or more of the at least one selected epitope sequence comprises a point mutation or a sequence encoded by a point mutation. In some embodiments, one or more of the at least one selected epitope sequence comprises a sequence encoded by a neoORF mutation. In some embodiments, one or more of the at least one selected epitope sequence comprises a sequence encoded by a gene fusion mutation. In some embodiments, one or more of the at least one selected epitope sequence comprises a sequence encoded by an indel mutation. In some embodiments, one or more of the at least one selected epitope sequence comprises a sequence encoded by a splice site mutation. In some embodiments, at least two of the at least one selected epitope sequence are from a same protein. In some embodiments, at least two of the at least one selected epitope sequence comprise an overlapping sequence. In some embodiments, at least two of the at least one selected epitope sequence are from different proteins. In some embodiments, the one or more peptides comprise at least 2, 3, 4, 5, 6, 7, 8, 9, or 10 or more peptides.

In some embodiments, cancer cells of the subject are cancer cells of a solid cancer. In some embodiments, cancer cells of the subject are cancer cells of a leukemia or a lymphoma.

In some embodiments, the mutation is a mutation that occurs in a plurality of cancer patients.

In some embodiments, the MHC is a Class I MHC. In some embodiments, the MHC is a Class II MHC.

In some embodiments, the T cell is a CD8 T cell. In some embodiments, the T cell is a CD4 T cell. In some embodiments, the T cell is a cytotoxic T cell. In some embodiments, the T cell t is a memory T cell. In some embodiments, the T cell is a naive T cell.

In some embodiments, the method further comprises selecting one or more subpopulation of cells from an expanded population of T cells prior to administering to the subject.

In some embodiments, eliciting an elicit an immune response in the T cell culture comprises inducing IL2 production from the T cell culture upon contact with the peptide. In some embodiments, eliciting an immune response in the T cell culture comprises inducing a cytokine production from the T cell culture upon contact with the peptide, wherein the cytokine is an Interferon gamma (IFN-γ), Tumor Necrosis Factor (TNF) alpha (a) and/or beta ((3) or a combination thereof. In some embodiments, eliciting an immune response in the T cell culture comprises inducing the T cell culture to kill a cell expressing the peptide. In some embodiments, eliciting an immune response in the T cell culture comprises detecting an expression of a Fas ligand, granzyme, perforins, IFN, TNF, or a combination thereof in the T cell culture.

In some embodiments, the one or more peptides comprising the at least one selected epitope sequence is purified. In some embodiments, the one or more peptides comprising the at least one selected epitope sequence is lyophilized. In some embodiments, the one or more peptides comprising the at least one selected epitope sequence is in a solution. In some embodiments, the one or more peptides comprising the at least one selected epitope sequence is present in a storage condition such that the integrity of the peptide is ≥99%.

In some embodiments, the method comprises stimulating T cells to be cytotoxic against cells loaded with the at least one selected epitope sequences according to a cytotoxicity assay. In some embodiments, the method comprises stimulating T cells to be cytotoxic against cancer cells expressing a protein comprising the at least one selected epitope sequences according to a cytotoxicity assay. In some embodiments, the method comprises stimulating T cells to be cytotoxic against a cancer associated cell expressing a protein comprising the at least one selected epitope sequences according to a cytotoxicity assay.

In some embodiments, the at least one selected epitope is expressed by a cancer cell, and an additional selected epitope is expressed by a cancer associated cell. In some embodiments, the additional selected epitope is expressed on a cancer associated fibroblast cell. In some embodiments, the additional selected epitope is selected from any one of Tables 2-XYX.

In some embodiments, a method provided herein is a method for treating cancer in a subject in need thereof comprising: selecting at least one epitope sequence from a library of epitope sequences, wherein each epitope sequence in the library is matched to a protein encoded by an HLA allele; and contacting a T cell from the subject or an allogeneic T cell with one or more peptides comprising the at least one selected epitope sequence, wherein each of the at least one selected epitope sequences; binds to a protein encoded by an HLA allele of the subject; is immunogenic according to an immunogenic assay; is presented by APCs according to a mass spectrometry assay, and stimulates T cells to be cytotoxic according to a cytotoxicity assay.

In some embodiments, the method comprises selecting the subject using a circulating tumor DNA assay. In some embodiments, the method comprises selecting the subject using a gene panel.

In some embodiments, the T cell is from a biological sample from the subject. In some embodiments, the T cell is from an apheresis or a leukapheresis sample from the subject.

In some embodiments, at least one of the one or more peptides a synthesized peptide or a peptide expressed from a nucleic acid sequence.

In some embodiments, the method comprises identifying a protein encoded by an HLA allele of the subject or identifying an HLA allele in the genome of the subject. In some embodiments, the method comprises identifying a protein encoded by an HLA allele of the subject that is expressed by the subject. In some embodiments, the method comprises contacting a T cell from the subject with one or more peptides selected from one or more peptides of a table provided herein. In some embodiments, the method comprises contacting a T cell from the subject with one or more peptides comprising an epitope selected from an epitope of a table provided herein. In some embodiments, the method further comprises expanding in vitro or ex vivo the T cell contacted with the one or more peptides to obtain a population of T cells. In some embodiments, the method further comprises administering the population of T cells to the subject at a dose and a time interval such that the cancer is reduced or eliminated.

In some embodiments, at least one of the one or more peptides is expressed by a cancer cell of the subject. In some embodiments, at least one of the epitopes of the one or more peptides comprises a mutation.

In some embodiments, at least one of the epitopes of the one or more peptides comprises a tumor specific mutation. In some embodiments, at least one of the epitopes of the one or more peptides is from a protein overexpressed by a cancer cell of the subject. In some embodiments, at least one of the epitopes of the one or more peptides is from a protein encoded by a gene selected from the group consisting of ANKRD30A, COL10A1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGE5, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53, KIF2B, ADAD1, SPATA8, CCDC70, TPD52L3, ACTL7B, DMRTB1, SYCN, CELA2A, CELA2B, PNLIPRP1, CTRC, AMY2A, SERPINI2, RBPJL, AQP12A, LAPP, KIRREL2, G6PC2, AQP12B, CYP11B1, CYP11B2, STAR, CYP11A1, and MC2R.

In some embodiments, the at least one of the one or more peptides is from a protein encoded by a tissue-specific antigen epitope gene that has an expression level in a target tissue of the subject that is at least 2 fold more than an expression level of the tissue-specific antigen gene in each tissue of a plurality of non-target tissues that are different than the target tissue.

In some embodiments, composition comprises an adjuvant.

In some embodiments, the composition comprises one or more additional peptides, wherein the one or more additional peptides comprise a third epitope. In some embodiments, the first and/or second epitope, and/or third epitope binds to an HLA protein with a greater affinity than a corresponding wild-type sequence. In some embodiments, the first and/or second epitope binds to an HLA protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second epitope binds to an HLA class I protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class II protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second epitope (or neoepitope) binds to a protein encoded by an HLA allele expressed by a subject. In some embodiments, the mutation is not present in non-cancer cells of a subject. In some embodiments, the first and/or second neoepitope is encoded by a gene or an expressed gene of a subject's cancer cells. In some embodiments, the composition comprises a first T cell comprising the first TCR. In some embodiments, the composition comprises a second T cell comprising the second TCR. In some embodiments, the first TCR comprises a non-native intracellular domain and/or the second TCR comprises a non-native intracellular domain. In some embodiments, the first TCR is a soluble TCR and/or the second TCR is a soluble TCR. In some embodiments, the first and/or second T cell is a cytotoxic T cell. In some embodiments, the first and/or second T cell is a gamma delta T cell. In some embodiments, the first and/or second T cell is a helper T cell. In some embodiments, the first T cell is a T cell stimulated, expanded or induced with the first neoepitope and/or the second T cell is a T cell stimulated, expanded or induced with the second neoepitope. In some embodiments, the first and/or second T cell is an autologous T cell. In some embodiments, the first and/or second T cell is an allogenic T cell. In some embodiments, the first and/or second T cell is an engineered T cell. In some embodiments, the first and/or second T cell is a T cell of a cell line. In some embodiments, the first and/or second TCR binds to an HLA-peptide complex with a K_{D} or an IC₅₀ of less than 1000 nM, 900 nM, 800 nM, 700 nM, 600 nM, 500 nM, 250 nM, 150 nM, 100 nM, 50 nM, 25 nM or nM. In some aspects, provided herein is a vector comprising a polynucleotide encoding a first and a second peptide described herein. In some embodiments, the polynucleotide is operably linked to a promoter. In some embodiments, the vector is a self-amplifying RNA replicon, plasmid, phage, transposon, cosmid, virus, or virion. In some embodiments, the vector is a viral vector. In some embodiments, the vector is derived from a retrovirus, lentivirus, adenovirus, adeno-associated virus, herpes virus, pox virus, alpha virus, vaccinia virus, hepatitis B virus, human papillomavirus or a pseudotype thereof. In some embodiments, the vector is a non-viral vector. In some embodiments, the non-viral vector is a nanoparticle, a cationic lipid, a cationic polymer, a metallic nanopolymer, a nanorod, a liposome, a micelle, a microbubble, a cell-penetrating peptide, or a liposphere.

In some aspects, provided herein is a pharmaceutical composition comprising: a composition described herein, or a vector described herein; and a pharmaceutically acceptable excipient.

In some embodiments, the plurality of cells is autologous cells. In some embodiments, the plurality of APC cells is autologous cells. In some embodiments, the plurality of T cells is autologous cells. In some embodiments, the pharmaceutical composition further comprises an immunomodulatory agent or an adjuvant. In some embodiments, the immunomodulatory agent is a cytokine.

In some embodiments, the method comprises: incubating one or more antigen presenting cell (APC) preparations with a population of immune cells from a biological sample depleted of cells expressing CD14 and CD25 for one or more separate time periods; incubating one or more APC preparations with a population of immune cells from a biological sample for one or more separate time periods, wherein the one or more APCs comprise one or more FMS-like tyrosine kinase 3 receptor ligand (FLT3L)-stimulated APCs; or incubating FLT3L and at least one peptide with a population of immune cells from a biological sample, wherein the FLT3L is incubated with the population of immune cells for a first time period and wherein the at least one peptide is incubated with the population of immune cells for a first peptide stimulation time period, thereby obtaining a first stimulated T cell sample, wherein the population of immune cells comprises at least one T cell and at least one APC; wherein at least one antigen specific memory T cell is expanded, or at least one antigen specific naïve T cell is induced.

In some embodiments, the method comprises incubating a population of immune cells from a biological sample with one or more APC preparations for one or more separate time periods of less than 28 days from incubating the population of immune cells with a first APC preparation of the one or more APC preparations. In some embodiments, the method comprises incubating a population of immune cells from a biological sample with 3 or less APC preparations for 3 or less separate time periods. In some embodiments, the method comprises incubating a population of immune cells from a biological sample with 2 or less APC preparations for 2 or less separate time periods. In some embodiments, the method comprises incubating a population of immune cells from a biological sample with one or more APC preparations for one or more separate time periods of less than 28 days from incubating the population of immune cells with a first APC preparation of the one or more APC preparations. In some embodiments, the total period of preparation of T cells stimulated with an antigen by incubating a population of immune cells from a biological sample with one or more APC preparations for one or more separate time periods is less than 28 days.

In some embodiments, at least two of the one or more APC preparations comprise a FLT3L-stimulated APC. In some embodiments, at least three of the one or more APC preparations comprise a FLT3L-stimulated APC. In some embodiments, incubating comprises incubating a first APC preparation of the APC preparations to the T cells for more than 7 days. In some embodiments, an APC of the APC preparations comprises an APC loaded with one or more antigen peptides comprising one or more of the at least one antigen peptide sequence. In some embodiments, an APC of the APC preparations is an autologous APC or an allogenic APC. In some embodiments, an APC of the APC preparations comprises a dendritic cell (DC). In some embodiments, the DC is a CD141⁺ DC. In some embodiments, the method comprises depleting cells expressing CD14 and CD25 from the biological sample, thereby obtaining the population of immune cells from a biological sample depleted of cells expressing CD14 and CD25. In some embodiments, the method further comprises depleting cells expressing CD19. In some embodiments, the method further comprises depleting cells expressing CD11b. In some embodiments, depleting cells expressing CD14 and CD25 comprises binding a CD14 or CD25 binding agent to an APC of the one or more APC preparations. In some embodiments, the method further comprises administering one or more of the at least one antigen specific T cell to a subject.

In some embodiments, incubating comprises incubating a first APC preparation of the one or more APC preparations to the T cells for more than 7, 8, 9, 10, 11, 12, 13, 14, 15, 16, 17, 18, 19, or 20 days. In some embodiments, the method comprises incubating at least one of the one or more of the APC preparations with a first medium comprising at least one cytokine or growth factor for a first time period. In some embodiments, the method comprises incubating at least one of the one or more of the APC preparations with a second medium comprising one or more cytokines or growth factors for a third time period, thereby obtaining a matured APC. In some embodiments, the method further comprises removing the one or more cytokines or growth factors of the second medium after the third time period. In some embodiments, an APC of the APC preparations is stimulated with one or more cytokines or growth factors. In some embodiments, the one or more cytokines or growth factors comprise GM-CSF, IL-4, FLT3L, TNF-α, IL-1β, PGE1, IL-6, IL-7, IFN-α, R848, LPS, ss-rna40, poly I: C, or a combination thereof.

In some embodiments, the antigen is a neoantigen, a tumor associated antigen, a viral antigen, a minor histocompatibility antigen or a combination thereof.

In some embodiments, the method is performed ex vivo.

In some embodiments, wherein the method comprises incubating the population of immune cells from a biological sample depleted of cells expressing CD14 and CD25 with FLT3L for a first time period. In some embodiments, the method comprises incubating at least one peptide with the population of immune cells from a biological sample depleted of cells expressing CD14 and CD25 for a second time period, thereby obtaining a first matured APC peptide loaded sample. In some embodiments, the method comprises depleting cells expressing CD14, cells expressing CD19 and cells expressing CD25 from the population of immune cells. In some embodiments, the method comprises depleting cells expressing CD14, cells expressing CD11b and cells expressing CD25 from the population of immune cells. In some embodiments, the method comprises depleting cells expressing CD14, cells expressing CD11b, cells expressing CD19 and cells expressing CD25. In some embodiments, the method comprises depleting at least CD14, CD11b, CD19 and CD25. In some embodiments, the method comprises depleting cells expressing at least one of CD14, CD11b, CD19 and CD25, and at least a fifth cell type expressing a fifth cell surface marker. In some embodiments, the method comprises selectively depleting CD14 and CD25 expressing cells from the population of immune cells, and any one or more of CD19, CD11b expressing cells, from the population of immune cells, at a first incubation period, at a second incubation period, and/or at a third incubation period.

In some embodiments of the method described herein, contacting a T cell from the subject or an allogeneic T cell with one or more peptides comprising the at least one selected epitope sequence comprises contacting the T cell with APCs presenting the epitope.

In some embodiments of the method described herein, the APCs presenting the epitope comprises one or more peptides comprising the at least one selected epitope sequence or a polynucleic acid that encodes one or more peptides comprising the at least one selected epitope sequence.

In some embodiments, the method comprises depleting CD14+ cells and CD25+ cells from a population of immune cells comprising APCs and T cells, thereby forming a CD14/CD25 depleted population of immune cells comprising a first population of APCs and T cells. In some embodiments, the population of immune cells is from a biological sample from the subject. In some embodiments of the method described herein, the method further comprises incubating the CD14/CD25 depleted population of immune cells comprising a first population of APCs and T cells for a first time period in the presence of FMS-like tyrosine kinase 3 receptor ligand (FLT3L), and a polypeptide comprising the at least one selected epitope sequences, or a polynucleotide encoding the polypeptide; thereby forming a population of cells comprising stimulated T cells. In some embodiments, the method further comprises expanding the population of cells comprising stimulated T cells, thereby forming an expanded population of cells comprising tumor antigen-specific T cells, wherein the tumor antigen-specific T cells comprise T cells that are specific to a complex comprising the at least one selected epitope sequences and an MHC protein expressed by the cancer cells or APCs of the subject.

In some embodiments of the method described herein, expanding comprises contacting the population of cells comprising stimulated T cells with a second population of mature APCs, wherein the second population of mature APCs have been incubated with FLT3L and present the at least one selected epitope sequence and expanding the population of cells comprising stimulated T cells for a second time period, thereby forming an expanded population of T cells. In some embodiments, the second population of mature APCs has been incubated with FLT3L for at least 1 day prior to contacting the population of cells comprising stimulated T cells with the second population of mature APCs. In some embodiments, the expanding further comprises contacting the expanded population of T cells with a third population of mature APCs, wherein the third population of mature APCs have been incubated with FLT3L and present the at least one selected epitope sequence; and expanding the expanded population of T cells for a third time period, thereby forming the expanded population of cells comprising tumor antigen-specific T cells. In some embodiments, the third population of mature APCs has been incubated with FLT3L for at least 1 day prior to contacting the expanded population of T cells with the third population of mature APCs. In some embodiments of the method described herein, the method further comprises harvesting the expanded population of cells comprising tumor antigen-specific T cells, cryopreserving the expanded population of cells comprising tumor antigen-specific T cells or preparing a pharmaceutical composition containing the expanded population of cells comprising tumor antigen-specific T cells. In some embodiments, the incubating comprises incubating the CD14/CD25 depleted population of immune cells comprising a first population of APCs and T cells for a first time period in the presence of FLT3L and an RNA encoding the polypeptide.

In some embodiments, the method further comprises administering a pharmaceutical composition comprising the expanded population of cells comprising tumor antigen specific T cells to a human subject with cancer. In some embodiments, the human subject with cancer is the human subject from which the biological sample was obtained. In some embodiments, the polypeptide is from 8 to 50 amino acids in length. In some embodiments, the polypeptide comprises at least two of the selected epitope sequences, each expressed by cancer cells of a human subject with cancer.

In some embodiments, depleting CD14+ cells and CD25+ cells from the population of immune cells comprising a first population of APCs and T cells comprises contacting the population of immune cells comprising a first population of APCs and T cells with a CD14 binding agent and a CD25 binding agent. In some embodiments, depleting further comprising depleting CD19+ cells from the population of immune cells comprising a first population of APCs and T cells. In some embodiments, the method further comprises contacting the population of immune cells with a CD19 binding agent. In some embodiments, depleting further comprising depleting CD11b+ cells from the population of immune cells comprising a first population of APCs and T cells. In some embodiments, the method further comprises contacting the population of immune cells with a CD11b binding agent.

In some embodiments, the method comprises incubating the first matured APC peptide loaded sample with at least one T cell for a third time period, thereby obtaining a stimulated T cell sample. In some embodiments, the method comprises incubating a T cell of a first stimulated T cell sample with a FLT3L-stimulated APC of a matured APC sample for a fourth time period, FLT3L and a second APC peptide loaded sample of a matured APC sample for a fourth time period or FLT3L and a FLT3L-stimulated APC of a matured APC sample for a fourth time period, thereby obtaining a stimulated T cell sample. In some embodiments, the method comprises incubating a T cell of a second stimulated T cell sample with a FLT3L-stimulated APC of a matured APC sample for a fifth time period, FLT3L and a third APC peptide loaded sample of a matured APC sample for a fifth time period, or FLT3L and a third APC peptide loaded sample of a matured APC sample for a fifth time period, thereby obtaining a stimulated T cell sample.

In some embodiments, the one or more separate time periods, the 3 or less separate time periods, the first time period, the second time period, the third time period, the fourth time period, or the fifth time period is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, or at least 40 hours.

In some embodiments, the one or more separate time periods, the 3 or less separate time periods, the first time period, the second time period, the third time period, the fourth time period, or the fifth time period is from 1 to 4 hours, from 1 to 3 hours, from 1 to 2 hours, from 4 to 40 hours, from 7 to 40 hours, from 4 to 35 hours, from 4 to 32 hours, from 7 to 35 hours or from 7 to 32 hours.

In some embodiments, the population of immune cells comprises the APC or at least one of the one or more APC preparations. In some embodiments, the population of immune cells does not comprise the APC and/or the population of immune cells does not comprise one of the one or more APC preparations.

In some embodiments, the method comprises incubating FLT3L and at least one peptide with a population of immune cells from a biological sample, wherein the FLT3L is incubated with the population of immune cells for a first time period and wherein the at least one peptide is incubated with the population of immune cells for a first peptide stimulation time period, thereby obtaining a first stimulated T cell sample, wherein the population of immune cells comprises at least one T cell and at least one APC. In some embodiments, the method comprises incubating FLT3L and at least one peptide with at least one APC, wherein the FLT3L is incubated with the at least one APC for a second time period and wherein the at least one peptide is incubated with the at least one APC for a second peptide stimulation time period, thereby obtaining a first matured APC peptide loaded sample; and incubating the first matured APC peptide loaded sample with the first stimulated T cell sample, thereby obtaining a second stimulated T cell sample. In some embodiments, the method comprises incubating FLT3L and at least one peptide with at least one APC, wherein the FLT3L is incubated with the at least one APC for a third time period and wherein the at least one peptide is incubated with the at least one APC for a third peptide stimulation time period, thereby obtaining a second matured APC peptide loaded sample; and incubating the second matured APC peptide loaded sample with the second stimulated T cell sample, thereby obtaining a third stimulated T cell sample.

In some embodiments, the method further comprises isolating the first stimulated T cell from the stimulated T cell sample. In some embodiments, isolating as described in the preceding sentence comprises enriching a stimulated T cell from a population of immune cells that have been contacted with the at least one APC incubated with the at least one peptide. In some embodiments, the enriching comprises determining expression of one or more cell markers of at least one the stimulated T cell and isolating the stimulated T cell expressing the one or more cell markers. In some embodiments the cell surface markers may be but not limited to one or more of TNF-α, IFN-γ, LAMP-1, 4-1BB, IL2, IL-17A, Granzyme B, PD-1, CD25, CD69, TIM3, LAG3, CTLA-4, CD62L, CD45RA, CD45RO, FoxP3, or any combination thereof. In some embodiments, the one or more cell markers comprise a cytokine.

In some embodiments, the method comprises administering at least one T cell of a first or a second or a third stimulated T cell sample to a subject in need thereof.

In some embodiments, the method comprises: obtaining a biological sample from a subject comprising at least one antigen presenting cell (APC); enriching cells expressing CD14 from the biological sample, thereby obtaining a CD14⁺ cell enriched sample; incubating the CD14⁺ cell enriched sample with at least one cytokine or growth factor for a first time period; incubating at least one peptide with the CD14⁺ cell enriched sample of for a second time period, thereby obtaining an APC peptide loaded sample; incubating the APC peptide loaded sample with one or more cytokines or growth factors for a third time period, thereby obtaining a matured APC sample; incubating APCs of the matured APC sample with a CD14 and CD25 depleted sample comprising T cells for a fourth time period; incubating the T cells with APCs of a matured APC sample for a fifth time period; incubating the T cells with APCs of a matured APC sample for a sixth time period; and administering at least one T cell of the T cells to a subject in need thereof.

In some embodiments, the method comprises: obtaining a biological sample from a subject comprising at least one APC and at least one T cell; depleting cells expressing CD14 and CD25 from the biological sample, thereby obtaining a CD14 and CD25 cell depleted sample; incubating the CD14 and CD25 cell depleted sample with FLT3L for a first time period; incubating at least one peptide with the CD14 and CD25 cell depleted sample of for a second time period, thereby obtaining an APC peptide loaded sample; incubating the APC peptide loaded sample with the at least one T cell for a third time period, thereby obtaining a first stimulated T cell sample; incubating a T cell of the first stimulated T cell sample with an APC of a matured APC sample for a fourth time period, thereby obtaining a second stimulated T cell sample; optionally, incubating a T cell of the second stimulated T cell sample with an APC of a matured APC sample for a fifth time period, thereby obtaining a third stimulated T cell sample; administering at least one T cell of the first, the second or the third stimulated T cell sample to a subject in need thereof.

In some embodiments, the method comprises: obtaining a biological sample from a subject comprising at least one APC and at least one T cell; depleting cells expressing CD14 and CD25 from the biological sample, thereby obtaining a CD14 and CD25 cell depleted sample; incubating the CD14 and CD25 cell depleted sample with FLT3L for a first time period; incubating at least one peptide with the CD14 and CD25 cell depleted sample of for a second time period, thereby obtaining an APC peptide loaded sample; incubating the APC peptide loaded sample with the at least one T cell for a third time period, thereby obtaining a first stimulated T cell sample; optionally, incubating a T cell of the first stimulated T cell sample with a FLT3L-stimulated APC of a matured APC sample for a fourth time period, thereby obtaining a second stimulated T cell sample; optionally, incubating a T cell of the second stimulated T cell sample with a FLT3L-stimulated APC of a matured APC sample for a fifth time period, thereby obtaining a third stimulated T cell sample; administering at least one T cell of the first, the second or the third stimulated T cell sample to a subject in need thereof.

In some embodiments, the method comprises: obtaining a biological sample from a subject comprising at least one APC and at least one T cell; depleting cells expressing CD14 and CD25 from the biological sample, thereby obtaining a CD14 and CD25 cell depleted sample; incubating the CD14 and CD25 cell depleted sample with FLT3L for a first time period; incubating at least one peptide with the CD14 and CD25 cell depleted sample of for a second time period, thereby obtaining a first APC peptide loaded sample; incubating the first APC peptide loaded sample with the at least one T cell for a third time period, thereby obtaining a first stimulated T cell sample; optionally, incubating a T cell of the first stimulated T cell sample with FLT3L and a second APC peptide loaded sample of a matured APC sample for a fourth time period, thereby obtaining a second stimulated T cell sample; optionally, incubating a T cell of the second stimulated T cell sample with FLT3L and a third APC peptide loaded sample of a matured APC sample for a fifth time period, thereby obtaining a third stimulated T cell sample; administering at least one T cell of the first, the second or the third stimulated T cell sample to a subject in need thereof.

In some embodiments, the method comprises: obtaining a biological sample from a subject comprising at least one APC and at least one T cell; depleting cells expressing CD14 and CD25 from the biological sample, thereby obtaining a CD14 and CD25 cell depleted sample; incubating the CD14 and CD25 cell depleted sample with FLT3L for a first time period; incubating at least one peptide with the CD14 and CD25 cell depleted sample of for a second time period, thereby obtaining a first APC peptide loaded sample; incubating the first APC peptide loaded sample with the at least one T cell for a third time period, thereby obtaining a first stimulated T cell sample; optionally, incubating a T cell of the first stimulated T cell sample with FLT3L and a FLT3L-stimulated APC of a matured APC sample for a fourth time period, thereby obtaining a second stimulated T cell sample; optionally, incubating a T cell of the second stimulated T cell sample with FLT3L and a FLT3L-stimulated APC of a matured APC sample for a fifth time period, thereby obtaining a third stimulated T cell sample; administering at least one T cell of the first, the second or the third stimulated T cell sample to a subject in need thereof.

In some embodiments, the method comprises: incubating FLT3L and at least one peptide with a population of immune cells from a biological sample, wherein the FLT3L is incubated with the population of immune cells for a first time period and wherein the at least one peptide is incubated with the population of immune cells for a first peptide stimulation time period, thereby obtaining a first stimulated T cell sample, wherein the population of immune cells comprises at least one T cell and at least one APC; optionally, incubating FLT3L and at least one peptide with at least one APC, wherein the FLT3L is incubated with the at least one APC for a second time period and wherein the at least one peptide is incubated with the at least one APC for a second peptide stimulation time period, thereby obtaining a first matured APC peptide loaded sample; and incubating the first matured APC peptide loaded sample with the first stimulated T cell sample, thereby obtaining a second stimulated T cell sample; optionally, incubating FLT3L and at least one peptide with at least one APC, wherein the FLT3L is incubated with the at least one APC for a third time period and wherein the at least one peptide is incubated with the at least one APC for a third peptide stimulation time period, thereby obtaining a second matured APC peptide loaded sample; and incubating the second matured APC peptide loaded sample with the second stimulated T cell sample, thereby obtaining a third stimulated T cell sample; and administering at least one T cell of the first stimulated T cell sample, the second stimulated T cell sample or the third stimulated T cell sample to a subject in need thereof.

In some embodiments, the method comprises generating cancer cell nucleic acids from a first biological sample comprising cancer cells obtained from a subject and generating non-cancer cell nucleic acids from a second biological sample comprising non-cancer cells obtained from the same subject.

In some embodiments, the method comprises sequencing cancer cell nucleic acids by whole genome sequencing or whole exome sequencing, thereby obtaining a first plurality of nucleic acid sequences comprising cancer cell nucleic acid sequences; and sequencing non-cancer cell nucleic acids by whole genome sequencing or whole exome sequencing, thereby obtaining a second plurality of nucleic acid sequences comprising non-cancer cell nucleic acid sequences. In some embodiments, the method comprises identifying a plurality of cancer specific nucleic acid sequences from a first plurality of nucleic acid sequences that are unique to cancer cells of the subject and that do not include nucleic acid sequences from a second plurality of nucleic acid sequences from non-cancer cells of the subject.

In some embodiments, the method further comprises selecting one or more subpopulation of cells from the expanded population of T cells prior to administering to the subject. In some embodiments, the selecting one or more subpopulation is performed by cell sorting based on expression of one or more cell surface markers provided herein. In some embodiments, the activated T cells may be sorted based on cell surface markers including but not limited to any one or more of the following: CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, CD45RO, CCR7, FLT3LG, IL-6 and others.

In some embodiments, the method further comprises depleting one or more cells in the subject prior to administering the population of T cells.

In some embodiments, the one or more subpopulation of cells expressing a cell surface marker provided herein.

In some embodiments, the amino acid sequence of a peptide provided herein is validated by peptide sequencing. In some embodiments, the amino acid sequence a peptide provided herein is validated by mass spectrometry.

Also provided herein is a pharmaceutical composition comprising a T cell produced by expanding the T cell in the presence of an antigen presenting cell presenting one or more epitope sequence of any of Tables 1-12.

Also provided herein is library of polypeptides comprising epitope sequences or polynucleotides encoding the polypeptides, wherein each epitope sequence in the library is matched to a protein encoded by an HLA allele; and wherein each epitope sequence in the library is pre-validated to satisfy at least two or three or four of the following criteria: binds to a protein encoded by an HLA allele of a subject with cancer to be treated, is immunogenic according to an immunogenic assay, is presented by APCs according to a mass spectrometry assay, and stimulates T cells to be cytotoxic according to a cytotoxicity assay. In some embodiments, the library comprises one or two or more peptide sequences comprising an epitope sequence of any of Tables 1-12.

The peptides and polynucleotides provided herein can be for preparing antigen-specific T cells and include recombinant peptides and polynucleotides and synthetic peptides comprising epitopes, such as a tumor-specific neoepitopes, that have been identified and validated as binding to one or more MHC molecules, presented by the one or more MHC molecules, being immunogenic and/or capable of activating T cells to become cytotoxic. The peptides can be prepared for use in a method to prime T cells ex vivo. The peptides can be prepared for use in a method to activate T cells ex vivo. The peptides can be prepared for use in a method to expand antigen-specific T cells. The peptides can be prepared for use in a method to induce de novo CD8 T cell responses ex vivo. The peptides can be prepared for use in a method to induce de novo CD4 T cell responses ex vivo. The peptides can be prepared for use in a method to stimulate memory CD8 T cell responses ex vivo. The peptides can be prepared for use in a method to stimulate memory CD4 T cell responses ex vivo. The T cells can be obtained from a human subject. The T cells can be allogeneic T cells. The T cells can be T cell lines.

The epitopes can comprise at least 8 contiguous amino acids of an amino acid sequence encoded by the genome of a cancer cell. The epitopes can comprise from 8-12 contiguous amino acids of an amino acid sequence encoded by the genome of a cancer cell. The epitopes can comprise from 13-25 contiguous amino acids of an amino acid sequence encoded by the genome of a cancer cell. The epitopes can comprise from 8-50 contiguous amino acids of an amino acid sequence encoded by the genome of a cancer cell. In some embodiments, an epitope is from about 8 and about 30 amino acids in length. In some embodiments, an epitope is from about 8 to about 25 amino acids in length. In some embodiments, an epitope is from about 15 to about 24 amino acids in length. In some embodiments, an epitope is from about 9 to about 15 amino acids in length. In some embodiments, an epitope is 8 amino acids in length. In some embodiments, an epitope is 9 amino acids in length. In some embodiments, an epitope is 10 amino acids in length.

In some embodiments, a peptide containing an epitope is at most 500, at most 250, at most 150, at most 125, or at most 100 amino acids in length In some embodiments, a peptide containing an epitope is at least 8, at least 50, at least 100, at least 200, or at least 300 amino acids in length. In some embodiments, a peptide containing an epitope is from about 8 to about 500 amino acids in length. In some embodiments, a peptide containing an epitope is from about 8 to about 100 amino acids in length. In some embodiments, a peptide containing an epitope is from about 8 to about 50 amino acids in length. In some embodiments, a peptide containing an epitope is from about 15 to about 35 amino acids in length. In some embodiments, a peptide containing an epitope is from about 8 and about 15 amino acids in length. In some embodiments, a peptide containing an epitope is from about 8 and about 11 amino acids in length. In some embodiments, a peptide containing an epitope is 9 or 10 amino acids in length. In some embodiments, a peptide containing an epitope is from about 8 and about 30 amino acids in length. In some embodiments, a peptide containing an epitope is from about 8 to about 25 amino acids in length. In some embodiments, a peptide containing an epitope is from about 15 to about 24 amino acids in length. In some embodiments, a peptide containing an epitope is from about 9 to about 15 amino acids in length.

In some embodiments, a peptide containing an epitope has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, or at least 500 amino acids. In some embodiments, a peptide containing an epitope has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, or at most 500 amino acids. In some embodiments, a peptide containing an epitope comprises a first neoepitope peptide linked to at least a second neoepitope.

In some embodiments, a peptide contains a validated epitope from one or more of: ABL1, AC011997, ACVR2A, AFP, AKT1, ALK, ALPPL2, ANAPC1, APC, ARID1A, AR, AR-v7, ASCL2, β2M, BRAF, BTK, C15ORF40, CDH1, CLDN6, CNOT1, CT45A5, CTAG1B, DCT, DKK4, EEF1B2, EEF1DP3, EGFR, EIF2B3, env, EPHB2, ERBB3, ESR1, ESRP1, FAM111B, FGFR3, FRG1B, GAGE1, GAGE10, GATA3, GBP3, HER2, IDH1, JAK1, KIT, KRAS, LMAN1, MABEB16, MAGEA1, MAGEA10, MAGEA4, MAGEA8, MAGEB17, MAGEB4, MAGEC1, MEK, MLANA, MLL2, MMP13, MSH3, MSH6, MYC, NDUFC2, NRAS, PAGE2, PAGE5, PDGFRa, PIK3CA, PMEL, pol protein, POLE, PTEN, RAC1, RBM27, RNF43, RPL22, RUNX1, SEC31A, SEC63, SF3B1, SLC35F5, SLC45A2, SMAP1, SMAP1, SPOP, TFAM, TGFBR2, THAP5, TP53, TTK, TYR, UBRS, VHL, XPOT an EEF1DP3:FRY fusion polypeptide, an EGFR:SEPT14 fusion polypeptide, an EGFRVIII deletion polypeptide, an EML4:ALK fusion polypeptide, an NDRG1:ERG fusion polypeptide, an AC011997.1:LRRC69 fusion polypeptide, a RUNX1(ex5)-RUNX1T1fusion polypeptide, a TMPRSS2:ERG fusion polypeptide, a NAB:STAT6 fusion polypeptide, a NDRG1:ERG fusion polypeptide, a PML:RARA fusion polypeptide, a PPP1R1B:STARD3 fusion polypeptide, a MAD1L1:MAFK fusion polypeptide, a FGFR3:TAC fusion polypeptide, a FGFR3:TACC3 fusion polypeptide, a BCR:ABL fusion polypeptide, a C11orf95:RELA fusion polypeptide, a CBFB:MYH11 fusion polypeptide, a CBFB:MYH11 fusion polypeptide, a CD74:ROS1 fusion polypeptide, a CD74:ROS1 fusion polypeptide, ERVE-4: protease, ERVE-4: reverse transcriptase, ERVE-4: reverse transcriptase, ERVE-4: unknown, ERVH-2 matrix protein, ERVH-2: gag, ERVH-2: retroviral matrix, ERVH48-1: coat protein, ERVH48-1: syncytin, ERVI-1 envelope protein, ERVK-5 gag, ERVK-5 env, ERVK-5 pol, EBV A73, EBV BALF3, EBV BALF4, EBV BALF5, EBV BARF0, EBV LF2, EBV RPMS1, HPV-16, HPV-16 E7, and HPV-16 E6. In some embodiments, a neoepitope contains a mutation due to a mutational event in β2M, BTK, EGFR, GATA3, KRAS, MLL2, a TMPRSS2:ERG fusion polypeptide, or TP53 or Myc.

In some embodiments, an epitope binds a major histocompatibility complex (MHC) class I molecule. In some embodiments, an epitope binds an MHC class I molecule with a binding affinity of about 500 nM or less. In some embodiments an epitope binds an MHC class I molecule with a binding affinity of about 250 nM or less. In some embodiments, an epitope binds an MHC class I molecule with a binding affinity of about 150 nM or less. In some embodiments, an epitope binds an MHC class I molecule with a binding affinity of about 50 nM or less.

In some embodiments, an epitope binds an binds MHC class I molecule and a peptide containing the class I epitope binds to an MHC class II molecule.

In some embodiments, an epitope binds an MHC class II molecule. In some embodiments, an epitope binds to human leukocyte antigen (HLA)-A, -B, -C, -DP, -DQ, or -DR. In some embodiments, an epitope binds an MHC class II molecule with a binding affinity of 1000 nM or less. In some embodiments, an epitope binds MHC class II with a binding affinity of 500 nM or less. In some embodiments an epitope binds an MHC class II molecule with a binding affinity of about 250 nM or less. In some embodiments, an epitope binds an MHC class II molecule with a binding affinity of about 150 nM or less. In some embodiments, an epitope binds an MHC class II molecule with a binding affinity of about 50 nM or less.

In some embodiments, a peptide containing a validated epitope further comprises one or more amino acids flanking the C-terminus of the epitope. In some embodiments, a peptide containing a validated epitope further comprises one or more amino acids flanking the N-terminus of the epitope. In some embodiments, a peptide containing a validated epitope further comprises one or more amino acids flanking the C-terminus of the epitope and one or more amino acids flanking the N-terminus of the epitope. In some embodiments, the flanking amino acids are not native flanking amino acids. In some embodiments, a first epitope used in a method described herein binds an MHC class I molecule and a second epitope binds an MHC class II molecule. In some embodiments, a peptide containing a validated epitope further comprises a modification which increases in vivo half-life of the peptide. In some embodiments, a peptide containing a validated epitope further comprises a modification which increases cellular targeting by the peptide. In some embodiments, a peptide containing a validated epitope further comprises a modification which increases cellular uptake of the peptide. In some embodiments, a peptide containing a validated epitope further comprises a modification which increases peptide processing. In some embodiments, a peptide containing a validated epitope further comprises a modification which increases MHC affinity of the epitope. In some embodiments, a peptide containing a validated epitope further comprises a modification which increases MHC stability of the epitope. In some embodiments, a peptide containing a validated epitope further comprises a modification which increases presentation of the epitope by an MHC class I molecule, and/or an MHC class II molecule.

In some embodiments, sequencing methods are used to identify tumor specific mutations. Any suitable sequencing method can be used according to the invention, for example, Next Generation Sequencing (NGS) technologies. Third Generation Sequencing methods might substitute for the NGS technology in the future to speed up the sequencing step of the method. For clarification purposes: the terms “Next Generation Sequencing” or “NGS” in the context of the present invention mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, for example, within 1-7 days or within less than 24 hours and allow, in principle, single cell sequencing approaches. Multiple NGS platforms which are commercially available or which are mentioned in the literature can be used in the context of the invention e.g. those described in detail in WO 2012/159643.

In some embodiments, a peptide containing a validated epitope is linked to the at least second peptide, such as by a poly-glycine or poly-serine linker. In some embodiments, the modification is conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. In some embodiments, a peptide containing a validated epitope further comprises a modification which increases cellular targeting to specific organs, tissues, or cell types. In some embodiments, a peptide containing a validated epitope comprises an antigen presenting cell targeting moiety or marker. In some embodiments, the antigen presenting cells are dendritic cells. In some embodiments, the dendritic cells are targeted using DEC205, XCR1, CD197, CD80, CD86, CD123, CD209, CD273, CD283, CD289, CD184, CD85h, CD85j, CD85k, CD85d, CD85g, CD85a, CD141, CD11c, CD83, TSLP receptor, Clec9a, or CD1a marker. In some embodiments, the dendritic cells are targeted using the CD141, DEC205, Clec9a, or XCR1 marker. In some embodiments, the dendritic cells are autologous cells. In some embodiments, one or more of the dendritic cells are bound to a T cell.

In some embodiments, the method described herein comprises large scale manufacture of and storage of HLA-matched peptides corresponding to shared antigens for treatment of a cancer or a tumor.

In some embodiments, the method described herein comprises treatment methods, comprising administering to a subject with cancer antigen-specific T cell that are specific to a validated epitope selected from the HLA matched peptide repertoire presented in any of Tables 1-12. In some embodiments, epitope-specific T cells are administered to the patient by infusion. In some embodiments, the T cells are administered to the patient by direct intravenous injection. In some embodiments, the T cell is an autologous T cell. In some embodiments, the T cell is an allogeneic T cell.

The methods of the disclosure can be used to treat any type of cancer known in the art. In some embodiments, a method of treating cancer comprises treating breast cancer, prostate cancer, ovarian cancer, cervical cancer, skin cancer, pancreatic cancer, colorectal cancer, renal cancer, liver cancer, brain cancer, lung cancer, metastatic melanoma, thymoma, lymphoma, sarcoma, mesothelioma, renal cell carcinoma, stomach cancer, gastric cancer, ovarian cancer, NHL, leukemia, uterine cancer, colon cancer, bladder cancer, kidney cancer or endometrial cancer. In some embodiments, the cancer is selected from the group consisting of carcinoma, lymphoma, blastoma, sarcoma, leukemia, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, melanoma, endometrial or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblasts leukemia, and post-transplant lymphoproliferative disorder (PTLD), abnormal vascular proliferation associated with phakomatoses, edema, Meigs' syndrome. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is prostate cancer. In some embodiments, a cancer to be treated by the methods of treatment of the present disclosure is colorectal cancer. In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

The pharmaceutical compositions provided herein may be used alone or in combination with conventional therapeutic regimens such as surgery, irradiation, chemotherapy and/or bone marrow transplantation (autologous, syngeneic, allogeneic or unrelated). In some embodiments, at least one or more chemotherapeutic agents may be administered in addition to the pharmaceutical composition comprising an immunogenic therapy. In some embodiments, the one or more chemotherapeutic agents may belong to different classes of chemotherapeutic agents. In practicing the methods of treatment or use provided herein, therapeutically effective amounts of the pharmaceutical compositions can be administered to a subject having a disease or condition. A therapeutically effective amount can vary widely depending on the severity of the disease, the age and relative health of the subject, the potency of the compounds used, and other factors.

In some embodiments, the methods for treatment include one or more rounds of leukapheresis prior to transplantation of T cells. The leukapheresis may include collection of peripheral blood mononuclear cells (PBMCs). Leukapheresis may include mobilizing the PBMCs prior to collection. Alternatively, non-mobilized PBMCs may be collected. A large volume of PBMCs may be collected from the subject in one round. Alternatively, the subject may undergo two or more rounds of leukapheresis. The volume of apheresis may be dependent on the number of cells required for transplant. For instance, 12-15 liters of non-mobilized PBMCs may be collected from a subject in one round. The number of PBMCs to be collected from a subject may be between 1×10⁸ to 5×10¹⁰ cells. The number of PBMCs to be collected from a subject may be 1×10⁸, 5×10⁸, 1×10⁹, 5×10⁹, 1×10¹⁰ or 5×10¹⁰ cells. The minimum number of PBMCs to be collected from a subject may be 1×10⁶/kg of the subject's weight. The minimum number of PBMCs to be collected from a subject may be 1×10⁶/kg, 5×10⁶/kg, 1×10⁷/kg, 5×10⁷/kg, 1×10⁸/kg, 5×10⁸/kg of the subject's weight.

A single infusion may comprise a dose between 1×10⁶ cells per square meter body surface of the subject (cells/m²) and 5×10⁹ cells/m². A single infusion may comprise between about 2.5×10⁶ to about 9 cells/m². A single infusion may comprise between at least about 2.5×10⁶ cells/m². A single infusion may comprise between at most 5×10⁹ cells/m². A single infusion may comprise between 1×10⁶ to 2.5×10⁶, 1×10⁶ to 5×10⁶, 1×10⁶ to 7.5×10⁶, 1×10⁶ to 1×10⁷, 1×10⁶ to 5×10⁷, 1×10⁶ to 7.5×10⁷, 1×10⁶ to 1×10⁸, 1×10⁶ to 2.5×10⁸, 1×10⁶ to 5×10⁸, 1×10⁶ to 1×10⁹, 1×10⁶ to 5×10⁹, 2.5×10⁶ to 5×10⁶, 2.5×10⁶ to 7.5×10⁶, 2.5×10⁶ to 1×10⁷, 2.5×10⁶ to 5×10⁷, 2.5×10⁶ to 7.5×10⁷, 2.5×10⁶ to 1×10⁸, 2.5×10⁶ to 2.5×10⁸, 2.5×10⁶ to 5×10⁸, 2.5×10⁶ to 1×10⁹, 2.5×10⁶ to 5×10⁹, 5×10⁶ to 7.5×10⁶, 5×10⁶ to 1×10⁷, 5×10⁶ to 5×10⁷, 5×10⁶ to 7.5×10⁷, 6 to 1×10⁸, 5×10⁶ to 2.5×10⁸, 5×10⁶ to 5×10⁸, 5×10⁶ to 1×10⁹, 5×10⁶ to 5×10⁹, 7.5×10⁶ to 1×10⁷, 7.5×10⁶ to 5×10⁷, 7.5×10⁶ to 7.5×10⁷, 7.5×10⁶ to 1×10⁸, 7.5×10⁶ to 2.5×10⁸, 7.5×10⁶ to 5×10⁸, 7.5×10⁶ to 1×10⁹, 7.5×10⁶ to 5×10⁹, 1×10⁷ to 5×10⁷, 1×10⁷ to 7.5×10⁷, 1×10⁷ to 1×10⁸, 1×10⁷ to 2.5×10⁸, 1×10⁷ to 5×10⁸, 1×10⁷ to 1×10⁹, 1×10⁷ to 5×10⁹, 5×10⁷ to 7.5×10⁷, 5×10⁷ to 1×10⁸, 5×10⁷ to 2.5×10⁸, 5×10⁷ to 5×10⁸, 5×10⁷ to 1×10⁹, 5×10⁷ to 5×10⁹, 7.5×10⁷ to 1×10⁸, 7.5×10⁷ to 2.5×10⁸, 7.5×10⁷ to 5×10⁸, 7.5×10⁷ to 1×10⁹, 7.5×10⁷ to 5×10⁹, 1×10⁸ to 2.5×10⁸, 1×10⁸ to 5×10⁸, 1×10⁸ to 1×10⁹, 1×10⁸ to 5×10⁹, 2.5×10⁸ to 5×10⁸, 2.5×10⁸ to 1×10⁹, 2.5×10⁸ to 5×10⁹, 5×10⁸ to 1×10⁹, 5×10⁸ to 5×10⁹, or 1×10⁹ to 5×10⁹ cells/m². A single infusion may comprise between 1×10⁶ cells/m², 2.5×10⁶ cells/m², 5×10⁶ cells/m², 7.5×10⁶ cells/m², 1×10⁷ cells/m², 5×10⁷ cells/m², 7.5×10⁷ cells/m², 1×10⁸ cells/m², 2.5×10⁸ cells/m², 5×10⁸ cells/m², 1×10⁹ cells/m², or 5×10⁹ cells/m².

The methods may include administering chemotherapy to a subject including lymphodepleting chemotherapy using high doses of myeloablative agents. In some embodiments, the methods include administering a preconditioning agent, such as a lymphodepleting or chemotherapeutic agent, such as cyclophosphamide, fludarabine, or combinations thereof, to a subject prior to the first or subsequent dose. For example, the subject may be administered a preconditioning agent at least 2 days prior, such as at least 3, 4, 5, 6, 7, 8, 9 or 10 days prior, to the first or subsequent dose. In some embodiments, the subject is administered a preconditioning agent no more than 10 days prior, such as no more than 9, 8, 7, 6, 5, 4, 3, or 2 days prior, to the first or subsequent dose.

In some embodiments, where the lymphodepleting agent comprises cyclophosphamide, the subject is administered between 0.3 grams per square meter of the body surface of the subject (g/m²) and 5 g/m² cyclophosphamide. In some cases, the amount of cyclophosphamide administered to a subject is about at least 0.3 g/m². In some cases, the amount of cyclophosphamide administered to a subject is about at most 5 g/m². In some cases, the amount of cyclophosphamide administered to a subject is about 0.3 g/m² to 0.4 g/m², 0.3 g/m² to 0.5 g/m², 0.3 g/m² to 0.6 g/m², 0.3 g/m² to 0.7 g/m², 0.3 g/m² to 0.8 g/m², 0.3 g/m² to 0.9 g/m², 0.3 g/m² to 1 g/m², 0.3 g/m² to 2 g/m², 0.3 g/m² to 3 g/m², 0.3 g/m² to 4 g/m², 0.3 g/m² to 5 g/m², 0.4 g/m² to 0.5 g/m², 0.4 g/m² to 0.6 g/m², 0.4 g/m² to 0.7 g/m², 0.4 g/m² to 0.8 g/m², 0.4 g/m² to 0.9 g/m², 0.4 g/m² to 1 g/m², 0.4 g/m² to 2 g/m², 0.4 g/m² to 3 g/m², 0.4 g/m² to 4 g/m², 0.4 g/m² to 5 g/m², 0.5 g/m² to 0.6 g/m², 0.5 g/m² to 0.7 g/m², 0.5 g/m² to 0.8 g/m², 0.5 g/m² to 0.9 g/m², 0.5 g/m² to 1 g/m², 0.5 g/m² to 2 g/m², 0.5 g/m² to 3 g/m², 0.5 g/m² to 4 g/m², 0.5 g/m² to 5 g/m², 0.6 g/m² to 0.7 g/m², 0.6 g/m² to 0.8 g/m², 0.6 g/m² to 0.9 g/m², 0.6 g/m² to 1 g/m², 0.6 g/m² to 2 g/m², 0.6 g/m² to 3 g/m², 0.6 g/m² to 4 g/m², 0.6 g/m² to 5 g/m², 0.7 g/m² to 0.8 g/m², 0.7 g/m² to 0.9 g/m², 0.7 g/m² to 1 g/m², 0.7 g/m² to 2 g/m², 0.7 g/m² to 3 g/m², 0.7 g/m² to 4 g/m², 0.7 g/m² to 5 g/m², 0.8 g/m² to 0.9 g/m², 0.8 g/m² to 1 g/m², 0.8 g/m² to 2 g/m², 0.8 g/m² to 3 g/m², 0.8 g/m² to 4 g/m², 0.8 g/m² to 5 g/m², 0.9 g/m² to 1 g/m², 0.9 g/m² to 2 g/m², 0.9 g/m² to 3 g/m², 0.9 g/m² to 4 g/m², 0.9 g/m² to 5 g/m², 1 g/m² to 2 g/m², 1 g/m² to 3 g/m², 1 g/m² to 4 g/m², 1 g/m² to 5 g/m², 2 g/m² to 3 g/m², 2 g/m² to 4 g/m², 2 g/m² to 5 g/m², 3 g/m² to 4 g/m², 3 g/m² to 5 g/m², or 4 g/m² to 5 g/m². In some cases, the amount of cyclophosphamide administered to a subject is about 0.3 g/m², 0.4 g/m², 0.5 g/m², 0.6 g/m², 0.7 g/m², 0.8 g/m², 0.9 g/m², 1 g/m², 2 g/m², 3 g/m², 4 g/m², or 5 g/m². In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide.

In some embodiments, where the lymphodepleting agent comprises fludarabine, the subject is administered fludarabine at a dose between or between about 1 milligrams per square meter of the body surface of the subject (mg/m²) and 100 mg/m². In some cases, the amount of fludarabine administered to a subject is about at least 1 mg/m². In some cases, the amount of fludarabine administered to a subject is about at most 100 mg/m². In some cases, the amount of fludarabine administered to a subject is about 1 mg/m² to 5 mg/m², 1 mg/m² to 10 mg/m², 1 mg/m² to 15 mg/m², 1 mg/m² to 20 mg/m², 1 mg/m² to 30 mg/m², 1 mg/m² to 40 mg/m², 1 mg/m² to 50 mg/m², 1 mg/m² to 70 mg/m², 1 mg/m² to 90 mg/m², 1 mg/m² to 100 mg/m², 5 mg/m² to 10 mg/m², 5 mg/m² to 15 mg/m², 5 mg/m² to 20 mg/m², 5 mg/m² to 30 mg/m², 5 mg/m² to 40 mg/m², 5 mg/m² to 50 mg/m², 5 mg/m² to 70 mg/m², 5 mg/m² to 90 mg/m², 5 mg/m² to 100 mg/m², 10 mg/m² to 15 mg/m², 10 mg/m² to 20 mg/m², 10 mg/m² to 30 mg/m², 10 mg/m² to 40 mg/m², 10 mg/m² to 50 mg/m², 10 mg/m² to 70 mg/m², 10 mg/m² to 90 mg/m², 10 mg/m² to 100 mg/m², 15 mg/m² to 20 mg/m², 15 mg/m² to 30 mg/m², 15 mg/m² to 40 mg/m², 15 mg/m² to 50 mg/m², 15 mg/m² to 70 mg/m², 15 mg/m² to 90 mg/m², 15 mg/m² to 100 mg/m², 20 mg/m² to 30 mg/m², 20 mg/m² to 40 mg/m², 20 mg/m² to 50 mg/m², 20 mg/m² to 70 mg/m², 20 mg/m² to 90 mg/m², 20 mg/m² to 100 mg/m², 30 mg/m² to 40 mg/m², 30 mg/m² to 50 mg/m², 30 mg/m² to 70 mg/m², 30 mg/m² to 90 mg/m², 30 mg/m² to 100 mg/m², 40 mg/m² to 50 mg/m², 40 mg/m² to 70 mg/m², 40 mg/m² to 90 mg/m², 40 mg/m² to 100 mg/m², 50 mg/m² to 70 mg/m², 50 mg/m² to 90 mg/m², 50 mg/m² to 100 mg/m², 70 mg/m² to 90 mg/m², 70 mg/m² to 100 mg/m², or 90 mg/m² to 100 mg/m². In some cases, the amount of fludarabine administered to a subject is about 1 mg/m², 5 mg/m², 10 mg/m², 15 mg/m², 20 mg/m², 30 mg/m², 40 mg/m², 50 mg/m², 70 mg/m², 90 mg/m², or 100 mg/m². In some embodiments, the fludarabine can be administered in a single dose or can be administered in a plurality of doses, such as given daily, every other day or every three days. For example, in some instances, the agent, e.g., fludarabine, is administered between or between about 1 and 5 times, such as between or between about 3 and 5 times. In some embodiments, such plurality of doses is administered in the same day, such as 1 to 5 times or 3 to 5 times daily.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 400 mg/m² of cyclophosphamide and one or more doses of 20 mg/m² fludarabine prior to the first or subsequent dose of T cells. In some examples, the subject is administered 500 mg/m² of cyclophosphamide and one or more doses of 25 mg/m² fludarabine prior to the first or subsequent dose of T cells. In some examples, the subject is administered 600 mg/m² of cyclophosphamide and one or more doses of 30 mg/m² fludarabine prior to the first or subsequent dose of T cells. In some examples, the subject is administered 700 mg/m² of cyclophosphamide and one or more doses of 35 mg/m² fludarabine prior to the first or subsequent dose of T cells. In some examples, the subject is administered 700 mg/m² of cyclophosphamide and one or more doses of 40 mg/m² fludarabine prior to the first or subsequent dose of T cells. In some examples, the subject is administered 800 mg/m² of cyclophosphamide and one or more doses of 45 mg/m² fludarabine prior to the first or subsequent dose of T cells.

Fludarabine and cyclophosphamide may be administered on alternative days. In some cases, fludarabine and cyclophosphamide may be administered concurrently. In some cases, an initial dose of fludarabine is followed by a dose of cyclophosphamide. In some cases, an initial dose of cyclophosphamide may be followed by an initial dose of fludarabine. In some examples, a treatment regimen may include treatment of a subject with an initial dose of fludarabine 10 days prior to the transplant, followed by treatment with an initial dose of cyclophosphamide administered 9 days prior to the cell transplant, concurrently with a second dose of fludarabine. In some examples, a treatment regimen may include treatment of a subject with an initial dose of fludarabine 8 days prior to the transplant, followed by treatment with an initial dose of cyclophosphamide administered 7 days prior to the transplant concurrently with a second dose of fludarabine.

In some aspects, provided herein is a composition comprising: (a) APCs expressing a protein encoded by an HLA-C03:04 allele, wherein the APCs comprise a peptide or a polynucleotide encoding the peptide; and (b) T cells stimulated with APCs of (a); wherein the peptide comprises an epitope with a sequence GACGVGKSA.

In some aspects, provided herein is a composition comprising: (a) APCs expressing a protein encoded by an HLA-C03:04 allele, wherein the APCs comprise a peptide or a polynucleotide encoding the peptide; and (b) T cells stimulated with APCs of (a); wherein the peptide comprises an epitope with a sequence GACGVGKSA, and further comprises (c) antigen presenting cells expressing a protein encoded by an HLA allele of a subject, wherein the APCs further comprise a peptide selected from Tables 1-12, that binds to the protein encoded by the HLA of the subject and (d) T cells stimulated with APCs of (c).

In some aspects, provided herein is a composition comprising: (a) APCs expressing a protein encoded by an HLA-C03:03 allele, wherein the APCs comprise a peptide or a polynucleotide encoding the peptide; (b) T cells stimulated with APCs of (a); wherein the peptide comprises an epitope with a sequence GAVGVGKSA.

In some embodiments, provided herein is a composition comprising: (a) APCs expressing a protein encoded by an HLA-C03:03 allele, wherein the APCs comprise a peptide or a polynucleotide encoding the peptide; and (b) T cells stimulated with APCs of (a); wherein the peptide comprises an epitope with a sequence GAVGVGKSA, and further comprises (c) antigen presenting cells expressing a protein encoded by an HLA allele of a subject, wherein the APCs further comprise a peptide selected from Table 1-12, that binds to the protein encoded by the HLA of the subject and (d) T cells stimulated with APCs of (c).

Approximately 9% of US population show positive expression for a protein encoded by HLA-C03:03. Approximately, 15% of US population show positive expression for a protein encoded by HLA-C03.04. In some embodiments the compositions described herein are required for treating a KRAS mutation-related cancer. KRAS mutations are particularly prevalent in pancreatic and colorectal cancers. Approximate frequencies of G12C, D, V and R mutations in the different forms of cancer are indicated below: (PDAC, pancreatic ductal adenocarcinoma, NSCLC, non-small cell lung cancer; CRC, colorectal cancer).

| | PDAC | NSCLC | CRC |
|---|---|---|---|
| G12C | 1.83% | 12.71% | 3.39% |
| G12D | 38.83% | 3.54% | 12.08% |
| G12V | 33.51% | 4.65% | 7.96% |
| G12R | 16.30% | 0.26% | 0.55% |

In some embodiments, the composition further comprises a peptide that comprises an epitope sequence according to any one of Tables 1-12, or an APC loaded with and expressing a peptide that comprises an epitope sequence according to any one of Tables 1-12 antigen presenting cells (APCs), or a T cell stimulated with an APC expressing a peptide that comprises an epitope sequence according to any one of Tables 1-12 antigen presenting cells (APCs). In some embodiments, a peptide comprises an epitope sequence according to Table 1. In some embodiments, a peptide comprises an epitope sequence according to Table 2. In some embodiments, a peptide comprises an epitope sequence according to Table 3. In some embodiments, a peptide comprises an epitope sequence according to Table 4. In some embodiments, a peptide comprises an epitope sequence according to Table 5. In some embodiments, a peptide comprises an epitope sequence according to Table 6. In some embodiments, a peptide comprises an epitope sequence according to Table 7. In some embodiments, a peptide comprises an epitope sequence according to Table 8. In some embodiments, a peptide comprises an epitope sequence according to Table 9. In some embodiments, a peptide comprises an epitope sequence according to Table 10. In some embodiments, a peptide comprises an epitope sequence according to Table 11. In some embodiments, a peptide comprises an epitope sequence according to Table 12.

TABLE 8

| | Amino | | | |
|---|---|---|---|---|
| | Acid | | Peptides (Binding HLA allele | Exemplary |
| Gene | Alteration | Mutation Sequence Context | example(s)) | Diseases |
| KRAS | G12C | MTEYKLVVVGACGVGKSA | KLVVVGACGV (A02.01) | BRCA, CESC, |
| | | LTIQLIQNHFVDEYDPTIEDS | LVVVGACGV (A02.01) | CRC, HNSC, |
| | | YRKQVVIDGETCLLDILDT | VVGACGVGK (A03.01, A11.01) | LUAD, PAAD, |
| | | AGQE | VVVGACGVGK (A03.01) | UCEC |
| KRAS | G12D | MTEYKLVVVGADGVGKSA | VVGADGVGK (A11.01) | BLCA, BRCA, |
| | | LTIQLIQNHFVDEYDPTIEDS | VVVGADGVGK (A11.01) | CESC, CRC, |
| | | YRKQVVIDGETCLLDILDT | KLVVVGADGV (A02.01) | GBM, HNSC, |
| | | AGQE | LVVVGADGV (A02.01) | KIRP, LIHC, |
| | | | | LUAD, PAAD, |
| | | | | SKCM, UCEC |
| KRAS | G12V | MTEYKLVVVGAVGVGKSA | KLVVVGAVGV (A02.01) | BRCA, CESC, |
| | | LTIQLIQNHFVDEYDPTIEDS | LVVVGAVGV (A02.01) | CRC, LUAD, |
| | | YRKQVVIDGETCLLDILDT | VVGAVGVGK (A03.01, | PAAD, THCA, |
| | | AGQE | A11.01) | UCEC |
| | | | VVVGAVGVGK (A03.01, | |
| | | | A11.01) | |
| KRAS | Q61H | AGGVGKSALTIQLIQNHFV | ILDTAGHEEY (A01.01) | CRC, LUSC, |
| | | DEYDPTIEDSYRKQVVIDGE | | PAAD, |
| | | TCLLDILDTAGHEEYSAMR | | SKCM, UCEC |
| | | DQYMRTGEGFLCVFAINNT | | |
| | | KSFEDIHHYREQIKRVKDSE | | |
| | | DVPM | | |
| KRAS | Q61L | AGGVGKSALTIQLIQNHFV | ILDTAGLEEY (A01.01) | CRC, GBM, |
| | | DEYDPTIEDSYRKQVVIDGE | LLDILDTAGL (A02.01) | HNSC, LUAD, |
| | | TCLLDILDTAGLEEYSAMR | | SKCM, UCEC |
| | | DQYMRTGEGFLCVFAINNT | | |
| | | KSFEDIHHYREQIKRVKDSE | | |
| | | DVPM | | |
| NRAS | Q61K | AGGVGKSALTIQLIQNHFV | ILDTAGKEEY (A01.01) | BLCA, CRC, |
| | | DEYDPTIEDSYRKQVVIDGE | | LIHC, LUAD, |
| | | TCLLDILDTAGKEEYSAMR | | LUSC, SKCM, |
| | | DQYMRTGEGFLCVFAINNS | | THCA, UCEC |
| | | KSFADINLYREQIKRVKDSD | | |
| | | DVPM | | |
| NRAS | Q61R | AGGVGKSALTIQLIQNHFV | ILDTAGREEY (A01.01) | BLCA, CRC, |
| | | DEYDPTIEDSYRKQVVIDGE | | LUSC, PAAD, |
| | | TCLLDILDTAGREEYSAMR | | PRAD, SKCM, |
| | | DQYMRTGEGFLCVFAINNS | | THCA, UCEC |
| | | KSFADINLYREQIKRVKDSD | | |
| | | DVPM | | |
| BTK | C481S | MIKEGSMSEDEFIEEAKV | EYMANGSLL (A24.02) | CLL |
| | | MMNLSHEKLVQLYGVCT | MANGSLLNY (A01.01, | |
| | | KQRPIFIITEYMANGSLLN | A03.01, A11.01) | |
| | | YLREMRHRFQTQQLLEMC | MANGSLLNYL (A02.01, | |
| | | KDVCEAMEYLESKQFLHR | B07.02, B08.01) | |
| | | DLAARNCLVND | SLLNYLREM (A02.01, | |
| | | | B07.02, B08.01) | |
| | | | YMANGSLLN (A02.01) | |
| | | | YMANGSLLNY (A01.01, | |
| | | | A03.01, A11.01) | |
| EGFR | S492R | SLNITSLGLRSLKEISDGDV | IIRNRGENSCK (A03.01) | CRC |
| | | IISGNKNLCYANTINWKKL | | |
| | | FGTSGQKTKIIRNRGENSC | | |
| | | KATGQVCHALCSPEGCW | | |
| | | GPEPRDCVSCRNVSRGRE | | |
| | | CVDKCNLL | | |
| EGFR | T790M | IPVAIKELREATSPKANKEI | CLTSTVQLIM (A01.01, | NSCLC, |
| | | LDEAYVMASVDNPHVCR | A02.01) | PRAD |
| | | LLGICLTSTVQLIMQLMPF | IMQLMPFGC (A02.01) | |
| | | GCLLDYVREHKDNIGSQY | IMQLMPFGCL (A02.01, | |
| | | LLNWCVQIAKGMNYLED | A24.02, B08.01) | |
| | | RRLVHRDLAA | LIMQLMPFG (A02.01) | |
| | | | LIMQLMPFGC (A02.01) | |
| | | | LTSTVQLIM (A01.01) | |
| | | | MQLMPFGCL (A02.01, | |
| | | | B07.02, B08.01) | |
| | | | MQLMPFGCLL (A02.01, | |
| | | | A24.02, B08.01) | |
| | | | QLIMQLMPF (A02.01, | |
| | | | A24.02, B08.01) | |
| | | | QLIMQLMPFG (A02.01) | |
| | | | STVQLIMQL (A02.01) | |
| | | | VQLIMQLMPF (A02.01, | |
| | | | A24.02, B08.01) | |
| ABL1 | E255K | VADGLITTLHYPAPKRNKP | GQYGKVYEG (A02.01) | Chronic |
| | | TVYGVSPNYDKWEMERT | GQYGKVYEGV (A02.01) | myeloid |
| | | DITMKHKLGGGQYGKVY | KLGGGQYGK (A03.01) | leukemia |
| | | EGVWKKYSLTVAVKTLK | KLGGGQYGKV (A02.01) | (CML), Acute |
| | | EDTMEVEEFLKEAAVMKE | KVYEGVWKK (A02.01, | lymphocytic |
| | | IKHPNLVQLLGVC | A03.01) | leukemia |
| | | | KVYEGVWKKY (A03.01) | (ALL), |
| | | | QYGKVYEGV (A24.02) | Gastrointestina |
| | | | QYGKVYEGVW (A24.02) | 1 stromal |
| | | | | tumors (GIST) |
| ABL1 | E255V | VADGLITTLHYPAPKRNKP | GQYGVVYEG (A02.01) | Chronic |
| | | TVYGVSPNYDKWEMERT | GQYGVVYEGV (A02.01) | myeloid |
| | | DITMKHKLGGGQYGVVY | KLGGGQYGV (A02.01) | leukemia |
| | | EGVWKKYSLTVAVKTLK | KLGGGQYGVV (A02.01) | (CML), Acute |
| | | EDTMEVEEFLKEAAVMKE | QYGVVYEGV (A24.02) | lymphocytic |
| | | IKHPNLVQLLGVC | QYGVVYEGVW (A24.02) | leukemia |
| | | | VVYEGVWKK (A02.01, | (ALL), |
| | | | A03.01) | Gastrointestina |
| | | | VVYEGVWKKY (A03.01) | 1 stromal |
| | | | | tumors (GIST) |
| ABL1 | M351T | LLGVCTREPPFYIITEFMTY | ATQISSATEY (A01.01) | Chronic |
| | | GNLLDYLRECNRQEVNAV | ISSATEYLEK (A03.01) | myeloid |
| | | VLLYMATQISSATEYLEK | SSATEYLEK (A03.01) | leukemia |
| | | KNFIHRDLAARNCLVGEN | TQISSATEYL (A02.01) | (CML), Acute |
| | | HLVKVADFGLSRLMTGDT | YMATQISSAT (A02.01) | lymphocytic |
| | | YTAHAGAKF | | leukemia |
| | | | | (ALL), |
| | | | | Gastrointestina |
| | | | | 1 stromal |
| | | | | tumors (GIST) |
| ABL1 | T315I | SLTVAVKTLKEDTMEVEE | FYIIIEFMTY (A24.02) | Chronic |
| | | FLKEAAVMKEIKHPNLVQ | IIEFMTYGNL (A02.01) | myeloid |
| | | LLGVCTREPPFYIIIEFMTY | IIIEFMTYG (A02.01) | leukemia |
| | | GNLLDYLRECNRQEVNAV | IIIEFMTYGN (A02.01) | (CML), Acute |
| | | VLLYMATQISSAMEYLEK | YIIIEFMTYG (A02.01) | lymphocytic |
| | | KNFIHRDLA | | leukemia |
| | | | | (ALL), |
| | | | | Gastrointestina |
| | | | | 1 stromal |
| | | | | tumors (GIST) |
| ABL1 | Y253H | STVADGLITTLHYPAPKRN | GQHGEVYEGV (A02.01) | Chronic |
| | | KPTVYGVSPNYDKWEME | KLGGGQHGEV (A02.01) | myeloid |
| | | RTDITMKHKLGGGQHGEV | | leukemia |
| | | YEGVWKKYSLTVAVKTL | | (CML), Acute |
| | | KEDTMEVEEFLKEAAVM | | lymphocytic |
| | | KEIKHPNLVQLLG | | leukemia |
| | | | | (ALL), |
| | | | | Gastrointestina |
| | | | | 1 stromal |
| | | | | tumors (GIST) |
| ALK | G1269A | SSLAMLDLLHVARDIACG | KIADFGMAR (A03.01) | NSCLC |
| | | CQYLEENHFIHRDIAARNC | RVAKIADFGM (A02.01, | |
| | | LLTCPGPGRVAKIADFGM | B07.02) | |
| | | ARDIYRASYYRKGGCAML | | |
| | | PVKWMPPEAFMEGIFTSK | | |
| | | TDTWSFGVLL | | |
| ALK | L1196M | QVAVKTLPEVCSEQDELD | FILMELMAGG (A02.01) | NSCLC |
| | | FLMEALIISKFNHQNIVRCI | ILMELMAGG (A02.01) | |
| | | GVSLQSLPRFILMELMAG | ILMELMAGGD (A02.01) | |
| | | GDLKSFLRETRPRPSQPSS | LMELMAGGDL (A02.01) | |
| | | LAMLDLLHVARDIACGCQ | LPRFILMEL (B07.02, | |
| | | YLEENHFI | B08.01) | |
| | | | LPRFILMELM (B07.02) | |
| | | | LQSLPRFILM (A02.01, | |
| | | | B08.01) | |
| | | | SLPRFILMEL (A02.01, | |
| | | | A24.02, B07.02, B08.01) | |
| BRAF | V600E | MIKLIDIARQTAQGMDYL | LATEKSRWS (A02.01, | CRC, GBM, |
| | | HAKSIIHRDLKSNNIFLHE | B08.01) | KIRP, LUAD, |
| | | DLTVKIGDFGLATEKSRW | LATEKSRWSG (A02.01, | SKCM, THCA |
| | | SGSHQFEQLSGSILWMAPE | B08.01) | |
| | | VIRMQDKNPYSFQSDVYA | | |
| | | FGIVLYELM | | |
| EEF1B2 | S43G | MGFGDLKSPAGLQVLNDY | GPPPADLCHAL (B07.02) | BLCA, KIRP, |
| | | LADKSYIEGYVPSQADVA | | PRAD, SKCM |
| | | VFEAVSGPPPADLCHALR | | |
| | | WYNHIKSYEKEKASLPGV | | |
| | | KKALGKYGPADVEDTTGS | | |
| | | GAT | | |
| ERBB3 | V104M | ERCEVVMGNLEIVLTGHN | | CRC, Stomach |
| | | ADLSFLQWIREVTGYVLV | | Cancer |
| | | AMNEFSTLPLPNLRMVRG | | |
| | | TQVYDGKFAIFVMLNYNT | | |
| | | NSSHALRQLRLTQLTEILS | | |
| | | GGVYIEKNDK | | |
| ESR1 | D538G | HLMAKAGLTLQQQHQRL | GLLLEMLDA (A02.01) | Breast Cancer |
| | | AQLLLILSHIRHMSNKGME | LYGLLLEML (A24.02) | |
| | | HLYSMKCKNVVPLYGLLL | NVVPLYGLL (A02.01) | |
| | | EMLDAHRLHAPTSRGGAS | PLYGLLLEM (A02.01) | |
| | | VEETDQSHLATAGSTSSHS | PLYGLLLEML (A02.01, | |
| | | LQKYYITGEA | A24.02) | |
| | | | VPLYGLLLEM (B07.02) | |
| | | | VVPLYGLLL (A02.01, | |
| | | | A24.02) | |
| ESR1 | S463P | NQGKCVEGMVEIFDMLLA | FLPSTLKSL (A02.01, | Breast Cancer |
| | | TSSRFRMMNLQGEEFVCL | A24.02, B08.01) | |
| | | KSIILLNSGVYTFLPSTLKS | GVYTFLPST (A02.01) | |
| | | LEEKDHIHRVLDKITDTLI | GVYTFLPSTL (A02.01, | |
| | | HLMAKAGLTLQQQHQRL | A24.02) | |
| | | AQLLLILSH | TFLPSTLKSL (A24.02) | |
| | | | VYTFLPSTL (A24.02) | |
| | | | YTFLPSTLK (A03.01) | |
| ESR1 | Y537C | IHLMAKAGLTLQQQHQRL | NVVPLCDLL (A02.01) | Breast Cancer |
| | | AQLLLILSHIRHMSNKGME | NVVPLCDLLL (A02.01) | |
| | | HLYSMKCKNVVPLCDLLL | PLCDLLLEM (A02.01) | |
| | | EMLDAHRLHAPTSRGGAS | PLCDLLLEML (A02.01) | |
| | | VEETDQSHLATAGSTSSHS | VPLCDLLLEM (B07.02) | |
| | | LQKYYITGE | VVPLCDLLL (A02.01, | |
| | | | A24.02) | |
| ESR1 | Y537N | IHLMAKAGLTLQQQHQRL | NVVPLNDLL (A02.01) | Breast Cancer |
| | | AQLLLILSHIRHMSNKGME | NVVPLNDLLL (A02.01) | |
| | | HLYSMKCKNVVPLNDLLL | PLNDLLLEM (A02.01) | |
| | | EMLDAHRLHAPTSRGGAS | PLNDLLLEML (A02.01) | |
| | | VEETDQSHLATAGSTSSHS | VPLNDLLLEM (B07.02) | |
| | | LQKYYITGE | | |
| ESR1 | Y537S | IHLMAKAGLTLQQQHQRL | NVVPLSDLL (A02.01) | Breast Cancer |
| | | AQLLLILSHIRHMSNKGME | NVVPLSDLLL (A02.01) | |
| | | HLYSMKCKNVVPLSDLLL | PLSDLLLEM (A02.01) | |
| | | EMLDAHRLHAPTSRGGAS | PLSDLLLEML (A02.01) | |
| | | VEETDQSHLATAGSTSSHS | VPLSDLLLEM (B07.02) | |
| | | LQKYYITGE | VVPLSDLLL (A02.01, | |
| | | | A24.02) | |
| FGFR3 | S249C | HRIGGIKLRHQQWSLVME | VLERCPHRPI (A02.01, | BLCA, HNSC, |
| | | SVVPSDRGNYTCVVENKF | B08.01) | KIRP, LUSC |
| | | GSIRQTYTLDVLERCPHRP | YTLDVLERC (A02.01) | |
| | | ILQAGLPANQTAVLGSDV | | |
| | | EFHCKVYSDAQPHIQWLK | | |
| | | HVEVNGSKVG | | |
| FRG1B | L52S | AVKLSDSRIALKSGYGKY | FQNGKMALS (A02.01) | GBM, KIRP, |
| | | LGINSDELVGHSDAIGPRE | | PRAD, SKCM |
| | | QWEPVFQNGKMALSASNS | | |
| | | CFIRCNEAGDIEAKSKTAG | | |
| | | EEEMIKIRSCAEKETKKKD | | |
| | | DIPEEDKG | | |
| HER2 | V777L | GSGAFGTVYKGIWIPDGE | VMAGLGSPYV (A02.01, | BRCA |
| | (Resistance) | NVKIPVAIKVLRENTSPKA | A03.01) | |
| | | NKEILDEAYVMAGLGSPY | | |
| | | VSRLLGICLTSTVQLVTQL | | |
| | | MPYGCLLDHVRENRGRLG | | |
| | | SQDLLNWCM | | |
| IDH1 | R132H | RVEEFKLKQMWKSPNGTI | KPIIIGHHA (B07.02) | BLCA, GBM, |
| | | RNILGGTVFREAIICKNIPR | | PRAD |
| | | LVSGWVKPIIIGHHAYGDQ | | |
| | | YRATDFVVPGPGKVEITYT | | |
| | | PSDGTQKVTYLVHNFEEG | | |
| | | GGVAMGM | | |
| IDH1 | R132C | RVEEFKLKQMWKSPNGTI | KPIIIGCHA (B07.02) | BLCA, GBM, |
| | | RNILGGTVFREAIICKNIPR | | PRAD |
| | | LVSGWVKPIIIGCHAYGDQ | | |
| | | YRATDFVVPGPGKVEITYT | | |
| | | PSDGTQKVTYLVHNFEEG | | |
| | | GGVAMGM | | |
| IDH1 | R132G | RVEEFKLKQMWKSPNGTI | KPIIIGGHA (B07.02) | BLCA, BRCA, |
| | | RNILGGTVFREAIICKNIPR | | CRC, GBM, |
| | | LVSGWVKPIIIGGHAYGDQ | | HNSC, LUAD, |
| | | YRATDFVVPGPGKVEITYT | | PAAD, PRAD, |
| | | PSDGTQKVTYLVHNFEEG | | UCEC |
| | | GGVAMGM | | |
| IDHI | R132S | RVEEFKLKQMWKSPNGTI | KPIIIGSHA (B07.02) | BLCA, BRCA, |
| | | RNILGGTVFREAIICKNIPR | | GBM, HNSC, |
| | | LVSGWVKPIIIGSHAYGDQ | | LIHC, LUAD, |
| | | YRATDFVVPGPGKVEITYT | | LUSC, PAAD, |
| | | PSDGTQKVTYLVHNFEEG | | SKCM, UCEC |
| | | GGVAMGM | | |
| KIT | T670I | VAVKMLKPSAHLTEREAL | IIEYCCYGDL (A02.01) | Gastrointestinal |
| | | MSELKVLSYLGNHMNIVN | TIGGPTLVII (A02.01) | stromal |
| | | LLGACTIGGPTLVIIEYCCY | VIIEYCCYG (A02.01) | tumors (GIST) |
| | | GDLLNFLRRKRDSFICSKQ | | |
| | | EDHAEAALYKNLLHSKES | | |
| | | SCSDSTNE | | |
| KIT | V654A | VEATAYGLIKSDAAMTVA | HMNIANLLGA (A02.01) | Gastrointestinal |
| | | VKMLKPSAHLTEREALMS | IANLLGACTI (A02.01) | stromal |
| | | ELKVLSYLGNHMNIANLL | MNIANLLGA (A02.01) | tumors (GIST) |
| | | GACTIGGPTLVITEYCCYG | YLGNHMNIA (A02.01, | |
| | | DLLNFLRRKRDSFICSKQE | B08.01) | |
| | | DHAEAALYK | YLGNHMNIAN (A02.01) | |
| MEK | C121S | ISELGAGNGGVVFKVSHK | VLHESNSPY (A03.01) | Melanoma |
| | | PSGLVMARKLIHLEIKPAI | VLHESNSPYI (A02.01) | |
| | | RNQIIRELQVLHESNSPYIV | | |
| | | GFYGAFYSDGEISICMEHM | | |
| | | DGGSLDQVLKKAGRIPEQI | | |
| | | LGKVSI | | |
| MEK | P124L | LGAGNGGVVFKVSHKPSG | LQVLHECNSL (A02.01, | Melanoma |
| | | LVMARKLIHLEIKPAIRNQI | B08.01) | |
| | | IRELQVLHECNSLYIVGFY | LYIVGFYGAF (A24.02) | |
| | | GAFYSDGEISICMEHMDG | NSLYIVGFY (A01.01) | |
| | | GSLDQVLKKAGRIPEQILG | QVLHECNSL (A02.01, | |
| | | KVSIAVI | B08.01) | |
| | | | SLYIVGFYG (A02.01) | |
| | | | SLYIVGFYGA (A02.01) | |
| | | | VLHECNSLY (A03.01) | |
| | | | VLHECNSLYI (A02.01, | |
| | | | A03.01) | |
| MYC | E39D | MPLNVSFTNRNYDLDYDS | FYQQQQQSDL (A24.02) | Lymphoid |
| | | VQPYFYCDEEENFYQQQQ | QQQSDLQPPA (A02.01) | Cancer; Burkitt |
| | | QSDLQPPAPSEDIWKKFEL | QQSDLQPPA (A02.01) | Lymphoma |
| | | LPTPPLSPSRRSGLCSPSYV | YQQQQQSDL (A02.01, | |
| | | AVTPFSLRGDNDGG | B08.01) | |
| MYC | P57S | FTNRNYDLDYDSVQPYFY | FELLSTPPL (A02.01, | Lymphoid |
| | | CDEEENFYQQQQQSELQP | B08.01) | Cancer |
| | | PAPSEDIWKKFELLSTPPLS | LLSTPPLSPS (A02.01) | |
| | | PSRRSGLCSPSYVAVTPFS | | |
| | | LRGDNDGGGGSFSTADQL | | |
| | | EMVTELLG | | |
| MYC | T58I | TNRNYDLDYDSVQPYFYC | FELLPIPPL (A02.01) | Neuroblastoma |
| | | DEEENFYQQQQQSELQPP | IWKKFELLPI (A24.02) | |
| | | APSEDIWKKFELLPIPPLSP | LLPIPPLSPS (A02.01, | |
| | | SRRSGLCSPSYVAVTPFSL | B07.02) | |
| | | RGDNDGGGGSFSTADQLE | LPIPPLSPS (B07.02) | |
| | | MVTELLGG | | |
| PDGFRa | T674I | VAVKMLKPTARSSEKQAL | IIEYCFYGDL (A02.01) | Chronic |
| | | MSELKIMTHLGPHLNIVNL | IIIEYCFYG (A02.01) | Eosinophilic |
| | | LGACTKSGPIYIIIEYCFYG | IYIIIEYCF (A24.02) | Leukemia |
| | | DLVNYLHKNRDSFLSHHP | IYIIIEYCFY (A24.02) | |
| | | EKPKKELDIFGLNPADEST | YIIIEYCFYG (A02.01) | |
| | | RSYVILS | | |
| PIK3CA | E542K | IEEHANWSVSREAGFSYSH | KITEQEKDFL (A02.01) | BLCA, BRCA, |
| | | AGLSNRLARDNELRENDK | | CESC, CRC, |
| | | EQLKAISTRDPLSKITEQEK | | GBM, HNSC, |
| | | DFLWSHRHYCVTIPEILPK | | KIRC, KIRP, |
| | | LLLSVKWNSRDEVAQMY | | LIHC, LUAD, |
| | | CLVKDWPP | | LUSC, PRAD, |
| | | | | UCEC |
| PIK3CA | E545K | HANWSVSREAGFSYSHAG | STRDPLSEITK (A03.01) | BLCA, BRCA, |
| | | LSNRLARDNELRENDKEQ | DPLSEITK (A03.01) | CESC, CRC, |
| | | LKAISTRDPLSEITKQEKDF | | GBM, HNSC, |
| | | LWSHRHYCVTIPEILPKLL | | KIRC, KIRP, |
| | | LSVKWNSRDEVAQMYCL | | LIHC, LUAD, |
| | | VKDWPPIKP | | LUSC, PRAD, |
| | | | | SKCM, UCEC |
| PIK3CA | H1047R | LFINLFSMMLGSGMPELQS | | BRCA, CESC, |
| | | FDDIAYIRKTLALDKTEQE | | CRC, GBM, |
| | | ALEYFMKQMNDARHGG | | HNSC, LIHC, |
| | | WTTKMDWIFHTIKQHALN | | LUAD, LUSC, |
| | | | | PRAD, UCEC |
| POLE | P286R | QRGGVITDEEETSKKIADQ | LPLKFRDAET (B07.02) | Colorectal |
| | | LDNIVDMREYDVPYHIRLS | | adenocarcinoma; |
| | | IDIETTKLPLKFRDAETDQI | | Uterine/ |
| | | MMISYMIDGQGYLITNREI | | Endometrium |
| | | VSEDIEDFEFTPKPEYEGPF | | Adenocarcinoma; |
| | | CVFN | | Colorectal |
| | | | | adenocarcinoma, |
| | | | | MSI+; |
| | | | | Uterine/ |
| | | | | Endometrium |
| | | | | Adenocarcinoma, |
| | | | | MSI+; |
| | | | | Endometrioid |
| | | | | carcinoma; |
| | | | | Endometrium |
| | | | | Serous |
| | | | | carcinoma; |
| | | | | Endometrium |
| | | | | Carcinosarcoma- |
| | | | | malignant |
| | | | | mesodermal |
| | | | | mixed tumor; |
| | | | | Glioma; |
| | | | | Astrocytoma; |
| | | | | GBM |
| PTEN | R130Q | KFNCRVAQYPFEDHNPPQ | QTGVMICAYL (A02.01) | BRCA, CESC, |
| | | LELIKPFCEDLDQWLSEDD | | CRC, GBM, |
| | | NHVAAIHCKAGKGQTGV | | KIRC, LUSC, |
| | | MICAYLLHRGKFLKAQEA | | UCEC |
| | | LDFYGEVRTRDKKGVTIPS | | |
| | | QRRYVYYYSY | | |
| RAC1 | P29S | MQAIKCVVVGDGAVGKT | AFSGEYIPTV (A02.01, | Melanoma |
| | | CLLISYTTNAFSGEYIPTVF | A24.02) | |
| | | DNYSANVMVDGKPVNLG | | |
| | | LWDTAGQEDYDRLRPLSY | | |
| | | PQTVGET | | |
| TP53 | G245S | IRVEGNLRVEYLDDRNTF | SMNRRPILT (A02.01, | BLCA, BRCA, |
| | | RHSVVVPYEPPEVGSDCTT | B08.01) | CRC, GBM, |
| | | IHYNYMCNSSCMGSMNR | YMCNSSCMGS (A02.01) | HNSC, LUSC, |
| | | RPILTIITLEDSSGNLLGRN | | PAAD, PRAD |
| | | SFEVRVCACPGRDRRTEEE | | |
| | | NLRKKGEP | | |
| TP53 | R175H | TYSPALNKMFCQLAKTCP | | BLCA, BRCA, |
| | | VQLWVDSTPPPGTRVRAM | | CRC, GBM, |
| | | AIYKQSQHMTEVVRHCPH | | HNSC, LUAD, |
| | | HERCSDSDGLAPPQHLIRV | | PAAD, PRAD, |
| | | EGNLRVEYLDDRNTFRHS | | UCEC |
| | | VVVPYEPPEV | | |
| TP53 | R248Q | EGNLRVEYLDDRNTFRHS | GMNQRPILT (A02.01) | BLCA, BRCA, |
| | | VVVPYEPPEVGSDCTTIHY | | CRC, GBM, |
| | | NYMCNSSCMGGMNQRPIL | | HNSC, KIRC, |
| | | TIITLEDSSGNLLGRNSFEV | | LIHC, LUSC, |
| | | RVCACPGRDRRTEEENLR | | PAAD, PRAD, |
| | | KKGEPHHE | | UCEC |
| TP53 | R248W | EGNLRVEYLDDRNTFRHS | GMNWRPILT (A02.01) | BLCA, BRCA, |
| | | VVVPYEPPEVGSDCTTIHY | | CRC, GBM, |
| | | NYMCNSSCMGGMNWRPI | | HNSC, LIHC, |
| | | LTIITLEDSSGNLLGRNSFE | | LUSC, PAAD, |
| | | VRVCACPGRDRRTEEENL | | SKCM, UCEC |
| | | RKKGEPHHE | | |
| TP53 | R273C | PEVGSDCTTIHYNYMCNS | LLGRNSFEVC (A02.01) | BLCA, BRCA, |
| | | SCMGGMNRRPILTIITLED | | CRC, GBM, |
| | | SSGNLLGRNSFEVCVCACP | | HNSC, LUSC, |
| | | GRDRRTEEENLRKKGEPH | | PAAD, UCEC |
| | | HELPPGSTKRALPNNTSSS | | |
| | | PQPKKKPL | | |
| ACVR2A | D96fs; | GVEPCYGDKDKRRHCFAT | | MSI+ CRC, |
| | +1 | WKNISGSIEIVKQGCWLD | | MSI+ Uterine/ |
| | | DINCYDRTDCVEKKRQP* | | Endometrium |
| | | | | Cancer, |
| | | | | MSI+ Stomach |
| | | | | Cancer, Lynch |
| | | | | syndrome |
| ACVR2A | D96fs; | GVEPCYGDKDKRRHCFAT | ALKYIFVAV (A02.01, | MSI+ CRC, |
| | −1 | WKNISGSIEIVKQGCWLD | B08.01) | MSI+ |
| | | DINCYDRTDCVEKKTALK | ALKYIFVAVR (A03.01) | Uterine/ |
| | | YIFVAVRAICVMKSFLIFR | AVRAICVMK (A03.01) | Endometrium |
| | | RWKSHSPLQIQLHLSHPIT | AVRAICVMKS (A03.01) | Cancer, |
| | | TSCSIPWCHLC* | CVEKKTALK (A03.01) | MSI+ Stomach |
| | | | CVEKKTALKY (A01.01) | Cancer, Lynch |
| | | | CVMKSFLIF (A24.02, | syndrome |
| | | | B08.01) | |
| | | | CVMKSFLIFR (A03.01) | |
| | | | FLIFRRWKS (A02.01, | |
| | | | B08.01) | |
| | | | FRRWKSHSPL (B08.01) | |
| | | | FVAVRAICV (A02.01, | |
| | | | B08.01) | |
| | | | FVAVRAICVM (B08.01) | |
| | | | IQLHLSHPI (A02.01) | |
| | | | KSFLIFRRWK (A03.01) | |
| | | | KTALKYIFV (A02.01) | |
| | | | KYIFVAVRAI (A24.02) | |
| | | | RWKSHSPLQI (A24.02) | |
| | | | TALKYIFVAV (A02.01, | |
| | | | B08.01) | |
| | | | VAVRAICVMK (A03.01) | |
| | | | VMKSFLIFR (A03.01) | |
| | | | VMKSFLIFRR (A03.01) | |
| | | | YIFVAVRAI (A02.01) | |
| C15ORF40 | L132fs; | TAEAVNVAIAAPPSEGEA | ALFFFFFET (A02.01) | MSI+ CRC, |
| | +1 | NAELCRYLSKVLELRKSD | ALFFFFFETK (A03.01) | MSI+ Uterine/ |
| | | VVLDKVGLALFFFFFETKS | AQAGVQWRSL (A02.01) | Endometrium |
| | | CSVAQAGVQWRSLGSLQP | CLANFCIFNR (A03.01) | Cancer, |
| | | PPPGFKLFSCLSFLSSWDY | CLSFLSSWDY (A01.01, | MSI+ Stomach |
| | | RRMPPCLANFCIFNRDGVS | A03.01) | Cancer, Lynch |
| | | PCWSGWS* | FFETKSCSV (B08.01) | syndrome |
| | | | FFFETKSCSV (A02.01) | |
| | | | FKLFSCLSFL (A02.01) | |
| | | | FLSSWDYRRM (A02.01) | |
| | | | GFKLFSCLSF (A24.02) | |
| | | | KLFSCLSFL (A02.01, | |
| | | | A03.01) | |
| | | | KLFSCLSFLS (A02.01, | |
| | | | A03.01) | |
| | | | LALFFFFFET (A02.01) | |
| | | | LFFFFFETK (A03.01) | |
| | | | LSFLSSWDY (A01.01) | |
| | | | LSFLSSWDYR (A03.01) | |
| | | | RMPPCLANF (A24.02) | |
| | | | RRMPPCLANF (A24.02) | |
| | | | SLQPPPPGFK (A03.01) | |
| | | | VQWRSLGSL (A02.01) | |
| CNOT1 | L1544fs; | LSVIIFFFVYIWHWALPLIL | FFFSVIFST (A02.01) | MSI+ CRC, |
| | +1 | NNHHICLMSSIILDCNSVR | MSVCFFFFSV (A02.01) | MSI+ Uterine/ |
| | | QSIMSVCFFFFSVIFSTRCL | SVCFFFFSV (A02.01, | Endometrium |
| | | TDSRYPNICWFK* | B08.01) | Cancer, |
| | | | SVCFFFFSVI (A02.01) | MSI+ Stomach |
| | | | | Cancer, Lynch |
| | | | | syndrome |
| CNOT1 | L1544fs; | LSVIIFFFVYIWHWALPLIL | FFCYILNTMF (A24.02) | MSI+ CRC, |
| | −1 | NNHHICLMSSIILDCNSVR | MSVCFFFFCY (A01.01) | MSI+ Uterine/ |
| | | QSIMSVCFFFFCYILNTMF | SVCFFFFCYI (A02.01) | Endometrium |
| | | DR* | | Cancer, |
| | | | | MSI+ Stomach |
| | | | | Cancer, Lynch |
| | | | | syndrome |
| EIF2B3 | A151fs; | VLVLSCDLITDVALHEVV | KQWSSVTSL (A02.01) | MSI+ CRC, |
| | −1 | DLFRAYDASLAMLMRKG | VLWMPTSTV (A02.01) | MSI+ Uterine/ |
| | | QDSIEPVPGQKGKKKQWS | | Endometrium |
| | | SVTSLEWTAQERGCSSWL | | Cancer, |
| | | MKQTWMKSWSLRDPSYR | | MSI+ Stomach |
| | | SILEYVSTRVLWMPTSTV* | | Cancer, Lynch |
| | | | | syndrome |
| EPHB2 | K1020fs; | SIQVMRAQMNQIQSVEGQ | ILIRKAMTV (A02.01) | MSI+ CRC, |
| | −1 | PLARRPRATGRTKRCQPR | | MSI+ Uterine/ |
| | | DVTKKTCNSNDGKKREW | | Endometrium |
| | | EKRKQILGGGGKYKEYFL | | Cancer, |
| | | KRILIRKAMTVLAGDKKG | | MSI+ Stomach |
| | | LGRFMRCVQSETKAVSLQ | | Cancer, Lynch |
| | | LPLGR* | | syndrome |
| ESRP1 | N512fs; | LDFLGEFATDIRTHGVHM | | MSI+ CRC, |
| | +1 | VLNHQGRPSGDAFIQMKS | | MSI+ Uterine/ |
| | | ADRAFMAAQKCHKKKHE | | Endometrium |
| | | GQIC* | | Cancer, |
| | | | | MSI+ Stomach |
| | | | | Cancer, Lynch |
| | | | | syndrome |
| ESRP1 | N512fs; | LDFLGEFATDIRTHGVHM | | MSI+ CRC, |
| | −1 | VLNHQGRPSGDAFIQMKS | | MSI+ Uterine/ |
| | | ADRAFMAAQKCHKKT* | | Endometrium |
| | | | | Cancer, |
| | | | | MSI+ Stomach |
| | | | | Cancer, Lynch |
| | | | | syndrome |
| FAM111B | A273fs; | GALCKDGRFRSDIGEFEW | RMKVPLMK (A03.01) | MSI+ CRC, |
| | −1 | KLKEGHKKIYGKQSMVDE | | MSI+ Uterine/ |
| | | VSGKVLEMDISKKKHYNR | | Endometrium |
| | | KISIKKLNRMKVPLMKLIT | | Cancer, |
| | | RV* | | MSI+ Stomach |
| | | | | Cancer, Lynch |
| | | | | syndrome |
| GBP3 | T585fs; | RERAQLLEEQEKTLTSKLQ | TLKKKPRDI (B08.01) | MSI+ CRC, |
| | −1 | EQARVLKERCQGESTQLQ | | MSI+ Uterine/ |
| | | NEIQKLQKTLKKKPRDICR | | Endometrium |
| | | IS* | | Cancer, |
| | | | | MSI+ Stomach |
| | | | | Cancer, Lynch |
| | | | | syndrome |
| JAK1 | P861fs; | VNTLKEGKRLPCPPNCPDE | LIEGFEALLK (A03.01) | MSI+ CRC, |
| | +1 | VYQLMRKCWEFQPSNRTS | | MSI+ Uterine/ |
| | | FQNLIEGFEALLKTSN* | | Endometrium |
| | | | | Cancer, |
| | | | | MSI+ Stomach |
| | | | | Cancer, Lynch |
| | | | | syndrome |
| JAK1 | K860fs; | CRPVTPSCKELADLMTRC | QQLKWTPHI (A02.01) | MSI+ CRC, |
| | −1 | MNYDPNQRPFFRAIMRDI | QLKWTPHILK (A03.01) | MSI+ Uterine/ |
| | | NKLEEQNPDIVSEKNQQL | IVSEKNQQLK (A03.01) | Endometrium |
| | | KWTPHILKSAS* | QLKWTPHILK (A03.01) | Cancer, |
| | | | QQLKWTPHI (A24.02) | MSI+ Stomach |
| | | | NQQLKWTPHIL (B08.01) | Cancer, Lynch |
| | | | NQQLKWTPHI (B08.01) | syndrome |
| | | | QLKWTPHIL (B08.01) | |
| LMAN1 | E305fs; | DDHDVLSFLTFQLTEPGKE | GPPRPPRAAC (B07.02) | MSI+ CRC, |
| | +1 | PPTPDKEISEKEKEKYQEE | PPRPPRAAC (B07.02) | MSI+ Uterine/ |
| | | FEHFQQELDKKKRGIPEGP | | Endometrium |
| | | PRPPRAACGGNI* | | Cancer, |
| | | | | MSI+ Stomach |
| | | | | Cancer, Lynch |
| | | | | syndrome |
| LMAN1 | E305fs; | DDHDVLSFLTFQLTEPGKE | SLRRKYLRV (B08.01) | MSI+ CRC, |
| | −1 | PPTPDKEISEKEKEKYQEE | | MSI+ Uterine/ |
| | | FEHFQQELDKKKRNSRRA | | Endometrium |
| | | TPTSKGSLRRKYLRV* | | Cancer, |
| | | | | MSI+ Stomach |
| | | | | Cancer, Lynch |
| | | | | syndrome |
| MSH3 | N385fs; | TKSTLIGEDVNPLIKLDDA | SAACHRRGCV (B08.01) | MSI+ CRC, |
| | +1 | VNVDEIMTDTSTSYLLCIS | | MSI+ Uterine/ |
| | | ENKENVRDKKKGQHFYW | | Endometrium |
| | | HCGSAACHRRGCV* | | Cancer, |
| | | | | MSI+ Stomach |
| | | | | Cancer, Lynch |
| | | | | syndrome |
| MSH3 | K383fs; | LYTKSTLIGEDVNPLIKLD | ALWECSLPQA (A02.01) | MSI+ CRC, |
| | −1 | DAVNVDEIMTDTSTSYLL | CLIVSRTLL (B08.01) | MSI+ Uterine/ |
| | | CISENKENVRDKKRATFLL | CLIVSRTLLL (A02.01, | Endometrium |
| | | ALWECSLPQARLCLIVSRT | B08.01) | Cancer, |
| | | LLLVQS* | FLLALWECS (A02.01) | MSI+ Stomach |
| | | | FLLALWECSL (A02.01, | Cancer, Lynch |
| | | | B08.01) | syndrome |
| | | | IVSRTLLLV (A02.01) | |
| | | | LIVSRTLLL (A02.01, | |
| | | | B08.01) | |
| | | | LIVSRTLLLV (A02.01) | |
| | | | LLALWECSL (A02.01, | |
| | | | B08.01) | |
| | | | LPQARLCLI (B08.01, | |
| | | | B07.02) | |
| | | | LPQARLCLIV (B08.01) | |
| | | | NVRDKKRATF (B08.01) | |
| | | | SLPQARLCLI (A02.01, | |
| | | | B08.01) | |
| NDUFC2 | A70fs; | LPPPKLTDPRLLYIGFLGY | FFCWILSCK (A03.01) | MSI+ CRC, |
| | +1 | CSGLIDNLIRRRPIATAGLH | FFFCWILSCK (A03.01) | MSI+ Uterine/ |
| | | RQLLYITAFFFCWILSCKT* | ITAFFFCWI (A02.01) | Endometrium |
| | | | LYITAFFFCW (A24.02) | Cancer, |
| | | | YITAFFFCWI (A02.01) | MSI+ Stomach |
| | | | | Cancer, Lynch |
| | | | | syndrome |
| NDUFC2 | F69fs; | SLPPPKLTDPRLLYIGFLGY | ITAFFLLDI (A02.01) | MSI+ CRC, |
| | −1 | CSGLIDNLIRRRPIATAGLH | LLYITAFFL (A02.01, | MSI+ Uterine/ |
| | | RQLLYITAFFLLDIIL* | B08.01) | Endometrium |
| | | | LLYITAFFLL (A02.01, | Cancer, |
| | | | A24.02) | MSI+ Stomach |
| | | | LYITAFFLL (A24.02) | Cancer, Lynch |
| | | | LYITAFFLLD (A24.02) | syndrome |
| | | | YITAFFLLDI (A02.01) | |
| RBM27 | Q817; | NQSGGAGEDCQIFSTPGHP | GSNEVTTRY (A01.01) | MSI+ CRC, |
| | +1 | KMIYSSSNLKTPSKLCSGS | MPKDVNIQV (B07.02) | MSI+ Uterine/ |
| | | KSHDVQEVLKKKTGSNEV | TGSNEVTTRY (A01.01) | Endometrium |
| | | TTRYEEKKTGSVRKANRM | | Cancer, |
| | | PKDVNIQVRKKQKHETRR | | MSI+ Stomach |
| | | KSKYNEDFERAWREDLTI | | Cancer, Lynch |
| | | KR* | | syndrome |
| RPL22 | K16fs; | MAPVKKLVVKGGKKKEA | | MSI+ CRC, |
| | +1 | SSEVHS* | | MSI+ Uterine/ |
| | | | | Endometrium |
| | | | | Cancer, |
| | | | | MSI+ Stomach |
| | | | | Cancer, Lynch |
| | | | | syndrome |
| RPL22 | K15fs; | MAPVKKLVVKGGKKRSK | | MSI+ CRC, |
| | −1 | F* | | MSI+ Uterine/ |
| | | | | Endometrium |
| | | | | Cancer, |
| | | | | MSI+ Stomach |
| | | | | Cancer, Lynch |
| | | | | syndrome |
| SEC31 | I462fs; | MPSHQGAEQQQQQHHVFI | | MSI+ CRC, |
| A | +1 | SQVVTEKEFLSRSDQLQQ | | MSI+ Uterine/ |
| | | AVQSQGFINYCQKKN* | | Endometrium |
| | | | | Cancer, |
| | | | | MSI+ Stomach |
| | | | | Cancer, Lynch |
| | | | | syndrome |
| SEC31 | I462fs; | MPSHQGAEQQQQQHHVFI | KKLMLLRLNL (A02.01) | MSI+ CRC, |
| A | −1 | SQVVTEKEFLSRSDQLQQ | KLMLLRLNL (A02.01, | MSI+ Uterine/ |
| | | AVQSQGFINYCQKKLMLL | A03.01, B07.02, B08.01) | Endometrium |
| | | RLNLRKMCGPF* | KLMLLRLNLR (A03.01) | Cancer, |
| | | | LLRLNLRKM (B08.01) | MSI+ Stomach |
| | | | LMLLRLNL (B08.01) | Cancer, Lynch |
| | | | LMLLRLNLRK (A03.01) | syndrome |
| | | | LNLRKMCGPF (B08.01) | |
| | | | MLLRLNLRK (A03.01) | |
| | | | MLLRLNLRKM (A02.01, | |
| | | | A03.01, B08.01) | |
| | | | NLRKMCGPF (B08.01) | |
| | | | NYCQKKLMLL (A24.02) | |
| | | | YCQKKLMLL (B08.01) | |
| SEC63 | K530fs; | AEVFEKEQSICAAEEQPAE | FKKKTYTCAI (B08.01) | MSI+ CRC, |
| | +1 | DGQGETNKNRTKGGWQQ | ITTVKATETK (A03.01) | MSI+ Uterine/ |
| | | KSKGPKKTAKSKKKETFK | KSKKKETFK (A03.01) | Endometrium |
| | | KKTYTCAITTVKATETKA | KSKKKETFKK (A03.01) | Cancer, |
| | | GKWSRWE* | KTYTCAITTV (A02.01, | MSI+ Stomach |
| | | | A24.02) | Cancer, Lynch |
| | | | TFKKKTYTC (B08.01) | syndrome |
| | | | TYTCAITTV (A24.02) | |
| | | | TYTCAITTVK (A03.01) | |
| | | | YTCAITTVK (A03.01) | |
| SEC63 | K529fs; | MAEVFEKEQSICAAEEQP | TAKSKKRNL (B08.01) | MSI+ CRC, |
| | −1 | AEDGQGETNKNRTKGGW | | MSI+ Uterine/ |
| | | QQKSKGPKKTAKSKKRNL | | Endometrium |
| | | * | | Cancer, |
| | | | | MSI+ Stomach |
| | | | | Cancer, Lynch |
| | | | | syndrome |
| SLC35F5 | C248fs; | NIMEIRQLPSSHALEAKLS | FALCGFWQI (A02.01) | MSI+ CRC, |
| | −1 | RMSYPVKEQESILKTVGK | | MSI+ Uterine/ |
| | | LTATQVAKISFFFALCGFW | | Endometrium |
| | | QICHIKKHFQTHKLL* | | Cancer, |
| | | | | MSI+ Stomach |
| | | | | Cancer, Lynch |
| | | | | syndrome |
| SMAP1 | K172fs; | YEKKKYYDKNAIAITNISS | | MSI+ CRC, |
| | +1 | SDAPLQPLVSSPSLQAAVD | | MSI+ Uterine/ |
| | | KNKLEKEKEKKKGREKER | | Endometrium |
| | | KGARKAGKTTYS* | | Cancer, |
| | | | | MSI+ Stomach |
| | | | | Cancer, Lynch |
| | | | | syndrome |
| SMAP1 | K171fs; | KYEKKKYYDKNAIAITNIS | LKKLRSPL (B08.01) | MSI+ CRC, |
| | −1 | SSDAPLQPLVSSPSLQAAV | SLKKVPAL (B08.01) | MSI+ Uterine/ |
| | | DKNKLEKEKEKKRKRKRE | RKISNWSLKK (A03.01) | Endometrium |
| | | KRSQKSRQNHLQLKSCRR | VPALKKLRSPL (B07.02) | Cancer, |
| | | KISNWSLKKVPALKKLRSP | | MSI+ Stomach |
| | | LWIF* | | Cancer, Lynch |
| | | | | syndrome |
| TFAM | E148fs; | IYQDAYRAEWQVYKEEIS | KRVNTAWKTK (A03.01) | MSI+ CRC, |
| | +1 | RFKEQLTPSQIMSLEKEIM | MTKKKRVNTA (B08.01) | MSI+ Uterine/ |
| | | DKHLKRKAMTKKKRVNT | RVNTAWKTK (A03.01) | Endometrium |
| | | AWKTKKTSFSL* | RVNTAWKTKK (A03.01) | Cancer, |
| | | | TKKKRVNTA (B08.01) | MSI+ Stomach |
| | | | WKTKKTSFSL (B08.01) | Cancer, Lynch |
| | | | | syndrome |
| TFAM | E148fs; | IYQDAYRAEWQVYKEEIS | | MSI+ CRC, |
| | −1 | RFKEQLTPSQIMSLEKEIM | | MSI+ Uterine/ |
| | | DKHLKRKAMTKKKS* | | Endometrium |
| | | | | Cancer, |
| | | | | MSI+ Stomach |
| | | | | Cancer, Lynch |
| | | | | syndrome |
| TGFBR2 | P129fs; | KPQEVCVAVWRKNDENIT | | MSI+ CRC, |
| | +1 | LETVCHDPKLPYHDFILED | | MSI+ Uterine/ |
| | | AASPKCIMKEKKKAW* | | Endometrium |
| | | | | Cancer, |
| | | | | MSI+ Stomach |
| | | | | Cancer, Lynch |
| | | | | syndrome |
| TGFBR2 | K128fs: | EKPQEVCVAVWRKNDENI | ALMSAMTTS (A02.01) | MSI+ CRC, |
| | −1 | TLETVCHDPKLPYHDFILE | AMTTSSSQK (A03.01, | MSI+ Uterine/ |
| | | DAASPKCIMKEKKSLVRL | A11.01) | Endometrium |
| | | SSCVPVALMSAMTTSSSQ | AMTTSSSQKN (A03.01) | Cancer, |
| | | KNITPAILTCC* | CIMKEKKSL (B08.01) | MSI+ Stomach |
| | | | CIMKEKKSLV (B08.01) | Cancer, Lynch |
| | | | IMKEKKSL (B08.01) | syndrome |
| | | | IMKEKKSLV (B08.01) | |
| | | | KSLVRLSSCV (A02.01) | |
| | | | LVRLSSCVPV (A02.01) | |
| | | | RLSSCVPVA (A02.01, | |
| | | | A03.01) | |
| | | | RLSSCVPVAL (A02.01) | |
| | | | SAMTTSSSQK (A03.01, | |
| | | | A11.01) | |
| | | | SLVRLSSCV (A02.01) | |
| | | | VPVALMSAM (B07.02) | |
| | | | VRLSSCVPVA (A02.01) | |
| THAP5 | K99fs; | VPSKYQFLCSDHFTPDSLD | KMRKKYAQK (A03.01) | MSI+ CRC, |
| | −1 | IRWGIRYLKQTAVPTIFSLP | | MSI+ Uterine/ |
| | | EDNQGKDPSKKNPRRKT | | Endometrium |
| | | WKMRKKYAQKPSQKNHL | | Cancer, |
| | | Y* | | MSI+ Stomach |
| | | | | Cancer, Lynch |
| | | | | syndrome |
| TTK | R854fs; | GTTEEMKYVLGQLVGLNS | FVMSDTTYK (A03.01) | MSI+ CRC, |
| | −1 | PNSILKAAKTLYEHYSGGE | FVMSDTTYKI (A02.01) | MSI+ Uterine/ |
| | | SHNSSSSKTFEKKGEKNDL | KTFEKKGEK (A03.01) | Endometrium |
| | | QLFVMSDTTYKIYWTVILL | LFVMSDTTYK (A03.01) | Cancer, |
| | | NPCGNLHLKTTSL* | MSDTTYKIY (A01.01) | MSI+ Stomach |
| | | | VMSDTTYKI (A02.01) | Cancer, Lynch |
| | | | VMSDTTYKIY (A01.01) | syndrome |
| XPOT | F126fs; | QQLIRETLISWLQAQMLNP | YLTKWPKFFL (A02.01) | MSI+ CRC, |
| | −1 | QPEKTFIRNKAAQVFALLF | | MSI+ Uterine/ |
| | | VTEYLTKWPKFFLTFSQ* | | Endometrium |
| | | | | Cancer, |
| | | | | MSI+ Stomach |
| | | | | Cancer, Lynch |
| | | | | syndrome |
| APC | V1352fs | AKFQQCHSTLEPNPADCR | FLQERNLPP (A02.01) | CRC, LUAD, |
| | SF1354fs | VLVYLQNQPGTKLLNFLQ | FRRPHSCLA (B08.01) | UCEC, STAD |
| | Q1378fs | ERNLPPKVVLRHPKVHLN | LIVLRVVRL (B08.01) | |
| | S1398fs | TMFRRPHSCLADVLLSVH | LLSVHLIVL (A02.01, | |
| | | LIVLRVVRLPAPFRVNHAV | B08.01) | |
| | | EW* | | |
| APC | S1421fs | APVIFQIALDKPCHQAEVK | EVKHLHHLL (B08.01) | CRC, LUAD, |
| | R1435fs | HLHHLLKQLKPSEKYLKIK | HLHHLLKQLK (A03.01) | UCEC, STAD |
| | T1438fs | HLLLKRERVDLSKLQ* | HLLLKRERV (B08.01) | |
| | P1442fs | | KIKHLLLKR (A03.01) | |
| | P1443fs | | KPSEKYLKI (B07.02) | |
| | V1452fs | | KYLKIKHLL (A24.02) | |
| | P1453fs | | KYLKIKHLLL (A24.02) | |
| | K1462fs | | LLKQLKPSEK (A03.01) | |
| | E1464fs | | LLKRERVDL (B08.01) | |
| | | | LLLKRERVDL (B08.01) | |
| | | | QLKPSEKYLK (A03.01) | |
| | | | YLKIKHLLL (A02.01, | |
| | | | B08.01) | |
| | | | YLKIKHLLLK (A03.01) | |
| APC | T1487fs | MLQFRGSRFFQMLILYYIL | ILPRKVLQM (B08.01) | CRC, LUAD, |
| | H1490fs | PRKVLQMDFLVHPA* | KVLQMDFLV (A02.01, | UCEC, STAD |
| | L1488fs | | A24.02) | |
| | | | LPRKVLQMDF (B07.02, | |
| | | | B08.01) | |
| | | | LQMDFLVHPA (A02.01) | |
| | | | QMDFLVHPA (A02.01) | |
| | | | YILPRKVLQM (A02.01, | |
| | | | B08.01) | |
| ARID1A | Q1306fs | ALGPHSRISCLPTQTRGCIL | APSPASRLQC (B07.02) | STAD, UCEC, |
| | S1316fs | LAATPRSSSSSSSNDMIPM | HPLAPMPSKT (B07.02) | BLCA, BRCA, |
| | Y1324fs | AISSPPKAPLLAAPSPASRL | ILPLPQLLL (A02.01) | LUSC, CESC, |
| | T1348fs | QCINSNSRITSGQWMAHM | LLLSADQQA (A02.01) | KIRC, UCS |
| | G1351fs | ALLPSGTKGRCTACHTAL | LPTQTRGCI (B07.02) | |
| | G1378fs | GRGSLSSSSCPQPSPSLPAS | LPTQTRGCIL (B07.02) | |
| | P1467fs | NKLPSLPLSKMYTTSMAM | RISCLPTQTR (A03.01) | |
| | | PILPLPQLLLSADQQAAPR | SLAETVSLH (A03.01) | |
| | | TNFHSSLAETVSLHPLAPM | TPRSSSSSS (B07.02) | |
| | | PSKTCHHK* | TPRSSSSSSS (B07.02) | |
| ARID1A | S674fs | AHQGFPAAKESRVIQLSLL | ALPPVLLSL (A02.01) | STAD, UCEC, |
| | P725fs | SLLIPPLTCLASEALPRPLL | ALPPVLLSLA (A02.01) | BLCA, BRCA, |
| | R727fs | ALPPVLLSLAQDHSRLLQC | ALPRPLLAL (A02.01) | LUSC, CESC, |
| | I736fs | QATRCHLGHPVASRTASCI | ASRTASCIL (B07.02) | KIRC, UCS |
| | | LP* | EALPRPLLAL (B08.01) | |
| | | | HLGHPVASR (A03.01) | |
| | | | HPVASRTAS (B07.02) | |
| | | | HPVASRTASC (B07.02) | |
| | | | IIQLSLLSLL (A02.01) | |
| | | | IQLSLLSLL (A02.01) | |
| | | | IQLSLLSLLI (A02.01, | |
| | | | A24.02) | |
| | | | LLALPPVLL (A02.01) | |
| | | | LLIPPLTCL (A02.01) | |
| | | | LLIPPLTCLA (A02.01) | |
| | | | LLSLLIPPL (A02.01) | |
| | | | LLSLLIPPLT (A02.01) | |
| | | | LPRPLLALPP (B07.02) | |
| | | | QLSLLSLLI (A02.01) | |
| | | | RLLQCQATR (A03.01) | |
| | | | RPLLALPPV (B07.02) | |
| | | | RPLLALPPVL (B07.02) | |
| | | | SLAQDHSRL (A02.01) | |
| | | | SLAQDHSRLL (A02.01) | |
| | | | SLLIPPLTCL (A02.01) | |
| | | | SLLSLLIPP (A02.01) | |
| | | | SLLSLLIPPL (A02.01, | |
| | | | B08.01) | |
| ARID1A | G414fs | PILAATGTSVRTAARTWV | AAATSAASTL (B07.02) | STAD, UCEC, |
| | Q473fs | PRAAIRVPDPAAVPDDHA | AAIPASTSAV (B07.02) | BLCA, BRCA, |
| | H477fs | GPGAECHGRPLLYTADSS | AIPASTSAV (A02.01) | LUSC, CESC, |
| | S499fs | LWTTRPQRVWSTGPDSIL | ALPAGCVSSA (A02.01) | KIRC, UCS |
| | P504fs | QPAKSSPSAAAATLLPATT | APLLTATGSV (B07.02) | |
| | Q548fs | VPDPSCPTFVSAAATVSTT | APVLSASIL (B07.02) | |
| | P549fs | TAPVLSASILPAAIPASTSA | ATLLPATTV (A02.01) | |
| | | VPGSIPLPAVDDTAAPPEP | ATVSTTTAPV (A02.01) | |
| | | APLLTATGSVSLPAAATSA | AVPANCLFPA (A02.01) | |
| | | ASTLDALPAGCVSSAPVSA | CLFPAALPST (A02.01) | |
| | | VPANCLFPAALPSTAGAIS | CPTFVSAAA (B07.02) | |
| | | RFIWVSGILSPLNDLQ* | FPAALPSTA (B07.02) | |
| | | | FPAALPSTAG (B07.02) | |
| | | | GAECHGRPL (B07.02) | |
| | | | GAISRFIWV (A02.01) | |
| | | | ILPAAIPAST (A02.01) | |
| | | | IWVSGILSPL (A24.02) | |
| | | | LLTATGSVSL (A02.01) | |
| | | | LLYTADSSL (A02.01) | |
| | | | LPAAATSAA (B07.02) | |
| | | | LPAAATSAAS (B07.02) | |
| | | | LPAAIPAST (B07.02) | |
| | | | LPAGCVSSA (B07.02) | |
| | | | LPAGCVSSAP (B07.02) | |
| | | | LYTADSSLW (A24.02) | |
| | | | QPAKSSPSA (B07.02) | |
| | | | QPAKSSPSAA (B07.02) | |
| | | | RFIWVSGIL (A24.02) | |
| | | | RPQRVWSTG (B07.02) | |
| | | | RVWSTGPDSI (A02.01) | |
| | | | SAVPGSIPL (B07.02) | |
| | | | SILPAAIPA (A02.01) | |
| | | | SLPAAATSA (A02.01) | |
| | | | SLPAAATSAA (A02.01) | |
| | | | SLWTTRPQR (A03.01) | |
| | | | SLWTTRPQRV (A02.01) | |
| | | | SPSAAAATL (B07.02) | |
| | | | SPSAAAATLL (B07.02) | |
| | | | TLDALPAGCV (A02.01) | |
| | | | TVSTTTAPV (A02.01) | |
| | | | VLSASILPA (A02.01) | |
| | | | VLSASILPAA (A02.01) | |
| | | | VPANCLFPA (B07.02) | |
| | | | VPANCLFPAA (B07.02) | |
| | | | VPDPSCPTF (B07.02) | |
| | | | VPGSIPLPA (B07.02) | |
| | | | VPGSIPLPAV (B07.02) | |
| | | | WVSGILSPL (A02.01) | |
| | | | YTADSSLWTT (A02.01) | |
| ARID1A | T433fs | PCRAGRRVPWAASLIHSRF | APAGMVNRA (B07.02) | STAD, UCEC, |
| | A441fs | LLMDNKAPAGMVNRARL | ASLHRRSYL (B08.01) | BLCA, BRCA, |
| | Y447fs | HITTSKVLTLSSSSHPTPSN | ASLHRRSYLK (A03.01) | LUSC, CESC, |
| | P483fs | HRPRPLMPNLRISSSHSLN | FLLMDNKAPA (A02.01) | KIRC, UCS |
| | P484fs | HHSSSPLSLHTPSSHPSLHI | HPRRSPSRL (B07.02, | |
| | P504fs | SSPRLHTPPSSRRHSSTPRA | B08.01) | |
| | S519fs | SPPTHSHRLSLLTSSSNLSS | HPSLHISSP (B07.02) | |
| | H544fs | QHPRRSPSRLRILSPSLSSP | HRRSYLKIHL (B08.01) | |
| | P549fs | SKLPIPSSASLHRRSYLKIH | HSRFLLMDNK (A03.01) | |
| | P554fs | LGLRHPQPPQ* | KLPIPSSASL (A02.01) | |
| | Q563fs | | KVLTLSSSSH (A03.01) | |
| | | | LIHSRFLLM (B08.01) | |
| | | | LLMDNKAPA (A02.01) | |
| | | | LMDNKAPAGM (A02.01) | |
| | | | LPIPSSASL (B07.02) | |
| | | | MPNLRISSS (B07.02, | |
| | | | B08.01) | |
| | | | MPNLRISSSH (B07.02) | |
| | | | NLRISSSHSL (B07.02, | |
| | | | B08.01) | |
| | | | PPTHSHRLSL (B07.02) | |
| | | | RAGRRVPWAA (B08.01) | |
| | | | RARLHITTSK (A03.01) | |
| | | | RISSSHSLNH (A03.01) | |
| | | | RLHTPPSSR (A03.01) | |
| | | | RLHTPPSSRR (A03.01) | |
| | | | RLRILSPSL (A02.01, | |
| | | | B07.02, B08.01) | |
| | | | RPLMPNLRI (B07.02) | |
| | | | RPRPLMPNL (B07.02) | |
| | | | SASLHRRSYL (B07.02, | |
| | | | B08.01) | |
| | | | SLHISSPRL (A02.01) | |
| | | | SLHRRSYLK (A03.01) | |
| | | | SLHRRSYLKI (B08.01) | |
| | | | SLIHSRFLL (A02.01) | |
| | | | SLIHSRFLLM (A02.01, | |
| | | | B08.01) | |
| | | | SLLTSSSNL (A02.01) | |
| | | | SLNHHSSSPL (A02.01, | |
| | | | B07.02, B08.01) | |
| | | | SLSSPSKLPI (A02.01) | |
| | | | SPLSLHTPS (B07.02) | |
| | | | SPLSLHTPSS (B07.02) | |
| | | | SPPTHSHRL (B07.02) | |
| | | | SPRLHTPPS (B07.02) | |
| | | | SPRLHTPPSS (B07.02) | |
| | | | SPSLSSPSKL (B07.02) | |
| | | | SYLKIHLGL (A24.02) | |
| | | | TPSNHRPRPL (B07.02, | |
| | | | B08.01) | |
| | | | TPSSHPSLHI (B07.02) | |
| ARID1A | A2137fs | RTNPTVRMRPHCVPFWTG | CVPFWTGRIL (B07.02) | STAD, UCEC, |
| | P2139fs | RILLPSAASVCPIPFEACHL | HCVPFWTGRIL (B07.02) | BLCA, BRCA, |
| | L1970fs | CQAMTLRCPNTQGCCSSW | ILLPSAASV (A02.01) | LUSC, CESC, |
| | V1994fs | AS* | ILLPSAASVC (A02.01) | KIRC, UCS |
| | | | LLPSAASVCPI (A02.01) | |
| | | | LPSAASVCPI (B07.02) | |
| | | | MRPHCVPF (B08.01) | |
| | | | RILLPSAASV (A02.01) | |
| | | | RMRPHCVPF (A24.02, | |
| | | | B07.02, B08.01) | |
| | | | RMRPHCVPFW (A24.02) | |
| | | | RTNPTVRMR (A03.01) | |
| | | | SVCPIPFEA (A02.01) | |
| | | | TVRMRPHCV (B08.01) | |
| | | | TVRMRPHCVPF (B08.01) | |
| | | | VPFWTGRIL (B07.02) | |
| | | | VPFWTGRILL (B07.02) | |
| | | | VRMRPHCVPF (B08.01) | |
| ARID1A | N756fs | TNQALPKIEVICRGTPRCPS | AMVPRGVSM (B07.02, | STAD, UCEC, |
| | S764fs | TVPPSPAQPYLRVSLPEDR | B08.01) | BLCA, BRCA, |
| | T783fs | YTQAWAPTSRTPWGAMV | AMVPRGVSMA (A02.01) | LUSC, CESC, |
| | Q799fs | PRGVSMAHKVATPGSQTI | AWAPTSRTPW (A24.02) | KIRC, UCS |
| | A817fs | MPCPMPTTPVQAWLEA* | CPMPTTPVQA (B07.02) | |
| | | | CPSTVPPSPA (B07.02) | |
| | | | GAMVPRGVSM (B07.02, | |
| | | | B08.01) | |
| | | | MPCPMPTTPV (B07.02) | |
| | | | MPTTPVQAW (B07.02) | |
| | | | MPTTPVQAWL (B07.02) | |
| | | | SLPEDRYTQA (A02.01) | |
| | | | SPAQPYLRV (B07.02) | |
| | | | SPAQPYLRVS (B07.02) | |
| | | | TIMPCPMPT (A02.01) | |
| | | | TPVQAWLEA (B07.02) | |
| | | | TSRTPWGAM (B07.02) | |
| | | | VPPSPAQPYL (B07.02) | |
| | | | VPRGVSMAH (B07.02) | |
| β2M | N62fs | RMERELKKWSIQTCLSAR | CLSARTGLSI (B08.01) | CRC, STAD, |
| | E67fs | TGLSISCTTLNSPPLKKMS | CTTLNSPPLK (A03.01) | SKCM, HNSC |
| | L74fs | MPAV* | GLSISCTTL (A02.01) | |
| | F82fs | | SPPLKKMSM (B07.02, | |
| | T91fs | | B08.01) | |
| | E94fs | | TLNSPPLKK (A03.01) | |
| | | | TTLNSPPLK (A03.01) | |
| | | | TTLNSPPLKK (A03.01) | |
| β2M | L13fs | LCSRYSLFLAWRLSSVLQR | LQRFRFTHV (B08.01) | CRC, STAD, |
| | S14fs | FRFTHVIQQRMESQIS* | LQRFRFTHVI (B08.01) | SKCM, HNSC |
| | | | RLSSVLQRF (A24.02) | |
| | | | RLSSVLQRFR (A03.01) | |
| | | | VLQRFRFTHV (A02.01, | |
| | | | B08.01) | |
| CDH1 | A691fs | RSACVTVKGPLASVGRHS | ASVGRHSLSK (A03.01) | ILC LumA |
| | P708fs | LSKQDCKFLPFWGFLEEFL | KFLPFWGFL (A24.02) | Breast Cancer |
| | L711fs | LC* | LASVGRHSL (B07.02) | |
| | | | LPFWGFLEEF (B07.02) | |
| | | | PFWGFLEEF (A24.02) | |
| | | | SVGRHSLSK (A03.01) | |
| CDH1 | H121fs | IQWGTTTAPRPIRPPFLESK | APRPIRPPF (B07.02) | ILC LumA |
| | P126fs | QNCSHFPTPLLASEDRRET | APRPIRPPFL (B07.02) | Breast Cancer |
| | H128fs | GLFLPSAAQKMKKAHFLK | AQKMKKAHFL (B08.01) | |
| | N144fs | TWFRSNPTKTKKARFSTA | FLPSAAQKM (A02.01) | |
| | V157fs | SLAKELTHPLLVSLLLKEK | GLFLPSAAQK (A03.01) | |
| | P159fs | QDG* | HPLLVSLLL (B07.02) | |
| | N166fs | | KAHFLKTWFR (A03.01) | |
| | N181fs | | KARFSTASL (B07.02) | |
| | F189fs | | KMKKAHFLK (A03.01) | |
| | P201fs | | KTWFRSNPTK (A03.01) | |
| | F205fs | | LAKELTHPL (B07.02, | |
| | | | B08.01) | |
| | | | LAKELTHPLL (B08.01) | |
| | | | NPTKTKKARF (B07.02) | |
| | | | QKMKKAHFL (B08.01) | |
| | | | RFSTASLAK (A03.01) | |
| | | | RPIRPPFLES (B07.02) | |
| | | | RSNPTKTKK (A03.01) | |
| | | | SLAKELTHPL (A02.01, | |
| | | | B08.01) | |
| | | | TKKARFSTA (B08.01) | |
| CDH1 | V114fs | PTDPFLGLRLGLHLQKVFH | GLRFWNPSR (A03.01) | ILC LumA |
| | P127fs | QSHAEYSGAPPPPPAPSGL | ISQLLSWPQK (A03.01) | Breast Cancer |
| | V132fs | RFWNPSRIAHISQLLSWPQ | RIAHISQLL (A02.01) | |
| | P160fs | KTEERLGYSSHQLPRK* | RLGYSSHQL (A02.01) | |
| | | | SQLLSWPQK (A03.01) | |
| | | | SRIAHISQL (B08.01) | |
| | | | WPQKTEERL (B07.02) | |
| | | | YSSHQLPRK (A03.01) | |
| CDH1 | L731fs | FCCSCCFFGGERWSKSPYC | CPQRMTPGTT (B07.02) | ILC LumA |
| | R749fs | PQRMTPGTTFITMMKKEA | EAEKRTRTL (B08.01) | Breast Cancer |
| | E757fs | EKRTRTLT* | GTTFITMMK (A03.01) | |
| | G759fs | | GTTFITMMKK (A03.01) | |
| | | | ITMMKKEAEK (A03.01) | |
| | | | RMTPGTTFI (A02.01) | |
| | | | SPYCPQRMT (B07.02) | |
| | | | TMMKKEAEK (A03.01) | |
| | | | TPGTTFITM (B07.02) | |
| | | | TPGTTFITMM (B07.02) | |
| | | | TTFITMMKK (A03.01) | |
| CDH1 | S19fs | WRRNCKAPVSLRKSVQTP | CPGATWREA (B07.02) | ILC LumA |
| | E24fs | ARSSPARPDRTRRLPSLGV | CPGATWREAA (B07.02) | Breast Cancer |
| | S36fs | PGQPWALGAAASRRCCCC | RSRCPGATWR (A03.01) | |
| | | CRSPLGSARSRSPATLALT | TPRATRSRC (B07.02) | |
| | | PRATRSRCPGATWREAAS | | |
| | | WAE* | | |
| GATA3 | P394fs | PGRPLQTHVLPEPHLALQP | HVLPEPHLAL (B07.02) | Breast Cancer |
| | P387fs | LQPHADHAHADAPAIQPV | RPLQTHVLPE (B07.02) | |
| | S398fs | LWTTPPLQHGHRHGLEPC | VLWTTPPLQH (A03.01) | |
| | H400fs | SMLTGPPARVPAVPFDLHF | | |
| | M401fs | CRSSIMKPKRDGYMFLKA | | |
| | S408fs | ESKIMFATLQRSSLWCLCS | | |
| | P409fs | NH* | | |
| | S408fs | | | |
| | P409fs | | | |
| | T419fs | | | |
| | H424fs | | | |
| | P425fs | | | |
| | S427fs | | | |
| | F431fs | | | |
| | S430fs | | | |
| | H434fs | | | |
| | H435fs | | | |
| | S438fs | | | |
| | M443fs | | | |
| | G444fs | | | |
| | *445fs | | | |
| GATA3 | P426fs | PRPRRCTRHPACPLDHTTP | APSESPCSPF (B07.02) | Breast Cancer |
| | H434fs | PAWSPPWVRALLDAHRAP | CPLDHTTPPA (B07.02) | |
| | P433fs | SESPCSPFRLAFLQEQYHE | FLQEQYHEA (A02.01, | |
| | T441fs | A* | B08.01) | |
| | | | RLAFLQEQYH (A03.01) | |
| | | | SPCSPFRLAF (B07.02) | |
| | | | SPPWVRALL (B07.02) | |
| | | | YPACPLDHTT (B07.02) | |
| MLL2 | P519fs | TRRCHCCPHLRSHPCPHHL | ALHLRSCPC (B08.01) | STAD, BLCA, |
| | E524fs | RNHPRPHHLRHHACHHHL | CLHHRRHLV (B08.01) | CRC, HNSC, |
| | P647fs | RNCPHPHFLRHCTCPGRW | CLHHRRHLVC (B08.01) | BRCA |
| | S654fs | RNRPSLRRLRSLLCLPHLN | CLHRKSHPHL (B08.01) | |
| | L656fs | HHLFLHWRSRPCLHRKSH | CLRSHACPP (B08.01) | |
| | R755fs | PHLLHLRRLYPHHLKHRP | CLRSHTCPP (B08.01) | |
| | L761fs | CPHHLKNLLCPRHLRNCP | CLWCHACLH (A03.01) | |
| | Q773fs | LPRHLKHLACLHHLRSHP | CPHHLKNHL (B07.02) | |
| | | CPLHLKSHPCLHHRRHLV | CPHHLKNLL (B07.02) | |
| | | CSHHLKSLLCPLHLRSLPE | CPHHLRTRL (B07.02, | |
| | | PHHLRHHACPHHLRTRLC | B08.01) | |
| | | PHHLKNHLCPPHLRYRAY | CPLHLRSLPF (B07.02, | |
| | | PPCLWCHACLHRLRNLPC | B08.01) | |
| | | PHRLRSLPRPLHLRLHASP | CPLPRHLKHL (B07.02, | |
| | | HHLRTPPHPHHLRTHLLPH | B08.01) | |
| | | HRRTRSCPCRWRSHPCCH | CPLSLRSHPC (B07.02) | |
| | | YLRSRNSAPGPRGRTCHP | CPRHLRNCPL (B07.02, | |
| | | GLRSRTCPPGLRSHTYLRR | B08.01) | |
| | | LRSHTCPPSLRSHAYALCL | FPHHLRHHA (B07.02, | |
| | | RSHTCPPRLRDHICPLSLR | B08.01) | |
| | | NCTCPPRLRSRTCLLCLRS | FPHHLRHHAC (B07.02, | |
| | | HACPPNLRNHTCPPSLRSH | B08.01) | |
| | | ACPPGLRNRICPLSLRSHP | GLRSRTCPP (B08.01) | |
| | | CPLGLKSPLRSQANALHLR | HACLHRLRNL (B08.01) | |
| | | SCPCSLPLGNHPYLPCLES | HLACLHHLR (A03.01) | |
| | | QPCLSLGNHLCPLCPRSCR | HLCPPHLRY (A03.01) | |
| | | CPHLGSHPCRLS* | HLCPPHLRYR (A03.01) | |
| | | | HLKHLACLH (A03.01) | |
| | | | HLKHRPCPH (B08.01) | |
| | | | HLKNHLCPP (B08.01) | |
| | | | HLKSHPCLH (A03.01) | |
| | | | HLKSLLCPL (A02.01, | |
| | | | B08.01) | |
| | | | HLLHLRRLY (A03.01) | |
| | | | HLRNCPLPR (A03.01) | |
| | | | HLRNCPLPRH (A03.01) | |
| | | | HLRRLYPHHL (B08.01) | |
| | | | HLRSHPCPL (B07.02, | |
| | | | B08.01) | |
| | | | HLRSHPCPLH (A03.01) | |
| | | | HLRSLPFPH (A03.01) | |
| | | | HLRTRLCPH (A03.01, | |
| | | | B08.01) | |
| | | | HLVCSHHLK (A03.01) | |
| | | | HPCLHHRRHL (B07.02, | |
| | | | B08.01) | |
| | | | HPGLRSRTC (B07.02) | |
| | | | HPHLLHLRRL (B07.02, | |
| | | | B08.01) | |
| | | | HRKSHPHLL (B08.01) | |
| | | | HRRTRSCPC (B08.01) | |
| | | | KSHPHLLHLR (A03.01) | |
| | | | KSLLCPLHLR (A03.01) | |
| | | | LLCPLHLRSL (A02.01, | |
| | | | B08.01) | |
| | | | LLHLRRLYPH (B08.01) | |
| | | | LPRHLKHLA (B07.02) | |
| | | | LPRHLKHLAC (B07.02, | |
| | | | B08.01) | |
| | | | LRRLRSHTC (B08.01) | |
| | | | LRRLYPHHL (B08.01) | |
| | | | LVCSHHLKSL (B08.01) | |
| | | | NLRNHTCPPS (B08.01) | |
| | | | PLHLRSLPF (B08.01) | |
| | | | RLCPHHLKNH (A03.01) | |
| | | | RLYPHHLKH (A03.01) | |
| | | | RLYPHHLKHR (A03.01) | |
| | | | RPCPHHLKNL (B07.02) | |
| | | | RSHPCPLHLK (A03.01) | |
| | | | RSLPFPHHLR (A03.01) | |
| | | | RTRLCPHHL (B07.02) | |
| | | | RTRLCPHHLK (A03.01) | |
| | | | SLLCPLHLR (A03.01) | |
| | | | SLRSHACPP (B08.01) | |
| | | | SPLRSQANA (B07.02) | |
| | | | YLRRLRSHT (B08.01) | |
| | | | YPHHLKHRPC (B07.02, | |
| | | | B08.01) | |
| PTEN | I122fs | SWKGTNWCNDMCIFITSG | FITSGQIFK (A03.01) | UCEC, PRAD, |
| | I135fs | QIFKGTRGPRFLWGSKDQ | IFITSGQIF (A24.02) | SKCM, STAD, |
| | A148fs | RQKGSNYSQSEALCVLL* | SQSEALCVL (A02.01) | BRCA, LUSC, |
| | L152fs | | SQSEALCVLL (A02.01) | KIRC, LIHC, |
| | D162fs | | | KIRP, GBM |
| | I168fs | | | |
| PTEN | L265fs | KRTKCFTFG* | | UCEC, PRAD, |
| | K266fs | | | SKCM, STAD, |
| | | | | BRCA, LUSC, |
| | | | | KIRC, LIHC, |
| | | | | KIRP, GBM |
| PTEN | A39fs | PIFIQTLLLWDFLQKDLKA | AYTGTILMM (A24.02) | UCEC, PRAD, |
| | E40fs | YTGTILMM* | DLKAYTGTIL (B08.01) | SKCM, STAD, |
| | V45fs | | | BRCA, LUSC, |
| | R47fs | | | KIRC, LIHC, |
| | N48fs | | | KIRP, GBM |
| PTEN | T319fs | QKMILTKQIKTKPTDTFLQ | ILTKQIKTK (A03.01) | UCEC, PRAD, |
| | T321fs | ILR* | KMILTKQIK (A03.01) | SKCM, STAD, |
| | K327fs | | KPTDTFLQI (B07.02) | BRCA, LUSC, |
| | A328fs | | KPTDTFLQIL (B07.02) | KIRC, LIHC, |
| | A333fs | | MILTKQIKTK (A03.01) | KIRP, GBM |
| PTEN | N63fs | GFWIQSIKTITRYTIFVLKD | ITRYTIFVLK (A03.01) | UCEC, PRAD, |
| | E73fs | IMTPPNLIAELHNILLKTIT | LIAELHNIL (A02.01) | SKCM, STAD, |
| | A86fs | HHS* | LIAELHNILL (A02.01) | BRCA, LUSC, |
| | N94fs | | MTPPNLIAEL (A02.01) | KIRC, LIHC, |
| | | | NLIAELHNI (A02.01) | KIRP, GBM |
| | | | NLIAELHNIL (A02.01) | |
| | | | RYTIFVLKDI (A24.02) | |
| | | | TITRYTIFVL (A02.01) | |
| | | | TPPNLIAEL (B07.02) | |
| PTEN | T202fs | NYSNVQWRNLQSSVCGLP | FLQFRTHTT (A02.01, | UCEC, PRAD, |
| | G209fs | AKGEDIFLQFRTHTTGRQV | B08.01) | SKCM, STAD, |
| | C211fs | HVL* | LPAKGEDIFL (B07.02) | BRCA, LUSC, |
| | I224fs | | LQFRTHTTGR (A03.01) | KIRC, LIHC, |
| | G230fs | | NLQSSVCGL (A02.01) | KIRP, GBM |
| | P231fs | | SSVCGLPAK (A03.01) | |
| | R233fs | | VQWRNLQSSV (A02.01) | |
| | D236fs | | | |
| PTEN | G251fs | YQSRVLPQTEQDAKKGQN | GQNVSLLGK (A03.01) | UCEC, PRAD, |
| | E256fs | VSLLGKYILHTRTRGNLRK | HTRTRGNLRK (A03.01) | SKCM, STAD, |
| | K260fs | SRKWKSM* | ILHTRTRGNL (B08.01) | BRCA, LUSC, |
| | Q261fs | | KGQNVSLLGK (A03.01) | KIRC, LIHC, |
| | L265fs | | LLGKYILHT (A02.01) | KIRP, GBM |
| | M270fs | | LRKSRKWKSM (B08.01) | |
| | H272fs | | SLLGKYILH (A03.01) | |
| | T286fs | | SLLGKYILHT (A02.01) | |
| | E288fs | | | |
| TP53 | A70fs | SSQNARGCSPRGPCTSSSY | CTSPLLAPV (A02.01) | BRCA, CRC, |
| | P72fs | TGGPCTSPLLAPVIFCPFPE | FPENLPGQL (B07.02) | LUAD, PRAD, |
| | A76fs | NLPGQLRFPSGLLAFWDS | GLLAFWDSQV (A02.01) | HNSC, LUSC, |
| | A79fs | QVCDLHVLPCPQQDVLPT | IFCPFPENL (A24.02) | PAAD, STAD, |
| | P89fs | GQDLPCAAVG* | LLAFWDSQV (A02.01) | BLCA, OV, |
| | W91fs | | LLAPVIFCP (A02.01) | LIHC, SKCM, |
| | S96fs | | LLAPVIFCPF (A02.01, | UCEC, LAML, |
| | V97fs | | A24.02) | UCS, KICH, |
| | V97fs | | LPCPQQDVL (B07.02) | GBM, ACC |
| | G108fs | | RFPSGLLAF (A24.02) | |
| | G117fs | | RFPSGLLAFW (A24.02) | |
| | S121fs | | SPLLAPVIF (B07.02) | |
| | V122fs | | SPRGPCTSS (B07.02) | |
| | C124fs | | SPRGPCTSSS (B07.02) | |
| | K139fs | | SQVCDLHVL (A02.01) | |
| | V143fs | | VIFCPFPENL (A02.01) | |
| TP53 | V173fs | GAAPTMSAAQIAMVWPLL | AMVWPLLSI (A02.01) | BRCA, CRC, |
| | H178fs | SILSEWKEICVWSIWMTET | AMVWPLLSIL (A02.01) | LUAD, PRAD, |
| | D186fs | LFDIVWWCPMSRLRLALT | AQIAMVWPL (A02.01, | HNSC, LUSC, |
| | H193fs | VPPSTTTTCVTVPAWAA* | A24.02) | PAAD, STAD, |
| | L194fs | | AQIAMVWPLL (A02.01) | BLCA, OV, |
| | E198fs | | CPMSRLRLA (B07.02, | LIHC, SKCM, |
| | V203fs | | B08.01) | UCEC, LAML, |
| | E204fs | | CPMSRLRLAL (B07.02, | UCS, KICH, |
| | L206fs | | B08.01) | GBM, ACC |
| | D207fs | | IAMVWPLLSI (A02.01, | |
| | N210fs | | A24.02, B08.01) | |
| | T211fs | | ILSEWKEICV (A02.01) | |
| | F212fs | | IVWWCPMSR (A03.01) | |
| | V225fs | | IVWWCPMSRL (A02.01) | |
| | S241fs | | IWMTETLFDI (A24.02) | |
| | | | LLSILSEWK (A03.01) | |
| | | | MSAAQIAMV (A02.01) | |
| | | | MSRLRLALT (B08.01) | |
| | | | MSRLRLALTV (B08.01) | |
| | | | MVWPLLSIL (A02.01) | |
| | | | RLALTVPPST (A02.01) | |
| | | | TLFDIVWWC (A02.01) | |
| | | | TLFDIVWWCP (A02.01) | |
| | | | TMSAAQIAMV (A02.01) | |
| | | | VWSIWMTETL (A24.02) | |
| | | | WMTETLFDI (A02.01, | |
| | | | A24.02) | |
| | | | WMTETLFDIV (A01.01, | |
| | | | A02.01) | |
| TP53 | R248fs | TGGPSSPSSHWKTPVVIY | ALRCVFVPV (A02.01, | BRCA, CRC, |
| | P250fs | WDGTALRCVFVPVLGETG | B08.01) | LUAD, PRAD, |
| | S260fs | AQRKRISARKGSLTTSCPQ | ALRCVFVPVL (A02.01, | HNSC, LUSC, |
| | N263fs | GALSEHCPTTPAPLPSQRR | B08.01) | PAAD, STAD, |
| | G266fs | NHWMENISPFRSVGVSAS | ALSEHCPTT (A02.01) | BLCA, OV, |
| | N268fs | RCSES* | AQRKRISARK (A03.01) | LIHC, SKCM, |
| | V272fs | | GAQRKRISA (B08.01) | UCEC, LAML, |
| | V274fs | | HWMENISPF (A24.02) | UCS, KICH, |
| | P278fs | | LPSQRRNHW (B07.02) | GBM, ACC |
| | D281fs | | LPSQRRNHWM (B07.02, | |
| | R282fs | | B08.01) | |
| | T284fs | | NISPFRSVGV (A02.01) | |
| | E285fs | | RISARKGSL (B07.02, | |
| | L289fs | | B08.01) | |
| | K292fs | | SPFRSVGVSA (B07.02) | |
| | P301fs | | SPSSHWKTPV (B07.02, | |
| | S303fs | | B08.01) | |
| | T312fs | | TALRCVFVPV (A02.01) | |
| | S314fs | | VIYWDGTAL (A02.01) | |
| | K319fs | | VIYWDGTALR (A03.01) | |
| | K320fs | | VLGETGAQRK (A03.01) | |
| | P322fs | | | |
| | Y327fs | | | |
| | F328fs | | | |
| | L330fs | | | |
| | R333fs | | | |
| | R335fs | | | |
| | R337fs | | | |
| | E339fs | | | |
| TP53 | S149fs | FHTPARHPRPRHGHLQAV | HPRPRHGHL (B07.02, | BRCA, CRC, |
| | P151fs | TAHDGGCEALPPP* | B08.01) | LUAD, PRAD, |
| | P152fs | | HPRPRHGHLQ (B07.02) | HNSC, LUSC, |
| | V157fs | | RPRHGHLQA (B07.02) | PAAD, STAD, |
| | Q165fs | | RPRHGHLQAV (B07.02, | BLCA, OV, |
| | S166fs | | B08.01) | LIHC, SKCM, |
| | H168fs | | | UCEC, LAML, |
| | V173fs | | | UCS, KICH, |
| | | | | GBM, ACC |
| TP53 | P47fs | CCPRTILNNGSLKTQVQM | GSLKTQVQMK (A03.01) | BRCA, CRC, |
| | D48fs | KLPECQRLLPPWPLHQQL | PPGPCHLLSL (B07.02) | LUAD, PRAD, |
| | D49fs | LHRRPLHQPPPGPCHLLSL | RTILNNGSLK (A03.01) | HNSC, LUSC, |
| | Q52fs | PRKPTRAATVSVWASCIL | SLKTQVQMK (A03.01) | PAAD, STAD, |
| | F54fs | GQPSL* | SLKTQVQMKL (B08.01) | BLCA, OV, |
| | E56fs | | TILNNGSLK (A03.01) | LIHC, SKCM, |
| | P58fs | | | UCEC, LAML, |
| | P60fs | | | UCS, KICH, |
| | E62fs | | | GBM, ACC |
| | M66fs | | | |
| | P72fs | | | |
| | V73fs | | | |
| | P75fs | | | |
| | A78fs | | | |
| | P82fs | | | |
| | P85fs | | | |
| | S96fs | | | |
| | P98fs | | | |
| | T102fs | | | |
| | Y103fs | | | |
| | G108fs | | | |
| | F109fs | | | |
| | R110fs | | | |
| | G117fs | | | |
| TP53 | L26fs | VRKHFQTYGNYFLKTTFC | CPPCRPKQWM (B07.02) | BRCA, CRC, |
| | P27fs | PPCRPKQWMI* | TTFCPPCRPK (A03.01) | LUAD, PRAD, |
| | P34fs | | | HNSC, LUSC, |
| | P36fs | | | PAAD, STAD, |
| | A39fs | | | BLCA, OV, |
| | Q38fs | | | LIHC, SKCM, |
| | | | | UCEC, LAML, |
| | | | | UCS, KICH, |
| | | | | GBM, ACC |
| TP53 | C124fs | LARTPLPSTRCFANWPRPA | CFANWPRPAL (A24.02) | BRCA, CRC, |
| | L130fs | LCSCGLIPHPRPAPASAPW | FANWPRPAL (B07.02, | LUAD, PRAD, |
| | N131fs | PSTSSHST* | B08.01) | HNSC, LUSC, |
| | C135fs | | GLIPHPRPA (A02.01) | PAAD, STAD, |
| | K139fs | | HPRPAPASA (B07.02, | BLCA, OV, |
| | A138fs | | B08.01) | LIHC, SKCM, |
| | T140fs | | HPRPAPASAP (B07.02) | UCEC, LAML, |
| | V143fs | | IPHPRPAPA (B07.02, | UCS, KICH, |
| | Q144fs | | B08.01) | GBM, ACC |
| | V147fs | | IPHPRPAPAS (B07.02) | |
| | T150fs | | RPALCSCGL (B07.02) | |
| | P151fs | | RPALCSCGLI (B07.02) | |
| | P152fs | | TPLPSTRCF (B07.02) | |
| | G154fs | | WPRPALCSC (B07.02) | |
| | R156fs | | WPRPALCSCG (B07.02) | |
| | R158fs | | | |
| | A161fs | | | |
| VHL | L178fs | ELQETGHRQVALRRSGRP | ALRRSGRPPK (A03.01) | KIRC, KIRP |
| | D179fs | PKCAERPGAADTGAHCTS | GLVPSLVSK (A03.01) | |
| | L184fs | TDGRLKISVETYTVSSQLL | KISVETYTV (A02.01) | |
| | T202fs | MVLMSLDLDTGLVPSLVS | LLMVLMSLDL (A02.01, | |
| | R205fs | KCLILRVK* | B08.01) | |
| | D213fs | | LMSLDLDTGL (A02.01) | |
| | G212fs | | LMVLMSLDL (A02.01) | |
| | | | LVSKCLILRV (A02.01) | |
| | | | QLLMVLMSL (A02.01, | |
| | | | B08.01) | |
| | | | RPGAADTGA (B07.02) | |
| | | | RPGAADTGAH (B07.02) | |
| | | | SLDLDTGLV (A02.01) | |
| | | | SLVSKCLIL (A02.01, | |
| | | | B08.01) | |
| | | | SQLLMVLMSL (A02.01) | |
| | | | TVSSQLLMV (A02.01) | |
| | | | TYTVSSQLL (A24.02) | |
| | | | TYTVSSQLLM (A24.02) | |
| | | | VLMSLDLDT (A02.01) | |
| | | | VPSLVSKCL (B07.02) | |
| | | | VSKCLILRVK (A03.01) | |
| | | | YTVSSQLLM (A01.01) | |
| | | | YTVSSQLLMV (A02.01) | |
| VHL | L158fs | KSDASRLSGA* | | KIRC, KIRP |
| | K159fs | | | |
| | R161fs | | | |
| | Q164fs | | | |
| VHL | P146fs | RTAYFCQYHTASVYSERA | FCQYHTASV (B08.01) | KIRC, KIRP |
| | I147fs | MPPGCPEPSQA* | | |
| | F148fs | | | |
| | L158fs | | | |
| VHL | S68fs | TRASPPRSSSAIAVRASCCP | CPYGSTSTA (B07.02) | KIRC, KIRP |
| | S72fs | YGSTSTASRSPTQRCRLAR | CPYGSTSTAS (B07.02) | |
| | I75fs | AAASTATEVTFGSSEMQG | LARAAASTAT (B07.02) | |
| | S80fs | HTMGFWLTKLNYLCHLS | MLTDSLFLP (A02.01) | |
| | P86fs | MLTDSLFLPISHCQCIL* | PPRSSSAIAV (B07.02) | |
| | P97fs | | RAAASTATEV (B07.02) | |
| | I109fs | | SPPRSSSAI (B07.02) | |
| | H115fs | | SPPRSSSAIA (B07.02) | |
| | L116fs | | SPTQRCRLA (B07.02) | |
| | G123fs | | TQRCRLARA (B08.01) | |
| | T124fs | | TQRCRLARAA (B08.01) | |
| | N131fs | | | |
| | L135fs | | | |
| | V137fs | | | |
| | G144fs | | | |
| | D143fs | | | |
| | I147fs | | | |
| VHL | K171fs | SSLRITGDWTSSGRSTKIW | KIWKTTQMCR (A03.01) | KIRC, KIRP |
| | P172fs | KTTQMCRKTWSG* | WTSSGRSTK (A03.01) | |
| | N174fs | | | |
| | L178fs | | | |
| | D179fs | | | |
| | L188fs | | | |
| VHL | V62fs | RRRRGGVGRRGVRPGRVR | ALGELARAL (A02.01) | KIRC, KIRP |
| | V66fs | PGGTGRRGGDGGRAAAA | AQLRRRAAA (B08.01) | |
| | Q73fs | RAALGELARALPGHLLQS | AQLRRRAAAL (B08.01) | |
| | V84fs | QSARRAARMAQLRRRAA | ARRAARMAQL (B08.01) | |
| | F91fs | ALPNAAAWHGPPHPQLPR | HPQLPRSPL (B07.02, | |
| | T100fs | SPLALQRCRDTRWASG* | B08.01) | |
| | P103fs | | HPQLPRSPLA (B07.02) | |
| | S111fs | | LARALPGHL (B07.02) | |
| | L116fs | | LARALPGHLL (B07.02) | |
| | H115fs | | MAQLRRRAA (B07.02, | |
| | D126fs | | B08.01) | |
| | | | MAQLRRRAAA (B07.02, | |
| | | | B08.01) | |
| | | | QLRRRAAAL (B07.02, | |
| | | | B08.01) | |
| | | | RAAALPNAAA (B07.02) | |
| | | | RMAQLRRRAA (B07.02, | |
| | | | B08.01) | |
| | | | SQSARRAARM (B08.01) | |
| AR-v7 | cryptic | SCKVFFKRAAEGKQKYLC | GMTLGEKFRV (A02: 01) | Prostate |
| | final | ASRNDCTIDKFRRKNCPSC | RVGNCKHLK (A03.01) | Cancer, |
| | exon | RLRKCYEAGMTLGEKFRV | | Castration- |
| | | GNCKHLKMTRP* | | resistant |
| | | | | Prostate |
| | | | | Cancer |
| AC011997.1: | AC011997.1: | MAGAPPPASLPPCSLISDC | GPSEPGNNI (B07.02) | LUSC, Breast |
| LRRC69 | LRRC69 | CASNQRDSVGVGPSEP:G: | KICNESASRK (A03.01) | Cancer, Head |
| | *out-of- | NNIKICNESASRK* | | and Neck |
| | frame | | | Cancer, LUAD |
| EEF1DP3 | EEF1DP3: | HGWRPFLPVRARSRWNRR | GIQVLNVSLK (A03.01) | Breast Cancer |
| | FRY | LDVTVANGR:S:WKYGWS | IQVLNVSLK (A03.01) | |
| | *out-of- | LLRVPQVNGIQVLNVSL | KSSSNVISY (A01.01, | |
| | frame | KSSSNVISYE* | A03.01) | |
| | | | KYGWSLLRV (A24.02) | |
| | | | RSWKYGWSL (A02.01) | |
| | | | SLKSSSNVI (B08.01) | |
| | | | SWKYGWSLL (A24.02) | |
| | | | TVANGRSWK (A03.01) | |
| | | | VPQVNGIQV (B07.02) | |
| | | | VPQVNGIQVL (B07.02) | |
| | | | VTVANGRSWK (A03.01) | |
| | | | WSLLRVPQV (B08.01) | |
| MAD1L1: | MAD1L1: | RLKEVFQTKIQEFRKACYT | HPGDCLIFKL (B07.02) | CLL |
| MAFK | MAFK | LTGYQIDITTENQYRLTSL | KLRVPGSSV (B07.02) | |
| | | YAEHPGDCLIFK::LRVPGS | KLRVPGSSVL (B07.02) | |
| | | SVLVTVPGL* | RVPGSSVLV (A02.01) | |
| | | | SVLVTVPGL (A02.01) | |
| | | | VPGSSVLVTV (B07.02) | |
| PPP1R1B: | PPP1R1B: | AEVLKVIRQSAGQKTTCG | ALLLRPRPPR (A03.01) | Breast Cancer |
| STARD3 | STARD3 | QGLEGPWERPPPLDESERD | ALSALLLRPR (A03.01) | |
| | | GGSEDQVEDPALS:A:LLL | | |
| | | RPRPPRPEVGAHQDEQA | | |
| | | AQGADPRLGAQPACRGL | | |
| | | PGLLTVPQPEPLLAPPSA | | |
| | | A* | | |
| BCR: ABL | BCR:ABL | ERAEWRENIREQQKKCFR | LTINKEEAL (A02.01, | CML, AML |
| | | SFSLTSVELQMLTNSCVKL | B08.01) | |
| | | QTVHSIPLTINKE::EALQR | | |
| | | PVASDFEPQGLSEAARW | | |
| | | NSKENLLAGPSENDPNLF | | |
| | | VALYDFVASG | | |
| BCR: ABL | BCR: ABL | ELQMLTNSCVKLQTVHSIP | IVHSATGFK (A03.01) | CML, AML |
| | | LTINKEDDESPGLYGFLNV | ATGFKQSSK (A03.01) | |
| | | IVHSATGFKQSS:K:ALQRP | | |
| | | VASDFEPQGLSEAARWN | | |
| | | SKENLLAGPSENDPNLFV | | |
| | | ALYDFVASGD | | |
| C11orf95: | C11orf95: | ISNSWDAHLGLGACGEAE | ELFPLIFPA (A02.01, | Supretentorial |
| RELA | RELA | GLGVQGAEEEEEEEEEEEE | B08.01) | ependyomas |
| | | EGAGVPACPPKGP:E:LFPL | KGPELFPLI (A02.01, | |
| | | IFPAEPAQASGPYVEIIEQ | A24.02) | |
| | | PKQRGMRFRYKCEGRSA | KGPELFPLIF (A24.02) | |
| | | GSIPGERSTD | | |
| CBFB: | (variant | LQRLDGMGCLEFDEERAQ | | AML |
| MYH11 | “type | QEDALAQQAFEEARRRTR | | |
| | a”) | EFEDRDRSHREEME::VHE | | |
| | | LEKSKRALETQMEEMKT | | |
| | | QLEELEDELQATEDAKL | | |
| | | RLEVNMQALKGQF | | |
| CD74: | (exon6: | KGSFPENLRHLKNTMETID | KPTDAPPKAGV (B07.02) | NSCLC, |
| ROS1 | exon32) | WKVFESWMHHWLLFEMS | | Crizotinib |
| | | RHSLEQKPTDAPPK::AGV | | resistance |
| | | PNKPGIPKLLEGSKNSIQ | | |
| | | WEKAEDNGCRITYYILEI | | |
| | | RKSTSNNLQNQ | | |
| EGFR | EGFRvIII | MRPSGTAGAALLALLAAL | ALEEKKGNYV (A02.01) | GBM |
| | (internal | CPASRALEEKK:G:NYVVT | | |
| | deletion) | DHGSCVRACGADSYEMEE | | |
| | | DGVRKCKKCEGPCRKVC | | |
| | | NGIGIGEFKD | | |
| EGFR: | EGFR: | LPQPPICTIDVYMIMVKCW | IQLQDKFEHL (A02.01, | GBM, Glioma, |
| SEPT14 | SEPT14 | MIDADSRPKFRELIIEFSKM | B08.01) | Head and Neck |
| | | ARDPQRYLVIQ::LQDKFE | QLQDKFEHL (A02.01, | Cancer |
| | | HLKMIQQEEIRKLEEEK | B08.01) | |
| | | KQLEGEIIDFYKMKAASE | QLQDKFEHLK (A03.01) | |
| | | ALQTQLSTD | YLVIQLQDKF (A02.01, | |
| | | | A24.02) | |
| EML4: | EML4: | SWENSDDSRNKLSKIPSTP | QVYRRKHQEL (B08.01) | NSCLC |
| ALK | ALK | KLIPKVTKTADKHKDVIIN | STREKNSQV (B08.01) | |
| | | QAKMSTREKNSQ:V:YRR | VYRRKHQEL (A24.02, | |
| | | KHQELQAMQMELQSPE | B08.01) | |
| | | YKLSKLRTSTIMTDYNPN | | |
| | | YCFAGKTSSISDL | | |
| FGFR3: | FGFR3: | EGHRMDKPANCTHDLYMI | VLTVTSTDV (A02.01) | Bladder |
| TACC3 | TACC3 | MRECWHAAPSQRPTFKQL | VLTVTSTDVK (A03.01) | Cancer, LUSC |
| | | VEDLDRVLTVTSTD::VKA | | |
| | | TQEENRELRSRCEELHG | | |
| | | KNLELGKIMDRFEEVVY | | |
| | | QAMEEVQKQKELS | | |
| NAB: | NAB: | RDNTLLLRRVELFSLSRQV | IMSLWGLVS (A02.01) | Solitary fibrous |
| STAT6 | STAT6 | ARESTYLSSLKGSRLHPEE | IMSLWGLVSK (A03.01) | tumors |
| | “” | LGGPPLKKLKQE::ATSKSQ | KLKQEATSK (A03.01) | |
| | | IMSLWGLVSKMPPEKVQ | QIMSLWGLV (A02.01) | |
| | | RLYVDFPQHLRHLLGDW | SQIMSLWGL (A02.01, | |
| | | LESQPWEFLVGSDAFCC | A24.02, B08.01) | |
| | | | SQIMSLWGLV (A02.01) | |
| | | | TSKSQIMSL (B08.01) | |
| NDRG1: | NDRG1: | MSREMQDVDLAEVKPLV | LLQEFDVQEA (A02.01) | Prostate |
| ERG | ERG | EKGETITGLLQEFDVQ::EA | LQEFDVQEAL (A02.01) | Cancer |
| | | LSVVSEDQSLFECAYGTP | | |
| | | HLAKTEMTASSSSDYGQ | | |
| | | TSKMSPRVPQQDW | | |
| PML: | PML: | VLDMHGFLRQALCRLRQE | | Acute |
| RARA | RARA | EPQSLQAAVRTDGFDEFK | | promyelocytic |
| | (exon3: | VRLQDLSSCITQGK:A:IET | | leukemia |
| | exon3) | QSSSSEEIVPSPPSPPPLPR | | |
| | | IYKPCFVCQDKSSGYHY | | |
| | | GVSACEGCKG | | |
| PML: | PML: | RSSPEQPRPSTSKAVSPPHL | | Acute |
| RARA | RARA | DGPPSPRSPVIGSEVFLPNS | | promyelocytic |
| | (exon6: | NHVASGAGEA:A:IETQSS | | leukemia |
| | exon3) | SSEEIVPSPPSPPPLPRIYK | | |
| | | PCFVCQDKSSGYHYGVS | | |
| | | ACEGCKG | | |
| RUNX1 | RUNX1 | VARFNDLRFVGRSGRGKS | GPREPRNRT (B07.02) | AML |
| | (ex5)- | FTLTITVFTNPPQVATYHR | RNRTEKHSTM (B08.01) | |
| | RUNX1 | AIKITVDGPREPR:N:RTEK | | |
| | T1(ex2) | HSTMPDSPVDVKTQSRL | | |
| | | TPPTMPPPPTTQGAPRTS | | |
| | | SFTPTTLTNGT | | |
| TMPRSS2: | TMPRSS2: | MALNS::EALSVVSEDQSLF | ALNSEALSV (A02.01) | Prostate |
| ERG | ERG | ECAYGTPHLAKTEMTASS | ALNSEALSVV (A02.01) | |
| | | SSDYGQTSKMSPRVPQQD | MALNSEALSV (A02.01, | Cancer |
| | | W | B08.01) | |

TABLE 9

| | Amino Acid | Mutation Sequence | Peptides (HLA allele | Exemplary |
|---|---|---|---|---|
| Gene | Alteration | Context | example(s)) | Diseases |
| AKT1 | E17K | MSDVAIVKEGWLH | KYIKTWRPRY (A24.02) | BRCA, CESC, |
| | | KRGKYIKTWRPRY | WLHKRGKYI (A02.01, B07.02, | HNSC, LUSC, |
| | | FLLKNDGTFIGYKE | B08.01) | PRAD, SKCM, |
| | | RPQDVDQREAPLN | WLHKRGKYIK (A03.01) | THCA |
| | | NFSVAQCQLMKTE | | |
| | | R | | |
| ANAPCI | T537A | TMLVLEGSGNLVL | APKPLSKLL (B07.02) | GBM, LUSC, |
| | | YTGVVRVGKVFIPG | GVSAPKPLSK (A03.01) | PAAD, PRAD, |
| | | LPAPSLTMSNTMPR | VSAPKPLSK (A03.01) | SKCM |
| | | PSTPLDGVSAPKPL | | |
| | | SKLLGSLDEVVLLS | | |
| | | PVPELRDSSKLHDS | | |
| | | LYNEDCTFQQLGT | | |
| | | YIHSI | | |
| FGFR3 | S249C | HRIGGIKLRHQQWS | CPHRPILQA (B07.02) | BLCA, HNSC, |
| | | LVMESVVPSDRGN | | KIRP, LUSC |
| | | YTCVVENKFGSIRQ | | |
| | | TYTLDVLERCPHRP | | |
| | | ILQAGLPANQTAVL | | |
| | | GSDVEFHCKVYSD | | |
| | | AQPHIQWLKHVEV | | |
| | | NGSKVG | | |
| FRG1B | I10T | MREPIYMHSTMVF | KLSDSRTAL (A02.01, B07.02, | KIRP, PRAD, |
| | | LPWELHTKKGPSPP | B08.01) | SKCM |
| | | EQFMAVKLSDSRT | KLSDSRTALK (A03.01) | |
| | | ALKSGYGKYLGINS | LSDSRTALK (A01.01, A03.01) | |
| | | DELVGHSDAIGPRE | RTALKSGYGK (A03.01) | |
| | | QWEPVFQNGKMAL | TALKSGYGK (A03.01) | |
| | | LASNSCFIR | | |
| FRGIB | L52S | AVKLSDSRIALKSG | ALSASNSCF (A02.01, A24.02, | GBM, KIRP, |
| | | YGKYLGINSDELVG | B07.02) | PRAD, SKCM |
| | | HSDAIGPREQWEPV | ALSASNSCFI (A02.01) | |
| | | FQNGKMALSASNS | FQNGKMALSA (A02.01, | |
| | | CFIRCNEAGDIEAK | B08.01) | |
| | | SKTAGEEEMIKIRS | | |
| | | CAEKETKKKDDIPE | | |
| | | EDKG | | |
| HER2 | L755S | AMPNQAQMRILKE | KVSRENTSPK (A03.01) | BRCA |
| | (Resistance) | TELRKVKVLGSGA | | |
| | | FGTVYKGIWIPDGE | | |
| | | NVKIPVAIKVSREN | | |
| | | TSPKANKEILDEAY | | |
| | | VMAGVGSPYVSRL | | |
| | | LGICLTSTVQLVTQ | | |
| | | LMPYGC | | |
| IDHI | R132G | RVEEFKLKQMWKS | KPIIIGGHAY (B07.02) | BLCA, BRCA, |
| | | PNGTIRNILGGTVF | | CRC, GBM, |
| | | REAIICKNIPRLVSG | | HNSC, LUAD, |
| | | WVKPIIIGGHAYGD | | PAAD, PRAD, |
| | | QYRATDFVVPGPG | | UCEC |
| | | KVEITYTPSDGTQK | | |
| | | VTYLVHNFEEGGG | | |
| | | VAMGM | | |
| KRAS | G12C | MTEYKLVVVGACG | KLVVVGACGV (A02.01) | BRCA, CESC, |
| | | VGKSALTIQLIQNH | LVVVGACGV (A02.01) | CRC, HNSC, |
| | | FVDEYDPTIEDSYR | VVGACGVGK (A03.01, A11.01) | LUAD, PAAD, |
| | | KQVVIDGETCLLDI | VVVGACGVGK (A03.01) | UCEC |
| | | LDTAGQE | | |
| KRAS | G12D | MTEYKLVVVGADG | VVGADGVGK (A11.01) | BLCA, BRCA, |
| | | VGKSALTIQLIQNH | VVVGADGVGK (A11.01) | CESC, CRC, |
| | | FVDEYDPTIEDSYR | KLVVVGADGV (A02.01) | GBM, HNSC, |
| | | KQVVIDGETCLLDI | LVVVGADGV (A02.01) | KIRP, LIHC, |
| | | LDTAGQE | | LUAD, PAAD, |
| | | | | SKCM, UCEC |
| KRAS | G12V | MTEYKLVVVGAVG | KLVVVGAVGV (A02.01) | BRCA, CESC, |
| | | VGKSALTIQLIQNH | LVVVGAVGV (A02.01) | CRC, LUAD, |
| | | FVDEYDPTIEDSYR | VVGAVGVGK (A03.01, A11.01) | PAAD, THCA, |
| | | KQVVIDGETCLLDI | VVVGAVGVGK (A03.01, | UCEC |
| | | LDTAGQE | A11.01) | |
| KRAS | Q61H | AGGVGKSALTIQLI | ILDTAGHEEY (A01.01) | CRC, LUSC, |
| | | QNHFVDEYDPTIED | | PAAD, SKCM, |
| | | SYRKQVVIDGETCL | | UCEC |
| | | LDILDTAGHEEYSA | | |
| | | MRDQYMRTGEGFL | | |
| | | CVFAINNTKSFEDI | | |
| | | HHYREQIKRVKDSE | | |
| | | DVPM | | |
| KRAS | Q61L | AGGVGKSALTIQLI | ILDTAGLEEY (A01.01) | CRC, GBM, |
| | | QNHFVDEYDPTIED | LLDILDTAGL (A02.01) | HNSC, LUAD, |
| | | SYRKQVVIDGETCL | | SKCM, UCEC |
| | | LDILDTAGLEEYSA | | |
| | | MRDQYMRTGEGFL | | |
| | | CVFAINNTKSFEDI | | |
| | | HHYREQIKRVKDSE | | |
| | | DVPM | | |
| NRAS | Q61K | AGGVGKSALTIQLI | ILDTAGKEEY (A01.01) | BLCA, CRC, |
| | | QNHFVDEYDPTIED | | LIHC, LUAD, |
| | | SYRKQVVIDGETCL | | LUSC, SKCM, |
| | | LDILDTAGKEEYSA | | THCA, UCEC |
| | | MRDQYMRTGEGFL | | |
| | | CVFAINNSKSFADI | | |
| | | NLYREQIKRVKDSD | | |
| | | DVPM | | |
| NRAS | Q61R | AGGVGKSALTIQLI | ILDTAGREEY (A01.01) | BLCA, CRC, |
| | | QNHFVDEYDPTIED | | LUSC, PAAD, |
| | | SYRKQVVIDGETCL | | PRAD, SKCM, |
| | | LDILDTAGREEYSA | | THCA, UCEC |
| | | MRDQYMRTGEGFL | | |
| | | CVFAINNSKSFADI | | |
| | | NLYREQIKRVKDSD | | |
| | | DVPM | | |
| PIK3CA | E542K | IEEHANWSVSREAG | AISTRDPLSK (A03.01) | BLCA, BRCA, |
| | | FSYSHAGLSNRLAR | | CESC, CRC, |
| | | DNELRENDKEQLK | | GBM, HNSC, |
| | | AISTRDPLSKITEQE | | KIRC, KIRP, |
| | | KDFLWSHRHYCVT | | LIHC, LUAD, |
| | | IPEILPKLLLSVKWN | | LUSC, PRAD, |
| | | SRDEVAQMYCLVK | | UCEC |
| | | DWPP | | |
| PTEN | R130Q | KFNCRVAQYPFED | QTGVMICAY (A01.01) | BRCA, CESC, |
| | | HNPPQLELIKPFCE | | CRC, GBM, |
| | | DLDQWLSEDDNHV | | KIRC, LUSC, |
| | | AAIHCKAGKGQTG | | UCEC |
| | | VMICAYLLHRGKF | | |
| | | LKAQEALDFYGEV | | |
| | | RTRDKKGVTIPSQR | | |
| | | RYVYYYSY | | |
| RAC1 | P29S | MQAIKCVVVGDGA | FSGEYIPTV (A02.01) | Melanoma |
| | | VGKTCLLISYTTNA | TTNAFSGEY (A01.01) | |
| | | FSGEYIPTVFDNYS | YTTNAFSGEY (A01.01) | |
| | | ANVMVDGKPVNL | | |
| | | GLWDTAGQEDYDR | | |
| | | LRPLSYPQTVGET | | |
| SF3B1 | K700E | AVCKSKKSWQARH | GLVDEQQEV (A02.01) | AML associated |
| | | TGIKIVQQIAILMGC | | with MDS; |
| | | AILPHLRSLVEIIEH | | Chronic |
| | | GLVDEQQEVRTISA | | lymphocytic |
| | | LAIAALAEAATPYG | | leukaemia-small |
| | | IESFDSVLKPLWKG | | lymphocytic |
| | | IRQHRGKGLAAFLK | | lymphoma; |
| | | AI | | Myelodysplastic |
| | | | | syndrome; AML; |
| | | | | Luminal NS |
| | | | | carcinoma of |
| | | | | breast; Chronic |
| | | | | myeloid |
| | | | | leukaemia; Ductal |
| | | | | carcinoma of |
| | | | | pancreas; Chronic |
| | | | | myelomonocytic |
| | | | | leukaemia; |
| | | | | Chronic |
| | | | | lymphocytic |
| | | | | leukaemia-small |
| | | | | lymphocytic |
| | | | | lymphoma; |
| | | | | Myelofibrosis; |
| | | | | Myelodysplastic |
| | | | | syndrome; PRAD; |
| | | | | Essential |
| | | | | thrombocythaemia; |
| | | | | Medullomyoblastoma |
| SPOP | F133L | YLSLYLLLVSCPKS | FVQGKDWGL (A02.01, B08.01) | PRAD |
| | | EVRAKFKFSILNAK | | |
| | | GEETKAMESQRAY | | |
| | | RFVQGKDWGLKKF | | |
| | | IRRDFLLDEANGLL | | |
| | | PDDKLTLFCEVSVV | | |
| | | QDSVNISGQNTMN | | |
| | | MVKVPE | | |
| SPOP | F133V | YLSLYLLLVSCPKS | FVQGKDWGV (A02.01) | PRAD |
| | | EVRAKFKFSILNAK | | |
| | | GEETKAMESQRAY | | |
| | | RFVQGKDWGVKKF | | |
| | | IRRDFLLDEANGLL | | |
| | | PDDKLTLFCEVSVV | | |
| | | QDSVNISGQNTMN | | |
| | | MVKVPE | | |
| TP53 | G245S | IRVEGNLRVEYLDD | CMGSMNRRPI (A02.01, | BLCA, BRCA, |
| | | RNTFRHSVVVPYEP | B08.01) | CRC, GBM, |
| | | PEVGSDCTTIHYNY | GSMNRRPIL (B08.01) | HNSC, LUSC, |
| | | MCNSSCMGSMNRR | MGSMNRRPI (B08.01) | PAAD, PRAD |
| | | PILTIITLEDSSGNLL | MGSMNRRPIL (B08.01) | |
| | | GRNSFEVRVCACP | SMNRRPILTI (A02.01, A24.02, | |
| | | GRDRRTEEENLRK | B08.01) | |
| | | KGEP | | |
| TP53 | R248Q | EGNLRVEYLDDRN | CMGGMNQRPI (A02.01, | BLCA, BRCA, |
| | | TFRHSVVVPYEPPE | B08.01) | CRC, GBM, |
| | | VGSDCTTIHYNYM | GMNQRPILTI (A02.01, B08.01) | HNSC, KIRC, |
| | | CNSSCMGGMNQRP | NQRPILTII (A02.01, B08.01) | LIHC, LUSC, |
| | | ILTITLEDSSGNLL | | PAAD, PRAD, |
| | | GRNSFEVRVCACP | | UCEC |
| | | GRDRRTEEENLRK | | |
| | | KGEPHHE | | |
| TP53 | R248W | EGNLRVEYLDDRN | CMGGMNWRPI (A02.01, | BLCA, BRCA, |
| | | TFRHSVVVPYEPPE | A24.02, B08.01) | CRC, GBM, |
| | | VGSDCTTIHYNYM | GMNWRPILTI (A02.01, B08.01) | HNSC, LIHC, |
| | | CNSSCMGGMNWR | MNWRPILTI (A02.01, A24.02, | LUSC, PAAD, |
| | | PILTIITLEDSSGNLL | B08.01) | SKCM, UCEC |
| | | GRNSFEVRVCACP | MNWRPILTII (A02.01, A24.02) | |
| | | GRDRRTEEENLRK | | |
| | | KGEPHHE | | |
| TP53 | R273C | PEVGSDCTTIHYNY | NSFEVCVCA (A02.01) | BLCA, BRCA, |
| | | MCNSSCMGGMNR | | CRC, GBM, |
| | | RPILTIITLEDSSGNL | | HNSC, LUSC, |
| | | LGRNSFEVCVCACP | | PAAD, UCEC |
| | | GRDRRTEEENLRK | | |
| | | KGEPHHELPPGSTK | | |
| | | RALPNNTSSSPQPK | | |
| | | KKPL | | |
| TP53 | R273H | PEVGSDCTTIHYNY | NSFEVHVCA (A02.01) | BRCA, CRC, |
| | | MCNSSCMGGMNR | | GBM, HNSC, |
| | | RPILTIITLEDSSGNL | | LIHC, LUSC, |
| | | LGRNSFEVHVCACP | | PAAD, UCEC |
| | | GRDRRTEEENLRK | | |
| | | KGEPHHELPPGSTK | | |
| | | RALPNNTSSSPQPK | | |
| | | KKPL | | |
| TP53 | Y220C | TEVVRRCPHHERCS | VVPCEPPEV (A02.01) | BLCA, BRCA, |
| | | DSDGLAPPQHLIRV | VVVPCEPPEV (A02.01) | GBM, HNSC, |
| | | EGNLRVEYLDDRN | | LIHC, LUAD, |
| | | TFRHSVVVPCEPPE | | LUSC, PAAD, |
| | | VGSDCTTIHYNYM | | SKCM, UCEC |
| | | CNSSCMGGMNRRP | | |
| | | ILTITLEDSSGNLL | | |
| | | GRNSF | | |
| MSH6 | F1088fs; +1 | YNFDKNYKDWQS | ILLPEDTPPL (A02.01) | MSI+ CRC, MSI+ |
| | | AVECIAVLDVLLCL | LLPEDTPPL (A02.01) | Uterine/Endometrium |
| | | ANYSRGGDGPMCR | | Cancer, MSI+ |
| | | PVILLPEDTPPLLRA | | Stomach Cancer, |
| | | | | Lynch syndrome |
| APC | F1354fs | AKFQQCHSTLEPNP | APFRVNHAV (B07.02) | CRC, LUAD, |
| | | ADCRVLVYLQNQP | CLADVLLSV (A02.01) | UCEC, STAD |
| | | GTKLLNFLQERNLP | FLQERNLPPK (A03.01) | |
| | | PKVVLRHPKVHLN | HLIVLRVVRL (A02.01, B08.01) | |
| | | TMFRRPHSCLADV | HPKVHLNTM (B07.02, B08.01) | |
| | | LLSVHLIVLRVVRL | HPKVHLNTMF (B07.02, | |
| | | PAPFRVNHAVEW* | B08.01) | |
| | | | KVHLNTMFR (A03.01) | |
| | | | KVHLNTMFRR (A03.01) | |
| | | | LPAPFRVNHA (B07.02) | |
| | | | MFRRPHSCL (B07.02, B08.01) | |
| | | | MFRRPHSCLA (B08.01) | |
| | | | NTMFRRPHSC (B08.01) | |
| | | | RPHSCLADV (B07.02) | |
| | | | RPHSCLADVL (B07.02) | |
| | | | RVVRLPAPFR (A03.01) | |
| | | | SVHLIVLRV (A02.01) | |
| | | | TMFRRPHSC (B08.01) | |
| | | | TMFRRPHSCL (A02.01, B08.01) | |
| | | | VLLSVHLIV (A02.01) | |
| | | | VLLSVHLIVL (A02.01) | |
| | | | VLRVVRLPA (B08.01) | |
| | | | VVRLPAPFR (A03.01) | |

Table 10 shows HLA affinity and stability of selected BTK peptides:

TABLE 10

| | HLA | Peptide | Affinity | Stability |
|---|---|---|---|---|
| Gene | Allele | Sequence | (nM) | (half-life (hr.)) |
| BTK, C481S | A01.01 | YMANGSLLNY | 13.24495 | 0.866167 |
| BTK, C481S | A01.01 | MANGSLLNY | 439.029 | 0.216408 |
| BTK, C481S | A03.01 | MANGSLLNY | 35.62463 | 0.237963 |
| BTK, C481S | A03.01 | YMANGSLLNY | 95.93212 | 0.279088 |
| BTK, C481S | A11.01 | MANGSLLNY | 535.6333 | NB |
| BTK, C481S | A11.01 | YMANGSLLNY | 974.2881 | NB |
| BTK, C481S | A24.02 | EYMANGSLL | 4.961145 | 5.716141 |
| BTK_C481S | A02.01 | SLLNYLREM | 67.69132 | 3.043604 |
| BTK_C481S | A02.01 | MANGSLLNYL | 1006.566 | 0 |
| BTK_C481S | A02.01 | YMANGSLLN | 3999.442 | 0 |
| BTK_C481S | B07.02 | SLLNYLREM | 865.8805 | 0 |
| BTK_C481S | B07.02 | MANGSLLNYL | 16474.59 | 0 |
| BTK_C481S | B08.01 | SLLNYLREM | 959.6542 | 0 |
| BTK_C481S | B08.01 | MANGSLLNYL | 18463.09 | 0 |

Table 11 shows HLA affinity and stability of selected EGFR peptides:

TABLE 11

| | HLA | Peptide | Affinity | Stability |
|---|---|---|---|---|
| Gene | Allele | Sequence | (nM) | (half-life (hr.)) |
| EGFR, T790M | A01.01 | LTSTVQLIM | 2891.111 | 0.103721 |
| EGFR_T790M | A01.01 | CLTSTVQLIM | 8276.876 | 0 |
| EGFR_T790M | A02.01 | MQLMPFGCLL | 16.26147 | 0.381118 |
| EGFR_T790M | A02.01 | MQLMPFGCL | 116.3352 | 0.368273 |
| EGFR_T790M | A02.01 | LIMQLMPFGC | 132.4766 | 0.381284 |
| EGFR_T790M | A02.01 | QLIMQLMPF | 192.8406 | 0.34067 |
| EGFR_T790M | A02.01 | CLTSTVQLIM | 537.1391 | 0 |
| EGFR_T790M | A02.01 | IMQLMPFGCL | 653.1065 | 0.515559 |
| EGFR_T790M | A02.01 | IMQLMPFGC | 1205.368 | 0.370112 |
| EGFR_T790M | A02.01 | LIMQLMPFG | 3337.708 | 0 |
| EGFR_T790M | A02.01 | VOLIMQLMPF | 4942.892 | 0 |
| EGFR_T790M | A02.01 | QLIMQLMPFG | 5214.668 | 0 |
| EGFR_T790M | A02.01 | STVQLIMQL | 7256.773 | 0 |
| EGFR_T790M | A24.02 | QLIMQLMPF | 2030.807 | 0.368673 |
| EGFR_T790M | A24.02 | VQLIMQLMPF | 4103.131 | 0 |
| EGFR_T790M | A24.02 | IMQLMPFGCL | 14119.38 | 0 |
| EGFR_T790M | A24.02 | MQLMPFGCLL | 18857.47 | 0 |
| EGFR_T790M | B07.02 | MQLMPFGCL | 1589.188 | 0 |
| EGFR_T790M | B08.01 | QLIMQLMPF | 330.1933 | 0 |
| EGFR_T790M | B08.01 | IMQLMPFGCL | 427.3913 | 0 |
| EGFR_T790M | B08.01 | MQLMPFGCL | 4931.727 | 0 |
| EGFR_T790M | B08.01 | MQLMPFGCLL | 11244.9 | 0 |
| EGFR_T790M | B08.01 | VQLIMQLMPF | 16108.18 | 0 |
| EGFR_T790M | B08.02 | QLIMQLMPF | 5590.3 | ND |

Tumor Antigens Associated with Tumor Microenvironment

In many cases, predominant antigens are expressed by cells in the tumor microenvironment that not only serve as excellent biomarkers for the disease, but also can be important vaccine candidates for immunotherapy. Such tumor associated antigens (TAAs) are not necessarily presented on the surface of tumor cells, but on cells that are juxtaposed to the tumor, which could be the stromal cells, connective tissue cells, fibroblasts etc. These are cells that often contribute to the structural integrity of the tumor, feed the tumor and support growth of the tumor. In most cases, TAAs are overexpressed antigens in the tumor microenvironment, however some antigens in the tumor microenvironment may also be unique in the tumor associated cells. As an example, telomerase reverse transcriptase (TERT) is a TAA that is not present in most normal tissues but is activated in most human tumors. Tissue kallikrein-related peptidases, or kallikreins (KLKs), on the other hand are overexpressed in various cancers and comprise a large family of secreted trypsin- or chymotrypsin-like serine proteases. Kallikreins are upregulated in prostrate ovarian and breast cancers. Some TAAs are specific to certain cancers, some are expressed in a large variety of cancers. Carcinoembryonic antigen (CEA) is overexpressed in breast, colon, lung and pancreatic carcinomas, whereas MUC-1 is breast, lung, prostate, colon cancers. Some TAAs are differentiation or tissue specific, for example, MART-1/melan-A and gp100 are expressed in normal melanocytes and melanoma, and prostate specific membrane antigen (PSMA) and prostate-specific antigen (PSA) are expressed by prostate epithelial cells as well as prostate carcinoma.

In some embodiments, T cells are developed for adoptive therapy that are directed to overexpressed tissue specific or tumor associated antigens, such as prostrate specific kallikrein proteins KLK2, KLK3, KLK4 in case of prostate cancer therapy, or transglutamase protein 4, TGM4 for adenocarcinoma.

In some embodiments, the antigenic peptides that are targeted for the adoptive therapy in the methods disclosed herein are effective in modulating the tumor microenvironment. T cells are primed with antigens expressed by cells in the TME, so that the therapy is directed towards weakening and/or breaking down the tumor facilitating TME, oftentimes, in addition to directly targeting the tumor cells for T cell mediated lysis.

Tumor microenvironment comprises fibroblasts, stromal cells, endothelial cells and connective tissue cells which make up a large proportion of cells that induce or influence tumor growth. Just as T cells can be stimulated and directed attack the tumor cells in a immunosuppressive tumor environment, certain peptides and antigens can be utilized to direct the T cells against cells in the tumor vicinity that help in tumor propagation CD8+ and CD4+ T cells can be generated ex vivo that are directed against antigens on the surface of non-tumor cells in the tumor microenvironment that promote tumor sustenance and propagation. Cancer/tumor associated fibroblasts (CAFs) are hallmark feature of pancreatic cancers, such as pancreatic adenocarcinoma (PDACs). CAFs express Col10a1 antigen. CAFs are cells that may help perpetuate a tumor. Col10A1 often confers negative prognosis for the tumor. In some embodiments Col10A1 may be considered as a biomarker for tumor sustenance and progression. It is a 680 amino acid long heterodimer protein associated with poor prognosis in breast cancer and colorectal cancers.

Activation of Col10a1 specific CD8+ T cells and CD4+ T cells may help attack and destruction of Col10A1 specific fibroblasts and help break down the tissue matrix of solid tumors.

T cells can be generated ex vivo using the method described herein, so that the T cells are activated against cancer-associated fibroblasts (CAFs). For this, Col10a1 peptides comprising epitopes that can specifically activate T cells were generated, and the HLA binding partner determined, using the highly reliable data generated from the in-house generated machine learning epitope presentation software described previously as described in Table 12.

TABLE 12

| | | Rank on HLA |
|---|---|---|
| Peptide | HLA Allele | allele |
| FTCQIPGIYY | HLA-A01:01 | 1 |
| GSDGKPGY | HLA-A01:01 | 2 |
| NAESNGLY | HLA-A01:01 | 3 |
| LTENDQVWL | HLA-A01:01 | 4 |
| GTHVWVGLY | HLA-A01:01 | 5 |
| TYDEYTKGY | HLA-A01:01 | 6 |
| YTYDEYTKGY | HLA-A01:01 | 7 |
| FTCQIPGIY | HLA-A01:01 | 8 |
| NAESNGLYSSEY | HLA-A01:01 | 9 |
| YLDQASGSA | HLA-A01:01 | 10 |
| FLLLVSLNL | HLA-A02:01 | 1 |
| FLLLVSLNLV | HLA-A02:01 | 2 |
| GLYKNGTPV | HLA-A02:01 | 3 |
| GLDGPKGNPGL | HLA-A02:01 | 4 |
| LLLVSLNLV | HLA-A02:01 | 5 |
| SLSGTPLVSA | HLA-A02:01 | 6 |
| GLYSSEYV | HLA-A02:01 | 7 |
| SLSGTPLV | HLA-A02:01 | 8 |
| MLPQIPFLL | HLA-A02:01 | 9 |
| GLPGPPGPSA | HLA-A02:01 | 10 |
| SAFTVILSK | HLA-A03:01 | 1 |
| AVMPEGFIK | HLA-A03:01 | 2 |
| GLYKNGTPVMY | HLA-A03:01 | 3 |
| AIGTPIPFDK | HLA-A03:01 | 4 |
| GLPGGPGAK | HLA-A03:01 | 5 |
| ILYNRQQHY | HLA-A03:01 | 6 |
| AGPPGPPGFGK | HLA-A03:01 | 7 |
| GIPGFPGSK | HLA-A03:01 | 8 |
| GTHVWVGLYK | HLA-A03:01 | 9 |
| GVPGQPGIK | HLA-A03:01 | 10 |
| AVMPEGFIK | HLA-A11:01 | 1 |
| SAFTVILSK | HLA-A11:01 | 2 |
| VSAFTVILSK | HLA-A11:01 | 3 |
| GTHVWVGLYK | HLA-A11:01 | 4 |
| AIGTPIPFDK | HLA-A11:01 | 5 |
| AVMPEGFIKA | HLA-A11:01 | 6 |
| SSFSGFLVA | HLA-A11:01 | 7 |
| PVSAFTVILSK | HLA-A11:01 | 8 |
| GIPGFPGSK | HLA-A11:01 | 9 |
| GVPGMNGQK | HLA-A11:01 | 10 |
| AYPAIGTPIPF | HLA-A24:02 | 1 |
| IGPPGIPGF | HLA-A24:02 | 2 |
| HYDPRTGIF | HLA-A24:02 | 3 |
| EYVHSSFSGF | HLA-A24:02 | 4 |
| AGPPGPPGF | HLA-A24:02 | 5 |
| YYFSYHVHV | HLA-A24:02 | 6 |
| AYPAIGTPI | HLA-A24:02 | 7 |
| PLPNTKTQF | HLA-A24:02 | 8 |
| MLPQIPFLL | HLA-A24:02 | 9 |
| CQIPGIYYF | HLA-A24:02 | 10 |
| RPSLSGTPL | HLA-B07:02 | 1 |
| LPQIPFLLL | HLA-B07:02 | 2 |
| IPFLLLVSL | HLA-B07:02 | 3 |
| LPGPPGPSAV | HLA-B07:02 | 4 |
| GPIGPPGIPGF | HLA-B07:02 | 5 |
| IPGPAGISV | HLA-B07:02 | 6 |
| YPAIGTPIPF | HLA-B07:02 | 7 |
| SPGPPGPAGI | HLA-B07:02 | 8 |
| LPGPPGPSA | HLA-B07:02 | 9 |
| SPGPPGPAG | HLA-B07:02 | 10 |
| TIKSKGIAV | HLA-B08:01 | 1 |
| IPFLLLVSL | HLA-B08:01 | 2 |
| HVHVKGTHV | HLA-B08:01 | 3 |
| LPNTKTQF | HLA-B08:01 | 4 |
| LPQIPFLL | HLA-B08:01 | 5 |
| PFLLLVSL | HLA-B08:01 | 6 |
| SLNLVHGV | HLA-B08:01 | 7 |
| LPQIPFLLL | HLA-B08:01 | 8 |
| TGMPVSAF | HLA-B08:01 | 9 |
| TPIPFDKIL | HLA-B08:01 | 10 |

Neoantigenic peptides provided herein are prevalidated for HLA binding immunogenicity (Tables 1-12). In some embodiments the neoantigenic peptides, prepared and stored earlier, are used to contact an antigen presenting cell (APC) to then allow presentation to a T cell in vitro for preparation of neoantigen-specific activated T cell. In some embodiments, between 2-80 or more neoantigenic peptides are used to stimulate T cells from a patient at a time.

In some embodiments the APC is an autologous APC. In some embodiments the APC is a non-autologous APC. In some embodiments the APC is a synthetic cell designed to function as an APC. In some embodiments the T cell is an autologous cell. In some embodiments, an antigen presenting cell is a cell that expresses an antigen. For example, an antigen presenting cell may be a phagocytic cell such as a dendritic cell or myeloid cell, which process an antigen after cellular uptake and presents the antigen in association with an MHC for T cell activation. For certain purposes, an APC as used herein is a cell that normally presents an antigen on its surface. In a non-binding or non-limiting example, relevant to certain cytotoxicity assays as described herein, a tumor cell is an antigen presenting cell, that the T cell can recognize an antigen presenting cell (tumor cell). Similarly, a cell or cell line expressing an antigen can be, for certain purposes as used herein, an antigen presenting cell.

In some embodiments, one or more polynucleotides encoding one or more neoantigenic peptides may be used to express in a cell to present to a T cell for activation in vitro. The one or more polynucleotides encoding one or more of the neoantigenic peptides are encoded in a vector. In some embodiments, the composition comprises from about 2 to about 80 neoantigenic polynucleotides. In embodiments, at least one of the additional neoantigenic peptide is specific for an individual subject's tumor. In embodiments, the subject specific neoantigenic peptide is selected by identifying sequence differences between the genome, exome, and/or transcriptome of the subject's tumor sample and the genome, exome, and/or transcriptome of a non-tumor sample. In embodiments, the samples are fresh or formalin-fixed paraffin embedded tumor tissues, freshly isolated cells, or circulating tumor cells. In embodiments, the sequence differences are determined by Next Generation Sequencing.

In some embodiments the method and compositions provided herein can be used to identify or isolate a T cell receptor (TCR) capable of binding at least one neoantigenic peptide described herein or an MHC-peptide complex comprising at least one neoantigenic peptide described herein. In embodiments, the MHC of the MHC-peptide is MHC class I or class II. In embodiments, TCR is a bispecific TCR further comprising a domain comprising an antibody or antibody fragment capable of binding an antigen. In embodiments, the antigen is a T cell-specific antigen. In embodiments, the antigen is CD3. In embodiments, the antibody or antibody fragment is an anti-CD3 scFv.

In some embodiments the method and compositions provided herein can be used to prepare a chimeric antigen receptor comprising: (i) a T cell activation molecule; (ii) a transmembrane region; and (iii) an antigen recognition moiety capable of binding at least one neoantigenic peptide described herein or an MHC-peptide complex comprising at least one neoantigenic peptide described herein. In embodiments, CD3-zeta is the T cell activation molecule. In embodiments, the chimeric antigen receptor further comprises at least one costimulatory signaling domain. The In embodiments, the signaling domain is CD28, 4-1BB, ICOS, OX40, ITAM, or Fc epsilon RI-gamma. In embodiments, the antigen recognition moiety is capable of binding the isolated neoantigenic peptide in the context of MHC class I or class II. In embodiments, the CD3-zeta, CD28, CTLA-4, ICOS, BTLA, KIR, LAG3, CD137, OX40, CD27, CD40L, Tim-3, A2aR, or PD-1 transmembrane region. In embodiments, the neoantigenic peptide is located in the extracellular domain of a tumor associated polypeptide. In embodiments, the MHC of the MHC-peptide is MHC class I or class II.

Provided herein is a T cell comprising the T cell receptor or chimeric antigen receptor described herein, optionally wherein the T cell is a helper or cytotoxic T cell. In embodiments, the T cell is a T cell of a subject.

Provided herein is a T cell comprising a T cell receptor (TCR) capable of binding at least one neoantigenic peptide described herein or an MHC-peptide complex comprising at least one neoantigenic peptide described herein, wherein the T cell is a T cell isolated from a population of T cells from a subject that has been incubated with antigen presenting cells and one or more of the at least one neoantigenic peptide described herein for a sufficient time to activate the T cells. In embodiments, the T cell is a CD8+ T cell, a helper T cell or cytotoxic T cell. In embodiments, the population of T cells from a subject is a population of CD8+ T cells from the subject. In embodiments, the one or more of the at least one neoantigenic peptide described herein is a subject-specific neoantigenic peptide. In embodiments, the subject-specific neoantigenic peptide has a different tumor neo-epitope that is an epitope specific to a tumor of the subject. In embodiments, the subject-specific neoantigenic peptide is an expression product of a tumor-specific non-silent mutation that is not present in a non-tumor sample of the subject. In embodiments, the subject-specific neoantigenic peptide binds to a HLA protein of the subject. In embodiments, the subject-specific neoantigenic peptide binds to a HLA protein of the subject with an IC50 less than 500 nM. In embodiments, the activated CD8+ T cells are separated from the antigen presenting cells. In embodiments, the antigen presenting cells are dendritic cells or CD40L-expanded B cells. In embodiments, the antigen presenting cells are non-transformed cells. In embodiments, the antigen presenting cells are non-infected cells. In embodiments, the antigen presenting cells are autologous. In embodiments, the antigen presenting cells have been treated to strip endogenous MHC-associated peptides from their surface. In embodiments, the treatment to strip the endogenous MHC-associated peptides comprises culturing the cells at about 26° C. In embodiments, the treatment to strip the endogenous MHC-associated peptides comprises treating the cells with a mild acid solution. In embodiments, the antigen presenting cells have been pulsed with at least one neoantigenic peptide described herein. In embodiments, pulsing comprises incubating the antigen presenting cells in the presence of at least about 2 μg/mL of each of the at least one neoantigenic peptide described herein. In embodiments, ratio of isolated T cells to antigen presenting cells is between about 30:1 and 300:1. In embodiments, the incubating the isolated population of T cells is in the presence of IL2 and IL-7. In embodiments, the MEW of the MHC-peptide is MEW class I or class II.

Provided herein is a method for activating tumor specific T cells comprising: isolating a population of T cells from a subject; and incubating the isolated population of T cells with antigen presenting cells and at least one neoantigenic peptide described herein for a sufficient time to activate the T cells. In embodiments, the T cell is a CD8+ T cell, a helper T cell or cytotoxic T cell. In embodiments, the population of T cells from a subject is a population of CD8+ T cells from the subject. In embodiments, the one or more of the at least one neoantigenic peptide described herein is a subject-specific neoantigenic peptide. In embodiments, the subject-specific neoantigenic peptide has a different tumor neo-epitope that is an epitope specific to a tumor of the subject. In embodiments, the subject-specific neoantigenic peptide is an expression product of a tumor-specific non-silent mutation that is not present in a non-tumor sample of the subject. In embodiments, the subject-specific neoantigenic peptide binds to a HLA protein of the subject. In embodiments, the subject-specific neoantigenic peptide binds to a HLA protein of the subject with an IC50 less than 500 nM. In embodiments, the method further comprises separating the activated T cells from the antigen presenting cells. In embodiments, the method further comprises testing the activated T cells for evidence of reactivity against at least one of neoantigenic peptide of described herein. In embodiments, the antigen presenting cells are dendritic cells or CD40L-expanded B cells. In embodiments, the antigen presenting cells are non-transformed cells. In embodiments, the antigen presenting cells are non-infected cells. In embodiments, the antigen presenting cells are autologous. In embodiments, the antigen presenting cells have been treated to strip endogenous MHC-associated peptides from their surface. In embodiments, the treatment to strip the endogenous MHC-associated peptides comprises culturing the cells at about 26° C. In embodiments, the treatment to strip the endogenous MHC-associated peptides comprises treating the cells with a mild acid solution. In embodiments, the antigen presenting cells have been pulsed with at least one neoantigenic peptide described herein. In embodiments, pulsing comprises incubating the antigen presenting cells in the presence of at least about 2 μg/ml of each of at least one neoantigenic peptide described herein. In embodiments, ratio of isolated T cells to antigen presenting cells is between about 30:1 and 300:1. In embodiments, the incubating the isolated population of T cells is in the presence of IL2 and IL-7. In embodiments, the MHC of the MHC-peptide is MHC class I or class II.

Provided herein is a composition comprising activated tumor specific T cells produced by a method described herein.

Provided herein is a method of treating cancer in a subject comprising administering to the subject a therapeutically effective amount of activated tumor specific T cell described herein, or produced by a method described herein. In embodiments, the administering comprises administering from about 10\hat{ }6 to 10\hat{ }12, from about 10\hat{ }8 to 10\hat{ }11, or from about 10\hat{ }9 to 10\hat{ }10 of the activated tumor specific T cells.

Provided herein is a nucleic acid comprising a promoter operably linked to a polynucleotide encoding the T cell receptor described herein. In embodiments, the TCR is capable of binding the at least one neoantigenic peptide in the context of major histocompatibility complex (MHC) class I or class II. In some embodiments, provided herein is a TCR comprising a TCR alpha chain and a TCR beta chain capable of binding to a mutated RAS epitope in complex with an MHC encoded by a C03:04 or a 03:03 HLA molecule. A TCR can be identified that specifically binds to an epitope, e.g., a mutant RAS epitope as described herein, for example, by isolating and sequencing TCRs from T cells that can bind to and are activated by the antigen-MHC complex. In some embodiments, provided herein are TCRs that can bind to an antigen comprising a GACGVGKSA epitope in complex with a protein encoded by an HLA-C03:04 allele. In some embodiments, provided herein are TCRs that can bind to an antigen comprising a GAVGVGKSA epitope in complex with a protein encoded by the HLA-C03:03 allele. In some embodiments, the TCR is modified. Also provided herein is a nucleic acid molecule comprising a sequence encoding a TCR alpha chain and/or a TCR beta chain that can bind to an antigen comprising a GACGVGKSA epitope in complex with a protein encoded by an HLA-C03:04 allele. Also provided herein is a nucleic acid molecule comprising a sequence encoding a TCR alpha chain and/or a TCR beta chain that can bind to an antigen comprising a GAVGVGKSA epitope in complex with a protein encoded by the HLA-C03:03 allele.

Provided herein is a nucleic acid comprising a promoter operably linked to a polynucleotide encoding the chimeric antigen receptor described herein. In embodiments, the antigen recognition moiety is capable of binding the at least one neoantigenic peptide in the context of major histocompatibility complex (MHC) class I or class II. In embodiments, the neoantigenic peptide is located in the extracellular domain of a tumor associated polypeptide. In embodiments, the nucleic acid comprises the CD3-zeta, CD28, CTLA-4, ICOS, BTLA, KIR, LAG3, CD137, OX40, CD27, CD40L, Tim-3, A2aR, or PD-1 transmembrane region.

In some embodiments the autologous immune cells from the peripheral blood of the patient constitute peripheral blood mononuclear cells (PBMC). In some embodiments the autologous immune cells from the peripheral blood of the patient are collected via an apheresis procedure. In some embodiments, the PBMCs are collected from more than one apheresis procedures, or more than one draw of peripheral blood.

In some embodiments, both CD25+ cells and the CD14+ cells are depleted prior to addition of peptides. In some embodiments, either of CD25+ cells or the CD14+ cells are depleted prior to addition of peptides. In some embodiments, CD25+ cells and not the CD14+ cells are depleted prior to addition of peptides.

In some embodiments, the depletion procedure is followed by the addition of FMS-like tyrosine kinase 3 receptor ligand (FLT3L) to stimulate the APCs, constituted by the monocytes, macrophages or dendritic cells (DCs) prior to addition of the peptides. In some embodiments, the depletion procedure is followed by selection of DC as suitable PACs for peptide presentation to the T cells, and mature macrophages and other antigen presenting cells are removed from the autologous immune cells from the patient. In some embodiments, the depletion procedure is followed by selection of immature DC as suitable PACs for peptide presentation to the T cells.

In some embodiments, a selection of ‘n’ number of neoantigenic peptides is contacted with the APCs for stimulation of the APCs for antigen presentation to the T cells.

In some embodiments, a first level selection of ‘n’ number of neoantigenic peptides is based on the binding ability of each of the peptides to at least on HLA haplotype that is predetermined to be present in the recipient patient. In order to determine HLA haplotype that is predetermined to be present in the recipient patient, as is known to one of skill in the art, a patient is subjected to HLA haplotyping assay form a blood sample prior to the commencement of the treatment procedure. In some embodiments, a first level selection of ‘n’ number of neoantigenic peptides is followed by a second level selection based on the determination of whether the mutation present in the neoantigenic peptide(s) match the neoantigens (or mutations leading to) known to be found in at least 5% of patients known to have the cancer. In some embodiments, the second level of the selection involves further determination of whether the mutation is evident in the patient.

In some embodiments, a first and the second level selection of ‘n’ number of neoantigenic peptides for contacting the APCs is followed by a third level of selection, based on the binding affinity of the peptide with the HLA that the peptide is capable of binding to and is at least less than 500 nM, with the determination that higher the binding affinity, the better the choice of the peptide to be selected. In some embodiments, the finally selected ‘n’ number of peptides can range from 1-200 peptides which are in a mix, for exposing APCs to the peptides in the culture media, and contacting with APCs.

In some embodiments the ‘n’ number of peptides can range from 10-190 neoantigenic peptides. In some embodiments the ‘n’ number of peptides can range from 20-180 neoantigenic peptides. In some embodiments the ‘n’ number of peptides can range from 30-170 neoantigenic peptides. In some embodiments the ‘n’ number of peptides can range from 40-160 neoantigenic peptides. In some embodiments the ‘n’ number of peptides can range from 50-150 neoantigenic peptides. In some embodiments the ‘n’ number of peptides can range from 60-140 neoantigenic peptides. In some embodiments the ‘n’ number of peptides can range from 70-130 neoantigenic peptides. In some embodiments the ‘n’ number of peptides can range from 80-120 neoantigenic peptides. In some embodiments the ‘n’ number of peptides can range from 50-100 neoantigenic peptides. In some embodiments the ‘n’ number of peptides can range from 50-90 neoantigenic peptides. In some embodiments the ‘n’ number of peptides can range from 50-80 neoantigenic peptides. In some embodiments the ‘n’ number of peptides comprise at least 60 neoantigenic peptides. In some embodiments the ‘n’ number of peptides comprise a mixture of (a) neoantigenic peptides that are short, 8-15 amino acids long, comprising the mutated amino acid as described previously, following the formula AxByCz; these peptides are interchangeably called shortmers or short peptides for the purpose of this application; and (b) long peptides that are 15, 30, 50, 60, 80, 100-300 amino acids long and any length in between, which are subject to endogenous processing by dendritic cells for better antigen presentation; these peptides are interchangeably called longmers or long peptides for the purpose of this application. In some embodiments the at least 60 neoantigenic peptides comprise at least 30 shortmers and at least 30 longmers or variations of the same. Exemplary variations of the same include, but are not limited to the following: in some embodiments the at least 60 neoantigenic peptides comprise at least 32 shortmers and at least 32 longmers or variations of the same. In some embodiments the at least 60 neoantigenic peptides comprise at least 34 shortmers and at least 30 longmers or variations of the same. In some embodiments the at least 60 neoantigenic peptides comprise at least 28 shortmers and at least 34 longmers or variations of the same.

In some embodiments, the ‘n’ number of peptides are incubated in the medium comprising APCs in culture, where the APCs (DCs) have been isolated from the PBMCs, and previously stimulated with FLT3L. In some embodiments, the ‘n’ number of peptides are incubated with APCs in presence of FLT3L. In some embodiments, following the step of incubation of the APCs with FLT3L, the cells are added with fresh media containing FL3TL for incubation with peptides. In some embodiments, the maturation of APCs to mature peptide loaded DCs may comprise several steps of culturing the DCs towards maturation, examining the state of maturation by analysis of one or more released substances, (e.g. cytokines, chemokines) in the culture media or obtaining an aliquot of the DCs in culture form time to time. In some embodiments, the maturation of DCs take at least 5 days in culture from onset of the culture. In some embodiments, the maturation of DCs take at least 7 days in culture from onset of the culture. In some embodiments, the maturation of DCs take at least 11 days in culture from onset of the culture, or any number of days in between.

In some embodiments, the DCs are contacted with T cells after being verified for presence of or absence of maturation factors and peptide tetramer assay for verifying the repertoire of antigens presented.

In some embodiments, the DCs are contacted with T cells in a T cell media for about 2 days for the first induction. In some embodiments, the DCs are contacted with T cells in a T cell media for about 3 days for the first induction. In some embodiments, the DCs are contacted with T cells in a T cell media for about 4 days for the first induction. In some embodiments, the DCs are contacted with T cells in a T cell media for at least about 2 days for the second induction. In some embodiments, the DCs are contacted with T cells in a T cell media for at least about 3 days for the second induction. In some embodiments, the DCs are contacted with T cells in a T cell media for at least about 4 days for the second induction. In some embodiments, the DCs are contacted with T cells in a T cell media for 5 days for the second induction. In some embodiments, the DCs are contacted with T cells in a T cell media for about 6 days for the second induction. In some embodiments, the DCs are contacted with T cells in a T cell media for about 7, 8, 9 or days for the second induction. In some embodiments, the DCs are contacted with T cells in a T cell media for about less than 1 days for the third induction. In some embodiments, the DCs are contacted with T cells in a T cell media for at least about 2 or 3 days for the third induction. In some embodiments, the DCs are contacted with T cells in a T cell media for at least about 4 days for the third induction. In some embodiments, the DCs are contacted with T cells in a T cell media for 5 days for the third induction. In some embodiments, the DCs are contacted with T cells in a T cell media for about 6 days for the third induction. In some embodiments, the DCs are contacted with T cells in a T cell media for about 7, 8, 9 or days for the second induction.

In some embodiments, the T cells are further contacted with one or more shortmer peptides during incubation with DCs (and in addition to the DCs) at either the first induction phase, the second induction phase or the third induction phase. In some embodiments, the T cells are further contacted with one or more shortmer peptides during incubation with DCs at the first induction phase and the second induction phase. In some embodiments, the T cells are further contacted with one or more shortmer peptides during incubation with DCs at the second induction phase and the third induction phase. In some embodiments, the T cells are further contacted with one or more shortmer peptides in all the three induction phases.

In some embodiments, the APCs and the T cells are comprised in the same autologous immune cells from the peripheral blood of the patient drawn at the first step from the patient. The T cells are isolated and preserved for the time of activation with the DCs at the end of the DC maturation phase. In some embodiments the T cells are cocultured in the presence of a suitable media for activation for the time of activation with the DCs at the end of the DC maturation phase. In some embodiments the T cells are prior cyropreserved cells from the patient, which are thawed and cultured for at least 4 hours to up to about 48 hours for induction at the time of activation with the DCs at the end of the DC maturation phase.

In some embodiments, the APCs and the T cells are comprised in the same autologous immune cells from the peripheral blood of the patient drawn at the different time periods from the patient, e.g. at different apheresis procedures. In some embodiments the time from apheresis of the patient to the time of harvest, takes between about 20 days to about less than 26 days. In some embodiments the time from apheresis of the patient to the time of harvest, takes between about 21 days to about less than 25 days. In some embodiments the time from apheresis of the patient to the time of harvest, takes between about 21 days to about less than 24 days. In some embodiments the time from apheresis of the patient to the time of harvest, takes between about 21 days to about less than 23 days. In some embodiments the time from apheresis of the patient to the time of harvest, takes about 21 days. In some embodiments the time from apheresis of the patient to the time of harvest, takes about less than 21 days.

In some embodiments the release criteria for the activated T cells (the drug substance) comprises any one or more of sterility, endotoxin, cell phenotype, TNC Count, viability, cell concentration, potency. In some embodiments the release criteria for the activated T cells (the drug substance) comprises each one of sterility, endotoxin, cell phenotype, TNC Count, viability, cell concentration, potency.

In some embodiments the total number of cells is 2×10\hat{ }10. In some embodiments the total number of cells is 2×10\hat{ }9. In some embodiments the total number of cells is 5×10\hat{ }8. In some embodiments the total number of cells is 2×10\hat{ }8. In some embodiments the final concentration of the resuspended T cells is 2×10\hat{ }5 cells/ml or more. In some embodiments the final concentration of the resuspended T cells is 1×10\hat{ }6 cells/ml or more. In some embodiments the final concentration of the resuspended T cells is 2×10\hat{ }6 cells/ml or more.

The following criteria of released cells are described as exemplary non-limiting conditions, particularly because of the reason that the criteria for the cell population and subpopulations in Drug substance (DS) can vary based on the cancer, the state of the cancer, the state of the patient, the availability of the matched HLA haplotype and the growth potential of the APCs and T cells in the presence of the peptide. In some embodiments the activated T cells (the drug substance) comprises at least 2% or at least 3% or at least 4% or at least 5% of CD8+ T cells reactive to a particular neoantigen by tetramer assay. In some embodiments, the activated T cells (the drug substance) comprises at least 2% or at least 3% or at least 4% or at least 5% of CD4+ T cells reactive to a particular neoantigen by tetramer assay. In some embodiments, the activated T cells (the drug substance) comprise at least 5% or at least 6% or at least 7% or at least 8% or at least 9% or at least 10% of cells that are positive for memory T cell phenotype.

In some embodiments, the activated T cells (the drug substance) are selected based on one or more markers. In some embodiments, the activated T cells (the drug substance) are not selected based on one or more markers. In some embodiments, an aliquot of the activated T cells (the drug substance) are tested for the presence or absence of one or more of the following markers, and the proportions of cells thereof exhibiting each of the tested markers, the one or more markers are selected from a group consisting of: CD19, CD20, CD21, CD22, CD24, CD27, CD38, CD40, CD72, CD3, CD79a, CD79b, IGKC, IGHD, MZB1, TNFRSF17, MS4A1, CD138, TNFRSR13B, GUSPB11, BAFFR, AID, IGHM, IGHE, IGHA1, IGHA2, IGHA3, IGHA4, BCL6, FCRLA CCR7, CD27, CD45RO, FLT3LG, GRAP2, IL16, IL7R, LTB, S1PR1, SELL, TCF7, CD62L, PLAC8, SORL1, MGAT4A, FAM65B, PXN, A2M, ATM, C20orf112, GPR183, EPB41, ADD3, GRAP2, KLRG1, GIMAP5, TC2N, TXNIP, GIMAP2, TNFAIP8, LMNA, NR4A3, CDKN1A, KDM6B, ELL2, TIPARP, SC5D, PLK3, CD55, NR4A1, REL, PBX4, RGCC, FOSL2, SIK1, CSRNP1, GPR132, GLUL, KIAA1683, RALGAPA1, PRNP, PRMT10, FAM177A1, CHMP1B, ZC3H12A, TSC22D2, P2RY8, NEU1, ZNF683, MYADM, ATP2B1, CREM, OAT, NFE2L2, DNAJB9, SKIL, DENND4A, SERTAD1, YPEL5, BCL6, EGR1, PDE4B, ANXA1, SOD2, RNF125, GADD45B, SELK, RORA, MXD1, IFRD1, PIK3R1, TUBB4B, HECA, MPZL3, USP36, INSIG1, NR4A2, SLC2A3, PER1, S100A10, AIM1, CDC42EP3, NDEL1, IDI1, EIF4A3, BIRC3, TSPYL2, DCTN6, HSPH1, CDK17, DDX21, PPP1R15B, ZNF331, BTG2, AMD1, SLC7A5 POLR3E, JMJD6, CHD1, TAF13, VPS37B, GTF2B, PAF1, BCAS2, RGPD6, TUBA4A, TUBA1A, RASA3, GPCPD1, RASGEF1B, DNAJA1, FAM46C, PTP4A1, KPNA2, ZFAND5, SLC38A2, PLIN2, HEXIM1, TMEM123, JUND, MTRNR2L1, GABARAPL1, STAT4, ALG13, FOSB, GPR65, SDCBP, HBP1, MAP3K8, RANBP2, FAM129A, FOS, DDIT3, CCNH, RGPD5, TUBA1C, ATP1B3, GLIPR1, PRDM2, EMD, HSPD1, MORF4L2, IL21R, NFKBIA, LYAR, DNAJB6, TMBIM1, PFKFB3, MED29, B4GALT1, NXF1, BIRC2, ARHGAP26, SYAP1, DNTTIP2, ETF1, BTG1, PBXIP1, MKNK2, DEDD2, AKIRIN1, HLA-DMA, HLA-DNB, HLA-DOA, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, CCL18, CCL19, CCL21, CXCL13, LAMP3, LTB, IL7R, MS4A1, CCL2, CCL3, CCL4, CCL5, CCL8, CXCL10, CXCL11, CXCL9, CD3, LTA, IL17, IL23, IL21, IL7, CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT, CD56, CCL2, CCL3, CCL4, CCL5, CXCL8, IFN, IL2, IL-12, IL-15, IL-18, NCR1, XCL1, XCL2, IL21R, KIR2DL3, KIR3DL1, KIR3DL2, NCAM1, HLA-DMA, HLA-DNB, HLA-DOA, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, and HLA-DRB5.

In some embodiments, at least 0.01% of naive T cells which were obtained from the obtaining of autologous immune cells from the peripheral blood of the patient were stimulated in response to a neoantigen, and was amplified at the end of the procedure and was harvested. In some embodiments, greater than 0.01% of naive T cells which were obtained from the obtaining of autologous immune cells from the peripheral blood of the patient were stimulated in response to a neoantigen, and was amplified at the end of the procedure and was harvested. In some embodiments, greater than 0.1% of naive T cells which were obtained from the obtaining of autologous immune cells from the peripheral blood of the patient were stimulated in response to a neoantigen, and was amplified at the end of the procedure and was harvested. In some embodiments, greater than 1% of naive T cells which were obtained from the obtaining of autologous immune cells from the peripheral blood of the patient were stimulated in response to a neoantigen, and was amplified at the end of the procedure and was harvested.

In some embodiments the total number of cells is harvested from 1, 2, or 3 cycles of the process of DC maturation and T cell activation.

In some embodiments the harvested cells are cryopreserved in vapor phase of liquid nitrogen in bags.

As is known to one of skill in the art, all applications described in the preceding paragraphs of this section from obtaining of autologous immune cells from the peripheral blood of the patient to the harvesting of cells is performed in an aseptic closed system, except the steps where aliquots of media or cells are taken out for examination by flow cytometry, mass spectroscopy, cell count, cell sorting or any functional assays, that are terminal to the cells or materials taken out as aliquots. In some embodiments the closed system for aseptic culture of up to the harvesting is proprietary to the applicant's process.

In some embodiments the T cells are method for culturing and expansion of activated T cells including the steps delineated above, starting from obtaining of autologous immune cells from the peripheral blood of the patient to harvesting, is scalable in an aseptic procedure. In some embodiments, at least 1 Liter of DC cell culture is performed at a time. In some embodiments, at least 1-2 Liters of T cell culture is performed at a time. In some embodiments, at least 5 Liters of DC cell culture is performed at a time. In some embodiments, at least 5-10 Liters of T cell culture is performed at a time. In some embodiments, at least 10 Liter of DC cell culture is performed at a time. In some embodiments, at least 10-40 Liters of T cell culture is performed at a time. In some embodiments, at least 10 Liter of DC cell culture is performed at a time. In some embodiments, at least 10-50 Liters of T cell culture is performed at a time. In some embodiments, simultaneous batch cultures are performed and tested in a system that is a closed system, and that can be manipulated and intervened from outside without introducing non-aseptic means. In some embodiments, a closed system described herein is fully automated.

When administration is by injection, the active agent can be formulated in aqueous solutions, specifically in physiologically compatible buffers such as Hanks solution, Ringer's solution, or physiological saline buffer. The solution can contain formulation agents such as suspending, stabilizing and/or dispersing agents. Alternatively, the active compound can be in powder form for constitution with a suitable vehicle, e.g., sterile pyrogen-free water, before use. In another embodiment, the drug product comprises a substance that further activates or inhibits a component of the host's immune response, for example, a substance to reduce or eliminate the host's immune response to the peptide.

The disclosure provided herein demonstrates that shared neoantigens can be used for ready therapeutic administration of a patient, thereby reducing the bench-to-bedside time lag considerably. The composition and methods described herein provide innovative advancements in the field of cancer therapeutics.

### EXAMPLES

### Example 1. Precision NEOSTIM Clinical Process

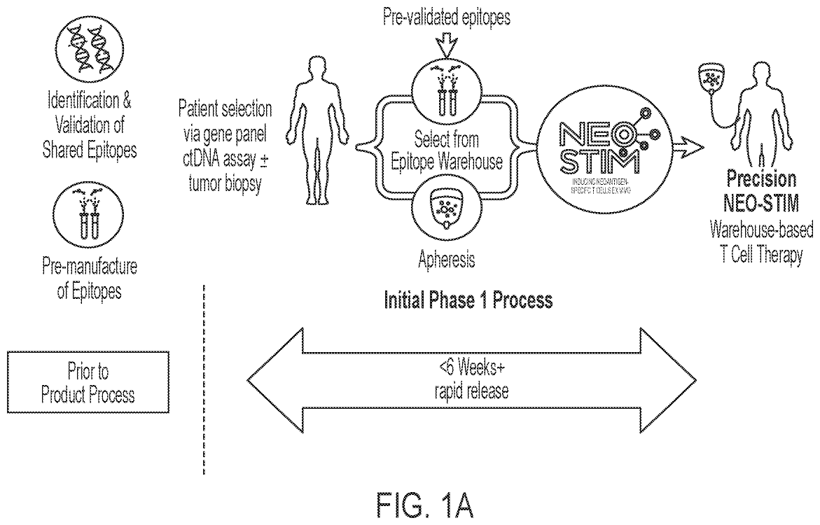

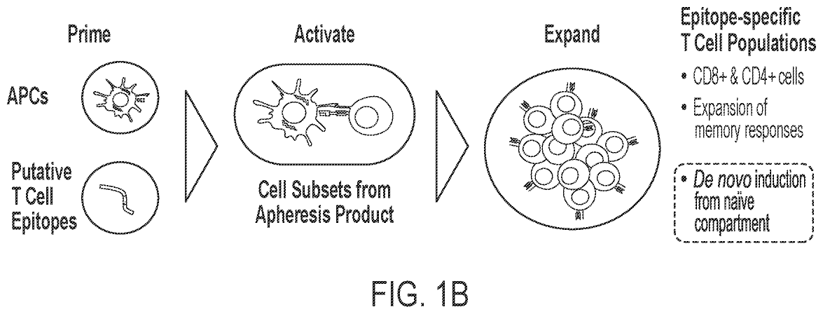

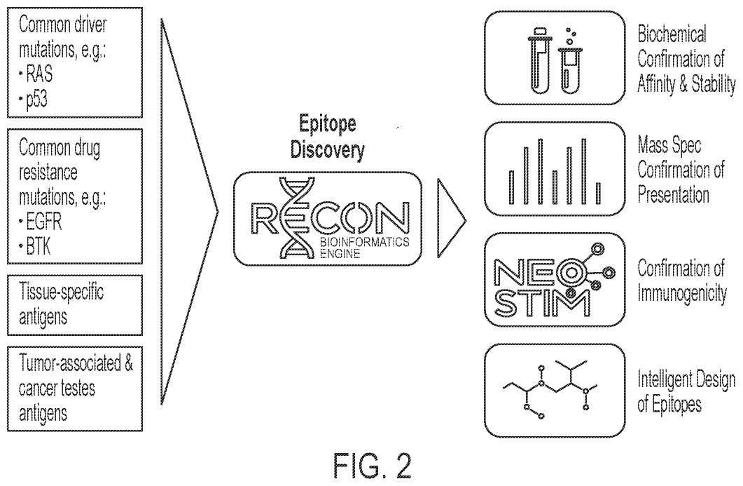

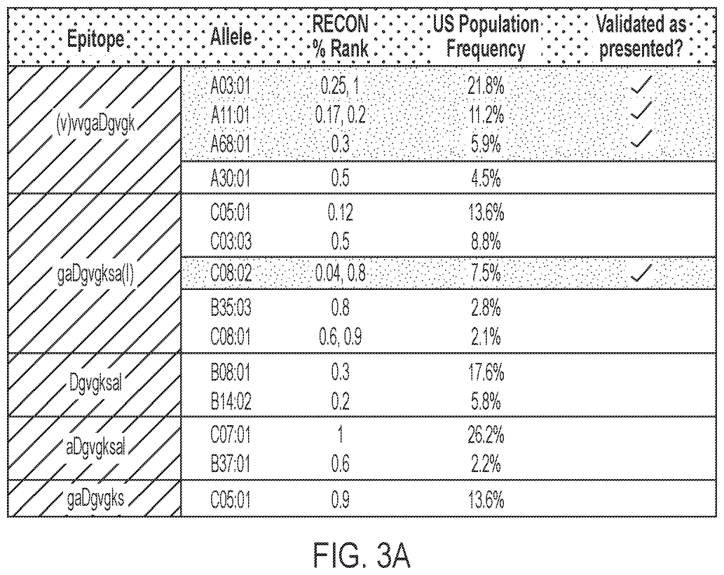

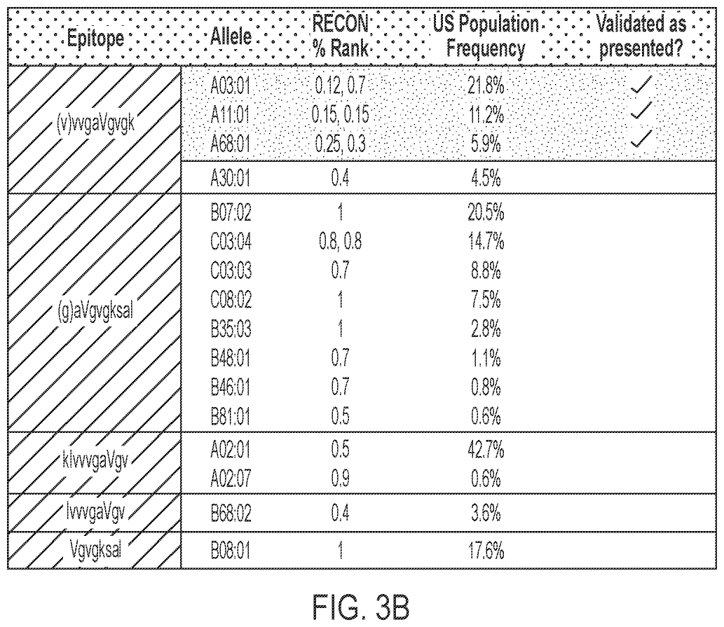

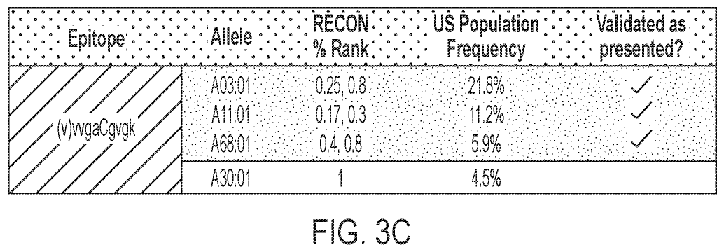

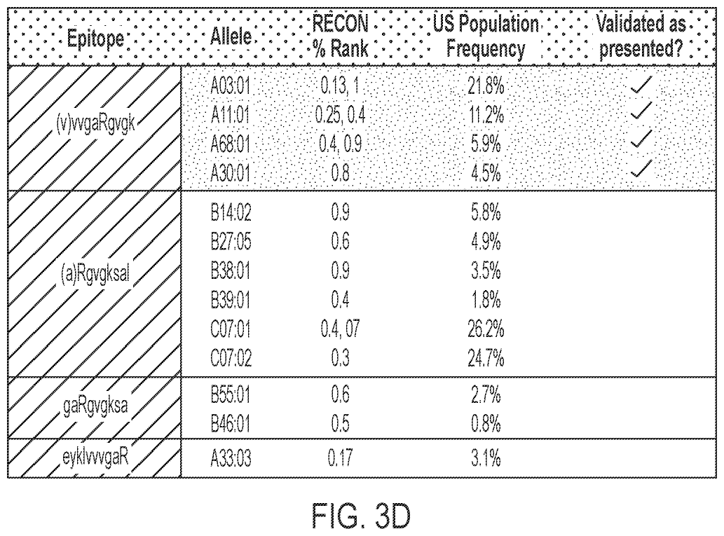

Provided herein is an adoptive T cell therapy where T cells primed and responsive against curated pre-validated, shelved, antigenic peptides specific for a subject's cancer is administered to the subject. Provided in this example is a method of bypassing lengthy sequencing, identification and manufacture of subject specific neoantigen peptides and thereafter generating T cells having the subject specific TCRs for cancer immunotherapy, at least for the time when a subject undergoes a process of such evaluation and preparations for the personalized therapy. Advantage of this process is that it is fast, targeted and robust. As shown in FIG. 1A, patient identified with a cancer or tumor can be administered T cells that are activated ex vivo with warehouse curated peptides having selected, validated collection of epitopes generated from a library of shared antigens known for the identified cancer. The process from patient selection to the T cell therapy may require less than 6 weeks. FIG. 1B illustrates the method of generating cancer target specific T cells ex vivo by priming T cells with APCs expressing putative T cell epitopes and expanding the activated T cells to obtain epitope-specific CD8+ and CD4+ including a population of these cells exhibiting memory phenotype (see, e.g., WO2019094642, incorporated by reference in its entirety). A library of prevalidated epitopes is generated in advance. Such epitopes are collected from prior knowledge in the field, common driver mutations, common drug resistant mutations, tissue specific antigens, and tumor associated antigens. With the help of an efficient computer-based program for epitope prediction, HLA binding and presentation characteristics, pre-validated peptides are generated for storage and stocking as shown in a diagram in FIG. 2. Exemplary predictions for common RAS G12 mutations are shown in FIG. 3A-3D. Validations are performed using a systematic process as outlined in Examples 2-5. Target tumor cell antigen responsive T cells are generated ex vivo and immunogenicity is validated using an in vitro antigen-specific T cell assay (Example 2). Mass spectrometry is used to validate that cells that express the antigen of interest can process and present the peptides on the relevant HLA molecules (Example 3). Additionally, the ability of these T cells to kill cells presenting the peptide is confirmed using a cytotoxicity assay (Example 4). Exemplary data provided herein demonstrate this validation process for RAS and GATA3 neoantigens, and can be readily applied to other antigens.

### Example 2. Generation of Target Tumor Cell Antigen Responsive T Cells Ex Vivo

Materials:
AIM V media (Invitrogen)
Human FLT3L, preclinical CellGenix #1415-050 Stock 50 ng/μL
TNF-α, preclinical CellGenix #1406-050 Stock 10 ng/μL
IL-1β, preclinical CellGenix #1411-050 Stock 10 ng/μL
PGE1 or Alprostadil—Cayman from Czech republic Stock 0.5 μg/μL
R10 media—RPMI 1640 glutamax+10% Human serum+1% PenStrep
20/80 Media—18% AIM V+72% RPMI 1640 glutamax+10% Human Serum+1% PenStrep
IL7 Stock 5 ng/μL
IL15 Stock 5 ng/μL

### Procedure:

Step 1: Plate 5 million PBMCs (or cells of interest) in each well of 24 well plate with FLT3L in 2 mL AIM V media
Step 2: Peptide loading and maturation—in AIMV
1. Mix peptide pool of interest (except for no peptide condition) with PBMCs (or cells of interest) in respective wells.
2. Incubate for 1 hr.
3. Mix Maturation cocktail (including TNF-α, IL-1β, PGE1, and IL-7) to each well after incubation.
   Step 3: Add human serum to each well at a final concentration of 10% by volume and mix.
   Step 4: Replace the media with fresh RPMI+10% HS media supplemented with IL7+IL15.
   Step 5: Replace the media with fresh 20/80 media supplemented with IL7+IL15 during the period of incubation every 1-6 days.
   Step 6: Plate 5 million PBMCs (or cells of interest) in each well of new 6-well plate with FLT3L in 2 ml AIM V media
   Step 7: Peptide loading and maturation for re-stimulation—(new plates)
1. Mix peptide pool of interest (except for no peptide condition) with PBMCs (or cells of interest) in respective wells
2. Incubate for 1 hr.
3. Mix Maturation cocktail to each well after incubation

### Step 8: Re-stimulation:

1. Count first stimulation FLT3L cultures and add 5 million cultured cells to the new Re-stimulation plates.
2. Bring the culture volume to 5 mL (AIM V) and add 500 μL of Human serum (10% by volume)
   Step 9: Remove 3 ml of the media and add 6 ml of RPMI+10% HS media supplemented with IL7+IL15.
   Step 10: Replace 75% of the media with fresh 20/80 media supplemented with IL7+IL15.
   Step 11: Repeat re-stimulation if needed.

### Analysis of Antigen-Specific Induction

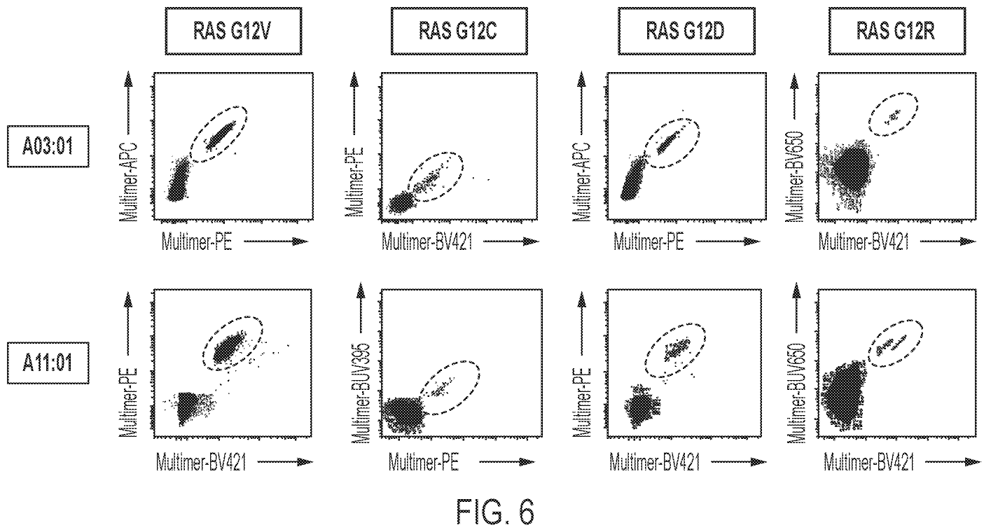

MHC tetramers are purchased or manufactured on-site according to methods known by one of ordinary skill, and are used to measure peptide-specific T cell expansion in the immunogenicity assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a LSR Fortessa (Becton Dickinson) instrument, and are analyzed by use of FlowJo software (Becton Dickinson). For analysis of tetramer positive cells, the lymphocyte gate is taken from the forward and side-scatter plots. Data are reported as the percentage of cells that were CD8⁺/tetramer⁺. Exemplary data for RAS neoantigens on HLA-A03:01 and HLA-A11:01 are shown in FIG. 6.

### Example 3. Evaluation of Presentation of Antigens

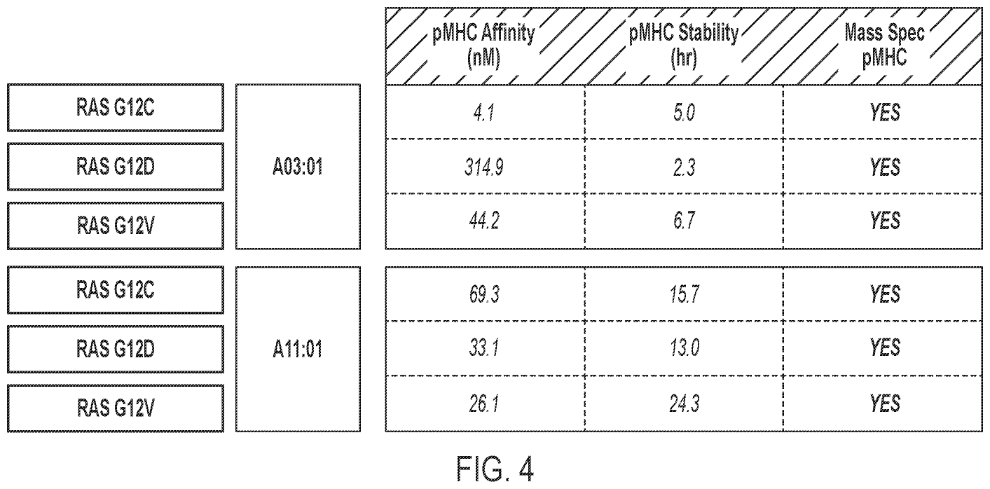

For a subset of predicted antigens, the affinity of the neoepitopes for the indicated HLA alleles and stability of the neoepitopes with the HLA alleles was determined. Exemplary data for a subset of RAS neoantigens and GATA3 neoantigens are shown in FIG. 4.

An exemplary detailed description of the protocol utilized to measure the binding affinity of peptides to Class I MHC has been published (Sette et al, Mol. Immunol. 31(11):813-22, 1994). In brief, MHCI complexes were prepared and bound to radiolabeled reference peptides. Peptides were incubated at varying concentrations with these complexes for 2 days, and the amount of remaining radiolabeled peptide bound to MHCI was measured using size exclusion gel-filtration. The lower the concentration of test peptide needed to displace the reference radiolabeled peptide demonstrates a stronger affinity of the test peptide for MHCI. Peptides with affinities to MHCI<50 nM are generally considered strong binders while those with affinities <150 nM are considered intermediate binders and those <500 nM are considered weak binders (Fritsch et al, 2014).

An exemplary detailed description of the protocol utilized to measure the binding stability of peptides to Class I MHC has been published (Harndahl et al. J Immunol Methods. 374:5-12, 2011). Briefly, synthetic genes encoding biotinylated MHC-I heavy and light chains are expressed in E. coli and purified from inclusion bodies using standard methods. The light chain (β2m) is radio-labeled with iodine (1251), and combined with the purified MHC-I heavy chain and peptide of interest at 18° C. to initiate pMHC-I complex formation. These reactions are carried out in streptavidin coated microplates to bind the biotinylated MHC-I heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts.

To assess whether antigens could be processed and presented from the larger polypeptide context, peptides eluted from HLA molecules isolated from cells expressing the genes of interest were analyzed by tandem mass spectrometry (MS/MS).

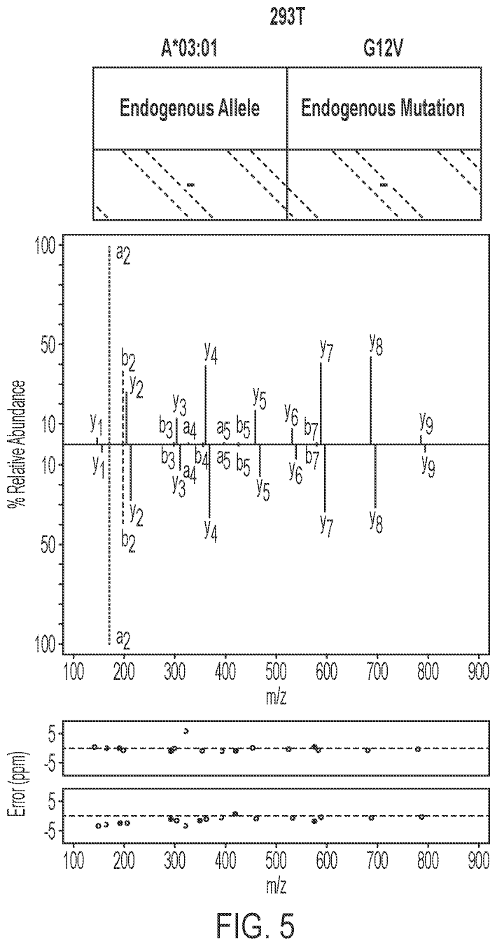

For analysis of presentation of RAS neoantigens, cell lines were utilized that have RAS mutations naturally or were lentivirally transduced to express the mutated RAS gene. HLA molecules were either isolated based on the natural expression of the cell lines or the cell lines were lentivirally transduced or transiently transfected to express the HLA of interest. 293T cells were transduced with a lentiviral vector encoding various regions of a mutant RAS peptide. Greater than 50 million cells expressing peptides encoded by a mutant RAS peptide were cultured and peptides were eluted from HLA-peptide complexes using an acid wash. Eluted peptides were then analyzed by targeted MS/MS with parallel reaction monitoring (PRM). For 293T cells lentivirally transduced with both a RAS^{G12V} mutation and an HLA-A*03:01 gene, the peptide with amino acid sequence vvvgaVgvgk was detected by mass spectrometry. Spectral comparison to its corresponding stable heavy-isotopically labeled synthetic peptide (FIG. 5) showed mass accuracy of the detected peptide to be less than 5 parts per million (ppm). Endogenous peptide spectrum is shown in the top panel and the corresponding stable heavy-isotopically labeled spectrum is shown in the bottom panel. For SW620 cells naturally expressing a RAS^{G12V} mutation and lentivirally transduced with the HLA-A*03:01 gene, the peptide with amino sequence vvvgaVgvgk was detected by mass spectrometry.

### HLA Class I Binding and Stability

A subset of the peptides used for affinity measurements were also used for stability measurements using the assay described (n=275). These data are shown in Table 3. Less than 50 nM was considered by the field as a strong binder, 50-150 nM was considered an intermediate binder, 150-500 nM was considered a weak binder, and greater than 500 nM was considered a very weak binder. The connection between the observed stability and observed affinity was evident by the decreasing median stability across these binned stability intervals. However, there is considerable overlap between the bins, and importantly there are epitopes in all bins with observed stability in the multiple hour range, including the very weak binders.

Immunogenicity assays are used to test the ability of each test peptide to expand T cells. Mature professional APCs are prepared for these assays in the following way. Monocytes are enriched from healthy human donor PBMCs using a bead-based kit (Miltenyi). Enriched cells are plated in GM-CSF and IL-4 to induce immature DCs. After 5 days, immature DCs are incubated at 37° C. with each peptide for 1 hour before addition of a cytokine maturation cocktail (GM-CSF, IL-1β, IL-4, IL-6, TNFα, PGE1β). Cells are incubated at 37° C. to mature DCs. In some embodiments the peptides, when administered into a patient is required to elicit an immune response.

Table 4A shows peptide sequences comprising RAS mutations, corresponding HLA allele to which it binds, and measured stability and affinity.

### Example 4. Assessment of Cytotoxic Capacity of Antigen-Specific T Cells In Vitro

Cytotoxicity activity can be measured with the detection of cleaved Caspase 3 in target cells by Flow cytometry. Target cancer cells are engineered to express the mutant peptide along and the proper MHC-I allele. Mock-transduced target cells (i.e. not expressing the mutant peptide) are used as a negative control. The cells are labeled with CFSE to distinguish them from the stimulated PBMCs used as effector cells. The target and effector cells are co-cultured for 6 hours before being harvested. Intracellular staining is performed to detect the cleaved form of Caspase 3 in the CFSE-positive target cancer cells. The percentage of specific lysis is calculated as: Experimental cleavage of Caspase 3/spontaneous cleavage of Caspase 3 (measured in the absence of mutant peptide expression)×100.

In some examples, cytotoxicity activity is assessed by co-culturing induced T cells with a population of antigen-specific T cells with target cells expressing the corresponding HLA, and by determining the relative growth of the target cells, along with measuring the apoptotic marker Annexin V in the target cancer cells specifically. Target cancer cells are engineered to express the mutant peptide or the peptide is exogenously loaded. Mock-transduced target cells (i.e. not expressing the mutant peptide), target cells loaded with wild-type peptides, or target cells with no peptide loaded are used as a negative control. The cells are also transduced to stably express GFP allowing the tracking of target cell growth. The GFP signal or Annexin-V signal are measured over time with an IncuCyte S3 apparatus. Annexin V signal originating from effector cells is filtered out by size exclusion. Target cell growth and death is expressed as GFP and Annexin-V area (mm 2) over time, respectively.

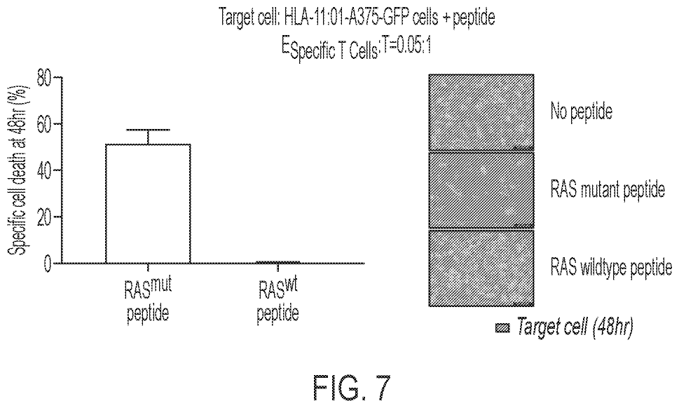

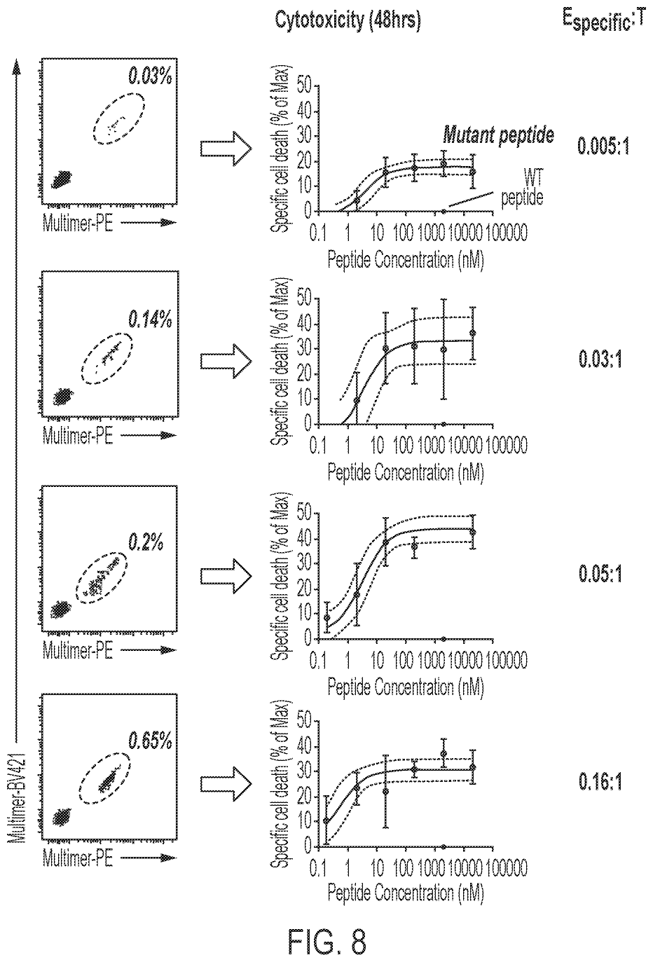

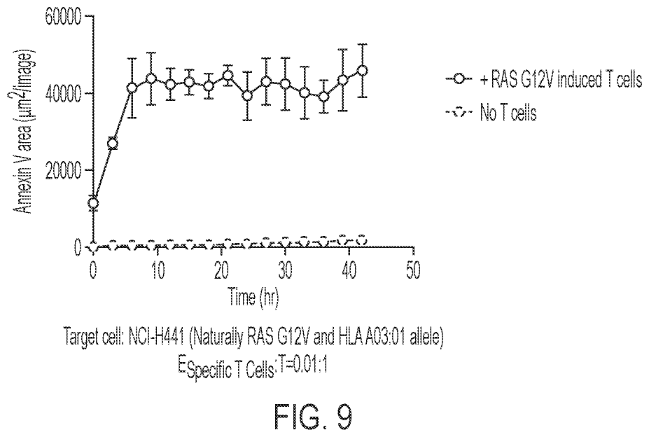

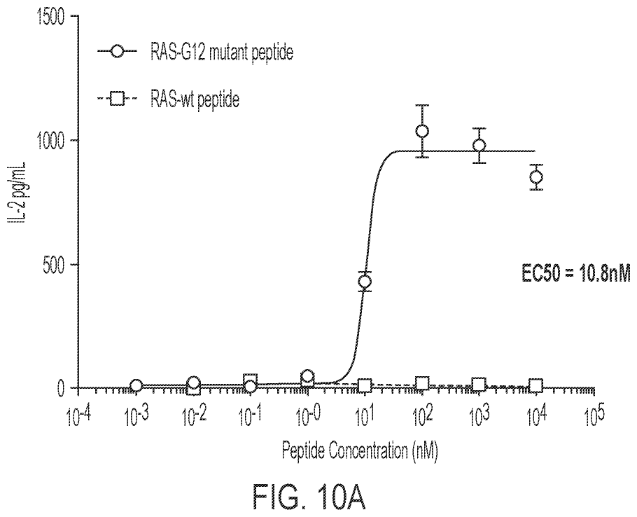

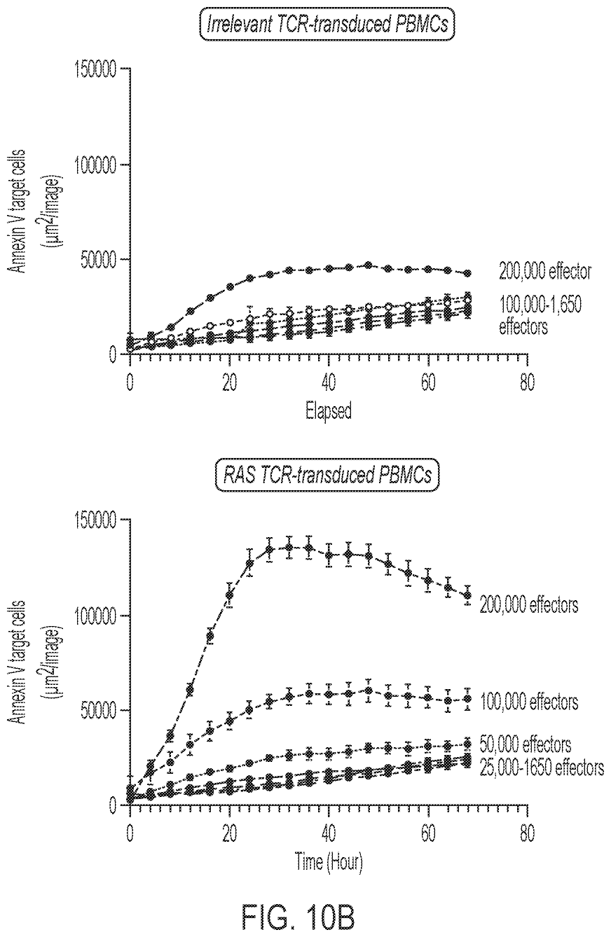

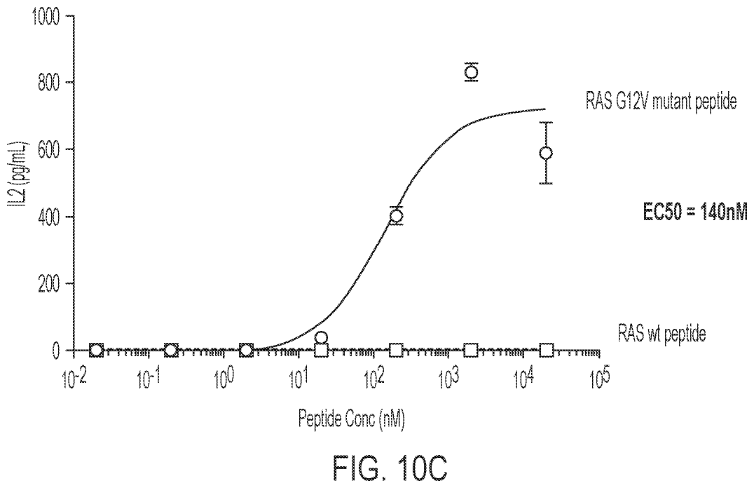

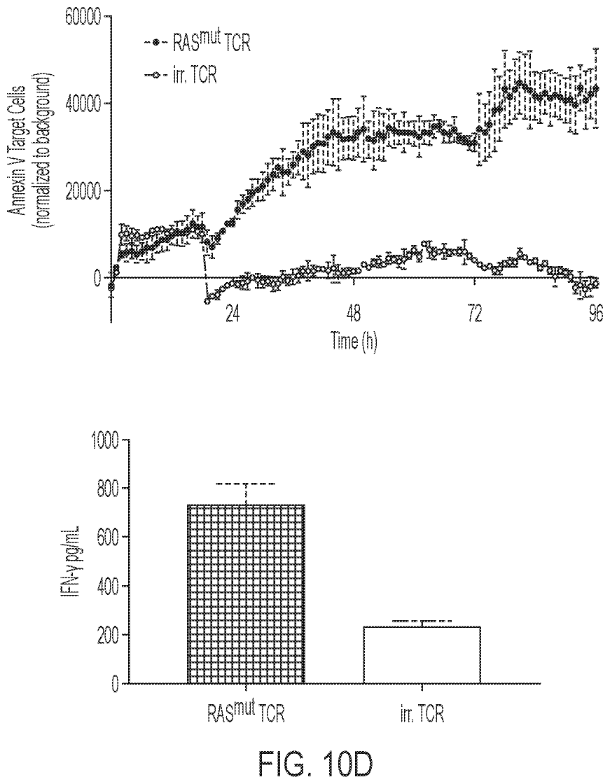

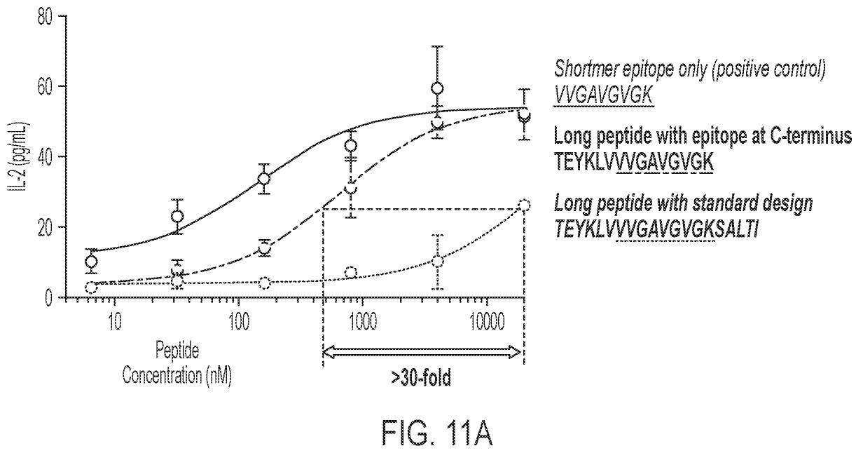

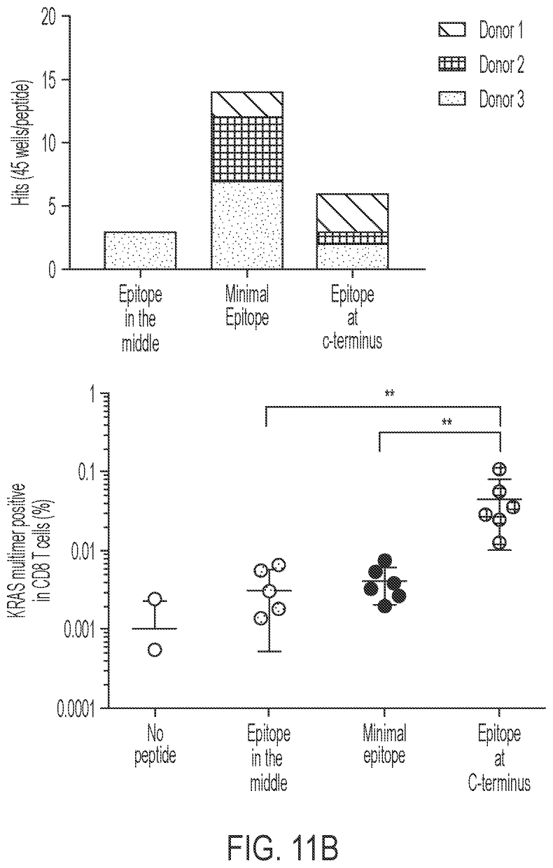

Exemplary data demonstrating that T cells stimulated to recognize a RAS^{G12V} neoantigen on HLA-A11:01 specifically recognize and kill target cells loaded with the mutant peptide but not the wild-type peptide is shown in FIG. 7. Exemplary data demonstrating that T cells stimulated to recognize a RAS^{G12V} neoantigen on HLA-A11:01 kill target cells loaded with nanomolar amounts of peptide at E:T ratios of <0.2:1 are shown in FIG. 8. Exemplary data demonstrating that T cells stimulated to recognize a RAS^{G12V} neoantigen on HLA-A03:01 kill NCI-H441 cells that naturally have the RASG12V mutation and HLA-A03:01 are shown in FIG. 9. IL2 secretion was found to be specific for target mutated peptides by Jurkat cells expressing peptide specific TCRs (FIG. 10A and FIG. 10C). Cytotoxicity measurements of target cells in presence of TCR-expressing Jurkat cells were found to exhibit high specificity FIG. 10B and FIG. 10D (upper panel). The TCR expressing cells exhibit high target specific cytokine production (IFN-gamma), as shown in FIG. 10D (lower panel). FIG. 11A depicts the effects of using short versus long peptides for stimulation on T cell activation, as indicated by IL2 release. FIG. 11B shows the effect of positioning the epitope within the peptide (in the middle; at C terminus, or using the minimal KRAS epitope) on generating T cells (upper panel), and shows antigen responsive T cell CD8 T cell percent (lower panel).

### Example 5. Enrichment of Target Antigen Activated T Cells

Tumor antigen responsive T cells may be further enriched. In this example, multiple avenues for enrichment of antigen responsive T cells are explored and results presented. After the initial stimulation of antigen-specific T cells (Example 2, Steps 1-5), an enrichment procedure can be used prior to further expansion of these cells. As an example, stimulated cultures and pulsed with the same peptides used for the initial stimulation on day 13, and cells upregulating 4-1BB are enriched using Magnetic-Assisted Cell Separation (MACS; Miltenyi). These cells can then be further expanded, for example, using anti-CD3 and anti-CD28 microbeads and low-dose IL2.

### Example 6. Immunogenicity Assays for Selected Peptides

After maturation of DCs, PBMCs (either bulk or enriched for T cells) are added to mature dendritic cells with proliferation cytokines. Cultures are monitored for peptide-specific T cells using a combination of functional assays and/or tetramer staining. Parallel immunogenicity assays with the modified and parent peptides allowed for comparisons of the relative efficiency with which the peptides expanded peptide-specific T cells. In some embodiments, the peptides elicit an immune response in the T cell culture comprises detecting an expression of a FAS ligand, granzyme, perforins, IFN, TNF, or a combination thereof in the T cell culture.

Immunogenicity can be measured by a tetramer assay. MHC tetramers are purchased or manufactured on-site, and are used to measure peptide-specific T cell expansion in the immunogenicity assays. For the assessment, tetramer is added to 1×10\hat{ }5 cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4 degrees Celsius for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that were CD8⁺/Tetramer⁺.

Immunogenicity can be measured by intracellular cytokine staining. In the absence of well-established tetramer staining to identify antigen-specific T cell populations, antigen-specificity can be estimated using assessment of cytokine production using well-established flow cytometry assays. Briefly, T cells are stimulated with the peptide of interest and compared to a control. After stimulation, production of cytokines by CD4+ T cells (e.g., IFNγ and TNFα) are assessed by intracellular staining. These cytokines, especially IFNγ, used to identify stimulated cells.

In some embodiments the immunogenicity is measured by measuring a protein or peptide expressed by the T cell, using ELISpot assay. Peptide-specific T cells are functionally enumerated using the ELISpot assay (BD Biosciences), which measures the release of IFNγ from T cells on a single cell basis. Target cells (T2 or HLA-A0201 transfected C1Rs) were pulsed with 10 μM peptide for one hour at 37 degrees C., and washed three times. 1×10\hat{ }5 peptide-pulsed targets are co-cultured in the ELISPOT plate wells with varying concentrations of T cells (5×10\hat{ }2 to 2×10\hat{ }3) taken from the immunogenicity culture. Plates are developed according to the manufacturer's protocol, and analyzed on an ELISPOT reader (Cellular Technology Ltd.) with accompanying software. Spots corresponding to the number of IFN gamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide. Some exemplary data are shown below in Table 13.

TABLE 13

| | | | Predicted | RECON | |
|---|---|---|---|---|---|
| | | | Affinity | Percent | Immunogenicity |
| Peptide | Allele | Gene | (nM) | Rank | (# donors/2) |
| SLQCVSLHL | HLA-A02:01 | KLK2 | 39.4 | 0.4 | |
| LVLSIALSV | HLA-A02:01 | KLK2 | 54.9 | 1.1 | |
| VILGVHLSV | HLA-A02:01 | KLK2 | 62.1 | 0.4 | |
| VLAPQESSV | HLA-A02:01 | KLK2 | 65.7 | 0.08 | |
| SLQCVSLHLL | HLA-A02:01 | KLK2 | 90.3 | 0.4 | |
| MLLRLSEPA | HLA-A02:01 | KLK2; KLK3 | 56 | 2.5 | |
| LTMPALPMV | HLA-A02:01 | KLK3 | 14.3 | 1.1 | |
| FLTLSVTWIA | HLA-A02:01 | KLK3 | 16.9 | 3.5 | |
| KLQCVDLHV | HLA-A02:01 | KLK3 | 21.2 | 0.3 | |
| *FLTPKKLQCV | HLA-A02:01 | KLK3 | 126.4 | 0.17 | 1 |
| FLRPGDDSTL | HLA-A02:01 | KLK3 | 982.7 | 0.4 | |
| *FLGYLILGV | HLA-A02:01 | KLK4 | 6.3 | 0.05 | 1 |
| *LLANDLMLI | HLA-A02:01 | KLK4 | 10.7 | 0.4 | 2 |
| *FQNSYTIGL | HLA-A02:01 | KLK4 | 15.1 | 1.6 | 2 |
| MLIKLDESV | HLA-A02:01 | KLK4 | 17.6 | 0.25 | |
| VLQCVNVSV | HLA-A02:01 | KLK4 | 19.2 | 0.1 | |
| *LLANGRMPTV | HLA-A02:01 | KLK4 | 25.9 | 0.25 | 2 |
| *ILNDTGCHYV | HLA-A02:01 | TGM4 | 17.2 | 0.1 | 1 |
| *FQYPEFSIEL | HLA-A02:01 | TGM4 | 21.2 | 1 | 1 |
| ILGKYQLNV | HLA-A02:01 | TGM4 | 22 | 0.3 | |
| LLGNSVNFTV | HLA-A02:01 | TGM4 | 27.8 | 0.7 | |
| *VLDCCISLL | HLA-A02:01 | TGM4 | 30.6 | 0.4 | 1 |
| ILGSFELQL | HLA-A02:01 | TGM4 | 31.2 | 0.25 | |
| *RLIWLVKMV | HLA-A02:01 | TGM4 | 64.4 | 0.17 | 1 |
| VLLGNSVNFTV | HLA-A02:01 | TGM4 | 83.7 | 0.6 | |
| TLAIPLTDV | HLA-A02:01 | TGM4 | 149.2 | 0.25 | |

CD10⁷a and CD10⁷b are expressed on the cell surface of CD8+ T cells following activation with cognate peptide. The lytic granules of T cells have a lipid bilayer that contains lysosomal-associated membrane glycoproteins (“LAMPs”), which include the molecules CD107a and b. When cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107a and b staining is reported on a single cell basis, the assay is used to functionally enumerate peptide-specific T cells. To perform the assay, peptide is added to HLA-A0201-transfected cells C1R to a final concentration of 20 NM, the cells were incubated for 1 hour at 37 degrees C., and washed three times. 1×10\hat{ }5 of the peptide-pulsed C1R cells were aliquoted into tubes, and antibodies specific for CD107a and b are added to a final concentration suggested by the manufacturer (Becton Dickinson). Antibodies are added prior to the addition of T cells in order to “capture” the CD107 molecules as they transiently appear on the surface during the course of the assay. 1×10\hat{ }5 T cells from the immunogenicity culture are added next, and the samples were incubated for 4 hours at 37 degrees C. The T cells are further stained for additional cell surface molecules such as CD8 and acquired on a FACS Calibur instrument (Becton Dickinson). Data is analyzed using the accompanying Cellquest software, and results were reported as the percentage of CD8+ CD107 a and b+ cells.

Cytotoxic activity is measured using a chromium release assay. Target T2 cells are labeled for 1 hour at 37 degrees C. with Na51Cr and washed 5×10\hat{ }3 target T2 cells were then added to varying numbers of T cells from the immunogenicity culture. Chromium release is measured in supernatant harvested after 4 hours of incubation at 37 degrees C. The percentage of specific lysis is calculated as: Experimental release-spontaneous release/Total release-spontaneous release×100

Immunogenicity assays were carried out to assess whether each peptide can elicit a T cell response by antigen-specific expansion. Though current methods are imperfect, and therefore negative results do not imply a peptide is incapable of inducing a response, a positive result demonstrates that a peptide can induce a T cell response. Several peptides from Table 3 were tested for their capacity to elicit CD8+ T cell responses with multimer readouts as described. Each positive result was measured with a second multimer preparation to avoid any preparation biases. In an exemplary assay, HLA-A02:01+ T cells were co-cultured with monocyte-derived dendritic cells loaded with TMPRSS2::ERG fusion neoepitope (ALNSEALSV; HLA-A02:01) for 10 days. CD8+ T cells were analyzed for antigen-specificity for TMPRSS2::ERG fusion neoepitope using multimers (initial: BV421 and PE; validation: APC and BUV396).

While antigen-specific CD8+ T cell responses are readily assessed using well-established HLA Class I multimer technology, CD4+ T cell responses require a separate assay to evaluate because HLA Class II multimer technology is not well-established. In order to assess CD4+ T cell responses, T cells were re-stimulated with the peptide of interest and compared to a control. In the case of a completely novel sequence (e.g., arising from a frame-shift or fusion), the control was no peptide. In the case of a point-mutation, the control was the WT peptide. After stimulation, production of cytokines by CD4+ T cells (e.g., IFNγ and TNFα) were assessed by intracellular staining. These cytokines, especially IFNγ, used to identify stimulated cells. Antigen-specific CD4+ T cell responses showed increased cytokine production relative to control.

### Example 7. Cell Expansion and Preparation

To prepare APCs, the following method was employed (a) obtain of autologous immune cells from the peripheral blood of the patient; enrich monocytes and dendritic cells in culture; load peptides and mature DCs.

### T Cell Induction (Protocol 1)

First induction: (a) Obtaining autologous T cells from an apheresis bag; (b) Depleting CD25+ cells and CD14+ cells, alternatively, depleting only CD25+ cells; (c) Washing the peptide loaded and mature DC cells, resuspending in the T cell culture media; (d) Incubating T cells with the matured DC.

Second induction: (a) Washing T cells, and resuspending in T cell media, and optionally evaluating a small aliquot from the cell culture to determine the cell growth, comparative growth and induction of T cell subtypes and antigen specificity and monitoring loss of cell population; (b) Incubating T cells with mature DC.

Third induction: (a) Washing T cells, and resuspending in T cell media, and optionally evaluating a small aliquot from the cell culture to determine the cell growth, comparative growth and induction of T cell subtypes and antigen specificity and monitoring loss of cell population; (b) Incubating T cells with mature DC.

To harvest peptide activated t cells and cryopreserve the T cells, the following method was employed (a) Washing and resuspension of the final formulation comprising the activated T cells which are at an optimum cell number and proportion of cell types that constitutes the desired characteristics of the Drug Substance (DS). The release criteria testing include inter alia, Sterility, Endotoxin, Cell Phenotype, TNC Count, Viability, Cell Concentration, Potency; (b) Filling drug substance in suitable enclosed infusion bags; (c) Preservation until time of use.

### Example 8. Methods of Functional Characterization of the CD4+ and CD8+ Neoantigen-Specific T Cells

Neoantigens, which arise in cancer cells from somatic mutations that alter protein-coding gene sequences, are emerging as an attractive target for immunotherapy. They are uniquely expressed on tumor cells as opposed to healthy tissue and may be recognized as foreign antigens by the immune system, increasing immunogenicity. T cell manufacturing processes were developed to raise memory and de novo CD4+ and CD8+ T cell responses to patient-specific neoantigens through multiple rounds of ex-vivo T cell stimulation, generating a neoantigen-reactive T cell product for use in adoptive cell therapy. Detailed characterization of the stimulated T cell product can be used to test the many potential variables these processes utilize.

To probe T cell functionality and/or specificity, an assay was developed to simultaneously detect antigen-specific T cell responses and characterize their magnitude and function. This assay employs the following steps. First T cell-APC co-cultures were used to elicit reactivity in antigen-specific T cells. Optionally, sample multiplexing using fluorescent cell barcoding is employed. To identify antigen-specific CD8+ T cells and to examine T cell functionality, staining of peptide-MHC multimers and multiparameter intracellular and/or cell surface cell marker staining were probed simultaneously using FACS analysis. The results of this streamlined assay demonstrated its application to study T cell responses induced from a healthy donor. Neoantigen-specific T cell responses induced toward peptides were identified in a healthy donor. The magnitude, specificity and functionality of the induced T cell responses were also compared. Briefly, different T cell samples were barcoded with different fluorescent dyes at different concentrations (see, e.g., Example 19). Each sample received a different concentration of fluorescent dye or combination of multiple dyes at different concentrations. Samples were resuspended in phosphate-buffered saline (PBS) and then fluorophores dissolved in DMSO (typically at 1:50 dilution) were added to a maximum final concentration of 5 μM After labeling for 5 min at 37° C., excess fluorescent dye was quenched by the addition of protein-containing medium (e.g. RPMI medium containing 10% pooled human type AB serum). Uniquely barcoded T cell cultures were challenged with autologous APC pulsed with the antigen peptides as described above.

The differentially labeled samples were combined into one FACS tube or well, and pelleted again if the resulting volume is greater than 100 μL. The combined, barcoded sample (typically 100 μL) was stained with surface marker antibodies including fluorochrome conjugated peptide-MHC multimers. After fixation and permeabilization, the sample was additionally stained intracellularly with antibodies targeting TNF-α and IFN-γ.

The cell marker profile and MHC tetramer staining of the combined, barcoded T cell sample were then analyzed simultaneously by flow cytometry on flow cytometer. Unlike other methods that analyze cell marker profiles and MHC tetramer staining of a T cell sample separately, the simultaneous analysis of the cell marker profile and MHC tetramer staining of a T cell sample described in this example provides information about the percentage of T cells that are both antigen specific and that have increased cell marker staining. Other methods that analyze cell marker profiles and MHC tetramer staining of a T cell sample, separately determine the percentage of T cells of a sample that are antigen specific, and separately determine the percentage of T cells that have increased cell marker staining, only allowing correlation of these frequencies.

The simultaneous analysis of the cell marker profile and MHC tetramer staining of a T cell sample described in this example does not rely on correlation of the frequency of antigen specific T cells and the frequency of T cells that have increased cell marker staining; rather, it provides a frequency of T cells that are both antigen specific and that have increased cell marker staining. The simultaneous analysis of the cell marker profile and MHC tetramer staining of a T cell sample described in this example allows for determination on a single cell level, those cells that are both antigen specific and that have increased cell marker staining.

To evaluate the success of a given induction process, a recall response assay was used followed by a multiplexed, multiparameter flow cytometry panel analysis. A sample taken from an induction culture was labeled with a unique two-color fluorescent cell barcode. The labeled cells were incubated on antigen-loaded DCs or unloaded DCs overnight to stimulate a functional response in the antigen-specific cells. The next day, uniquely labeled cells were combined prior to antibody and multimer staining according to Table 14 below.

TABLE 14

| Marker | Fluorochrome | Purpose |
|---|---|---|
| CD19/CD16/CD14 | BUV395 | Cell exclusion |
| Live/Dead | Near-IR | Dead cell exclusion |
| CD3 | BUV805 | Lineage gating |
| CD4 | Alexa Fluor 700 | Lineage gating |
| CD8 | PerCP-Cy5.5 | Lineage gating |
| Barcode 1 | CFSE | Sample multiplexing |
| Barcode 2 | TagIT Violet | Sample multiplexing |
| Multimer 1 | PE | CD8+ antigen specificity |
| Multimer 2 | BV650 | CD8+ antigen specificity |
| IFNγ | APC | Functionality |
| TNFα | BV711 | Functionality |
| CD107a | BV786 | Cytotoxicity |
| 4-1BB | PE/Dazzle 594 | Activation |

Patient-specific neoantigens were predicted using bioinformatics engine. Synthetic long peptides covering the predicted neoantigens were used as immunogens in the stimulation protocol to assess the immunogenic capacity. The stimulation protocol involves feeding these neoantigen-encoding peptides to patient-derived APCs, which are then co-cultured with patient-derived T cells to prime neoantigen specific T cells.

Multiple rounds of stimulations are incorporated in the stimulation protocol to prime, activate and expand memory and de novo T cell responses. The specificity, phenotype and functionality of these neoantigen-specific T cells was analyzed by characterizing these responses with the following assays: Combinatorial coding analysis using pMHC multimers was used to detect multiple neoantigen-specific CD8+ T cell responses. A recall response assay using multiplexed, multiparameter flow cytometry was used to identify and validate CD4+ T cell responses. The functionality of CD8+ and CD4+ T cell responses was assessed by measuring production of pro-inflammatory cytokines including IFN-γ and TNFα, and upregulation of the CD107a as a marker of degranulation. A cytotoxicity assay using neoantigen-expressing tumor lines was used to understand the ability of CD8+ T cell responses to recognize and kill target cells in response to naturally processed and presented antigen. The cytotoxicity was measured by the cell surface upregulation of CD107a on the T cells and upregulation of active Caspase3 on neoantigen-expressing tumor cells. The stimulation protocol was successful in the expansion of pre-existing CD8+ T cell responses, as well as the induction of de novo CD8+ T cell responses (Table 15).

TABLE 15

| Patient | HUGO Symbol | Full Gene Name | Type |
|---|---|---|---|
| NV10 | SRSF1_{E>K} | Serine And Arginine Rich Splicing | CD8 |
| | | Factor 1 | |
| | ARAP1_{Y>H} | Ankyrin Repeat And PH Domain | |
| | PKDREJ_{G>R} | Polycystin Family Receptor For Egg | |
| | | Jelly | |
| | MKRN1_{S>L} | Makorin Ring Finger Protein 1 | CD4 |
| | CREBBP_{S>L} | CREB Binding Protein | |
| | TPCN1_{K>E} | Two Pore Segment Channel 1 | |
| NV6 | AASDH_{neoORF} | Aminoadipate-Semialdehyde | CD8 |
| | | Dehydrogenase | |
| | ACTN4_{K>N} | Actinin Alpha 4 | |
| | CSNK1A1_{S>L} | Casein Kinase 1 Alpha 1 | |
| | DHX40_{neoORF} | DEAH-Box Helicase 40 | |
| | GLI3_{P>L} | GLI Family Zinc Finger 3 | |
| | QARS_{R>W} | Glutaminyl-TRNA Synthetase | |
| | FAM178B_{P>L} | Family With Sequence Similarity 178 | |
| | | Member B | |
| | RPS26_{P>}L | Ribosomal Protein S26 | |

Using PBMCs from a melanoma patient a clinical study performed by Applicant's group, expansion of a pre-existing CD8+ T cell response was observed from 4.5% of CD8+ T cells to 72.1% of CD8+ T cells (SRSF1E_{>K}). Moreover, the stimulation protocol was effective in inducing two presumed de novo CD8+ T cell responses towards patient-specific neoantigens (exemplary de novo CD8+ T cell responses: ARAP1_{Y>H}: 6.5% of CD8+ T cells and PKDREJ_{G>R}: 13.4% of CD8+ T cells; no cells were detectable prior to the stimulation process). The stimulation protocol successfully induced seven de novo CD8+ T cell responses towards both previously described and novel model neoantigens using PBMCs from another melanoma patient, NV6, up to varying magnitudes (ACTN4_{K>N} CSNK1A1_{S>L} DHX40neoORF 7, GLI3_{P>L}, QARS_{R>W}, FAM178B_{P>L} and RPS26_{P>L}, range: 0.2% of CD8+ T cells up to 52% of CD8+ T cells). Additionally, a CD8+ memory T cell response towards a patient-specific neoantigen was expanded (AASDHneoORF, up to 13% of CD8+ T cells post stimulation).

The induced CD8+ T cells from the patient was characterized in more detail. Upon re-challenge with mutant peptide loaded DCs, neoantigen-specific CD8+ T cells exhibited one, two and/or all three functions (16.9% and 65.5% functional CD8+ pMHC+ T cells for SRSF1E>K and ARAP1Y>H, respectively. When re-challenged with different concentrations of neoantigen peptides, the induced CD8+ T cells responded significantly to mutant neoantigen peptide but not to the wildtype peptide. In said patient, CD4+ T cell responses were identified using a recall response assay with mutant neoantigen loaded DCs. Three CD4+ T cell responses were identified (MKRN1S>L, CREBBPS>L and TPCN1K>E) based on the reactivity to DCs loaded with mutant neoantigen peptide. These CD4+ T cell responses also showed a polyfunctional profile when re-challenged with mutant neoantigen peptide. 31.3%, 34.5% & 41.9% of CD4+ T cells exhibited one, two and/or three functions; MKRN1S>L, CREBBPS>L and TPCN1K>E responses, respectively.

The cytotoxic capacity of the induced CD8+ responses from said patient was also assessed. Both SRSF1E>K and ARAP1Y>H responses showed a significant upregulation of CD107a on the CD8+ T cells and active Caspase3 on the tumor cells transduced with the mutant construct after co-culture.

Using the stimulation protocol, predicted patient-specific neoantigens, as well as model neoantigens, were confirmed to be immunogenic by the induction of multiple neoantigen-specific CD8+ and CD4+ T cell responses in patient material. The ability to induce polyfunctional and mutant-specific CD8+ and CD4+ T cell responses proves the capability of predicting high-quality neoantigens and generating potent T cell responses. The presence of multiple enriched neoantigen-specific T cell populations (memory and de novo) at the end of the stimulation process demonstrates the ability to raise new T cell responses and generate effective cancer immunotherapies to treat cancer patients.

Exemplary materials for T cell culture are provided below:

Materials: AIM V media (Invitrogen) Human FLT3L; preclinical CellGenix #1415-050 Stock 50 ng/μL TNFα; preclinical CellGenix #1406-050 Stock 10 ng/μL; IL-1β, preclinical CellGenix #1411-050 Stock 10 ng/μL; PGE1 or Alprostadil—Cayman from Czech republic Stock 0.5 μg/μL; R10 media—RPMI 1640 glutamax+10% Human serum+1% PenStrep; 20/80 Media-18% AIM V+72% RPMI 1640 glutamax+10% Human Serum+1% PenStrep; IL7 Stock 5 ng/μL; IL15 Stock 5 ng/μL; DC media (Cellgenix); CD14 microbeads, human, Miltenyi #130-050-201, Cytokines and/or growth factors, T cell media (AIM V+RPMI 1640 glutamax+serum+PenStrep), Peptide stocks—1 mM per peptide (HIV A02—5-10 peptides, HIV B07-5-10 peptides, DOM—4-8 peptides, PIN—6-12 peptides).

### Example 9. Exemplary KRAS Mutations

Exemplary KRAS mutations, sequences comprising the mutated residue and exemplary diseases are listed below (Table 16).

TABLE 16

| | Exemplary | | |
|---|---|---|---|
| | Protein | Mutation Sequence Context | Exemplary |
| Gene | Change | (Mutated non-native residue underlined) | Diseases |
| KRAS | G12C | MTEYKLVVVGACGVGKSALTIQLIQNHFVD | BRCA, CESC, CRC, |
| | | EYDPTIEDSYRKQVVIDGETCLLDILDTAGQE | HNSC, LUAD, PAAD, |
| | | | UCEC |
| KRAS | G12D | MTEYKLVVVGADGVGKSALTIQLIQNHFVD | BLCA, BRCA, CESC, |
| | | EYDPTIEDSYRKQVVIDGETCLLDILDTAGQE | CRC, GBM, HNSC, KIRP, |
| | | | LIHC, LUAD, PAAD, |
| | | | SKCM, UCEC |
| KRAS | G12V | MTEYKLVVVGAVGVGKSALTIQLIQNHFVD | BRCA, CESC, CRC, |
| | | EYDPTIEDSYRKQVVIDGETCLLDILDTAGQE | LUAD, PAAD, THCA, |
| | | | UCEC |
| KRAS | Q61H | AGGVGKSALTIQLIQNHFVDEYDPTIEDSYRK | CRC, LUSC, PAAD, |
| | | QVVIDGETCLLDILDTAGHEEYSAMRDQYMR | SKCM, UCEC |
| | | TGEGFLCVFAINNTKSFEDIHHYREQIKRVKD | |
| | | SEDVPM | |
| KRAS | Q61L | AGGVGKSALTIQLIQNHFVDEYDPTIEDSYRK | CRC, GBM, HNSC, |
| | | QVVIDGETCLLDILDTAGLEEYSAMRDQYMR | LUAD, SKCM, UCEC |
| | | TGEGFLCVFAINNTKSFEDIHHYREQIKRVKD | |
| | | SEDVPM | |

1.-50. (canceled)

51. An ex vivo method for preparing antigen-specific T cells, the method comprising contacting T cells with:
(a) antigen presenting cells (APCs) comprising one or more peptides containing an epitope with a sequence GACGVGKSA, wherein the APCs express a protein encoded by an HLA-C03:04 allele; or
(b) APCs comprising one or more peptides containing an epitope with a sequence GAVGVGKSA, wherein the APCs express a protein encoded by an HLA-C03:03 allele.

52. The method of claim 51, wherein the T cells or APCs are from a subject with cancer.

53. The method of claim 51, wherein the T cells or APCs are allogeneic.

54. The method of claim 51, the method comprising contacting T cells with antigen presenting cells (APCs) comprising one or more peptides containing an epitope with a sequence GACGVGKSA, wherein the APCs express a protein encoded by an HLA-C03:04 allele, wherein the method further comprises administering the T cells to a subject in need thereof, wherein the subject expresses a protein encoded by the HLA-C03:04 allele, and the T cells have been contacted with APCs comprising one or more peptides containing the epitope GACGVGKSA.

55. The method of claim 51, the method comprising contacting T cells with APCs comprising one or more peptides containing an epitope with a sequence GAVGVGKSA, wherein the APCs express a protein encoded by an HLA-C03:03 allele, wherein the method further comprises administering the T cells to a subject in need thereof, wherein the subject expresses one or more protein encoded by the HLA-C03:03 allele and the T cells have been contacted with APCs comprising a peptide containing the epitope GAVGVGKSA.

56. The method of claim 51, wherein the method comprises obtaining a biological sample comprising T cells or APCs from a subject, and wherein the biological sample is peripheral blood mononuclear cell (PBMC) sample.

57. The method of claim 56, wherein the method comprises depleting CD14+ cells from the biological sample.

58. The method of claim 56, wherein the method comprises depleting CD25+ cells from the biological sample.

59. The method of claim 51, wherein the method comprises incubating the T cells and APCs in the presence of FMS-like tyrosine kinase 3 receptor ligand (FLT3L).

60. The method of claim 51, wherein the method comprises stimulating or expanding the T cells in the presence of the APCs for at least 3, 4, 5, 6, 7, 8, 9, 10, 11, 12, 13, 14, 15, 16, 7, 18, 19 or or more days.

61. The method of claim 51, wherein the method comprises expanding the T cells at least 5-fold, 10-fold, 20, fold, 50-fold, 100-fold, 500-fold or 1,000-fold or more in the presence of the APCs.

62. The method of claim 51, wherein the antigen-specific T cells are prepared in less than 28 days.

63. The method of claim 52, wherein the epitope:
(i) binds to a protein encoded by an HLA allele of the subject,
(ii) is immunogenic according to an immunogenicity assay,
(iii) is presented by APCs according to a mass spectrometry assay, or
(iv) stimulates T cells to be cytotoxic according to a cytotoxicity assay.

64. The method of claim 51, wherein the method comprises assaying the T cells for expression of a T cell activation marker or for cytokine production.

65. A pharmaceutical composition comprising:
(a) T cells comprising a population of T cells expressing a T cell receptor (TCR) that binds to a complex of (i) an MHC protein encoded by an HLA-C03:04 allele and (ii) an epitope with a sequence GACGVGKSA;
(b) T cells comprising a population of T cells expressing a T cell receptor (TCR) that binds to a complex of (i) an MHC protein encoded by an HLA-C03:03 allele and (ii) an epitope with a sequence GAVGVGKSA;
(c) antigen presenting cells (APCs) expressing an MHC protein encoded by an HLA-C03:04 allele, wherein the APCs comprise (i) a peptide having an epitope with a sequence GACGVGKSA or (ii) a polynucleotide encoding the peptide; or
(d) APCs expressing an MEW protein encoded by an HLA-C03:03 allele, wherein the APCs comprise (i) a peptide having an epitope with a sequence GAVGVGKSA or (ii) a polynucleotide encoding the peptide.

66. A TCR comprising a TCR alpha chain and a TCR beta chain that binds to a complex comprising:
(a) a mutated RAS epitope with the sequence GACGVGKSA and an MEW protein encoded by C03:04 allele, or
(b) a mutated RAS epitope with the sequence GAVGVGKSA and an MEW protein encoded by C03:03 allele.

67. A method of treating a subject with cancer comprising administering to the subject
(a) a peptide,
(b) a polynucleotide encoding the peptide,
(c) antigen presenting cells (APCs) comprising (a) or (b), or
(d) T cells stimulated with APCs comprising (a) or (b);
wherein:
(i) the peptide comprises an epitope with a sequence GACGVGKSA and the subject expresses a protein encoded by an HLA-C03:04 allele, or
(ii) the peptide comprises an epitope with a sequence GAVGVGKSA and the subject expresses a protein encoded by an HLA-C03:03 allele.

68. The method of claim 67, wherein the cancer is selected from the group consisting of pancreatic ductal adenocarcinoma, non-small cell lung cancer, colorectal cancer, and cholangiocarcinoma.

69. The method of claim 67, wherein the peptide comprises one or more additional epitopes, and wherein the one or more additional epitopes comprise one or more epitopes of any one of Tables 2-12.

70. The method of claim 67, wherein the method further comprises administering an additional anti-cancer therapy to the subject, wherein the additional anti-cancer therapy comprises an additional peptide, a polynucleotide encoding the additional peptide, APCs comprising the additional peptide or the polynucleotide, or T cells stimulated with the APCs, wherein the additional peptide comprises one or more epitopes of any one of Tables 2-12.
